SEQUENCE LISTING (179 sequences)

SEQ ID NO: 1 - protein, 506 aa, Lactuca sativa
MESLQIPIAFYAIIAILTFFFLSWVRRKPLPPGPMGWPIIGNMLMMDQLTHRGLARLAEKYGGILHLKMGFSHTIAVSSPEMARIILQEKDNIFANRPATIAITYLTYNGVDLAFANYGPFWRQMRKLCVMKLFSRKRAESWDSVRDEVDTMVKATAINSGTPVNLGELVFGLTHDIIYRAAFGSISHEGKEEFIRILQEYTKLFGAFNLADFIPFLGFIDPAGLNTRLPAARAALDGFIDKIIDEHLRKGKKTGDEGLDNDMVDEMLAFYSEEGKVNEGGDLQNAINLTRDNIKAIIMDVMFGGTETVASAIEWAMTELMHTPEALKRVQQEMANVVGLDRRVEESDLEKLTYFKCVIKETLRLHPPIPVLLHQSSEATEVSGYHIPKGTRVMVNAYAINRDKNSWEDPDTFNPSRFLQNGAPDFRGSNYEFLPFGSGRRSCPGMQLGLYAMEMAVAHLLHCFTWELPDGMKPSEIDMGDVFGLTAPKAIRLVAVPTPRLLCPLY

SEQ ID NO: 2 - protein, 503 aa, Lactuca sativa
MDPKSILLYVVLPLLTFFLLSRLRRKPLPPGPRGWPLIGNMLMMDQLTHRGLARLGEKYGGLLHLKMGFSHTVAVSSPEIARQVLQVQDNIFANRPATIAISYLTYDRQDMAFANYGPFWRQMRKLCVMKLFSRKRAESWDSVRDEVVSMVKITAASSGTAVNLGELVFGLTHDIIYRAAFGSISHEGKEEFIRILQEYTKLFGAFNLADFVPWLGFIDPAGLNTRLPKARAALDRFIDKIIDEHLAKERKTGDEEDNDMVDEMLAFYSEEGKVNEGEDLQNAIRLTRNNIKAIIMDVMFGGTETVASAIEWALTELMHTPESLKRAQQELADVVGLDRRVEESDFEKLTYFKCVIKETLRLHPPIPVLLHQSSEATSVAGYHIPKGTRVMVNAFAINRDKNSWKDPHTFNPSRFLQDGAPDFKGSNYEFLPFGSGRRSCPGMQLGLYAMEMAVAHLLHSFTWQLPDGMKPSEIDMNDVFGLTAPKAIRLVAVPTPRLLCPLY

SEQ ID NO: 3 - protein, 506 aa, Cichorium endivia
  UNSURE 328: note = Unknown
  UNSURE 405: note = Unknown
MDYLQIPIPIYAIIAILTFFFLAWLRRKPLPPGPMGWPIIGNMLMMDQLTHRGLASLANKYGGILHLKMGFSHTIAVSSPEIARQILQEKDNIFANRPATIAITYLTYNRVDMAFANYGPFWRQMRKLCVMKLFSRKRAESWDSVRDEVDTMVKATATNSGLPVNLGELVFGLTHDIIYRAAFGSISHEGKEEFIRILQEYTKLFGAFNLADFIPMLGFIDPAGLNTRLPAARAALDGFIDKIIDEHLSKEKKTGDENVDNDMVDEMLAFYSEDGRINEGGDLQNAINLTRDNIKAIIMDVMFGGTETVASAIEWTMTELMHTPEALXRVQQELTNVVGLDRRVEESDFEKLTYFKCVIKETLRMHPPIPVLLHQSSEATEVSGYHIPKGTRVMVNAYAINRDKXSWEDPDTFNPSRFLQNGAPDFRGSNYEFLPFGSGRRSCPGMQLGLYAMEMAVAHLLHCFTWDLPDGMKPSEIDMGDVFGLTAPKAIRLVAVPTPRLLCPLY

SEQ ID NO: 4 - protein, 506 aa, Cichorium endivia
MDSLQIPIPVYAIIAILTFFFLAWLRRKPLPPGPIGWPIIGNMLMMDQLTHRGLASLAKKYGGILHLKMGFSHTIAVSSPEIARQILQEKDNIFANRPATIAITYLTYNRVDMAFANYGPFWRQMRKLCVMKLFSRKRAESWDSVRDEVDTMVKATAINSGLPVNLGELVFGLTHDIIYRAAFGSISHEGKEEFIRILQEYTKLFGAFNLADFIPMLGFIDPAGLNTRLPAARAALDGFIDKIIDEHLSKEKKTGDENVDNDMVDEMLAFYSEDGRVNEGGDLQNAINLTRDNIKAIIMDVMFGGTETVASAIEWTMTELMHTPEALKRVQQELTNVVGLDRRVEESDFEKLTYFKCVIKETLRMHPPIPVLLHQSSEATEVSGYHIPKGTRVMVNAYAINRDKNSWEDPDTFNPSRFLQNGAPDFRGSNYEFLPFGSGRRSCPGMQLGLYAMEMAVAHLLLCFTWELPDGMKPSEIDMGDVFGLTAPKAIRLVAVPTPRLLCPLY

SEQ ID NO: 5 - protein, 503 aa, Cichorium endivia
MDPMSILLYVVLPLFTFFLLSRFRRKPLPPGPRGWPVIGNMLMMDQLTHRGLARLGEKYGGLLHLKMGFSHTVAVSSPEIARQVLQVQDNIFANRPATIAISYLTYDRQDMAFANYGPFWRQMRKLCVMKLFSRKRAESWDSVRDEVVSMIKITAASSGSAVNLGELVFGLTHDIIYRAAFGSISHEGKEEFIRILQEYTKLFGAFNLADFIPWLGFIDPAGLNNRLPKARAALDGFIDKIIDEHLRKEKKTGDEEDNDMVDEMLAFYSEEGKVNEGEDLQNAIRLTRNNIKAIIMDVMFGGTETVASAIEWALTELMHTPESLKRAQQELVDVVGLDRRVEESDFEKLTYFRCVIKETLRLHPPIPVLLHQSSEATEVAGYHIPKGTRVMVNAFAINRDKNSWKDPHTFNPSRFLQDGAPDFKGSNYEFLPFGSGRRSCPGMQLGLYAMEMAVAHLLHSFTWQLPDGMKPSEIDMSDVFGLTAPKAIRLVAVPTPRLLCPLY

SEQ ID NO: 6 - protein, 506 aa, Cichorium intybus
MDSLQIPIPVYAIIAVLTFVFVAWLRRKPLPPGPMGWPIIGNMLMMDQLTHRGLASLAKKYGGILHLKMGFSHTIAVSSPEIARQILQEKDNIFANRPATIAITYLTYNRVDMAFANYGPFWRQMRKLCVMKLFSRKRAESWDSVRDEVDTMVKATATNSGLPVNLGELVFGLTHDIIYRAAFGSISHEGKEEFIRILQEYTKLFGAFNLADFIPMLGFIDPAGLNTRLPAARAALDGFIDKIIDEHLSKEKKTGDENVDNDMVDEMLAFYSEDGRINEGGDLQNAINLTRDNIKAIIMDVMFGGTETVASAIEWTMTELMHTPEALKRVQQELTNVVGLDRRVEESDFEKLTYFKCVIKETLRMHPPIPVLLHQSSEATEVSGYHIPKGTRVMVNAYAINRDKNSWEDPDTFNPSRFLQNGAPDFRGSNYEFLPFGSGRRSCPGMQLGLYAMEMAVAHLLHCFTWELPDGMKPSEIDMGDVFGLTAPKAIRLVAVPTPRLLCPLY

SEQ ID NO: 7 - protein, 506 aa, Cichorium intybus
MDSLQIPIPVYAIIAILTFFFLAWLRRKPLPPGPMGWPIIGNMLMMDQLTHRGLASLAKKYGGILHLKMGFSHTIAVSSPEIARQILQEKDNIFANRPATIAITYLTYNRVDMAFANYGPFWRQMRKLCVMKLFSRKRAESWDSVRDEVDTMVKATAINSGLPVNLGELVFGLTHDIIYRAAFGSISHEGKEEFIRILQEYTKLFGAFNLADFIPMLGFIDPAGLNTRLPAARAALDGFIDKIIDEHLSKEKKTGDENVDNDMVDEMLAFYSEDGRVNEGGDLQNAINLTRDNIKAIIMDVMFGGTETVASAIEWTMTELMHTPEALKRVQQELTNVVGLDRRVEESDFEKLTYFKCVIKETLRMHPPIPVLLHQSSEATEVSGYHIPKGTRVMVNAYAINRDKNSWEDPDTFNPSRFLQNGAPDFRGSNYEFLPFGSGRRSCPGMQLGLYAMEMAVAHLLHCFTWELPDGMKPSEIDMGDVFGLTAPKAIRLVAVPTPRLLCPLY

SEQ ID NO: 8 - protein, 503 aa, Cichorium intybus
MDPMSILLYVVLPLFTFFLLSRLRRKPLPPGPRGWPVIGNMLMMDQLTHRGLARLGEKYGGLLHLKMGFSHTVAVSSPEIARQVLQVQDNIFANRPATIAISYLTYDRQDMAFANYGPFWRQMRKLCVMKLFSRKRAESWDSVRDEVISMIKITAASSGSAVNLGELVFGLTHDIIYRAAFGSISHEGKEEFIRILQEYTKLFGAFNLADFIPWLGFIDPAGLNTRLPKARAALDGFIDKIIDEHLSKEKKTGDEEDNDMVDEMLAFYSEEGKVNEGEDLQNAIRLTRNNIKAIIMDVMFGGTETVASAIEWALTELMHTPESLKRAQQELADVVGLDRRVEESDFEKLTYFRCVIKETLRLHPPIPVLLHQSSEATEVAGYHIPKGTRVMVNAFAINRDKNSWKDPHMFNPSRFLQDGAPDFKGSNYEFLPFGSGRRSCPGMQLGLYAMEMAVAHLLHSFTWQLPDGMKPSEIDMSDVFGLTAPKAIRLVAVPTPRLLCPLY

SEQ ID NO: 9-10 - skipped (2 entries)

SEQ ID NO: 11 - protein, 502 aa, Apium graveolens
MSLLFILLLTIISMIFFISRFGGKPYPPGPRRLPVIGNTLMMACLTHRGLAKLASRYGGLFYLRMGAQDIVVVSTPDMARQILQTHQDQISNRPTSIALDYLSYGRANMAFADYSPWWRQMRKICVMKLFSRARVESWNSVRDELNHMLRDVASNANQAINLGELVFGFTEKIIYRAAFGSRLSDGGTEFIKIMQEFSKLFGSFNVCDFVPWMSRADPQGLKARLRKARGSLDTFIDSIIDQHIIKRKGKNIGDKDMVDELLAFYTEEGEAKAESDDLQATIKLTKNNIKGIIMDIMFGGTETVAAAIEWAMSELLKNPEELRKTQEELSNVVGLHRCVEEGDLEKLTYLKCVLKETLRLHPPLPFLPRAAAEDVYVAGYYIPAGSRVIINLWAMGHDGKCWNDEPEAFKPSRFLDVGAPDYRNNNFEFIPFGTGRRSCPGMQLGLHAFEMGLAHLLHCFNWELPDGMKPSQVDMSDMYGLSAPKATRLIAVPTPRLLCPIC

SEQ ID NO: 12 - protein, 501 aa, Apium graveolens
MTTIFFFLLLLCLFLARRKPYPPGPKGWPIIGNMLIMDKLTHRGLAKIATQYGGIVHLRMGFLHMVTVSTPDMVREVLQIQDTVFANRPATMNISYLTYNRADMAFANYGPFWRQMRKISIIKLFSRKRAESWDSVRDQVDDMLGKVVSNSGLSVNIGELVFGLTRNIIYRAAFGSISGQGQDEFIKIMQEFSKLFGAFNICDFVPGITWLDPQGFKVRLVKARESLDKFIDSILDEHIANQKSNLNGSTDEGNGDMVYQLLAFYSEEQSKVNHSDDTNNALKLTRDNIKAIIMDVMFGGTETVASAIEWAMSELMGSPEDLKRVQQELIDVVGLHRRVEENDFDKLIYLKCCIKETLRLHPPIPLLLHETAQEAVVAGYHIPAKSRVMINSWAVNRDPNSWDDPEKFKPSRFLKQGMPDFKGSNFEFIPFGSGRRSCPGMQLGLYAFEMAVAHLLHSFNWELPDGMKPSELDTNDVFGLTAPRATRLVAVPTPRLLCSIC

SEQ ID NO: 13 - protein, 511 aa, Apium graveolens
METNTTAMTILFFILPLLSFFLLSSFRRKRYPPGPKGWPIIGNLLMMDKLSHRGLAKLAAQYGGLVHLRMGFLHMFTVSTPDMAREVLQIQDNIFANRPATMNISYLTYDRADMAFANYGPFWRQMRKISVMKLFSRKRAESWDSVREEVDDMVKIVLSKTGCSVNIGELVFGLTRNIIYRAAFGTLSHEGQDEFIKILQEFSKLFGAFNICDFVPGLTWADPQGFMGRVVKARASLDGFIDSIIDAHIEKKKSSKNGIIDEGNSDMVYELLDFYGEEKAKVSEFEDQNSSLKLTRDNIKAIIMDVMFGGTETVASAIEWAMSELMRSPKDLKKVQQELVNVVGLHRRVEESDFDKLTYLKCCIKETLRLHPPIPLLLHETAQDAEVAGYHIPARSRVIINSWAINRDPNSWTDPDTFKPSRFLQEGMPDFKGSNFEFIPFGSGRRSCPGMQLGLYALEIAVAHLLHCFNWELPDGMKPSEVDTDDVFGLTAPRATRLVAVPTPRLLCPIS

SEQ ID NO: 14 - protein, 520 aa, Brassica oleracea
MESSISQTLSQVLDPTTGILIVVSLFIFIGLITRGRRPPYPPGPRGWPIIGNMSMMDQLTHRGLANLAKKYGGLCHLRMGFLHMYAVSSPDVARQVLQVQDSVFSNRPATIAISYLTYDRADMAFAHYGPFWRQMRKVCVMKVFSRKRAESWASVRDEVDRMIRSVSSNVGKSINVGEQIFALTRNITYRAAFGSACEKGQDEFIRILQEFSKLFGAFNVADFIPYFGWIDPQGINKRLVKARNDLDGFIDDIIDEHIKKKENQNSIDAGDVVDTDMVDDLLAFYSEEAKLVSETADLQNSIKLTRDNIKAIIMDVMFGGTETVASAIEWALTELLRSPEDLKRVQQELAEVVGLDRRVEESDIEKLTFLKCTLKETLRLHPPIPLLLHETAEDTEIDGYFVPKKSRVMINAFAIGRDKNSWVDPETFRPSRFLEPGVPDFKGSNFEFIPFGSGRRSCPGMQLGLYALELAVAHILHCFTWKLPDGMKPSELDMSDVFGLTAPKATRLYAVPSTRLICSV

SEQ ID NO: 15 - protein, 519 aa, Brassica oleracea
MESSVSQTLSQVLDPTTAILIVVSLFIFIGLITRRRRSYPPGPRGWPIIGNMLMMDQLTHRGLANLAKKYGGLCHLRMGFLHMYAVSSPDVARQVLQVQDSIFSNRPATIAISYLTYDRADMAFAHYGPFWRQMRKVCVMKVFSRKRAESWASVRDEVDKMIRSVSSNVGKSINVGEQIFALTRNITYRAAFGSACEKGQDEFIRILQEFSKLFGAFNVADFIPYFGWIDPQGINKRLVKARNDLDGFIDDIIDEHMKKKENQNTVDDGYVGDTDMVDDLLAFYSEEAKLVSETTDLQNSIKLTRDNIKAIIMDVMFGGTETVASAIEWALTELLRSPEDLKRVQQELAEVVGLDRRVEESDIEKLTFLKCTLKETLRLHPPIPLLLHETAEDTEIDGYFVPKRSRVMINAFAIGRDPKSWPDAETFRPSRFLEPGVADFKGSNFEFIPFGSGRRSCPGMQLGLYALELAVAHILHCFTWKLPDGMKPSELDMNDVFGLTAPKATRLFAVPSTRLICAV

SEQ ID NO: 16 - protein, 512 aa, Brassica oleracea
MDCLLCSPILYVVLIFLWYLVRVLLTRSNPFPPGPKGYPIIGNMKLKNQLNHRGLAELAKQYGGLLHLQMGRIHIVAASTAEMAREILQVQDVVFANRPANVAISYLTYNRADMAFANYGPLWRQMRKVCVMKLFSRKRAESWASVRDEINTMVQTLTKQTGSPVNVGELVFALTRNITYRAAFGSFARDGQDEFVKILQEFSKLFGAFDITEFLPWMKWFSNRDFSKRLENARKSLDGFIDRIIDAHIEKKNSRKQDDDGLEDDMVDELMAFYSGESGENGGKSNDSLSSFKLTRDNIKALVMDVMFGGTETVASAIEWAMTELMKNPHELVKLQQELADVIGLNREFHESDLENLPYFRCAMKETLRLHPPIPLLLHEAAADSVVSGYSIPRDSRVMINVYAIGRDGSVWTEPDAFRPGRFMDSKAPDFKGSDFEFLPFGSGRRSCPGMQLGLYAMELAVAHMLHSFDWELPEGGSSDDLDMTDMFGLTAPRATRLIAVPSYRLKCPMVI

SEQ ID NO: 17 - protein, 514 aa, Brassica oleracea
MESLLSQTLNQVIDPTPSVLLITISLLVVVYLISQWFKPLYPPGPKGLPVIGNMLMMDQLTHHGLAKLAHKYGGLFHLRMGFRHVFAITSPDVARQVLQVQDISFSNRPVNVAINYLTYDLADMAFAPYGPFWREMRKVCVMKVFSRKRTESWASVREEVNNMVRSFSSNVGKPVNVGELIFTLTRNITYRAAFGAACETEQDEFIRILQEFSKLFGAFNIADFIPFLGWFDFQGINKRLVKARNDLDGFIDEVIDEHMKKRETVNVDEDTDMVDDLLAFYSEDSSTNRNKNTVKLTRDNIKALVMDVMFGGTETMASGIEWALTELLRNPAELKRLQQELTEVVGLDRRVDDTHLEQLTFLKCTLKETMRLHPPIPLILHEAIEDRKLQGFFVPKGSRLMINAFAIARDPKLWVDPEAFRPSRFMEPGMPDFMGTNFEFIPFGAGRRSCPGMQLGLYAMEVAVANIIHCFTWKLPDGMKPSELDMSDVMGLTAPRATRLIAVPDTRLICPVYP

SEQ ID NO: 18 - protein, 517 aa, Brassica oleracea
  UNSURE 401: note = Unknown
MYTLMTLILLVPLLLFLFRHLLSRRLRQRKPYPPGPKGLPIIGNILMMNQFNHRGLAKLSRTYGGLLHLRLGISHLFVVSSPQIARQVLQVQDHVFSNRPTTIAIRYLTYGQSDLAFCNYGPFWRRMRKLYVMMLFSRKRAESWASVDEEVHKAVRSVAANVGKPLNVCKVAFSLTRDITFRAAFGSSSSSSNEGRLDEFLEIIQEFSKLFGEFNVADYVPSWLSWIDPQGINKRVEKARKSLDCFIESIINDHLDKKKTEKNVNVDEVTDMVDQLLAFYKEEVKVKDSETKINLDNIKGIIMDVMFGGTETVALAIEWVLTELLRSPENMKRVQDELATVVGLERWSVEDTHLEKLSFLKCVLKDTLRLHPPFPLLLHETVEEAEVSGYFIPKGSRVMVXTYALGRDPASWSDPEIFNPSRFLDPGAPDLKGNSFEFIPFGSGRRSCPGMQLGMYAFELAVAHLLHCFTWRLPDGVKFGDVDTIEGPGLTVAKANSLVAVPIKRLLCPMVLESHNV

SEQ ID NO: 19 - protein, 524 aa, Solanum melongena
MKEMVQKNINSILEALQANPMLLFLFIIPLFFLYLFSTSSRRRYPPGPRGWPLIGNMMIMDQLTHRGLAKLAEKYGGVMHLKMGYIHKIVISGPEEARQVLQVQDNIYSNRPATVAISYLTYDRADMAFADYGPFWRQMRKLCVMKLFSRKRAESWDSVRDEVDSLIKIVTTNAGTSVNLGELVFGLTRDIIYRAAFGTSSAEGQEEFIKILQEFSKLFGAFNMVDFIPWLGWIGQQGLNVRLANARASLDGFIDSIIDDHIERKKANVTIDNGDRETDMVDELLAFYSEEATVNESEDNLQNAIRLTRDNIKAIIMDVMFGGTETVASAIEWVMAELVKNPEELKKVQQELANVVGLNRRVEESDLEKLTYLKCCLKETLRLHPPIPLLLHETAEESTIFGYHIPAGSHVVINSFAIGRDKNSWEDADSYKPSRFLKQGVPDFKGGNFEFLPFGSGRRSCPGMQLGLYALEMAVAHLLHCFTWELPDGMKPSELKMDDTFGLTAPLANRLVVVPSPRLLCTLY

SEQ ID NO: 20 - protein, 524 aa, Solanum melongena
MKEIIQNNTFFILTTKETNLMMPLYFIFPIFIFFFFTISRLNKKNFPPGPKGWPIIGNMMMMDQLTHHGLAKLAKKYGGILHLQMGYLQMTVVSSPAEAREVLQLQDTIFANRPTTIAVEYLSYARADMAFANYGPFWRQMRKLCVMKLFSRKRAESWDSIRDEVESMTMAVATRVGSSVNIGELVFGVAKNIIYRAAFGTCSNRGQDELLNIMQEFSKLFGAFNLADFIPWLGWVDPQGLNKMIIKARASLDGFIDTIIDDHIQRKKTNNYNDEGNNNDMVDELLAFYGEKTKLNDSDDLTNALRLTRDNIKSIIMDIMFGATETVASAIEWAMAELMKSPEDLKMVQQELANVVGLHRKVEEMDFEKLIFLKCCIKETLRLRPPIPLLVHESVEDTTINNYYIPAKSRVIVNAWAIGRDKNSWDDPESFKPSRFLKEGVADFKGGDFEFLPFGSGRRSCPGMQMGLYAFEMTLAHLLHCFNWELPNGMKPSDIDMNDVFGLTVPKATRLVAVPTPRLLCQLY

SEQ ID NO: 21 - protein, 522 aa, Solanum tuberosum
MKEMVLNNINSTLEALQAQPMLLFFFIIPLFFLYLFSTSRRKRYPPGPLGWPLIGNMMMMDQLTHRGLAKLAQKYGGVFHLKMGYVHKIVISGPEEARQVLQVQDNIYSNRPKTVAISYLTYDRADMAFADYGPFWRQMRKLCVMKLFSRKRAESWDSVRDEVDSMVKIVTTNTGTSINLGELVFCLTRNIIYRAAFGTSSDEGQDDFIKILQEFSKLFGAFNMADFIPWLRWIGQQGLNVRLAKARASLDGFIDSIIDDHIERKKANVINDDGYRESDMVDELLAFYSEETKVNESEDLQNSIRLTRDNIKAIIMDVMFGGTETVASAIEWAMAELMKSPEDLKKVQQELANVVGLNRKVDESDFEKLTYLKCCLKETLRLHPPIPLLLHETAEESTVSGYYIPAKSHVIINSFAIGRDKNSWEDPDSFKPSRFLKEGVPDFKGGNFEFLPFGSGRRSCPGMQLGLYALEMAVAHLLHCFTWELPDGMKPSELKMDDIFGLTAPLANRLVAVPTPRLLCNY

SEQ ID NO: 22 - protein, 516 aa, Malus domestica
  UNSURE 318: note = Unknown
MDYLLQSLQPLQSMTPLLLIIPLLFLLPLIFRFRRPPPYPPGPKGLPLIGNMLLMDQLTHRGLAKLAKKYGGIFHLRMGFLHMVAISNPDVARQVLQVQDNIFSNRPATIAISYLTYDRADMAFAHYGPFWRQMRKLCVMKLFSRKRAESWESVRDEVDSSVRTVTVHVGSAVNIGELVFSLTKNIIYRAAFGTSSQEGQDEFIGILQEFSKLFGAFNIADFIPSLGWVDPQGLNNRLAKARESLDRFIDTIIDDHMEKKRNGKGVSDGETDMVDELLAFYSEEAKVYESEDNLQNAIKLTRDNIKAIIMDVMFGGTXTVASAIEWAMSELMKSPEDLKRVQQELADVVGLDRRPEETDFEKLTYLKCALKETLRLHPPIPLLLHETSEDAVVAGYRIPKRSRVMINAWAIGRDKDSWEDAESFKPSRFLKEGVPDFKGSNFEFIPFGSGRRSCPGMQLGLYALEMAVAHLLHCFTWELPDGMKPSELDMNDVFGLTAPRASRLIAVPSKRVVCPL

SEQ ID NO: 23 - protein, 517 aa, Malus domestica
MDSLLQSLQPLKSMTPLVFIIPLLFLLPLIFCFRRRPPYPPGPKGLPLFGNMLMMDQLTHRGLAKLAKQYGGIFHLRMGFLHMVAISNPDVARQVLQVQDNIFSNRPATIAISYLTYDRADMAFAHYGPFWRQMRKLCVMKIFSRKRAESWESVRDEVDTAVRTVTVHVGSAVNIGELVFSLTKNIIYRAAFGTSSQEGQDEFIGILQEFSKLFGAFNIADFIPSLGWVDPQGLNNRLAKARESLDRFIDTIIDDHMEKKKKNNKGLNDGETDMVDDLLAFYSEEAKVNESEDNLQNAIKLTRDNIKAIIMDVMFGGTETVASAIEWAMSELMKNPEDLKRVQQELLNVVGLDRRPEEADFEKLTYLKCALKETLRLHPPIPLLLHETSEDAVVAGYHIPKKSRVMINAWAIGRDKDSWEDPESFKPSRFLKEGVPDFKGSNFEFIPFGSGRRSCPGMQLGLYALEMAVAHMLHCFTWELPDGMKPSELDMNDVFGLTAPRASRLVAVPSKRVVCPL

SEQ ID NO: 24 - protein, 519 aa, Malus domestica
MELLHQALQSLQSSPMLLILLLLLLIISFIFLFNSRRKPPYPPGPKGWPIIGNMLMTDQLTHRGLAHLAKQYGGLLHLQMGVIHVMAVSTPDMAREILQSQDSLFANRPANVAISYLTYDRADMAFANYGPFWRRMRKICVINLFSRKRAESWASVREEVDEMVQTVAGKTGSPVNIGQLVFALTRNITYRAAFGSSSHEGQGEFVQILQEFSKLFGAFNMQDFLPWLGWVHAQGFQDRMARARKSLDVFIDKIIDDHMAKRKANMEKDDSEAADTDMVDELIAYFSDDAGKEGDDPNSGFKLTRDNIKALIMDVMFGGTETVASVIEWTMAELMKSPEDLKRVQQELTDVVGLNRRLQETDLENLTYLKCAVKESLRLHPPIPLLLHETVEDTSVAGYSFPAGSRVWINAWAIARDPTAWDEPETFKPSRFLEDSSPDFKGSNFEFIPFGSGRRSCPGMALGLYGLEMAVAHLLHCFAWELPGGMKPSELDMNDVFGLTAPKAVQLVAVPTYRLNCPL

SEQ ID NO: 25 - protein, 516 aa, Pyrus x bretschneideri
MDSLLQSLQALQSMTPLLLIIPLLFLLPLIFRFRRPPPYPPGPKGLPLIGNMLLMDQLTHRGLAKLAKKYGGIFHLRMGFLHMVAISNPDVARQVLQVQDNIFSNRPATIAISYLTYDRADMAFAHYGPFWRQMRKLCVMKLFSRKRAESWESVRDEVDSAVRTVTVHVGSAVNIGELVFSLTKNIIYRAAFGTSSQEGQDEFIGILQEFSKLFGAFNIADFIPSLGWVDPQGLNNRLAKARESLDRFIDTIIDDHMEKKRNGKGVSDSETDMVDELLAFYSEEAKVNESEDNLQSAIKLTRDNIKAIIMDVMFGGTETVASAIEWAMSELMKSPEDLKRVQQELADVVGLDRRPEETDFEKLTYLKCALKETLRLHPPIPLLLHETSEDAVVAGYRIPKRSRVMINAWAIGRDKDSWEDAESFKPSRFLKEGVPDFKGSNFEFIPFGSGRRSCPGMQLGLYALEMAVAHLLHCFTWELPDGMKPSELDMNDVFGLTAPRASRLIAVPSKRVVCPL

SEQ ID NO: 26 - protein, 516 aa, Pyrus x bretschneideri
MDSLLQSLQPLQSMTPLLLIIPLLFLLPLIFRFRRPPPYPPGPKGLPLIGNMLLMDQLTHRGLAKLAKKYGGIFHLRMGFLHMVAISSPDVARQVLQVQDNIFSNRPATIAISYLTYDRADMAFAHYGPFWRQMRKLCVMKLFSRKRAESWESVRDEVDSAVRTVTVHVGSAVNIGELVFSLTKNIIYRAAFGTSSQEGQDEFIGILQEFSKLFGAFNIADFIPSLGWVDPQGLNNRLAKARESLDRFIDTIIDDHMEKNRNGKGVSDSETDMVDELLAFYSEEAKVNESEDNLQSAIKLTRDNIKAIIMDVMFGGTETVASAIEWAMSELMKSPEDLKRVQQELADVVGLDRRPEETDFEKLTYLKCALKETLRLHPPIPLLLHETSEDAVVAGYRIPKRSRVMINAWAIGRDKDSWEDAESFKPSRFLKEGVPDFKGSNFEFIPFGSGRRSCPGMQLGLYALEMAVAHLLHCFTWELPDGMKPSELDMNDVFGLTAPRASRLIAVPSKRVVCPL

SEQ ID NO: 27 - protein, 516 aa, Pyrus x bretschneideri
MDSLLQSLQPLKSMTPLVFIIPLLFLLPLIFRFRRLPPYPPGPKGLPLIGNMLMMDQLTHRGLAKLAKQYGGIFHLRMGFLHMVAVSNPDVARQVLQVQDNIFSNRPATIAISYLTYDRADMAFAYYGPFWRQMRKLCVMKLFSRKRAESWESVRDEVDSAVRTVTVHVGSAVNIGELVFSLTKNIIYRAAFGTSSQEGQDEFIAILQEFSKLFGAFNIADFIPSLGWVDPQGLNNRLAKARESLDRFIDTIIDDHMEKKKNNKGLNDGETDMVDELLAFYSEEAKVNESEDNLQSAIKLTRDNIKAIIMDVMFGGTETVASAIEWAMSELMKSPEDLKRVQQELADVVGLDRRPEETDFEKLTYLKCALKETLRLRPPIPLLLHETSEDAVVAGYRIPKRSRVMINAWAIGRDKDSWEDAESFKPSRFLKEGVPDFKGSNFEFIPFGSGRRSCPGMQLGLYALEMAVAHLLHCFTWELPDGMKPSELDMNDVFGLTAPRASRLIAVPSKRVVCPL

SEQ ID NO: 28 - protein, 517 aa, Pyrus x bretschneideri
MDSLLQSLQPFTSMTPLVFIIPLLFLLPLIFRFRRLPPYPPGPKGLPLIGNMLMMDQLTHRGLAKLAKQYGGIFHLRMGFLHMVAVSNPDVARQVLQVQDNIFSNRPATIAISYLTYDRADMAFAHYGPFWRQMRKLCVMKLFSRKRAESWESVRDEVDTAVRTVTVHVGSAVNIGELVFSLTKNIIYRAAFGTSSQEGQDEFIAILQEFSKLFGAFNIADFIPSLGWVDPQGLNNRLAKARESLDRFIDTIIDDHMEKKKNNNKGLNDGETDMVDDLLAFYSEEAKVNESEDNLQNAIKLTRDNIKAIIMDVMFGGTETVASAIEWAMSELMKSPEDLKRVQQELFNVVGLDRRPEEADFEKLTYLKCALKETLRLHPPIPLLLHETSEDAVVSGYHIPKQSRVMINAWAIGRDKDSWEDPESFKPSRFLKDGVPDFKGSNFEFIPFGSGRRSCPGMQLGLYALEMAVAHMLHCFTWELPDGMKPSELDMNDVFGLTAPRASRLVAVPSKRVVCPL

SEQ ID NO: 29 - protein, 519 aa, Pyrus x bretschneideri
MELLRQALQSLQSSPMLLILLLLLVISLIFLFNSRRKLPYPPGPKGWPIIGNMLMTDQLTHRGLAHLAKQYGGLLHLQMGAIHVIAVSTPDTAREILQVQDSSFANRPANIALSYLTYDRADMAFANYGPFWRRMRKICVINLFSRKRAESWASVREEVNEMVQTVAGKTGSPVNIGQLVFALTRNITYRAAFGSRSHEGQGEFVKILQEFSKLFGAFNMQDFLPWLGWVHAQGFQDRMARARKSLDVFIDKIIDDHMAKRKANTENKDDSEAADTDMVDELIAYFSDDAGKEGDDPNSGFKLTRDNIKALIMDVMFGGTETVASVIEWTMAELMKSPEDLKRVQQELTDVVGLNRRLQETDLENLTYLKCAVKESLRLHPPIPLLLHETVEDTSVAGYSFPAGSRVWINAWAIARDPTAWDEPETFKPSRFLDDGSPDFKGSNFEFIPFGSGRRSCPGMALGLYGLEMAVAHLLHCFAWELPGGMKPSELDMNDVFGLTAPKAVQLVAVPTYRLNCPL

SEQ ID NO: 30 - protein, 520 aa, Prunus persica
MDDLLLHQALESLQSSPMLFILLFLLIISWFVLFMSRRKLPYPPGPRGWPIIGNMLMMDQLTHRGLAQLAKQYGGLLHLQMGVLHIMVVSSPKVAREILQVQDSSFANRPANAAISYLTYDRADMAFANYGPFWRRMRKICVINLFSRKRAESWASVREEVEEMVRHVATKTSSPVNIGQLVFTLTKNITYRAAFGSSSHEGQGEFVKILQEFSKLFGAFNMQDFLPWLGWVHAQAFKDRMAKARRSLDVFIDKIIDDHMAKRNTNKAKKDDNEAETDMVDELIAFFSDDAAKESDDPNSTFRLTRDNIKAIIMDVMFGGTETVASVIEWTMAELMKSPEDLQKVQQELINVVGLNRRVQETDLENLTYLKCAVKESLRLHPPIPLLLHETAEETSVAGYSFPVGSRVYINAWAIARDPTAWDEPETFKPSRFLKDGSPDFKGSDFEFLPFGSGRRSCPGMQLGLYGLEMAVAHLLHCFAWELPEGMKPNELDMNDVFGLTAPKAVQLVAVPSYRLNCPL

SEQ ID NO: 31 - protein, 519 aa, Prunus persica
MDSLLQALQPLQPMTLFFIIPFLFLSGLVFLYRSRRRSPYPPGPTGLPIIGNMLMMDQLTHRGLAKLAKQYGGIFHLRMGFLHMVGISNPDVARQVLQVQDNIFSNRPATIAISYLTYDRADMAFAHYGPFWRQMRKLCVMKLFSRKRAESWDAVRDEVDTAVRTVAVQAGSAVNIGELVFSLTKNIIYRAAFGTSSEEGQDEFIGILQEFSKLFGAFNIADFIPCLGWVDPQGLNNRLARARQSLDRFIDSIIDDHIQKKKKKKSEGSNGGETDMVDELLAFYSDEAKVNESDDNLQNAINLTRDNIKAIIMDVMFGGTETVASAIEWAMAELMRNPEELKRVQQELADVVGLDRRPEEGDFEKLTYLKCALKETLRLHPPIPLLLHETAEDAEVAGYHIPKKSRVMINSWAIGRDKDSWEDAESFKPSRFLKEGVPDFKGSNFEFLPFGSGRRSCPGMQLGLYSLELAVGHLLHCFTWELPDGMKPSELDMNDVFGLTAPRASRLIAVPSKRVVCPL

SEQ ID NO: 32 - protein, 516 aa, Musa acuminata
MEWSEEVTPLHFMVCFALPLVLLYVVATRRRGKLPFPPGPPQLPVIGNMLMMDQLTHRGLAKLGEHYGGLCHLRLGFLHAFAVSTPEIARQVLQVQDNVFSNRPATIAISYLTYNRADMAFAHYGPFWRQMRKLCVMKLFSKKRAESWASVREEVDVAVRSLADRAGSAVNVGELLFNLTKNIIFRAAFGTQSHENQNEFISILQEFSKLFGSFNIGDFIPWLSWMDPQGINKRLKVARASLDRFIDKIIDEHMANRKEADASDADMVDDMLAFLDESGYRCQAGERDDLQGTLKLTRNNIKAIIMDVMFGGTETVASAIEWAMAELMKSPEDMKRVQQELAHVVGLDRKVHESDLDKLSFLKCVTKETLRLHPPIPLLLHETAEDCEVAGYTVPARSRVMINVWAIGRDKSSWEDADAFRPSRFTPGGCAASLDFKGNYFEFLPFGSGRRSCPGMQLGLHALELAVAQLIHCFTWTLPDGMKPSELDMGDVFGLTAPRAVRLAAVPAPRLSCPLY

SEQ ID NO: 33 - protein, 516 aa, Musa acuminata
MEWLEEVTSMRFVVCVVVPVTLLLAASTRWRRKLPFPPGPTPLPIVGNMLMMGQLTHRGLAKLSERFGGLCHLRLGFVHVFAVSTSEIARQVLQVQDAVFSNRFATIAITYLTYDRADMAFAHYGPFWRQMRKLCVMKLFSKKRAVSWASVREEVDAAVRAVTDGAGAAVNLGELMFNLTKNITFRAAFGTQSHENQEEFIAILQEFSLLFGAFNIGDFIPWVSWMDLQGINKRFKVAREALDGFIDKIIEEHMANPKEADAEDSDMVDEMLAFFEESRDRTKENEADELQRTLRLTRNNIKAIIMDVMFGGTETVASAIEWAMAELMKNPEDMRRVQEELASVVGLHRKVRESDLDKLPHLKCAVKETLRLHPPIPILLHETAEDCQLTGYAVPARSRIMINVWAIGRDKSAWEDAEVFRPSRFAPGGEAAALDFKGGCFEFLPFGSGRRSCPGMQLGLHALELAVAQLTHCFSWELPDGMKPGELDMGDMFGLTAPRAVRLVAVPTPRLTCPLY

SEQ ID NO: 34 - protein, 515 aa, Musa acuminata
MVWVEEVTSMHLILLCLMLPLTLLLIVNIAARRRRRLPLPPGPTPLPIIGNMLLMNQLTHRGLARLAKLYGGLLHLRLGFVHHFVVSTPDVARQVLQVQDSVFSDRPATTAIVYLTYNRSDLAFAQCGPYWRQMRKLCVTKLFSRKHAESWLSIPEEVDAAVCTVAKHAGSAFNVRDLAFTLTKNIVFRSAFGKRSDENQEEYIAVVQEIATLLGAFSVGDFIPWLSWMDPQGINKRLRVARATLDLFIDRIIDEHMATDAANADMVGVMLAFLEESSHHHRQEEGDDLKGTLRLSRANIRAVMMDVMFGGTETVAIAIEWALADLLTSPDDLKRVQEELAMVVGLDRKVHESHLDKLSFLKCAIKETLRLHPPFPLLLHQTADHCEVAGYSIPARSPVMINVWAIGRDESAWKDADAYRPSRFAPGGDAAALDFKGNCFEFLPFGSGRRSCPGMQLGMHELELAVAQLLHCFTWALPDGMKPTELDMGDVFGLSAPKAVPLVAVPTPRLSCPLN

SEQ ID NO: 35 - protein, 511 aa, Musa acuminata
MDWFHQLSFMVASVFIPLALLSFFCMRSGRKLLLPPGPQPLPIIGNMLMMDQLTHRGLARLAERYGGLFHLRLGSLHAVVVSTPEMARLVLQVQDASFCNRPVTAAIAYLTYDRADMAFANYGPFWRQTRKLCVMKLFSRRRLQSWASVRQEVDSAVRFAARRSGSSVDVGDLAFTLAKNVTFMAAFGAQSHGNQGEFAGILQEYSKLFGEFNISDFLPWLRWMDLQGIDKRLKVARQAIDRYIDVIIDDHLANPKEADAQDADMVDGMLAFLGDSGDTNEGGDLHGDLSLTRSNIKAIIMDVMFGGTETVALGIEWAMAELLKSPEELKRTQQELASVVGLHRKVDDSDLDKLPYLKCAVKEMLRLHPPLPLLQHQATQDCELAGYFIPVGTRVFVNAWGIGRDRDAWKSPNAFRPSRFALGGDAAAFDFRGSCFELLPFGSGRRSCPGMQLGLYVLELAVAQLLHCFDWSLPAGTKPGDLDMGDVFGLTAPKAVRLMAVPTPRLTCPLL

SEQ ID NO: 36 - protein, 509 aa, Triticum aestivum
MATFAKIAMELLADPLMWLFLASLALVAMQRRRLGSAPFPPGPKPLPVIGNMTLVDQLTHRGLAALAKQFGGLLHLRFGWLHVLAVSTPEYAREVLHAQDGVFSNRPATIAVVYLTYGRDMAFAHNGAYWRQMRKLCVTKIFSRRRAETWLAVREGYGALAREVGRRSGEAVNLGELIFNLTVSVIFRAAFSTCDEDGLIEFIAILQEFSRLLGLFHIGDFFPWLAWVGRRGFNRRLSTARGALDRFIDKIVDEHMRRGKDPADPDADLVDGLLGFLADANPREDALRFTRDNVKAMIMDMLFGGPETVGSTTEWAMAEMMRSPDELERLQQELADVVGLDRAVEESDLDKLPFLRCVVKEALRMHPPIPVLLHEAAKDCVVGGYSIPRGSRVLVIAWAINRDCGAWKDGDTFRPARFIPGEGEAAGLDLKGSCYEFLPFGSGRRSCPAQGLGQHAVEFAVAQLAHGFNWKLPDGMKPAELDMGDIFGLTASRSTRLYAVPTPRLTCPV

SEQ ID NO: 37 - protein, 515 aa, Triticum aestivum
MAAYAKVGTEFLKDPLIWLFLASLAFVILQRRRLGSAPFPPGPKPLPVIGNMALVDQLTHRGLAALAKQYGGLLHLRLGRLHVYAVSTPEYAREVLHVQDAALSNRPATIAVVYLTYGRSDMAFAHNGAYWRQMRKLCVTKIFSRRRAETWLAVREGYGALACAVSRRCGEAVNLGELIFNLTVSVIFRAAFSTRDEDGLDEFIAILQEFSSLLGLFHIGDFFPWLGWVGRRGFNRRLRTARGALDKFIDRIIDEHMKRGKNAADPEADLVDGLLAFLAEANPISGKHREDALRFTRDNAKAMIMDMLFGGPETVGSMTEWAMAEMMRSPDDLRRLQRELANVVGLHRTVDETDLDKLPFLRCVVKEALRMHPPIPLLLHEAAKDCFVGGYSVPKGSRVLVNAWAINRDPGAWKDGDTFRPSRFMPGEGEAAELDLNGGCYEFLPFGSGRRSCPAQGLGQHAVEFAVAQLAHGFNWELPDGMKPAELDMGDIFGLTALRATRLYAVPTPRLTCPM

SEQ ID NO: 38 - protein, 535 aa, Triticum aestivum
MVGLAKIAMDWLQEPLSWLFVASFVFVVLQRHRQRLRGKAPPLPPGPSPLPIVGNMFMMDQLTHRGLAALARQYGGILHLRLGQVHAVVLSTPEYAQEVLQAQDVAFSNRPATVAAIYLTYDRADMAFAHYGPFWRQMRKLCVMKLFSRRRAGTWLAVRDESAALVRAVARRSGESVNLGELIFNLTKNVTFRAAFGADAAGDAGKRDEFIAIMQEFSQLFGGSSIGDFIPWLGWVDQGLNVRARTARAALDEFIDKIIDEHMRRGKNPDDVDADMVDDMLAFLPEAKPKKDAGDDLQNSLHLTRDNIKAMIMDVMFGGTETVASGIEWAMTEMMHSPDDLRRLQQELADVVGLDRNVDESDLDKLPFLKCVIKETLRLHPPIPILHHENAEDCVVGGYSVPRGSSVMINVFAIGRDAKVWKDADTFRPSRFMTGEGEAARVDFKGSCFEFLPFGSGRRSCPGMALGIYSLEFAVAQLAHGFSWALPDGMKPSELDMTDIFGLTAPRATRLCAVPTPRLTYPLISDVDATHKGNT

SEQ ID NO: 39 - protein, 535 aa, Triticum aestivum
MVGLTKIAMEWLQDPLSWLFVASVVFVMLQRRRRRGRAPPLPPGPNPLPIVGNMSMMDQLTHRGLTALAKKYGGFLHLRLGKVHAFAVSTPEYAQEVLQVQDAAFSNRPASLAATYLTYDRADMAFAHHGPFWRQMRKLCVMKLFSRRRPETWLAVRNESAALVRAVARRSGETVNLGELIFNLAKNVTFRAAFGAGAAGDAGKQEEFIAILQEFSKLFVEFCIGDFIPWLSWADPQGINVRLRAARAALDQFIDKIIDEHMKRGRNPDDVDADMVDDMLAFLPEARTKKAAGDRGDDLQNTLRLTRDNIKAMIMDVMFGGTETVASAIEWAMSEMMHCPDDLRRLQQELADTVGLDQNVDESDLDKLPFLKCVIKETLRLHPPIPLLNHENAEDCVVGGYSVPRGSRVMINVFAIGRDASTWKDADAFRPSRFMEGEGEAAGVDFKGGCFEFLPFGSGRRSCPGMALGLYSLELIVAQLAHGFNLALPDGMAPSELDMRDVFGLTVPRATRLCVVPTPRLTCSLVADDDAAHQA

SEQ ID NO: 40 - protein, 526 aa, Triticum aestivum
MVDLSMIDMEWLQEPLSWLFVASVIFVLLQRRRGKAPPLPPGPYSPPIVGNIFMMDQLTHRGFAALAKQYGGLLHLRLGKVHTFAVSTPEYAQQVLQAQDAAFSHRPATIATTYLTYDRADMVFARYGPFWRQMRKLCVMKLFSRRRPGTWLAVRDESAALVRAVARRSGEPVNLGDLIFNLSMNVTFRAAFGAEAAGDGDGRKQHEFIAIMQEFSKLFGAFSIGDFIPWLGWADPQGIKVRLRAARTALDEFIDKIIDEHIKRGRNPDDMDADMVDGMLAFLPEAKPDKAAGDDLHHTLRLNRDNIKAIIMDVMFGGTETVASAIEWAMAEMMHSPNDLLQLQQELADTVGIDRNVDEPDLNKLSFLKCVIKETLRLHPPIPLLHRENAEDCVLGGYSVPQGSSVNINVFAMGRDTKVWKDADTFRPSRFMEGEGEAAGVDFKGGCFQFLPFGSGRRSCPGMALGLYSLELVIAQLAHGFNWALPNGEKPSVLDMSDIFGLTAPRATRLWVVPTPRLTCPLVVDV

SEQ ID NO: 41 - protein, 519 aa, Oryza sativa
MANGVAEYLLMDPWLVLWLVLASMAFALLHLRRRARRGAPPLPPGPRPLPIIGNMLMMDQLTHRGLAAMAARYGGLLHLRLGRVHMVVVSSPEHAREVLQVQDGDFSNRPASIAIAYLTYGRADMAFSHYGHFWRQVRKLSAVRLFSRRRAQSWRAVRDESAKLVGAIARRAGEAVDLGELIFGLTKDVIFRAAFGTRDGGGHGELEVLLQEFSKLFGAFNVGDFIPWLAWLDPHGINRRLRAARAALDSVIDRIIDEHVSNPAGDEDADMVDDMLAFLDEAGRDQTGGGGELQGTLRLTRDNIKAIIMDFVFGGTETVASAIEWAMAELLHSPGDLRRLQAELADVVGLGRGVEEGDLEKLPFLRCVAMETLRLHPPIPLLLHEAAADCVVGGYSVPRGARVVVNVWSVGRDAGAWKGDAGAFRPARFMAGGEAAGMDLRGGCFELLPFGSGRRACPAIVLGMYELELVVARLVHAFGWAPPGGVAPEELDMADGFGLTAPRAARLRAVPTPRLTCPM

SEQ ID NO: 42 - protein, 530 aa, Oryza sativa
MADMVKFTMEWLQDPLSLAIVVTVAVLIMRMQRRRAAPFPPGPKPLPIVGNMAMMDQLTHRGLAALAKEYGGLMHLRLGRLHAFAVSTPEYAREVLQAQDGAFSNRPATTAIAYLTYDRADMAFAHYGPFWRQMRKLCVVKLFSRRRAETWLAVRDESAALVRAVAASRGEAAVNLGELIFNLTKNVIFRAAFGTRDGEGHDEFIAILQEFSKLFGAFNIGDFIPWLSWADTNGINARLVAARTALDRFIDKIIDEHMERGKNPDDADADMVDDMLAFLAEAKPHAGKAAAAAAGAGDGADDLQNTLRLTRDNIKAIIMDVMFGGTETVASAIEWAMAEMMHSPDDLRRVQEELAAVVGLGRDVAESDLDKLPFLRCVIKETLRLHPPIPILLHETAADCLVAGYSVPRGSRVMVNVWAIARDRAAWGPDADAFRPSRFAAGAAAEGLDFRGGCFEFLPFGSGRRSCPGMALGLYALELAVARLAHGFNWSLPDGMKPSELDMSDIFGLTAPRATRLSAVATPRLTCPLY

SEQ ID NO: 43 - protein, 526 aa, Zea mays
MVTVAKIAMEWLQDPLSWVFLGTLALVVLQLRRRGKAPLPPGPKPLPIVGNMAMMDQLTHRGLAALAERYGGLLHLRLGRLHAFAVSTPEYAREVLQAQDGAFSNRPATIAIAYLTYDRADMAFAHYGPFWRQMRKLCVMKLFSRRRAETWVAVRDECAALVRAVASGGGGGGEAVNLGELIFNLTKNVTFRAAFGTRDGEDQEEFIAILQEFSKLFGAFNVVDFLPWLSWMDLQGINRRLRAARSALDRFIDKIIDEHVRRGKNPDDADADMVDDMLAFFAEAKPPKKGPAAAADGDDLHNTLRLTRDNIKAIIMDVMFGGTETVASAIEWAMAEMMHSPDDLRRLQQELADVVGLDRNVNESDLDKLPFLKCVIKETLRLHPPIPLLLHETAGDCVVGGYSVPRGSRVMVNVWAIGRHRASWKDADAFRPSRFTPEGEAAGLDFKGGCFEFLPFGSGRRSCPGTALGLYALELAVAQLAHGFNWSLPDGMKPSELDMGDVFGLTAPRATRLYAVPTPRLNCPLY

SEQ ID NO: 44 - protein, 532 aa, Zea mays
MAAAVANIGMEWLQDPLSWVFLGTVCLVVLQQLRRRRGKAPLPPGPKPLPIVGNMGMMDQLTHRGLAALAETYGGLLHLRLGRLHAFAVSTPEYAREVLQAQDGAFSNRPATAAIAYLTYDRADMAFAHYGPFWRQMRKLCVMKLFSRRRAETWAAVRDECAALVRAVAVGGGSGGEAVNLGELIFSLTKNVTFRAAFGTRDGEGQEEFIAILQEFSKLFGAFNVGDFLPWLGWMDLQGINRRLRAARSALDRFIDKIIDEHVRRGKSPDDADADMVDDMLAFFVEATPGKATGAAAAADGGDDLHNTLRLTRDNIKAIIMDVMFGGTETVASAIEWAMAEMMHSPDDLRRVQQELADVVGLDRNVSESDLDRLPFLRCVIKETLRLHPPIPLLLHETADDCVVAGYSVPRGSRVMVNVWAIGRHRASWKDADAFRPSRFAAPEGEAAGLDFKGGCFEFLPFGSGRRSCPGMALGLYALELAVAQLAHAFNWSLPDGMKPSEMDMGDIFGLTAPRATRLYAVPTPRLNCPLY

SEQ ID NO: 45 - protein, 515 aa, Glycine max
MANLDLDPFQTSILILVPIALLVALLSRTRRRAPYPPGPKGLPIIGNMLMMEQLTHRGLANLAKHYGGIFHLRMGFLHMVAISDPVAARQVLQVQDNIFSNRPATIAISYLTYDRADMAFAHYGPFWRQMRKLCVMKLFSRKRAESWQSVRDEVDAAVRAVASSVGKPVNIGELVFNLTKNIIYRAAFGSSSQEGQDEFIKILQEFSKLFGAFNIADFIPYLGCVDPQGLNSRLARARGALDSFIDKIIDEHVHKMKNDKSSEIVDGETDMVDELLAFYSEEAKLNNESDDLQNSIRLTKDNIKAIIMDVMFGGTETVASAIEWAMAELMRSPEDQKRVQQELADVVGLDRRAEESDFEKLTYLKCALKETLRLHPPIPLLLHETAEDATVGGYFVPRKARVMINAWAIGRDKNSWEEPETFKPARFLKPGVPDFKGSNFEFIPFGSGRRSCPGMVLGLYALELAVAHLLHCFTWELPDGMKPSEMDMGDVFGLTAPRSTRLIAVPTKRVVCPLF

SEQ ID NO: 46 - protein, 515 aa, Glycine max
MANLDLDPFQTSILILVPIALLVALLSRTRRRAPYPPGPKGLPIIGNMLMMEQLTHRGLANLAKHYGGIFHLRMGFLHMVAISDPVAARQVLQVQDNIFSNRPATIAISYLTYDRADMAFAHYGPFWRQMRKLCVMKLFSRKRAESWQSVRDEVDAAVRAVASSVGKPVNIGELVFNLTKNIIYRAAFGSSSQEGQDEFIKILQEFSKLFGAFNIADFIPYLGCVDPQGLNSRLARARGALDSFIDKIIDEHVHKMKNDKSSEIVDGETDMVDELLAFYSEEAKLNNESDDLQNSIRLTKDNIKAIIMDVMFGGTETVASAIEWAMAELMRSPEDQKRVQQELADVVGLDRRAEESDFEKLTYLKCALKETLRLHPPIPLLLHETAEDATVGGYLVPKKARVMINAWAIGRDKNSWEEPESFKPARFLKPGVPDFKGSNFEFIPFGSGRRSCPGMVLGLYALELAVAHLLHCFTWELPDGMKPSEMDMGDVFGLTAPRSTRLIAVPTKRVVCPLF

SEQ ID NO: 47 - protein, 520 aa, Glycine max
MDWQSMMGNLDPFQRTILILVPLTLLLLLLLSRTRPRPPYPPGPKGFPIIGNMFMMDQLTHRGLANLAKHYGGIFHLRMGFLHMVAISDPDAARQVLQVQDNIFSNRPATIAISYLTYDRADMAFAHYGPFWRQMRKLCVMKLFSRKRAESWQSVRDEVDAAVRAVASSVGKPVNIGELVFNLTKNIIYRAAFGSSSQEGQDEFIKILQEFSKLFGAFNIADFIPYLGCVDPQGLNSRLARARGALDSFIDKIIDEHVHKMKNDKSSEIVDGETDMVDELLAFYSEEAKLNNESDDLQNSIRLTKDNIKAIIMDVMFGGTETVASAIEWAMAELMRSPEDQKRVQQELADVVGLDRRAEESDFEKLTYLKCALKETLRLHPPIPLLLHETAEDATVGGYFVPRKARVMINAWAIGRDKNSWEEPETFKPARFLKPGVPDFKGSNFEFIPFGSGRRSCPGMVLGLYALELAVAHLLHCFTWELPDGMKPSEMDMGDVFGLTAPRSTRLIAVPTKRVVCPLF

SEQ ID NO: 48 - protein, 518 aa, Glycine max
MDLLLELKTALEPFRETLLFTIPLTLLLLGIVSRIRRKTAPYPPGPKGLPLIGNMNIMNQLTHKGLANLAKQYGGVLHLRIGFLHMVAISNAEAAREVLQVQDNIFSNRPATIAISYLTYDRADMAFAHYGPFWRQMRKICVMKLFSRKRAESWNTVRDEVDFIIRSVTNNLGSPVNVGELVFNLTKNIIYRAAFGSSSQEGQDEFISILQEFSKLFGAFNVADFVPFLGWVDPQGLNKRLVKARASLDSFIDKIIDEHVQKRRSGHDGDEESDMVDELLNFYSHEAKLNDESDELLNSISLTRDNIKAIIMDVMFGGTETVASGIEWAMAELMRSPDDLRRVQQELADVVGLDRRVEESDLEKLVYLKCAVKETLRLHPPIPLLLHETAEDAAVCGYHVPKGSRVMINAWAIGRDKSAWEDAEAFKPSRFLNPHVPDFKGSNFEFIPFGSGRRSCPGMQLGLYTLELAMAHLLHCFTWELPDGMKPSELDTSDVFGLTAPRASRLVAVPFKRVLCPL

SEQ ID NO: 49 - protein, 521 aa, Glycine max
MDWQSMMGNLDPFQRTILILVPLTLLLLLLLSRTRPRPPYPPGPKGFPIIGNMFMMDQLTHRGLANLAKHYGGIFHLRMGFLHMVAISDPDAARQVLQVQDNIFSNRPATIAISYLTYDRADMAFAHYGPFWRQMRKLCVMKLFSRKRAESWQSVRDEVDSAVRAVANSVGKPVNIGELVFNLTKNIIYRAAFGSSSQEGQDDFIKILQEFSKLFGAFNIADFIPYLGRVDPQGLNSRLARARGALDSFIDKIIDEHVQKKNNYQSSEIGDGETDMVDELLAFYGEEAKLNNESDDNLQNSIRLTKDNIKAIIMDVMFGGTETVASAIEWVMSELMRSPEDQKRVQQELADVVGLDRRVEESDFEKLTYLKCALKETLRLHPPIPLLLHETAEDATVGGYFVPRKARVMINAWAIGRDKNSWEEPETFKPARFLKPGVPDFKGSNFEFIPFGSGRRSCPGMVLGLYALELAVAHLLHCFTWELPDGMKPSEMDMGDVFGLTAPRSTRLIAVPTKRVVCPLF

SEQ ID NO: 50 - protein, 512 aa, Cynara cardunculus var. scolymus
MDVLQLQGLLHPMPILFYVALPLLTFFLLSSFRRKPLPPGPRGWPLIGNMLMMDQLTHRGLASLADKYGGLLHLKMGFSHTVAVSSPEMARQVLQVQDNVFANRPATIAITYLTYDREDMAFANYGPFWRQMRKLCVMKLFSRKRAESWDSVRDEVVSMIKITAASSGSAVNLGELVFGLTHDIIYRAAFGSISHEGKEEFIRILQEYTKLFGAFNLADFIPWLGFIDPAGLNTRLPKARGELDGFIDKIIDEHLRKEKKTGDDADNDMVDEMLAFYSEEGKVNEGEDLQNAIRLTRNNIKAIIMDVMFGGTETVASAIEWALTELMHTPEALKRAQQELADVVGLDRRVEESDFEKLTYFKCIIKETLRLHPPIPVLLHQSSEATEVAGYHIPKGTRVMVNAYAINRDKNAWKDPNTFNPSRFLENGAPDFKGSNYEFLPFGSGRRSCPGMQLGLYAMEMAVAHLLHSFTWQLPDGMKPSEIDMTDVFGLTAPKATRLVAVPTPRLLCPLY

SEQ ID NO: 51 - protein, 506 aa, Cynara cardunculus var. scolymus
MDSIQIPISIYVIIALLTFSFLAWLRRKNLPPGPMGWPVIGNMLMMDQLTHRGLARLAEKYGGILHLKMGFRHTIAVSSPEIARQILQVQDNIFANRPATIAITYLTYDRVDMAFADYGPFWRQMRKLCVMKLFSRKRAESWDSVRDEVDSMVTTTATNSGLPVNLGELVFGLTHDIIYRAAFGSTSHEGKEEFIRILQEYTKLFGAFNLADFIPWLGFIDPAGLNTRLPAARAALDGFIDKIIDQHLAKEKKVGDENVDNDMVDEMLAFYSEEGKTNEAEDLQNAIKLTRDNIKAIIMDVMFGGTETVASAIEWAMTELMHTPEALKLVQQELTNVVGLDRRVEESDFEKLPYFKCVIKETLRLHPPIPVLLHQSSEATEVSGYHIPKGTRVMVNSYAINRDKNAWTDPNTFNPSRFLQNGAPDFRGSNYEFLPFGSGRRSCPGMQLGLYAMEMAVVHLLHCFNWELPDGMKPSEIDMGDVFGLTAPKAIRLVAVPTPRLLCPLY

SEQ ID NO: 52 - protein, 520 aa, Raphanus sativus
MESSISQTLGQVLDPTTGILIVVSIFIFIGLITRRRRPPYPPGPRGWPIIGNMSMMDQLTHRGLANLAKKYGGLCHLRMGFLHMYAVSSPDVAKQVLQVQDSVFSNRPATIAISYLTYDRADMAFAHYGPFWRQMRKVCVMKVFSRKRAESWASVRDEVDKMIRSVSNNVGKSINVGEQIFALTRNITYRAAFGSACEKGQDEFIRILQEFSKLFGAFNVADFIPYFGWIDPQGINKRLVKARNDLDGFIDDIIDEHMKKKENQNTADDGEVVDTDMVDDLLAFYSEEAKLVSEATELQNSIKLTRDNIKAIIMDVMFGGTETVASAIEWALTELLRSPEDLKRVQQELAEVVGLDRHVEESDIEKLTFLKCTLKETLRLHPPIPLLLHETAEDTEIDGYFVPKKSRVMINAFAIGRDKNSWVDPETFRPSRFLEPGVPDFKGSNFEFIPFGSGRRSCPGMQLGLYALELAVAHILHCFTWKLPDGMKPSELDMSDVFGLTAPKATRLYAVPSTRLICAV

SEQ ID NO: 53 - protein, 518 aa, Raphanus sativus
MESSVSQTLSQVFDPTTTILIVVSVFIFIDLITRRRRSYPPGPRGWPIIGNMLMMDQLTHRGLANLAKKYGGLCHLRMGFLHMYAVSSPDVARQVLQVQDSIFSNRPATIAISYLTYDRADMAFAHYGPFWRQMRKVCVMKVFSRKRAESWASVRDEVDKMIRSVSSNVGKSINVGEQIFALTRNITYRAAFGSACEKGQDEFIRILQEFSKLFGAFNVADFIPYFGWIDPQGINKRLVKARNDLDGFIDDIIDEHMKKKENQNTVDDGDVDTDMVDDLLAFYSEEAKLVSETTDLQNSIKLTRDNIKAIIMDVMFGGTETVASAIEWALTELLRSPEDLKRVQQELAEVVGLDRHVEESDIEKLTFLKCTLKETLRLHPPIPLLLHETAEDTEIDGYFVPKKSRVMINAFAIGRDPKSWPDAETFRPSRFLEPGVADFKGSNFEFIPFGSGRRSCPGMQLGLYALELAVAHILHCFTWKLPDGMKPSELDMNDVFGLTAPRATRLFAVPSTRLICAV

SEQ ID NO: 54 - protein, 515 aa, Raphanus sativus
MESLLSQNLNHVIDPIPSALLITISLLVVVYLISQWLKPLYPPGPKGLPVIGNMLMVNQLTHHGLAKLANRYGGLFHLRMGFRHVFAITSPDVARQVLQVQDISFSNRPVTVAINYLTYDLADMAFAPYGPFWRQMRKVCVMKVFSRKRAESWASVREEVNNMVRSLSSNDVGKPVNVGDLIFTLTRNITYRAAFGAACETEQDEFIRILQEFSKLFGAFNIADFIPFLGWLDLQGINKRLVKARNDLDGFIDEVIDEHMKKRETINGDEDTDMVDDLLAFYSEDSSPNRSKNAVKLTRDNIKALVMDVMFGGTETMASGIEWALTELLRNPAELKRLQQELTEVVGLDQRVDDTHLEKLTFLKCTLKETMRLHPPIPLILHEAIEDTKLQGFSVPKGSRLMINAFAIARDPKLWVDPETFRPCRFMEPGMPDFMGTNFEFIPFGAGRRSCPGMQLGLYAMEVAVANIIHCFTWKLPDGMKPSELDMSDVMGLTAPRATRLIAVPDTRLICSVWP

SEQ ID NO: 55 - protein, 509 aa, Raphanus sativus
MDCLLCSPILYVVLIFLWYLVRVLVTRGNPFPPGPKGYPIIGNMKLKNQLNHRGLAELAKQYGGLLHLQMGKIHIVAASTAEMAREILQVQDVVFANRPANVAISYLTYNRADMAFANYGPLWRQMRKVCVMKLFSRKRAESWASVRDEINTMVQTLTKQTGSPVNVGELVFALTRNITYRAAFGSFARDGQDEFVKILQEFSKLFGAFDITEFLPWMKWFSNRDFSKRLENARKSLDGFIDRIIDAHIEKKNSRKQEDDGLEDDMVDELMAFYSGENGGKSNDSLSTFRLTRDNIKALVMDVMFGGTETVASAIEWAMTELMKNPHELVKLQQELADVIGLNRQFHESDLENLPYFRCAMKETLRLHPPIPLLLHEAAADSVVSGYSIPRDSRVMINVYAIGRDGSVWTEPDAFRPGRFMEDKAPDFKGSDFEFLPFGSGRRSCPGMQLGLYAMELAVAHMLHSFDWKLPEGVSSGDLDMTDMFGLTAPRATRLIAVPSYRLKCPMVI

SEQ ID NO: 56 - protein, 517 aa, Raphanus sativus
MYTPMTLILLVPLLLFLYWHLLSRRLRLRKSYPPGPKGLPIIGNILMMNQVNHRGLAKLSRTYGGLLHLRLGLSHLFVVSSPQIARQVLQVQDHVFSNRPTTIAIRYLTYGQSDLAFGNYGPFWRRMRKLYVMMLFSRKRAESWASVDEEVHKAVRFVAANVGKPLNICKVAFSLTRDITFGAAFGSSSSTSDEGRLDEFLEIIQEFSKLFGEFNVADYVPSWLSWIDPQGINKRVERARKSLDCFIESIINDHLDKKKTENNVDVDEETDMVDQLLAFYKEEVKVKDSETKINLNNIKGIIMDVMFGGTETVALAIEWVLTELLRSPENMKRVQEELATVVGLERWSVEDTHLEKLTFLKCVLKETLRLHPPFPLLLHETVEDAEVSGYSIPKGSRVMVNTYALGRNPNSWSDPEIFNPSRFLDPGAPDLKGNSFEFIPFGSGRRSCPGMQLGLYAFELAVAHLLHCFTWKLPNGVKPGDVDTIEGPGLTVPKANSLVAVPTTRLLSPIVLESHNV

SEQ ID NO: 57 - protein, 505 aa, Allium cepa
MMDMQSILIFTLPFVTLLFLVITSRRRPKLPLPPGPRPLPIIGNLNLIDKLTHRGLAHLANQYGGIFHLKLGSVHTFSISTPEIAKEVLQTQDLAFSNRPATIAITYLTYDRADMAFTHYGPFWRQIRKLCVMKLFSRKRAESWASVREEIEKAVSTAAAGAGTVVNVGELVFNLTKNITFRAAFGAKSGEEQDEFLGILQEISKLFGAFNVGDFVPGLRYLDPQGIGRRMRKVRKELDGFIDRIIDEHVQNRKEVDDVEADMVDEMLAFVGQGKSIGRDSDELRLTRNNIKAIIMDVMFGGTETVASAIEWAMAELLKSPEDLKRLQQELTSVVGLDRKVQDSDLDKLPYLKCVIKETLRLHPPIPLLLHETAEDCEIQGYSIPKKSRVMINVWAIGRDKSAWKDADQFKPSRFVKGGEYEQVDFKGNFFELLPFGAGRRSCPGMQLGLYALDLTVANMAHCFDWELPDGMKPGEMDMSDVFGLTAPRAVRLAAVPSPRLTCRI

SEQ ID NO: 58 - DNA, 5782 bp, Lactuca sativa
  misc_feature 1..5782: note = source = /organism="Lactuca sativa"/mol_type="unassigned DNA"
  source 1..5782: mol_type = unassigned DNA
gaaatatatttcatttttagcacatatttaccacataaacacaagaaatcacacataacatacaaatgcacatagatcttcacaattttacttgtattctcccccataaatcatatgaaaaccgaaaatagaggggtataaactcaccttgagtttgtggattcagtttttgaagaagatgtggaaagatgaaatgatttttcggccctagcaactcccttgagtagatctcgaactcaagaagatttcacaagtatgtatcttggagtgattcttggatgaaaaccttcttgaatgcccttaatcttgaaaattttgtaggaggaatgaaggtgttcttgagaggatttggaagtgaattaagtgtgtgtgtgtgtgtgtttggtgatcctcggtcaagagtagaagaagagatgggagggagtgacgtgagttgacatgcatagagatatgagaatggtgcatggatatcctatgtacatttatgaggtggcaccacaaacctcatttcatcttttcttttatttatttttattttttttttgttttacttgggccgaaatgggctaaggaaggatgaaggaatcactttggcccattctttccttggctcgagttcatgaggcccataaggaaaacttacggcccgttgggcccttagggtagctcacgacccaaatctttatataagagaatggatttgagtccatcaaagctaattggatttgttatggcccaataaggcctattaaagataaactagtccattttaggcttataaggcccaaaaaggttaagtctcatgagagtgggtccaatgagagcccatttaagagttctaagcccaaaatagtcaaattgaaagcttattggcccattaggcccaataaggaaattctagttcatatatgacttaaaccggaatttctagggtttttcaactttacttgatgaatctaggtttgaatgtgtttccataggttgaacatcaagcataacatgaattagggttacgagtttacaatagataatgttccaagagtacagaatttcctagctagaatgctatttgtgacatctgtgcctaatagatactttgatttttaattatacttaactcaagtaagttgaataaatatttttgttaattataatttattgtattaacttgaagcatttgaataatagaaaatatttttggtttctatacataaatgtaagtcatatcgaataacaaacttgaggtaggaagataaaaaataaatcgaaatttgcatgaggagaacaaaaccaaaacagaataattaaatattattaaaaaaaacatgtgagtggttggtaaagtaaccaatggttaaactcaaatactctctattttaatctaataataagtaataacaatgtaaatattttacatcttaacaagtggtacataaaagagaattatttctgacatgtcagattaatttccaaaattacttgtctttgttcatttattatatttaaaaaagaatatacttgacgcaattagtgtaacaagttatccaaaaatccattaccaccaaaatcaaaacaaatctaaacatatttactcttacaatctaaacataatcattaaaaaaactaaattaaataaacaataacctaaacaaataatttatctcatatgattacaagaatgtaaaattaattggtcgaccaagtcacttgtgcagtagccagcaactagcaacgttatgctgacatacaattctatcagatctaagccgaataaaaaatcaaactatatttatgtgtaaataattgaccggcactctacgtctttgtctcaagacaaaagaattagtcaaacatttcctgtaattctcatttttaatatttataataaataaaataaaagaaaaaaaaaatagtaatatccatacgacttcccacctttcaaacagggtgtgcccgtttatcttgtatataaacacccctttatctcactctttaggtagcaacattcatccagttgccacttaaaacctccatttgaatcaaaatggaatcacttcaaatccccatagcattctacgctataatagctatcttaactttcttctttctttcatgggtccgccggaaaccactcccgccggggccaatggggtggccaatcatcggcaacatgttgatgatggaccaacttacccaccgtggcttagcccgtttggcagaaaaatacggtggtatccttcatctaaagatgggtttcagccacaccattgcagtgtcctcgccggagatggcgaggataatacttcaagaaaaagataacatctttgccaaccgtccggcaaccatcgccatcacttacctgacttacaacggcgtagatttggcttttgctaattatggacctttctggcgacaaatgcgaaagctttgtgtcatgaagctgttcagccggaaacgagcggagtcatgggactccgtcagggatgaggtggacaccatggtgaaagccaccgccattaactccggtacgccggtaaacttgggtgagcttgtttttgggttgacccatgatattatctaccgagcagcttttgggtcgatttcacatgaagggaaagaagagtttatcagaatccttcaagaatacaccaaactttttggcgcattcaatttggctgactttatcccgttcctcgggtttattgatccggcggggttgaacacacgtttaccggcggccagggcggcgttggacggattcattgacaaaatcatcgacgagcatttgcgtaaaggaaagaaaaccggcgatgaaggtttggataacgatatggttgatgagatgttggcgttttacagcgaggaaggaaaagtcaacgaaggtggtgatttgcaaaacgccattaaccttactcgagataacatcaaagccataatcatggtaagtaatgaaaccatgaatacattctttttataaatcattataacattccaaattctaaaagttaatccaaaacttttgtaggatgtaatgttcggtggaactgagacagtggcgtccgccatagaatgggccatgacggagctaatgcatacaccggaggcactaaagcgcgtgcaacaggagatggcaaatgtcgtcggacttgaccggcgcgtggaggagtctgacttggagaagctgacgtacttcaaatgcgtcatcaaggaaaccctccgactacaccctccgatcccagttctcctccaccagtcttcggaggcgacagaagtttccggctaccatatacctaaaggaacacgtgtcatggtgaacgcgtatgctattaatcgtgataagaactcttgggaagatccggatacgtttaacccgtcacgttttttacaaaacggagctccggattttagaggaagcaactatgagtttctgccatttggttctggtcggaggtcgtgtccggggatgcaactagggttgtatgcgatggagatggcggtggcccaccttttgcattgtttcacgtgggaattgccggatggaatgaagccaagtgaaatcgacatgggtgatgtgtttggactcacagcaccaaaagcaataagattggtagcagtgccaactccgcgtttattatgcccattgtattgaaatggttttatcatttatatgtatttatatgtaggttgcatacggtttcaaattgtataagattaagatatataataacacggaaatatttttcatatcatatatatggtatcaccataagccttatttgaactcaatgggattgtatcgatctttatttttaaaattcataggcttaaggccggtccaagaaaacttgaagcccggggcaaaacacaaaaatgggattgtagggattatatggatctttattgtatggatctttatttttaaaattcataaagatatgtgaaaactcagaaacgaactatatataaaaagatatttgcacctttaatattcggtagtaaaaaaatgtctctatttgtatgttgtttcatccaacacacctttattacaaagatatacatcaccaaaacttacccttcaacttaaggttcatagctaaactatacttctgaacttaagtttctatcaaaaacaaagaaataaaaaactaaaaatgattgatttttccaaaagataacatgatacacagtgtagtagatcattgcacattgcataaattgccaacaaatactaacgtctacttctatcacatttttcttttttcacttatagtgctttaaaaacctctttcgctatcatctataacaacatcatcattaattcattaacatgcatacctataatcataattaaaaatacaaaaaacttgcctaaaatgaaaatgcatcgggttcttgttctaggtttgcacttaaattcagccaaaaatgtagtatgatctgaataccattcacaatcagagaaggtcggatactctaggggtgagcaaaaccgaacccggaaaccgaaaaaccgacaaaaaccggcaaaaccgaaaccgaaaaaccgaaaccgaagcataaaccgacggtttgggtttatattttttgaaaaccgaaaatttcggtttgggttgggtttttgggtttggtaaaaccgatagaaaccgaaccgaaccgattatatatactatttgtaaaaatatataatttatatacaaatataaatatgtttatagaagtaatgtcaaaaggatgcgctggttgaggtgggtaacgctcttgcttgtgtttctgaaggtttcaagttcgaaacctgtggccaaaattttacttcttttattttcgcagctatataaacctatcccacatcgacacatgatcgaaactttgctacatatgctcgcctacataagctaaacatacatcctcttcaggtttattcagctattagctttgtcccacatcgcttgagggatcaaagtagaagccattttcacttctataaaaatgaggatacgaaatgatagaaaaacagttttaggtttggtttagttcaaaaaccgaaaccgaaaacccgatttggaaaccgataatccataaaaccgaacccatgggtttagggtcggttagggttcaaatttttagaaaccgatggtcatcggtttgggttcggttttagggcaaaaccgacccaaaccgacccatgctcagccctaggatactcctttgattctatacaacacaaaattccgaagctaaaaaaaagattcataaaaaaaaaatcgtatttcaaacaagaataaaaaatttcaagaaggctatgataaactagaacttataaagacatataatcagtttcgtctcaatgtcaagtataatcgattttgtctcaatttaaagaagattttgagtctgagtttgtaacactcgtgtttctagcctagacatttatatcgaggtgatagtctaggttaacctttgtaactcattttgaagaaataaaaagaaattatgtgaatattatgtgaattatgtgttttatgcttaattattagggcttaattaattaagaataaaaataagcgtcaaaattaaagtgtgagataagcccgatatctttacataaagttgtagtggtccaaacaaggatttcagggatataaagaatatcaaaatccgagttataaagaagaagttatgacctgtcgaagtttcgcgacaaaaccggcacggtgctgaatgtcgtaaaaagtgagtttttgataaactactttttagccttagtaatctaaacgaaagtcat

SEQ ID NO: 59 - DNA, 6306 bp, Lactuca sativa
  misc_feature 501..1883: note = /note="n = a or t or c or g"
  misc_feature 1..6306: note = source = /organism="Lactuca sativa"/mol_type="unassigned DNA"
  source 1..6306: mol_type = unassigned DNA
ttttattttgtttaatttaattaatttttatttagataaaataaatgacgaaaataatattttaaaaaaattcaaaattttcatttatttttcattttaaaatatattttttttatccctaaaaggaatgacaacgattgcgttctcatgtcttacaaaatataataatatcacatgactgtaactatatatatatagttttaggttatctggataacaaaaaaccctaaagaaaaatcctaaaaatcataaaaatgtatgaaaaaaaaaatatttaaagatcacaaatattttttttgactttatatttttaaaagtggtaactttttatacaattttgctgaatttttttttgaaaaaaaaaaacctatgatttttaagaatataaagtcaaaaaaagatttgtgatctctaagtattttttatacattattatgattttaaattttttttcgttatccatataaccttctatatatctatatatatatatatatatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatatatgtgtgtgtgtgtgtgaaaatagtcaactttccaggattaaatataaaatgatataaataaatgcaaactcattcaaacagaggtgccgctgcatctcatttcaaccactgcccaccctgtatatatacacatcacttgatcttcctttacctagcagcgttttctagttgcacttcattacgctcatactccaatagacttactgaaccgaaactctttgccggaaaatatggatcctaagtccatcttactttacgttgtactccctctcttaaccttcttccttctctcccgattacgccgaaaacctcttccgcctggtccaagagggtggccgctgatcggtaacatgttaatgatggaccaactcacccaccgtggccttgctcgtttgggagaaaaatacggtggtcttcttcatctgaagatgggtttcagccataccgtcgctgtctcgtcccccgaaatagccaggcaagtactccaagttcaagacaacatcttcgccaaccgcccggccaccatcgccattagttacctcacctacgaccggcaagacatggcgttcgccaactacggtcccttttggcgtcagatgcgtaagctttgcgtcatgaagctgttcagcagaaagcgagctgagtcttgggactccgtcagagacgaagttgtctccatggtcaaaatcaccgctgcaagctccggcaccgctgttaaccttggagagcttgttttcgggttaacccatgatatcatttaccgagcagctttcgggtctatctctcatgaaggaaaagaagaattcatcagaattctacaagaatacacaaagctttttggtgctttcaatttggcagattttgtcccgtggcttggatttatcgaccctgccggactgaatacccgtttaccgaaggccagggcggcgcttgacagattcattgataaaatcatcgacgagcaccttgcaaaagagaggaaaacgggcgatgaggaagataatgatatggtggatgagatgttggctttttacagtgaagaaggaaaggtaaacgaaggcgaggatttgcagaacgcgattagactcacccgaaacaatatcaaagccattattatggtaagtaattgttgtttacattaatatataaaaaacaaaacaaaacaagttttctgaaaatagttgttttcgtagtcattttgttgaatactaacaaagtcgtaaatactttagctacttctatacgatacataaaacaattttctgttgtttttgggttttttaatattctatccaataaaaaagtataaaacatatctatttgatgtcatgttcaataaaaaaaatgtattaggtagaatccaaaaagttgtcaatattaattaagtattcgagttgatcacactgggatatggatagaaattgactaattgattacacatgattaattccagtccaacacaccaatacaattatgttcttatcattggttcacaactatagttctttgtttcatgtatttcttgaactatattattacaatgaatttattatgtattgattatatcattgtttatatgtaggatgtaatgtttggtgggactgaaactgttgcttctgctatcgaatgggctttaactgagctaatgcacaccccagaatccttaaaacgtgcacaacaagagctcgctgatgttgttggccttgatcgtcgtgtagaagaatcagatttcgagaagctaacttacttcaaatgtgtcatcaaagaaaccttacgtctccaccctccgatccctgtccttttgcaccaatcatcagaagccacgtcggttgctggctaccacatacctaaagggacacgtgtcatggttaacgcattcgccattaatcgtgataagaactcatggaaggatccacacacgttcaacccatcacgtttcttgcaagatggggcacccgactttaaaggaagcaattatgagtttcttccatttggatctggacgtagatcatgtcctggaatgcaacttggattgtacgcaatggagatggcagtggctcaccttcttcattcattcacatggcagttgcctgatggaatgaaaccaagtgagattgacatgaatgatgtgtttggactcactgcaccaaaagcgattcgacttgttgctgtgccaactcctcggttgttgtgtccgctgtattgagaaacggcgaaaagtttgatattgtgaaaatttgtttatgttgcatcatgcatcaaatcagattgcttttcttttacatctttctagtcatcttccataaagaaagatatacacaaattgaataatgcgtaaagattatattttcatgtacattttgaaatgttcaaactccttgtgtttcttgtgttcatattatattttcttcctcagagaattacacgcatgctttgtgttcatatttgggaaaaaaaaaacatataagttttgctatcaatgtcattcctggtaaaaatgaatgttgttctttatgatgtagcctaaatgtcaacttttttaggtttttctccaactacaactgatatgatatgctgcctgacgagtgactgggcttcttccaagtgggaccttcccattcacaaatttttattatgtttattgtcacaataatgatacttttagtaatataaatattacaaataaataaacaaacaaatatacattctctctctatcattagcatatagataaaaccgcaattacatgcataaaaaaaatataaaccgcattatatgaacaccgaattcgacccgtcagctagagccaacgtgcgaatcatggaaatcaataatattttgggcatgttacgctttgaacatttcacatctcaaccaatatgttcctatcattattattttgggcatgataatgacttaggagggtaactacctcttatctgtgttcacatattggcaacttcttcttttttgtacataagaaagaaactaacttgttttaactgtgtaacatccaaaaaatcatgaccaaaaattttgtttttaatttaatactaaaaccataattgtcaaaccattaatttaatactccagcatcgggcctcgcccgacatcaggccccgcctggcatcgagccccgctcggtatacaaatttgtctctcttaggccccgcctgtctccgggccccgcccgctaaacacatacaatcatctaacatgctaacacataaatcaacatactctactgtatccctgtcctaagaaacatactttgctaataatgagatacaaaatttcgtccgtacccaactagttccagccagggcttcggcagtgcaaggtgagtctcctcactctgtgaatgggtctaaggccacaatgccagcccatgtagtctatgagtaggaagactcgggggggttagccctaggcattgtttgctagtatcttattctagaccaaggactgatgtcgagcgagtgcctgaggaatgttttcatgttagattatacttgttgtatgtgtgatacttgtatgtgcctggtaaagaggtgagtgtgggcgagctcccgtatctcatcactaactgagtacgaacgatgttctgtatctcattattaccagagtatgtttcttaggacagggatacagtaaagtatgtttctttatgtgttagcatgttataggattgtatgtgtttagcgggcgggccccggagacaggcggggcctaagagagacatatatgtataccgagcggggctcgatgcccggcggggcccgatgacggacgggggcnattaaattaatggtttgacaattatggttttagtattaaattaaaaaagaaattttttggtcatgatttttgggatgttacaaagttaaaacaagttagttgctttcttatatacaaaaaataagaagttgtcaatatgtgaacacggataagaggtagttacactcctaatttattttccctttgagttaccggttaaaacagatattagccggtaatattggtgtaatttaaacagttaaaacaagttagttgctttattatgtacaaaaaagaagaagttgctaatacgtgaacatggataagaggtagttaccctcctaagtcattttcctaacatttcacatctcaactaataggttcctatcatattacattataatattatatacaaacgtgtatatgtgttagtacatggataatgacttaggagggtaataacgttttgtggttgtccattttaggtccctaacgtattttttgtgtgtattacaccattaaggttattataaccgtttacaaatagtccttttaacaggaagaaatcggtaaaaaggattatttctatacggttataacaaccttaatggtgtaatacacacaaaaaatacgttagtgac

SEQ ID NO: 60 - DNA, 5618 bp, Cichorium endivia
  misc_feature 1..5618: note = source = /organism="Cichorium endivia"/mol_type="unassigned DNA"
  source 1..5618: mol_type = unassigned DNA
caatacgtccaactattggcttcatatctaatggatacatttattctcttttttttcgtgatttgaggagtgctttcctcgttgctttctttttaatcatgcttccttcatcatcaaatgtaacattattcctactctcgccgtaaatttcttcacttaatccaatttccaaatgtgattggtgaggcttgaaagtttccccacagacaactattggtacatcatcacatttcccgacgttctgcatcatcaaaatgaataaacttttcatattagaaaccacaaccattattatttaataacataacgcacattaaaagtctgtcttcaacgctgttactcttggttaccacaacatcgttaattgtattcccgtcgtcctacataatcattaagtcatcaacatgttaaacttttcatattagcaaaccacaaccactatatcacattgcgtaccttgtcaggatgctcattagaactttgacttttggtaaccagataatccatggagaaactgtcctgcattagaaacaaaatcgaatataaagaaatatttttattcatgtgacaacttcaatataaaaattagacaccttgattacctcaatatcatcgacagctatcttgcatacatcctcatcggaccttcgcttacacagtagaccccccatgtataccgtatcctccaactacgtaaactcataaaacaacatctgttatatctatgtataagcaggaacatcaacccagacaaaatgaaaaccgtttagttccatgaaaatcgtcaacctcccatacgtgggtaactgaagccacgacctgaagattccccacaaaaagcaaaccctccaaataaccgtcacttctaaatgtatatggtgatttaacaccacttctaaacgtatatggtgatttaacacggttgagattgaaaattgggaagatcgaacggccagaattattggagaaaaaataggactcgttaggaacattaaagacataatttgacaatccctccgtaattttataactttctaacgttttgttagcattataaattaagacgttcaaacaaaaaaaaggcactttaaagtaaagtatttctaccgcatgatattattttccaacatcacctttctttctttgtcgaatattatattaaacaaaacaatgtttatgatataatcattctctgttctataaaaatagttagtcttctatatacttgatgtgattagtgtagtaatttatacgaagaactattttaacctgtatgaaacaaaaacaattatggtttttgatggtggactaattaacataatatttaaatatagaaagttaataagcaacaaccttaactaagaaattatgtcaattgaatacatgaacagaaaattaattcgaagaccaattcacttggtgtagcaactagtaactagtaacattatgctgacatttaatcttattatataaaataaaaaaataaaactctttatatgataatttagaccggcactcgtttgcaagacataagaattagtcaacatttcatgcatgtgattctcacttataataacacaaagtaaaaaggagtaaattgcaaaactggtccttaaattttacttaggattaaaaattcagttaaaaattttaaattatcacgatgtcgtccataaatttgaattttgttgatatgctctttgggattttctcaacagtacacgtttattcttgattttaaagtcatattatatgtattcggaaccatatttgcgacaaaatacaaacttaaagaatgatattattataaataatttgtaaaattgcaatatttggtaaagcacatggacatgatttgtaatttatatagtaatatctatttgaattccggccattcaaacggggtgtgcccgcttaacgtgtatataaacacccctttatttcactctttaggtagcaacattcaaccagttgccacttaactctccatttgaatcaatatggattatcttcaaatccctataccaatctacgctataatcgccatcttaaccttcttctttctggcatggctccgccgtaagccactcccgccaggcccaatggggtggccaatcatcggcaacatgttgatgatggaccaacttacccaccgtggtttagctagtttggcgaacaaatatggtggcatcctccatttaaaaatgggtttcagccacaccattgccgtgtcatcgccggagatagctcggcaaatacttcaagaaaaagataacatctttgccaaccgtccggccaccatcgccatcacgtatcttacctacaaccgcgtagatatggcgtttgcaaactacggaccattctggcggcagatgcggaagctttgtgtcatgaagctgttcagccgtaaacgagcggagtcatgggactccgtcagggatgaggtggataccatggtgaaggccaccgccactaactccgggttgccggtaaacttgggtgaactcgtatttgggttgacccatgatatcatttacagggcggccttcgggtcgatttcccacgaaggtaaagaagaattcatcagaatcctgcaagaatacaccaaactctttggcgcattcaacttggccgactttattccaatgctcgggttcattgacccggcagggttgaatacacgtttaccggcggccagggcggcgttggacggattcatcgacaaaatcatcgacgagcatttgagtaaagaaaagaaaaccggcgaygaaaatgtrgataacgacatggttgacgagatgttggcgttttacagcgaagacggaagaatcaacgaaggcggtgatttgcaaaacgctattaacttgacacgagataacatcaaagccataatcatggtacgtaacttatgtaactcgaataaactaataactatagatctttctaatatttaaaactattgttctaaagaaaaactatacgtaaattttgcaggatgtaatgtttggtggcactgagacggtggcttccgccatagaatggaccatgacggagctaatgcatacaccggaagcactaargcgcgttcaacaagagttgactaacgttgtcggtcttgaccgccgtgtcgaagagtctgacttygagaagctaacctacttcaaatgtgtcatcaaagaaaccctccggatgcaccctccgatcccagttctcctccaccaatcatcggaggcgacagaagtttccggctaccacatccctaaagggacacgtgtgatggtcaacgcrtatgccatcaatcgtgacaaaarctcttgggaagatcccgatactttcaacccatcccgttttttacaaaacggagctcctgattttaggggaagcaactatgaatttcttccgtttggttctggtcggaggtcgtgtccgggaatgcaacttgggttrtatgcgatggagatggcggtggcccaccttcttcattgtttcacgtgggacctgcccgacggaatgaaaccaagtgaaatcgacatgggtgatgtgttcggactcaccgcaccaaaagcaataaggctagtggctgtgccaactccgcgtttattatgtccattgtattgaaataattgtatgtaagttttttagagtttcattttataccagatatatataataacatggaaatatttttcttattgatctttgctaatatattactaaatcaacaaacctagttggaacttgaagtgtttactggatctttatttcccagattgtgacgacttgtaaaagtccgtgtaatccatgtgtgaaacatttggcgatgaaaaactcattggtgaatataaattagtagaaaaaagtcttttcatgacacacaattttgtcataaatggccacaaatgtctgtaattgaaaacacagtcctaaatgaaagacgacagacttttttatcataaatttacgatatacattatgatagataattacgacgtacaatgactgttgtaaacgactaaagatgacaaagtctatcattgaaaactccgtcgaaaatctaagatgacagacattttccgtaaaaatacgacatacatttttgacaaacaattacgacagacattcaaggcatacatctatcacatatattaatgactgaccttttgtatgtcgtaatttgacaaacttttttgacatacattttagtatttcataattttacgttttttaagaatatatttttttctctaaaatgtaccaatattaatcaaaataaacacacatttaatatatgataatacaaattaagataaatatacaaaaagtcaatccatcgcacaaattctagtatgtcaaatacaaagtaccatatgtttatcaaaaagtaattgtcaaatacacaatatcatcggcggtaaataaaggttgtgacataagtagccaaatcttcacgcacctcatcaagctcaacgttcgtatattcattcttcccccaaaaatgaaacaaaagttaatatattagccataaaaaacaattttaaacaattacttgaaaattgatctattttaaacaattttacttgcgttgttatccaacactttgactccttcatcgacaactgctttcatgaatttaagacataataccggcattttgtgccacctggctgcatcggacactatataaatacataataaatggttagtcaaaaaatatattagacaaatcatgaagttaaagctgtagtttgtgctcaccaagacatttatccacctcacactaatccttttgttggaatcacattgtgttgcatataaagataatgctctacaataaattggatgaaatatataacaacaaaaataaaataatgtgtgataatttatattacttatgctttaatcatttgcttcaattttgtattaacatttgcagtccttatagaatcaaggtaatagcaggtggttttttgcatgcttagtacgactagaacccaatagccactgcagaccaaaaagccaaataaaaattaaggcaaatacctctaatgattaagtcaactgagatagtattactaaaagaagacatgtccaaattaaacatgtcttcataaattaagacgtttttttacttaacccattaagtcattccatccacaccaggtaaactagaaacatacaagtataattgtttggatctagttactaaaatagtttagtactttttgtattcatagtcaaatagaaaatctaccccgggattctaaggtaataagatgatgtggtagtcctttcttgtgaacaaccgagatgttatgtttctactctcttgatcgccttttatgaagttttccagtcgtccatctttagatattgcactcgatatcacgaaacagattccatgagctgtttttcctccattcttgatttcttcatataagtgcctgttaagatacaaaagctaaattcgttaaatacacaaaatatagtaaattagtcaaaattaagcacttacaccatataactagatattatagaactggtcaacttatgcattgtaaacaccacttcaaaaccatcccaaaatatatatttgcaacaatcagactcaacaaacagtc

SEQ ID NO: 61 - DNA, 1521 bp, Cichorium endivia
  misc_feature 1..1521: note = source = /organism="Cichorium endivia"/mol_type="unassigned DNA"
  source 1..1521: mol_type = unassigned DNA
atggattctcttcaaatccctataccagtctacgctataatcgctatcttaaccttcttctttctggcatggctccgccgtaaaccactcccgccaggcccaattgggtggccaatcatcggcaacatgttgatgatggaccaacttacccaccgtggtttagctagtttggcgaaaaaatatggtggcatcctccatctaaagatgggtttcagccacaccattgccgtatcatcaccggagatagctcggcaaatacttcaagaaaaagataacatctttgccaaccggccggccacgatcgccataacctacctcacctacaaccgcgtagacatggcgtttgccaactacggacctttctggcggcagatgcggaagctttgtgtcatgaagctgttcagccgtaaacgagcggagtcatgggactccgtcagggatgaggtggataccatggtgaaggccaccgccattaactccggtttgccagtaaacttgggtgagctcgtatttgggttgacccatgatattatttatcgggctgctttcgggtcgatttcccatgaaggtaaagaagaatttatcagaatcctgcaagaatacaccaaactttttggcgcattcaacttggctgactttatcccaatgctcggttttattgacccggctgggttgaatacacgtttaccggcggccagggcggcgttggatgggttcatcgacaaaatcatcgacgagcatttgagtaaagaaaagaaaaccggcgacgaaaatgtggataacgacatggttgacgagatgttggcgttttacagcgaggacggaagagtcaacgaaggtggagacttgcagaacgccattaacctgactcgagataacatcaaagccataatcatggatgtgatgttcggtggcactgagacggtggcttccgccatagaatggaccatgacggagctaatgcatacaccggaagcactaaagcgcgttcaacaagagttgactaacgttgtcggtcttgaccgccgtgtcgaagagtctgactttgagaagctaacctacttcaagtgcgtcatcaaagaaaccctccggatgcaccctccgatcccagttctcctccaccaatcatcggaggcgacagaagtttccggctaccacatccctaaagggacacgtgtgatggtcaacgcatatgccatcaatcgtgacaaaaactcttgggaagatcccgatactttcaacccatcccgttttttacaaaacggagctcctgattttaggggaagcaactatgaatttcttccgtttggttctggtcggaggtcgtgtccgggaatgcaacttgggttgtatgcgatggagatggcggtggcccaccttttgctttgtttcacgtgggaactacccgacggaatgaaaccgagtgaaatcgacatgggtgatgtgttcggactcaccgcaccaaaagcaataaggctagtggcagtgccaactccgcgtttattatgtccattgtattga

SEQ ID NO: 62 - DNA, 5830 bp, Cichorium endivia
  misc_feature 1..5830: note = source = /organism="Cichorium endivia"/mol_type="unassigned DNA"
  source 1..5830: mol_type = unassigned DNA
ttccttaaaaggatgtatggaatatgaaattccaattcaggaactttgttttaaaaataaagtgcatcagacctgacctaacatgacttggtattcgtaataatagacatcaagaatcagaaaacatattttctgaaagtacattgctatcgaaagcaccatttcataaaaaaattaaaagaacttgcagactcagacctgacttggaaagaataatcataaaaaaaatacaaaaatgaaataaaatctctcaaaatacattgctattagaattaccattttagagtgatctttttccaaaaagaattgcatcccactcaggtaaaactggattgaacctacttctacagatgactataaaattgatgtaattttttgtctgacttttgtcacatattatgaaaatgataggatggagttctgcttggcctaaaatttttcagttgttgaatttgaatgttatttagctaccgtattcaagcattaataaagtgaatttgattctttacaatacctttttgactcctgtatacattggtcccaaaatagctcctctcttttaaaaaacaatgacaaaactatgaatcaattaccagaaagaggaaatggggttgaaattgttcattcatgaaaccaagttaagtcatacgcctccataatctagataagtaagtaactgaaaccacagtagtcaccaatggaattcaaacaagaaacaatattataaaaacaaattaaaaaagaatgaatgaaaaagaagatgacagtaaagtgtcgttgctacaagaacttaccaattgaaccagaaccatcagtccgctaccaccttaccaaggtatcccttgaaagggttgaatcaaactcggacttgattcatggatttgctaatttatatacactgaagccaaattagataatttactccatatatttggttgttcaacttcaactgtgcataaaaatgtcgaaaccaaatcaaataaaaaccctaaatggtgggatatttaggggatacgaatgatattggtggaaaaagaaaatgtgaatgaaacctgaatccattcatcgatcattcaaaatgaaaccccgattcaagcattgagagtaaaacctcaagaataccatatagtaagatgaagacgtagttgcaccggatggtttaacgatggaagcaacaacagggatgaaaaaaataggctaaaccggaaggcgtagatggaggctgcgaattatagatgttgattgctgttttgtggatgccgtttgagatttgtgcgttcaatatctaattgaatgcattctgtatctaatggctgatttatcgcctaacattcctgatttattggagatttggatggaactagttaattgtgatttaatatgataatagtacaatagaggttttgagcgggaaattaaaaaattggctacacaagaggaggacatgtggcactttttttactcttttattagaagcaaagatatatatactagttatatatatacatgagcagaaattaaaaatatacagcagtaatagcacagaaattattttcctgtatgatagaggcattgtgagattggattattggaataagctaaattttttttttccagatgtgtggtatattgttattttcgttgtagcaccggtaaaataaaaagaatttataaattttaccaattttcgaagaaaattccagtcacgagggcgccatgtgccctatagcaattcagcccctagttatctatgtaaaagcggaaatacgaaaatagtcaaccttacggcttcaagtatatataattatataataaaatgctaactcgttcaaacagagctatgcgctgcatcccctttcaagttcaactactgcgcaccctgtgtttttatatatatacactcgtgtgctctttctatacttggcaaccttctctcagttgcacttcattacccatatactttaagatactcactgaacataaattctttcccggatactatggatcccatgtccatcttactttacgttgtactccctctctttaccttcttccttctctcccgttttcgccggaaacctcttccgccaggtccaagaggatggccagtgatcgggaatatgttaatgatggaccaactcacccaccgtggcctagctcgtttaggtgaaaaatacggtggtcttcttcacctcaaaatgggtttcagccacacggttgctgtctcgtcgccggaaatagccaggcaagtgctccaagtgcaagacaacatctttgccaaccgtccggcaaccatagccatcagttatctgacctacgaccgccaagacatggcgttcgccaactacggtcccttctggcgtcagatgcgtaagctctgcgtcatgaagctgttcagccgaaaacgagcggagtcatgggactccgtcagagacgaagttgtctccatgatcaaaatcaccgctgcaagctccggttccgctgtcaacctcggcgagctggtgtttgggttaacccatgacatcatttaccgggcagctttcggttctatatcccatgaaggaaaagaagaattcataagaattctacaagaatacacaaagctttttggtgccttcaacttagccgattttatcccatggctaggatttatcgaccccgctggactgaacaaccgattacccaaggcccgagcagcgttagatgggttcatcgataaaatcattgatgagcacctaaggaaggagaagaaaaccggcgacgaagaagataatgatatggtggatgagatgttggcgttttacagtgaagaaggaaaagtgaacgaaggcgaggatttgcaaaacgcaattagactcacccgaaataatatcaaagccattatcatggtaagtaatttgttgattaatataatatatttaaaaacaaaacaagataaattttcagaaaaccatagatcttctatatgatttctagaaaaggcataaaataattttatgttggtttaggtaattatccgcttccattttagtcgttattcaattcaatgagtatgaaagcatgatattcgatatgattttcaataaaagaattgtgttaggtgaagtggcaaaatatgacaatactgatcaaatcacactgggatatggatagtttaatactttaattaatttgactaattgattgagttacatatgattacttattcaacacactaatacattatgtttttttattattagttcaggaaaacatgaatctttgtttaatatatctttaactattaatctagtattggtataaacttatgattaaatcattatctatatctatatctataggatgtgatgtttggtggcactgaaactgttgcttctgctatcgaatgggctttgacggagctcatgcataccccagaatccctcaagcgtgcacaacaagaacttgttgatgttgttggccttgatcgtcgtgtagaagagtccgattttgagaagctaacctacttcagatgtgtcatcaaagaaactctccgtctccaccctccgatccctgtcctattgcaccaatcatctgaagccacggaagttgctggctaccacatacctaaagggacacgtgtcatggttaatgcattcgccattaatcgtgacaagaactcttggaaggatccacacacgttcaacccatcacgattcttacaagatggggcccccgactttaaaggaagcaactatgagttccttccttttggatctggacgtagatcatgtcctggaatgcaacttggattgtatgcaatggagatggcggtggctcaccttcttcattctttcacgtggcagttgcccgatggaatgaaaccaagcgagatcgacatgagtgatgtgtttggactcactgcaccaaaagcgatccgacttgttgctgtgccaactcctcgtctgttgtgcccgctgtattgatcagaaagggccggtttgtgattgtgagaatttgttaatgttgcattgaaacaaattgtttttctttttcttcttatttcggtcattatccataaagatagatataaacaagatcaataaccgtaaagacttttattgtcatgtacatgttgaaagtaaaatttcttacgtttcttgtgttcatattatgttttcttcttccgagaattttacgccagctttgtgttgaattgctgaaaaagacattaagttcgactatattaatactcaagttgaaattgaaattagttgcccttttgtcaaattaaaaatgtcaaatatttataggttcttcaactacaactgatatgaaatgatacccgatgagtgacggggcttcttccaagtgggaccttcgcatcctccaatttcagatatatgtctattgttcacaataatgatatattaaataaagaaatattacaaaaataaccacaaatatatattatgtctccgtatcactcgcatatagataaccacatattacatcaatgatatataaaaacaaaatgctatatctttattacaaaaactaggttagaaacccgtgtgttacacggttgacaattcataaaacaaacgtaaacaaaattattttgtaaggcacttatatgccaaccacttgaacatttattacaacatccgacagtctccccgttaactgccacacaaacacaacttgtttttggcaatgcactttactcctttaacccgtaatacactatttttacccataatagcaaggtacttacacatcttttccgaaattgactatatataccctaaaacttgtttaattgttacaagttcaagttacttattgttagtattagtgaagaaatgtaaatacattgctatatggttaaaaaaaagtgcaaagagatggtatactaaaaagtcaaaatgtcgcctaacagctcaccctttacgttaagggtgatagtctttcttacaatcttaaatgtaattttcgtgacgagggacgtgtcaaccgatgctaaaaaaatttgatgatgctttattggtatgcattctttccatttctataatattaattatttaatgtttttatatgttttcttaaaatggtccaaagttgtttaaaactacattttctttactcacataatccgtctatttaacattgtagcatctgtccgacaaccaaaatcatatattaatgagagtaaaataactaatagctaaaatgttccaagtattgaaaaattaaagataaaacgtgataataggatagactatttggagtaaaattcccgataaaaaaattatgggtaaactttgtcctagggttaactgatttttttttcagttttgttcccaaaaaggaatcagatatttctaaaatggcctttagctattacaaacccaaatgacagttctacaacaaataaaaccaaaatgacaaaaaaattacgaaaaggaaaaattttctgatatgatacttaaatattgtttttatttaacctgattttagtggctgctttgaagaaactaataatttattttgggtctgagttcaaagctaaacttgaccatctgacaccagcacttctgactgatgcatctttgtggtgaaactaaggcagaggaagagtaatcaaagccatcaagtattggtccatcatctttcttaacaccttaacctcttgggtacatgagaaagaattaatatacggataccctataacactacaaggattagtggttttagcggcgacataagtcgcccctaatagtccagaaagtcgcccctaaaggcattagcggcgacgagtcgcccctaaaagtgtcgctcctaatgcctc

SEQ ID NO: 63 - DNA, 5618 bp, Cichorium intybus
  misc_feature 1..5618: note = source = /organism="Cichorium intybus"/mol_type="unassigned DNA"
  source 1..5618: mol_type = unassigned DNA
gtattgtcaacatcaacaacctcatccgacttccaattagcaagtgaccgagtatgcttcacctttttgtgattaaacccctagtataaagaaaacacattcagaatacacattttcttataaaatataaccacaataatacctcttcacttaatccaatttccaaatgtgatcgataaggcttgaaactttccccacagacaactattggtacatcatcacatttcctgacgtcctgtatcatcataaggtcatcaagatgatcaaacttttcatattagaaaccacaaccattattatttaataacataacgtacattaaaagcctgtcttcaacactgtatctcttggttaccagaacatcgttaattgtattctcgtcgtcctacataatcagcaaatcatcaacatggttaaacttttcatattagtaaaccacaactactattattcaatcacattgcgtaccttgtcaagaggctctttagaactttgacttttgtcaaccacataatccaacagagaaactgtccttcattagaaagaaaatagaatataaacaaatattcgtattcatgtgacaacttcaatataaaaattagacacattgattacctcaatatcattgataactatcttgcatacatcctcatcggaccttcgcttgcagagtagaccccccatgtatgccatatcctccaactacgtaaactcataaaacaacatctgttatatctatgtattaacaggaacatcaacccagacaaaacgaaaaccatttagttccatgaaaatcgtcaacctcccatacgtgggtctaaactgaagccacgacctgaagattctccacaaaaagtgaaccctccaaataactgtcacttctaaccgtatatgatgatttaacacggttgagattgaaagttgggaagatcgaacggccataattattggagaaaaagtaggacttattaggaacattagagatataatttgacaatccctccgtaattttataactttctaacgttttgttttgttagctttataaattaagacgttcaaacaaaaaaaaaaaaaaaaaaaaagcactttaaagtaaagtatttctaccgcatgatattattttccaacatcacctttctttctttgtccaatattatattaaacaaaacaatgtttatgatataatcattctccgttctataaaaagaattagtcttctatatacttgatgtgattagtgtagtaatttatacgaagaactattttcacctgtatgaaacaaaaacaatgatggtttttgatggtggactaattaacataatatttaaatatagaaagttaataagcaacaaccttaactaagaaattatgtcaattaaatacatgaacagaaaattaattcgaagaccaattcacttggtgtagtaactagtaactcgtaacattatgctgacatttaatcttattatataaaataaaaaacaaaactctttatatgataatttagaccggcactcgtttgcaagacataagaattagtcaacatttcatgtgattctcacttataataaacaaagtaaaaaggagtaaattgcaaaactggtccttgaattttacttaggattaaaaattcagttaaaattttgaaattatcacgatgtcgtccataaatttgaattttgttgatatgctctctgggattttctcaacagtacacgtttattctagattttaaagtcatattatatgtattaggatttaggaaccatatttgcgacaaaatacaaacttaaagaaggatattataacaatttgtataattgcaatatttggtaaagcacatggacatatttgtaaactataatagtaatatctatttgaattccggccattcaaacggggtgtgcccgcttaacgtgtatataaacacccctttatttcactctttaggtagcaacattcaaccagttgccacttaactctccatttgaatcaatatggattctcttcaaatccctataccagtctacgctataatcgctgtcttaaccttcgtctttgtggcatggctccgccgtaaaccactcccgccaggcccaatggggtggccaatcatcggcaacatgttgatgatggaccaacttacccaccgtggcttagctagtttggcgaaaaaatatggtggcatcctccatctaaaaatgggtttcagccacaccattgccgtgtcatcgccggagattgcccggcaaatacttcaagaaaaagataacatctttgccaaccggccggccaccatcgccatcacgtatcttacctacaaccgcgtagatatggcgtttgcaaactacggaccattctggcggcagatgcggaagctttgtgtcatgaagctgttcagccgtaaacgagcggagtcatgggactccgtcagggatgaggtggataccatggtgaaggccacagccactaactccggtttgccggtaaacttgggtgaactcgtatttgggttgacccatgatatcatctacagggcggccttcgggtcgatttcccacgaaggtaaagaagaattcatcagaatcttgcaagaatacaccaaactctttggcgcattcaacttggccgactttattccaatgctcgggttcattgacccggctgggttgaatacacgtttaccggcggctagggcggcgttggacggattcatcgacaaaatcatcgacgagcatttgagtaaagaaaagaaaaccggcgatgaaaatgtagataacgacatggttgacgagatgttggcgttttacagcgaggacggaagaatcaacgaaggcggtgatttgcaaaacgctattaacttgacacgagataacatcaaagccataatcatggtacgtaacttatgtaactcgaataaactaataactatagaactttctaatatttaaaactattgttctaaagaagaactatacgtaaattttgcaggatgtaatgtttggtggcactgagacagtggcttccgccatagaatggaccatgacggagctaatgcatacaccggaagcactaaagcgcgttcaacaagagttgactaacgttgtcggtcttgaccgccgtgtcgaagagtccgacttcgagaagctaacctacttcaaatgcgtcatcaaagaaaccctccggatgcaccctccgatcccagttctcctccaccaatcgtcggaggcgacagaagtttccggctatcacatccctaaagggacacgtgtcatggtcaacgcgtacgccatcaatcgtgacaaaaactcttgggaagatcctgatactttcaacccatctcgttttttgcaaaacggagctcctgattttaggggaagcaactacgaatttctgccgtttggttctggtcggaggtcgtgtccgggaatgcaacttgggttgtatgcgatggagatggcggtggcccaccttcttcattgtttcacgtgggaactacccgacggaatgaaaccgagtgaaatcgacatgggtgatgtgttcggactcaccgcaccaaaagcaataaggctagtggcagtgccaactccgcgtttattatgtccattgtattgaaataattgtatgtaagttttttagagtttcattttataaaagatatatataaataataacatggaaatatttttcttattgatcgttgctaatatattcctaaatcaacaaacctagttggaacttgaagtgtttacgggatctttgcttcccagattgtgacgaattgtaaaagtccatatatcacaatggagaatatcgaaaggattattagcaacattgttgaaagaaacaaaaggtaatctctttgtttagctaatggacacatatgacaaacagagtcattgtcatctttattgctaatacaaagatcattgcgtattatcctaattttgtcattagagggatgaccaaatcttgaatgccattgatgaagagaaacagccacatttgcacacacagtggtggatgggatagcatgaaccttacccttgccaattattttcttggggaaaatctgtttgataacagcataatcatgatagaatttgactgttagtaactgtgattcggttaaacaggtgactgatatcagattcagttcaaattgtggaacaaatagtacatctctaagaaacaactcatccgtgagctgtatgtctcctttataatgaacctggatgagactatgatttggtagtcttactctagtgcctctaataaagtgaatatttgtgaaatgcctttgtcattgcaaatatgttatgtagcccccgaatcaagaatccatacatttgataagtaatgtccagaattaaaacttaggattcggtatttacctctagatgctcagggacattggtagtaaccatattagtggaagcaagttttcctgttaacaaggaaataagaccttcacactgttgctgagtaagaccagtaacacatcagtatgtacagaaggttgattcatttcttctttatcaataatagattgttgttgatcaacatcaacagagtttgcacgaggttgattagactgagttctgtatccaggagggaagccatgcaagcgataacacttgtcttgtgtgtgaccgatgcgattacagtgcgtatggagaggagaatcttttcaaaagtttttgaaagactggttagatctagttttcttgttcttgatgggttttagtgcaaacaatgagctaaaatcctaagtttacatgcaccttataaggatctatgtttttctcagattgaacctataactaagaccaaaaataaaaatacaaaagaataattttatacctttttacctttggaagctagcaccacaattctcgtgcctctagtggctgacacccagaatcaggcaagctagatgagcaagagagaggaaggaggagactcctttttggcgtgagaaataggggagtcaggggcctctatttatagtctctaggtgataagcactaaggagtataaatgtcttatttaagatcatgatatgataatgtttttatcagataaaaggatctgcttaccattgctataaaaacccttatgggtttcggaatccaccaggctccaagcctgttatttcgtccaacccattaggttaaccagattgactgttacccacctattgaacaattacaatttaaccactgtacttttaactaatccaattaatcttaaaaataattccaattaatttttgattaattattaattatatattattatttctaattaatatattattattattttatattaatatatcattatttctctctctttattggtcatctaatcatttgctagttatgagagcaacccaaatagactgtgtgtaacacccaaaaattttcgcaacatatttgaacttttaaaaacaacatttataagagaaactatgattttacaaaagataagtatcacaaaaccaaattatatcgaaataaacttaataaagtgcggaagcaaaataataataataatgtcccaaaatggaaattcttctaatagtggtatcccggtgctccaagtcacacctc

SEQ ID NO: 64 - DNA, 5614 bp, Cichorium intybus
  misc_feature 4395..5406: note = /note="n = a or t or c or g"/note="n = a or t or c or g"
  misc_feature 1..5614: note = source = /organism="Cichorium intybus"/mol_type="unassigned DNA"
  source 1..5614: mol_type = unassigned DNA
atggtccggtgttatcccacttattttatgctgatgatgcaatttttgtaggtgaatggtctttgtcaaacatgaagaatctgtcccgcatccttaagtgttttcatgctgcatcaggacttaaggtcaattttagtaaatccaaagtattcggcattagtgtgaacaatgatctagtctcgaggtgtgctcgcgtccttggttgcgaggcgggctgttttccgttcaagtttcttggagttcctgtgggggctaatatgaaccgaataaaaaattgggacgtaatcgtggacaagttcaatgccaaattatcattgtggaaagcaaaaaacctatcattcggcgggaggatcacactaattaaggcagtactcagcagcttacctgtttattatttttccatttttaaagtgcctgcaggtatacttaagaagcttgagcggatacgaagaagctttttgtggggtggcgggggtcgaaatcatggaataccgtgggtggcttggaacacagtcatctgtgacaagagttatggcggccttggagtcggatcgctacgtagcctcaacattggactcatgttaaagcggtggtggcgtctcaaatgcaaaccggaaacattatggtgcagaataattagtagtatacacaacttggtgaggaaaccggctcatcatcttgcaaagaggtcggctacaggtgcttggcttaatatcgtcaacatccatggtgacctagaggagtttggattcagttttgacagtttattcaggaagacagttggttccggcaacaatactttattttggcatgataaatggctgaatgatacacctttgaaagattcatatccagccctttatgatttagacaaacggaaaatgtgttatgtttcagatagaatcacgatgcaagggaggcagtgggcttggagatctaatccatgcgctgctgatgagattgaccagatgtacaatttggaccaggctttaaacagttattatccgtccaatcgagaagatacctggaggtgtgtgcttaacagtacagggtcgttcacggtgggggcaattagggaaattatcgataattatggtgttacaagatcaagtataaatgttcaatggctaaaaaaataccattaaaagtaacatgttttgtatggagagcccttttagggaaaatcccgtcagcggaggctttgattaggaggggagtacggcttgattcctcgatttgttcattatgtaaatcaaatgttgaatgctacaatcatattctagttgggtgcagctttgctaaggaagtgtgggggaagatactgcagtggtgtggggacccaatctcgataggagaatcggtggagggactgttacagaacgtggcttattggggaaggtgtccgaagaaaaggagactcttgacggttgtatcttacggtacgttgtggtgtctttggaagtcaatgaatgatcagatctttaaaggtgtatcttgtaattctgacacaatttgtgatcaaattaaagcgttgctgtatacatggataaagtttagaggtggtaagttagacattaatcaggaggggtggaatttatctccattgaatactttgtaatttgactttgtatcggctgtccggtcttgcatctcgctgatcggttttgccttttattttatttataaattgtgttgccggttcaaaaaaaaactgtatgcatttaatgttgcattaaatgcatagattagatattatatgtgttaggaaacatatttgcaacaaaatacaaacttaatgaatgatattataataatttctaaaattgtcatatttggtaaagcacatggacataatttgtaatttatatagtaatatctatttgaattccggccattcaaacggggtgtgcccgctcaacttgtatataaacaaccctttatttcactcttaaggtagcaacattcaaccagttgccacttaactctccatttgaatcaatatggattctcttcaaatccctataccagtctacgctataatcgctatcttaaccttcttctttctggcatggctccgccgtaaaccactcccgccgggcccaatggggtggccaatcatcggcaacatgttgatgatggaccaacttacccaccgtggcttagctagtttggcgaaaaaatatggtggcatcctccatctaaaaatgggtttcagtcacaccattgccgtatcatcgccggagatagctcggcaaatacttcaagaaaaagacaacatctttgccaaccggccggccaccatcgctataacctaccttacctacaaccgcgtagacatggcgtttgcaaactacggacctttctggcggcagatgcggaagctttgtgtcatgaagctgtttagccgtaaacgagcggagtcatgggactccgtcagggatgaggtggataccatggtgaaggccaccgccattaactccggtttgccagtaaacttgggtgagctcgtatttgggttgacccatgatattatttatcgggctgctttcgggtcaatttcccatgaaggtaaagaagaattcatcagaatcctgcaagaatacaccaaactttttggcgcattcaacttggctgattttattccaatgctcggttttattgacccggctgggttgaatacacgtttaccggcggccagggcggcgttggatgggttcatcgacaaaatcatcgacgagcatttgagtaaagaaaagaaaaccggcgatgaaaatgttgataatgacatggttgacgagatgttggcgttttacagcgaggacggaagagtcaacgaaggtggagacttgcagaacgccattaacctgactcgagataacatcaaagccataatcatggtacgtaatgtaattcgaataaattaatttctatagatctttataatatttaaaactattattctaaacaaaagcaatacgtatattttgcaggatgtgatgttcggtggcactgagacggtagcttccgctatagaatggaccatgacggagctaatgcatacaccggaagcactaaagcgcgttcaacaagagttgactaatgttgtcggtcttgaccgccgtgtcgaagagtctgacttcgagaagctaacctacttcaaatgtgtcatcaaagaaaccctgcggatgcaccctccgatcccggttctcctccaccaatcatcggaggcgacagaagtttccggctaccacatccctaaagggacacgtgtcatggttaacgcgtacgccataaatcgtgacaaaaactcttgggaagatcccgacactttcaacccatcccgttttttgcaaaacggagctcctgattttaggggaagcaactatgaatttcttccgtttggttctggtcggaggtcgtgtccgggaatgcaacttgggttgtatgcgatggagatggcggtggcccaccttcttcattgtttcacgtgggaactacccgacggaatgaaaccgagtgaaatcgacatgggtgatgtgttcggactcaccgcaccaaaagcaataaggctagtggcagtgccaactccgcgtttattatgtccattgtattgaaataattgtatctaagttttttatagtttcattttatacaagatataaataataacatggaaatattttacttattgatctctgttaatatattcccaaatcaataaatctagttcaaactcaaagtgtttacgggattttttcttggcagattatgacgaattgtaaaagtccgtgtaatccaggtgtagtttagccatgaaaaactcaatggtcaatataaatggctaatttttacgaaatatctcgattcatgtacgtaattatcggtcgctattcttatactcttcaattttccaccaactttgaaacagacattaaataacaaaaaagggctatttgtaatggattggcgaaggaagtttagctacgaaaataacgacaatgtatttatgattagctagttagcgatggatgatattagcgacggttgcgacaaattatattggtgatggatgtttttcgaatgatgaccaccgtgttagcgaagaattttagggtctgtttggtaacatacaatcatgaaagaatgtaatagacaacgtaaaatggaatgaaatgtaatagaataaattattacatttaattgacatgttgattgactcgaataaatgaaatgagtcattcgaacatattatttgttattaatatataaaaaataaagtaaggtaaatttgataataaattaattcaagtaattttataaaataaattctattatatgaagatataacatactttatttcgttatatatatatatatatatatatatatatatannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntatatatatatatatatatatatatatatatagtaaattagacgaatggtcactgtggtttgggataatttgtgcgcttgatccttaactgtttttttaactcggacggtcccagagttgatgtgggatgctcgtgtcgttttacaaaccctagtcgatgcaaagcttcatggcgactttgatgatatgtcattttggttcctgagtt

SEQ ID NO: 65 - DNA, 5938 bp, Cichorium intybus
  misc_feature 1..1096: note = /note="n = a or t or c or g"
  misc_feature 1..5938: note = source = /organism="Cichorium intybus"/mol_type="unassigned DNA"
  source 1..5938: mol_type = unassigned DNA
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatatatatatatataaactagttatctacaggggcagaaattaaaattgtactgcagtaatagcaccatatattattttcctgtatgttaggggctttgtgagattgtaccattggaataagctaatttttttcaagatgtgtggtatattgttatttttgtagcagcacccgtagtataaaaaaaaatccataaattttgccaattttcgaagaaaatttgaccagtgacgaggccaccatgtgccctatagcaattcagcccctagttatctatgtaaggccattgggagtgggtatcacggagggaccgtgatggccacatcactaccccatcacggcgtgcacaccgccccgggtgctccgtggtgggcgtgatcgtgataggaagtgatggagatcacggctcatcacacccaatagggttttttccccctttttttcttttttttttaattctcactcaaccaattagaagcttggattttttatttttatggaattatttttatttgaaaaagatgatgtggtgtgatgggtataaagtgtgtgttgtgatggagtgtgatggagtgtgataggggtaaaatcctatgtggcgatgacgtggcgcccatcacatttgtgcaatgtgatggatactcccactagcttaaaaacggaaatacgaaaatagtcaactttccggcttcaagtatataattataatattatataataaaatgcaaactcgttcaaacagagctatgcgctgcatcgcctttcaagttcaactactgcgcaccctgtgtttttatatatatacactcgtgcgctctttctatacctggcaaccttctctcattgcacttcattacccatatactttaagatactcactgaacataaattctttcccggatactatggatcccatgtccatcttactttacgttgtactccctctgtttaccttcttccttctctcccgtttacgccggaaacctcttcctccaggtccaagagggtggccagtgatcgggaatatgttaatgatggaccaactcacccaccgtggcctagctcggttaggtgaaaaatacggtggtcttcttcacctcaaaatgggtttcagccacacggtcgctgtctcgtcgccggaaatagccaggcaagtactccaagtgcaagacaacatcttcgccaaccgtccggcaaccatagccatcagttatctgacctacgaccgccaagacatggcgttcgccaactacggtcccttctggcggcagatgcgtaagctctgcgtcatgaagctgttcagccgaaaacgagcggagtcatgggactccgtcagagacgaagttatctccatgatcaaaatcaccgctgcaagttccggttccgctgtcaacctcggcgagctggtgtttgggttaacccatgacatcatttaccgggcagctttcggttctatatctcatgaaggaaaagaagaattcataagaattctacaagaatacacaaagctttttggtgccttcaacttagccgattttatcccatggctaggatttatcgaccctgctggactgaacacccgattacccaaggcccgagcagcgttagatgggttcatcgataaaatcattgatgagcacctgagcaaggagaagaaaaccggcgacgaagaagataatgatatggtggatgagatgttggcgttttacagtgaagaaggaaaagtgaacgaaggcgaggatttgcaaaacgcaattagactcacacgaaataatatcaaagcaattatcatggtaagtaatttgttgtttaatataatatatttaaaaactaaacaagatagattttcagaaaaacatagatcttcaataacatttctagaaaaggcataaaataattttctattggtttaggtaattatcctcttccatttaagtcgttatccaattcaatgatcagtatgaaaacatgaaattcgatatgattttcaacaaaagaattgtgttaggtgaaatggcaaaacatgacaatattgatcaagtcacactgggatatggatagcttaatactttaatttgactaattgattgattacatatgattactttcaacacactaatacattatgattttttattattagttcaggaaaacatgaatctttgtttaatatatctttaactattaatctagtattgctataaaatcattatctataggatgtgatgtttggtggcacggaaactgttgcttctgctatcgaatgggctttgacggagctcatgcataccccagaatccctcaagcgtgcacaacaagaacttgctgatgttgttggccttgatcgtcgtgtagaagagtccgattttgagaagctaacttacttcagatgtgtcatcaaagaaactctccgtctacaccctccgatccctgtcctattgcaccaatcatctgaagccacggaagttgctggctaccacatacctaaagggacacgtgtcatggttaatgcattcgccattaatcgtgacaagaactcttggaaggatccacacatgttcaacccatcacgattcttacaagatggggcccccgactttaaaggaagcaactatgagttccttccttttggatctggacgtagatcatgtcctggaatgcaacttggattgtatgcaatggagatggcggtggctcaccttcttcattctttcacgtggcagttgcccgatggaatgaaaccaagcgagatcgacatgagtgatgtgtttggactcactgcaccaaaagcgatccgacttgttgctgtgccaactcctcgtctgttgtgcccgctgtattgatcagaaagggccgggtttgagattgtgagaatttgttaatgttgcattgaaacaaattgttctttttcttctttcggtcatttcccataaagatagatataaacaagatcaataaccgtaaagacttttattgtcatgtacatgttgaaagtaaaatttcttgtgtttcttgtgttcatattatgttttcttgttcagagaattttacgccagctttgggttgaattgctgaaaaagacattaagttcgactctattaatattcaagttgaaattagttgtccttttgtcaagttaaaaatgtcaaatatttgtaggttgctcaactacaactgatatgaaatgatacccgatgaatgacggggcttcttccaagtgggaccttggcgtcctacaatttccgatatatctattgttcacaataatgatatattaaataaagatatattacaaaaataacaacaaatatatattatgtctccctttcacttgcatatagataaccatgatatataaaaacaaaatgttatatttttattacaaaaataacaagaaatatatattatgttcttattataaatataacatttaccaaatgatattcattgtttacaaggtcagatcccatatatagtatttatattggggattgaaagaaactcacgcttcccatttcttgtatgtctttttggaaccatatctaattcctttgctctctttcccaccatcatcactcactttacgttatctgtcatggttctccttcaccaagccctggatttcaggtttcttggtccaaccataatttcatgattatcttggtctaaaattcgctctgctcctaccactctctaaccctaaagtcaagcatccacaccactactcaactacaccgtcaccatcatcatccattcattgcctttaaccacctaccgccaggcgccagtggtgccaatatttgaaaaagatggtgcttctgcccttcatcttctcctggtctttgccactcaaatctttatctattccatgcaaacaaaaaatctgatcgatcggggctttcgttttttaaatcgtgtttggttatttagattaggtttttgtggggtttgcacaatatatcgcagaacaaagagaaaggcgtggtgtatggacctctgactttctgtgaatgcattccttcaaactttgaccaagcttttcatatttagctatcatgcagtcttcctagaccatgcttgtttgctgctgcatttcttcaaaaatatacaactcaagcttagaattttttgtggttgttcatatttgtttttcaacaattacaaccacaaatttcctttgattcacacatgaagattgccctataaaagggagtagaaatcagcacatcaaaccacaccaacaaaacaaaaatctcacatcttctcttgtaaatacataagagtttttaaactcttagtatttttattcttcaaacattttgtaaacaccacaaaatatattgaattgtttctcggcttccttcgcccgtggtttttcccgatttgggttttccatgtaaatcctcgtgtctctattttatttactttatttgtatttttgttgtgattataaccgtttgataattttgatcccattccgcattgagctaatattgtctaaagtcaattcggatccgtcgtattttacaacaattggtatcatagcgtccgggttttgatattaacttgttgggatcattttatcaagtaataaatcatgtcttcggtgaaatacgatattccgctgctggatcggaatacccaatttccgctgtggcaagtaaagatgagagatattctcattcagatggatttacacaaggcgctcttgggcttcgaaaggatgcccaatacatggaccgaagaagaaaatgagatgaaagatctgaaggctttatctcaaattcgtttgcacttgtcaaacgatgttttacaagatgttctaaaagaaacatcggcggct

SEQ ID NO: 66-67 - skipped (2 entries)

SEQ ID NO: 68 - DNA, 4690 bp, Apium graveolens
  misc_feature 1..4690: note = source = /organism="Apium graveolens"/mol_type="unassigned DNA"
  source 1..4690: mol_type = unassigned DNA
gagaatgatttaaggttgggttgctgtgattgttgcttgtggtgggtctgctggctgtgtgtttgtgtttgtgtttgcaggctgcggctgtgtgtttatgtatataggtttgagagagactgtgtttgtcaagaattagttaaacaaaaaacgctaccaaccgctgaaaatttccgcggtccgtgcggagggtattttggtaattaccacggaccgctgaaaatttccgtggtccctttgctgctgttttaattaccaaccgttagatccaccatccaacggtggagaaactgtttgttgtataccggtctacaacaaatgtttgttgtagaccggcccccatatatataaagtcggtatagtggagagaagtagtggatgtagttattttaaaaattataaaaactttactatttttggaaatttttgaaatgtaaataattggaaggggtatgaaaaaaaagtgtaaacaaatgagaggtaagaagggtagcaagtcaaaatttaaaattattttaataaacatactaagaacttcctaaacactactgaactcgaaaaatatccggtccgagtgaccagtgtacttgaattaggtaagttgttggcaatgttttattactctaaacatgattatgattgatccaagttggccgtgatgcaaaattagacgtgtcagccccgctaccatacttagtcatggatgacaccaaaatgtattacaatttaaagtccgttgcaaacaagttagctagactagacttgttttgttctgatttccaggccggctcttgtcaatttgtgccccaagaggcaatagcatcttcatcatacaaattactatttttacattacactttttcgagatgtttaaaaatttggaggccctagaccatcaccttactcgccgccctgctgattccatagtagtatctgttgcttgcctgattattaaaagatttaaaataaaaagttgttataataaattgtatatttaaggaatctgtaggctatcgcatttgtagattcattaaaattcagtaggaacatcaaacctgtctctttctttcttaatccacttacagacctctaatcccatgtcacttctcttcattttgctcctaacaattatctcaatgatatttttcatatcacgttttgggggtaaaccttatccaccagggccgagacgcttgccagtgattggcaatacgctgatgatggcctgtctaactcaccgtgggctcgccaaacttgcgagccgttatggtggtctattttacctccgcatgggtgcccaagacattgtagtcgtgtcgacgcctgatatggcacgtcaaattcttcaaactcatcaagaccaaatctccaaccgccctactagtattgctctggactacctttcatatggcagggccaacatggcctttgctgactacagcccttggtggcgtcaaatgcgcaaaatttgtgtcatgaagctctttagccgtgcacgggttgagtcctggaactctgtccgtgacgagttaaaccatatgctccgagatgttgcatcaaatgctaatcaagctattaatctaggcgaattagtttttgggtttacggagaaaatcatctatcgggctgcgttcggatcaaggttgagcgacgggggcactgaatttataaaaataatgcaggaattttccaaattatttggttctttcaatgtatgtgatttcgttccatggatgagtagggctgatcctcaagggcttaaagctagacttcgcaaggctcgtggatcactagatacgttcatcgattccattatagaccaacacataataaaaaggaagggaaaaaatataggtgacaaggacatggtggacgagttactggctttttacactgaggaaggagaagctaaagctgaatctgatgatttacaggccactataaaactcacaaaaaataatatcaagggcattattatggtaagtggactttcattatgccattgttagaaattacagtgtactgacaaaccagtctatatcataataatttgttaatttcaggatataatgtttggggggacagagactgtagctgccgctatagagtgggctatgtcagagctattgaaaaacccggaggagctcagaaagactcaggaagagctttccaacgtagtaggacttcatcgctgtgtcgaagagggagacttggagaaactcacttaccttaagtgtgtactcaaagagaccctacggctccatcctcccctcccgtttctaccccgtgctgcagccgaggatgtatatgttgctggctactatattccagcgggatccagggtcataattaacctttgggcaatggggcacgacgggaagtgttggaatgatgagccagaagcatttaagccctctaggtttcttgatgtgggagcgccagactacagaaacaacaactttgagttcataccgtttgggacaggtcgcagatcatgtccaggcatgcagctgggccttcatgcatttgaaatgggcttagcacaccttcttcactgttttaactgggaattgcctgatggtatgaagccgagccaagttgacatgagtgacatgtatggactctcagctccaaaggcaacccggctcattgctgtgccgactccgcgcctactgtgtccgatctgttaacaaacttgcaaaataatgtacttgaattatatatctgaataataacagaatcagtttcattaagcgatatgattaagacattcattgagaatcctgttaataaatgaacctctaggtagctgcaaaataatgagaattcctgttatgcctgtttctaacacctgtaacgagatggaccgaatgaatgctaccaagttaaaaagctcatgctatttcggagtttggtgacttctgcgttctaattatgcagaagcattaggatttaggggattctcgaaaggagcgagcagaatatccttagtcaaattgtacttcaattttaagatcatgtaaagcatgctatattagtaagctactcttcataataaaaactgcatggtgcttgtaagattctatgacaaatgaaactggatgatgcaaatgatacaaacacatactaataaaatcataaggtaaatctggtacataaatgaaataagataaagagaaaggtaggtgtaacaagatcaacagcaatgataaaaatgaatgggcaagttgccggtttcttcaatttatgtatagggaaggaacccaaccagtgggtgcagctaggattataattcgggtcgagccgggttagcttataaacgagtcaagttcaggtcagcattgcacgacagctgaggcaaacatcgttcacttcatattaactactctttccctatactggctctattgatgttgtcaatgagcatgtggaaaaaaaagttactctcatataaagctttaaaacaatatcgcccaccgtggggctcgaacccacgaccacaaggttaagagccttgcgctctaccaactgagctagacgggcttgtgctttaatttaccaaacgtaatttataaaggtaatttcttagaaagttttgttttctagtaaagtttaatttttattatattttgacaccgtgactcttctggtacaaagaaaacacatgagctaatactgaaactaatgcaataaaagttgctaagggaaaatagtaccctcttgcttttttcagctgaaatttattttggtataaagtgagcaaatagggataaatataacagtttgcaaggaagatcacaaataaatacaaaaaaaaacaaaaacaaagttgcacccagcaaagttcatgctactccattgccttgttcttctcttttatcttgtcacgcagagatacttattactttgggggtcggcggtatgggtagaggatgtcatcgacacttgcttttacttgattgcctactttccttacatttttcgcaacccctgctgctaccaggcctgcatttcttgtaaacctgaaatgtttgttcaaaattttttaatcgtaagctgaatgtattacatttggataagaactgaaatagaggagaaatttaagaactaaagaacagcacttcagcagtgattgggttttacctttttaagaactcgtcccctacagacactgcaggtttatgtgtctgaggctgtggcgatctacgattatctatataaaacaacagttcgacaacaactaattagaaataaatacaaataacaatgctaaaaacgtacaccaccaacaacaaacagaactatttggagaagattcaatccacaggaacaatgaccatattatatagatacaacagttcgacaactaataattagaaacaaatacatataacaatgctaaaaatataaaccaccaacaaggaacaaaactataaggcgaagattcaatctacaggaacagttacaatattaactatgtccgtattctatattaagtcttcacatcccaaatttcaaacacctaatctttgaaaatttgtttatttacaaatttcatattcctttaggtaatattttatgaaacacaccaccaagaaacaacagcggcgccattttctttccatcagatgtatccccactttgatcacagctactagtcgaaaccacaatatagaatttacccgtatac

SEQ ID NO: 69 - DNA, 4155 bp, Apium graveolens
  misc_feature 1..4155: note = source = /organism="Apium graveolens"/mol_type="unassigned DNA"
  source 1..4155: mol_type = unassigned DNA
ttaataattattagttatgtgtttggagattctcttgctaatttataactaccataaacaaacaaatcatcatcttctcaatcagacaaaccaaaggtatgatgaccagcatggtaatacaaatcctcgtaatttgaactaatccgcatagataaagattagtaaaatattttttaaaaattaatggaatattgtgaacactatggactgcgaatttggaagggaatacgtaataagcaacatgcgttccaaattcacagaatgaaaaatgagtatataaagaggtacacaccacaagaaactgtagcggtagcaacaaatacaaaattaaatatggaacacgatcatattacattggttcaaagtatactacgatctcctactgccaaaatgacaaccatcttcttctttcttctattactgtgtttgttccttgcccgtcgtaaaccataccctccgggaccgaaagggtggcctatcatcggcaacatgttaatcatggacaagttaactcatcgcgggctcgcgaaaatcgcaactcagtacggtggtattgtccatcttcgtatgggcttcctccacatggtcaccgtatccactcccgatatggtccgagaagtacttcaaattcaagacaccgtttttgctaatcgtcctgcaacgatgaatattagctatcttacttataatcgtgctgatatggcattcgcaaattatgggcctttttggcgtcaaatgcgaaaaatatctattatcaagttatttagtcgtaaacgggcagagtcatgggactcggtccgtgaccaggttgatgacatgctcggaaaggtggtgtctaattcaggcttgtctgttaatattggggaactggtgtttgggctcactaggaacatcatttaccgggcggctttcggatcaatatcaggccagggtcaagatgaattcatcaagatcatgcaggagttttcgaaattgtttggtgcttttaatatatgtgattttgttcctgggataacctggcttgatccacaagggtttaaggttcggcttgtcaaggctcgagagtcgcttgataagtttatagactcgattctagacgaacacatagctaaccagaagagcaatctaaacggttcaactgacgagggcaatggtgatatggtgtaccaattattggctttttacagtgaagagcaatctaaagtcaaccattctgatgatacgaacaacgccctaaagctcacaagagataatatcaaagccattatcatggtcagtgcaaccttgacgcatctattagcatgcatatctattttcatatatatgttaatatgttaaaagatgcacacatacaaagtctgctcaagtaccagttttcctttataacatgatgtaaaggatagagctgaaaagagtatatttctagagtaatgtgtagcaattgcgttcactattggttatgcacatatccaaacatggtccctctagacaacaattagatgttttcctcatggtttaattacatgttcaggatgtaatgtttggagggactgagacagttgcgtctgcaattgagtgggcaatgtccgagctcatgggaagccctgaagacctaaaaagggttcaacaagagctcatagatgttgttgggctacatcgtcgtgtagaagaaaatgattttgacaagcttatttacttgaaatgttgtatcaaagagactctcaggctccacccgcccattccattactccttcatgagacggcccaggaagcggtggttgctggatatcacattccagcaaagtcacgggtcatgatcaactcatgggcagttaaccgagacccaaactcgtgggatgacccagaaaagtttaagccatcaagatttttgaaacaaggtatgcctgactttaaggggagcaactttgagttcataccatttgggtcaggtcgaaggtcctgccccgggatgcaactaggactatatgcatttgagatggctgtggctcatcttctgcatagtttcaactgggagttgcctgatggtatgaaaccaagtgaattggatactaatgatgtgtttggactcactgctcctagggccactcggctcgttgctgtaccaactccacgcttgttgtgttccatctgttgatacatatctatctatcgagagagctgagagaaattctataaatatagttccaacaaaatatgtgttaaatttcctgacaaaataaaaagaaatgattgttaaatttggaaatattgttttaaaatgtgtaatgttggctagccataagtgatttttaatgaaattttacatgttcaatctctggttttagctatagcggaaattcatgtatatgtgcgattctcttcttttatagtggagtcgtgtaataattattacataaacttttattatgatatatttctccaactctgtatgttatgctttctgaatgtatgtgagttactataaagtttgagtaagacaatgcttaaatcttttcgtcaatggtattgattacctcccttgtgaacaaaattatatggctcattcaaaccttcatctcattccagttctcactcggggcggtcagaatcactgagaattaatcaacctcacaacatcatgcaagttcagcaagttgcaggattaggaagtaatgcttctaattattaggtaatttcagctgtagtaagggtagcttggtaccttcataaacgtataatgttggctagctacaagtggttttgaataacaagttatatgtgaattatctggattcagatatagctttatgaatggtatgaacttccctgtatttgccattctcttttttctggcggtaataagctatatctgttttgctttctgactgtttgtgagtgggtaataactctcaaatccgtttccaaaacttgtcatgaatgacgcgtgacatttgtaacattttaatttgtgggcgcacatggagtccatctcttgttattaacaaaaaagttaccaagtatttgcataaactgttgtatcttaaaattgattgaagtttggcgctaccaagacccttgctttgaccattcctgtaaatttttatgtaacactttggagtgaaaaaaaaatgaaataaatgcccggagaaacaaatatttgatcctcacaagcttcttcctggtaatatattggttaacaaacggtgagaaccttttcctaagcatagttcattacctctcggattgacaactaacattttcgctgatttcggtctcttaacgttgatgcagacacactaaccttctgaatacttagtaataacaccagatgaaggataaaaattagatattagcaactactcagaagagagataagagttaacatacacaaaagtttcttggtgcatgctttcacgatcaaactcgttggtgaggtccaaagcttttcggcccgattcactaattaatcacaagctctctagaatctagaaactcccattgcctactactacaaagttataacaagcaatagtcaacagacatgacaagaaatttcttgattgaaatttcaagtttgaaccttcaacaaactatacaaagagatagtctgcacgccatgtaacttactggatctcttgacacttctaagcgtcacaaactcaagatttttattttaatattgctttcttgaattttcttcttaacatgctgggacatgctcagtttcaaccttcaacaagcttatttatagctttcaacgtctaacaagtaacattatgaatataatatcatatcaattccataataacctcatgtgtatttatcatattacagggctgacacaacaaaaaaatttcgacaaggttaatgtcatattcccacagtttaccagttaacctctacagaggtgctttgccgatgatactgaaaaggagaagaagcttaaatgtgccttaaacttgtaaaagccttcttctccttttcagtatcatctgcaaagaatctctatcgaggttaactggtgattcacattgaaaattttatctacagatgtgaccgatgatgaaatctgcatatgtgttcgataaaatcatcaaattctctatgagtatcacctccttccgccactgatcttttgacagcagtccccaactc

SEQ ID NO: 70 - DNA, 1536 bp, Apium graveolens
  misc_feature 1..1536: note = source = /organism="Apium graveolens"/mol_type="unassigned DNA"
  source 1..1536: mol_type = unassigned DNA
atggaaaccaacactaccgccatgacaattctcttcttcattcttccgctgcttagtttctttctcttgtccagctttcgacgtaaacgttaccctccaggcccgaaaggttggcccatcatcggcaacttgttgatgatggacaagctatctcatcgtggactggctaaacttgctgctcaatatggcggccttgtccacctccgtatgggttttcttcacatgttcaccgtttcgactcccgatatggcccgagaagttcttcaaattcaagacaacatttttgccaaccgtcctgctaccatgaatattagctacttaacttatgaccgagcggatatggctttcgcaaattacgggccgttttggcgccaaatgcggaaaatatcggtcatgaagttatttagccgtaaaagggcagagtcgtgggactctgtccgtgaagaggttgatgacatggtgaaaatcgtgttgtcgaagacggggtgttcggttaatattggagagcttgtgttcgggttaactaggaacattatttatcgggcagctttcgggacgttgtcgcacgaaggccaagacgagtttatcaagatattgcaggagttttcgaaactgtttggtgcattcaatatctgtgattttgttccgggattaacttgggcagatccgcaagggttcatgggtcgggtggttaaagctagagcatcgcttgatggattcatagactcaataatagatgcacacattgaaaaaaagaagagcagtaaaaatggtattatcgacgagggaaacagtgatatggtgtatgaattgctggatttttacggtgaagaaaaggctaaagtcagcgagtttgaagatcagaacagctccttgaagctcacaagagataacatcaaagccattatcatggatgtaatgtttggtgggacggagacggtagcatctgcaatagagtgggccatgtcagagctaatgaggagcccaaaagacctcaaaaaagtccaacaagaactcgtcaatgttgttgggcttcaccgtcgtgttgaagaaagtgatttcgacaagctcacttacctcaaatgctgcataaaagagactcttagactccaccctcccatcccactacttttacacgagacggcccaagatgcggaggttgctggatatcacattccggcaaggtctcgagtcatcataaactcatgggccatcaacagagacccaaactcgtggactgacccggacacattcaagccttctaggtttctacaagagggtatgcctgactttaaaggaagcaactttgagttcataccatttgggtcgggtcggaggtcttgtccaggcatgcaacttggactttatgcacttgagatagctgtggctcaccttctgcattgttttaactgggagttacctgatggtatgaagccaagtgaagttgatactgatgatgtgtttggtctcactgctccgagggcgactcgacttgtggctgtgccaactccacgcttgttgtgtcctatctcctga

SEQ ID NO: 71 - DNA, 6688 bp, Brassica oleracea
  misc_feature 1..6688: note = source = /organism="Brassica oleracea"/mol_type="unassigned DNA"
  source 1..6688: mol_type = unassigned DNA
acaatctggattcatgtgtgatttttttaaaatatagatccaatgaacctattcctagaaagtcatattacatttaatctataatcttatcatttaaattttggatctaccagaaacttttattggactatcaatagttagatttaaacagtggatgatctattggatttatagataatataattaaatagatataatttaatgttgtaatattatacctctatatgttaaatacttaaatatttgtctatgttattttttaaaattataaaaatcttttttttaaataataaaaatcatattatctaacaatgattaatctttactaccttaaaccaatgaaaacaaattttaaactatatagtttattttaaaaattaaacaaaaactaaatgtttaattatttactcgataatataaatctatgaagagaaaagtttaatttcttaataactttctaaatttgtgaaatgttacaatatctttgaatatgacaatacaataatattttactaatctttatatatatagttacgattttaataatgaaataataatccgaaaatatatatatataagaagattcaaatacatgtgaaagtttgaaacaatctattcaatgaaaaaaaaatactgtaaacttattatgttttaaaaattgatagacacatatatattataatatatatcaatttagaattgaaaacaaaatatttatataaaaataaatgaaaacaaaaacccgcgcggtttagcggatcgagatctaatcttatatattaaaacagaagtcaaaacattgattcatgtgcgattttttaaaaaatggacactcaatagaaatcaattatcattcatttattactaataatatggattaataatatatcattcctaatataacatcgactcctaaaaattcggattggttaacaaactcaccttgattcatgtgtgatattttaatttggatcatcatttaaattttttattaaatgtatttacttaatgttaatatataatcttttaactacttaaatcatgatataatttgatatcttttaatttaaaatataaatatatatatttattttttttaacaaaatgtgttgaaaaacattataacaatatcttaattatcaaaaattgtatataaatatttttactaattttgtaattagtaatgaaattaatgttattatacattaatatatatactcagttatcttttataaacattatatttaactatttttactattttatagttatatctaacattttaaaataaaatattgtatttaactaaatatgataaaattattttaaactgataaattaatatattttattttcacaaacattaaaaaatttatgctagaaatataatatgttggtagaacgggttaacattagtaaattagataattcatgtataaaattaacttatcttaaatttttatatatttatagttattatcttaaatgaataaacataaaaaatattgataaaagaaatctagcgctttgaattacggatcatgatcataataattgaataaaaataattttaaaatatatataataaaaaataccaaatatatgtgatgatttgaaataatcaactaacttacagcataaaaactatcaaatttttatatttaaaataattatatataaacatttaaatatatatgtaagtaacttaaaaaatatattctaattatgtaatatatatataaacatgaaaattaatacccgcatagttgtgcggatccaaatctagtaataattaaaaagaggtgtatgtatacatatatatgtgtcacgaaaatgaagttcccatataaaattagaaatatgcaataagtgggaccacactttgattataaaaccaacctttcgtaacacatcccaaaatggcgaactttgcctgatcaattcctgagaccttccacaaacaatagaaaaaacatttcccagaaaaaagaaaaactaatatggagtcttctatatcacaaacactaagccaagtattagatcccaccacgggtattctcatcgttgtctcacttttcatcttcatcggcctcatcacacggggacgaaggcctccgtacccacccggtccacgtggttggcccatcataggcaatatgtcaatgatggaccaactcactcaccgtggtttagccaacttagctaaaaagtacggtggcttgtgccatctccgcatgggatttctccacatgtacgccgtttcctcgccagacgtggctaggcaagtccttcaagtccaggacagcgtcttctcaaaccgaccagcaactatagctataagctatttgacttatgaccgagccgacatggcgtttgctcactacggaccgttttggagacagatgaggaaagtttgtgtcatgaaggtgtttagccgtaaacgagccgagtcatgggcttctgttcgtgatgaagtggacagaatgatccggtcggtttctagtaacgttggtaagttcgtcactctctatgtgcggttaaatattttaaaagtgaacaatcatttttgttagtttattatgcatttgttttctgtgatatatgtgtattttaatagtcaaccaaaaatatcgtaggtaagtctatcaacgttggtgagcaaatttttgcactgacccgaaacataacttaccgggcagcgttcgggtcagcatgtgaaaagggacaagacgagttcataagaattttacaagagttctctaagctttttggagccttcaacgtagcggatttcataccgtatttcgggtggatcgatcctcagggaataaacaagcggctcgtgaaggcccgtaatgacctagacggatttattgacgatatcatcgatgaacacataaagaagaaagagaatcaaaacagtattgatgctggagatgttgttgataccgatatggttgatgatcttcttgctttttacagtgaagaggcgaaattagtgagcgagacagcggatcttcagaactccatcaaacttacccgtgacaatatcaaagcaatcatcatggtaaatatttttcacgaggcactagtaacagtcattattcttaatgcgttacgtcataaaactcatctattaaaccggttgttttctctgcacgtatatattttctgtttttacaagtagtctaatataatattagagaacttgcgtatgcagtcgttttattgatagtagaaaaatacaaaggttttacgtatatcacacgtaaataaaatcaccgtattggaaaacatactataagaacatgattatagagaggttcttggggtgaagtttttagcggaatataagaccatgattatccctaagggtttcttaacaattttttttttacacatcttttatctaaataaaaaaaaaaattaaaaaacataccaatcgcgggtcgtcgcgtatcggtgggacccgcgaacagtccaccaaactcatcacaatcgatccttatcctgcatcttttgtgaccgttttttaaactttttgtgggccccagggctaagaaacacttacaaaactaccgatatttatgctctaagaatccgtctcttaacttttaactaaaaaaattataaaaaaattaagaactagttcttaaataagagttttaagagacggtttttatcgtttttgattaaaaattaatagatgagtttcttatatttcgctaagaactccaccctaagaatctccaggtaatcatgctctaacgtcatgtgaattgacttattttactttttcgataggacgttatgtttggaggaacggaaacggtagcgtcagcgatagagtgggcattgactgagttattacggagcccagaggatctaaaacgagtccaacaagaactcgctgaagttgtcggacttgaccgacgtgtggaagaatcagacatcgagaagttgacttttctgaaatgcacactcaaagaaaccctaaggctacacccaccgatcccactcctcctccacgaaaccgcagaggacactgagatcgacggttacttcgttcccaagaaatctcgcgttatgatcaacgcgtttgcgattggacgcgacaagaactcttgggttgatcccgaaacgtttagaccgtcgaggtttttggaaccgggcgtaccagatttcaaagggagtaacttcgagtttataccattcgggtcgggtcgtcggtcgtgcccgggtatgcagctcgggttatacgcgcttgaactagccgtggcccatatattacattgcttcacgtggaaattacctgatggcatgaaaccaagcgagcttgatatgagcgacgtgtttggtctgacggctcctaaagccacgcgtctctacgctgtcccgagcacgcgccttatttgttctgtttaagttatggttcgaagcacgtggcgggtgaaatgaaaggtggttggtatggttgatggttcttgaaagtggtgtgagaagtcaaacgaagccctgaaaatttgttgatgttatataatatatgtttatgtatttgttgtgtacacacgtgtgttctggatgaaacataaagtggctctttgtttcgtttttcatcttcttttgtgggaattttttccttgcatgaaatgtaaacgctgaaaaataagatttttttttacaactaatttgcatattacaaaagtatactaatgatccaaggattttgcaaacataagcatttccatcactatgagtccagaaaagagtttggcaaccggtgaactaagtgaaaactcatcaactttactatttgaggaacctaatgttagagaaatttggaggcgatagctttcaatttttagttttgaaaagccaattattttcgtgtagttttttaaaaagtcagatcacttgatagaaaaagttaaattctaaactcttcacgtattttcttatttaattttattttgttaaggtcgcgtcttctctattgaaaagttttatagtatatcttatatactcattcatatggaaaaacattgtagtctttattgaaaacgcacctatcatttatgtatcatgaatgtatgtgttgattacttaattttaacaccatcgacgatcagatttctagatggatggcaaaatccatgtttaaactactcacttaaaaaataatgtaacaaccagaatgcatcaagtactaaacgatccctacttcaactaccttgacgaaatcaataaattacaattacttagatttttcgtagctacacgtagcacattcattaacatatatgtagtactatacgtggactatcgaatatcacaaaataattgcaaacatatggttatcagtattaaatatgccccctgaaaaatattcaatcatatagaaaatctccgaaagataagaaaatgacaaatttatagacaagctacaagctgtgaattatgaagcagtcatcgctaatattagctcaaagccaaacctaacatttaaggcttgaatatctaagccatagctttgtaaagaagtttgttcgagtagggtttgtcattgtcttctagataagacacacattttaaattaaaataaataatatattataagtagaattattataaagtataatattatttacctcattaaaacctttttactgctgacaaattttttttttttactactattattaatcataataacgcaatgtaaataaaattaattttaaatgtatagaacaagaagtttatataatatattataagaatattattataaactataattttattcgactcataaaattatatttataatttatataattttttaaaaaattactaatattattaatcataacaacacactgtaaataaaattaattttaaatgtatagaaaaataagtttatataatattatgtcaatttatactacataatttttacttatcatatcataaagaactaaattctaaagtgttaatgaaaatgaatagattgtttactattttaaaattaactaatgttttatttactagcaattcataatatgaattttaaagacaatggatatatctatacgttgtttggaaaaagtaaacttaaagatgaaaatataatggaaagaaataaggaaagataaaagccagttcaaacaaaaaaaattaagaaaatatagataaaaaaaataagtatggaaaaaggtaaaaataataaaactaaagcttgtaaattgccacacagaacttaaattaaattcacataaaataatttgtgagtagatcgaactctcaacactggagccagacgggagcacttttaccaacgaagctacaaaaaattactagaacaactctaactgacaaaaattaaaaagtagtcggaacacgtgcccccgtagcttacactgtagatccgctcctggcagtaccaccaaaaattgagaaggcgcttaatttttgacttttaaagcatttccaacccaactccaatttttactctaaaatgaaataaaagtagatataaaacaataattggcggtgcctccttgacttcacattatttcgtggtggaaggataagaaaatgacaaatttatagacaagctacaagctgtgaattatgaagcagtcatcgctaatattagctccaagccaaacctaacatttaaggcttgaat

SEQ ID NO: 72 - DNA, 6254 bp, Brassica oleracea
  misc_feature 1..1557: note = /note="n = a or t or c or g"
  misc_feature 1..6254: note = source = /organism="Brassica oleracea"/mol_type="unassigned DNA"
  source 1..6254: mol_type = unassigned DNA
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaagggtttagggtttagtgattaggatttagggtttagtgttattaaaatttagtttttaatgtatgatttagggtttaagatattccaacggtttagagtttatccaaagtttaaggtttaacgtttagggtttaggatttagggtatagggtttagtattttgctgaagacttaacaatattaattaatttatttttttgtaactatttttatgtatttttattattttattttaaaaatataatctaatttggatatttaattttatttctttttttaaaaaatatcaaatatcaaatattcaaacactattggttggttcagaggtgaacccaaaaatttgaccacaacttgattataaaacccacctctctaatcacatcccaaaatggcgaactttgcctgatagacctcttacagaaaaagaaaaagaaaaatcatggagtcttctgtatcacaaacactaagtcaagtattagatcccacaacggctattctcatcgtcgtctcacttttcatattcatcggcctcatcacacgacggcgaaggtcttacccacccggtccacgtggttggcccatcataggaaatatgttaatgatggaccaactcacccaccgtggtttagccaacttagctaaaaaatatggcggcttgtgccatctccgcatgggcttcctccatatgtatgccgtctcatcacctgatgtggctcgacaagtcctccaagtccaagacagcatcttctcgaaccggccggcaacgatagctataagctatttgacttatgaccgagccgacatggcgttcgctcactacggaccgttttggagacagatgaggaaagtgtgtgtcatgaaggtgtttagccgtaaacgtgcggagtcatgggcttctgttcgagatgaagtggacaaaatgatccggtcggtatctagtaacgttggtaagttagctatgtgcggtaaaatatgtaaaaagtaaaaacaacaaacaatgtttctctcttttatacgtattttaacagccataccaaaaaaaatgtaggtaagtctataaatgtcggggagcaaattttcgccctgacccgaaacataacttaccgggcagcgttcgggtcagcttgcgaaaagggacaagacgagttcataagaatcttacaagagttctctaaactttttggagccttcaacgtagcagatttcataccatatttcgggtggatcgatccacaagggataaacaagcggctcgtgaaggcccgtaatgatctagacggatttattgacgatatcatcgacgaacatatgaagaagaaagagaatcaaaacactgttgatgatggatatgttggtgataccgatatggttgatgatcttcttgctttttacagtgaagaggccaaattagtgagcgagacaacggatcttcagaattctatcaaacttacccgtgacaatatcaaagcaatcatcatggtaatttctttttcaaaagaccactaggcacattcattattcttaatgcgtcacaccatcgaacttatccatcaaaccactagattcatatttttgtctaaatgaggaaggtaactttctgtatttacaagtagtccatatgatattagagattttgcgtaagcattaggggaaaatgacgtcatttactgatggtaaaaactcataaaaatataaagttttagaaaagacctaaaatacgaaacatgtaaaaaacacgtacaaaggtaatataaatcacacgtaaatataatctgaccgtctttgagctaacaatttatttctgtttacattttaaaatctaatttgaattgatttattgaatttaataggacgtcatgttcggaggaacggaaacggtagcgtcggcaatagagtgggccttaacggagttattacggagccccgaggatctaaaacgggtccaacaagaactcgcagaagttgtcggacttgaccgacgtgtggaagaatcagacatcgagaagttgacttttctgaaatgcacactcaaagaaaccctaaggctacacccaccgatcccactcctcctccacgaaaccgcagaggacactgagatcgatggttacttcgttcccaagagatctcgcgttatgatcaacgcgttcgcgattggacgtgaccctaaatcttggcctgacgccgaaacgtttagaccgtcgaggtttttagaaccgggagtagctgattttaaaggaagtaacttcgagtttataccattcgggtcgggtcgtagatcgtgcccgggtatgcaactcgggttatacgcgcttgagttagccgttgctcatatattacactgcttcacatggaaattacctgatgggatgaaaccgagcgagcttgacatgaacgacgtgtttggtctcacggctcctaaagccactcgtcttttcgccgtgcctagcacacgcctgatttgtgctgtctaagttatggttcgtagcacgtggcgggtgtaaaaccaaacgaagggttggttcttggaaagagtttgaaaagtcaaatgtagcactgaacatttgtggatgttattatatgtatgtatgtgtatcacgtgtggtctggatgaaaacataaatggctctttgtttcgattttccttttcttttgttgggatttttccttgaatgaaatgtaactggtcaaatatagtttttttttgtttatctacattacctttgcatattacgtaggtaaactattaagagtattgcattcgttagaaattttataacctaccgacaaatttccatcaccatgagtctatgacttaaaagagtttggcaacctgtaaaaccaagttaaagctcgatttgtattttaaaccagaagttaacccgcgctacgcgcatagttattttgattttcaaattttaactatatagatattcatttggaccttgtattctgaacaacaacaaaattttgaattgtatgtttgagtgaatacatatttacttagaaaaaacaaaaaattatatatatttttcatatagatatttgataaagtttttcatttattatattgtatatttacttattatttagtttttcttatattgtagggcaattctatcaaataacatttttaagttttttcacaaaatagccatcaataaagaaaatgaccaaaatagccccttttattttgaaaatttattttaattttaattttttttaaatttaaaaccatatcctcaaaactccacctcttaactctaaccctaagtatatattatttaaccttaggatataaatgtatttttactctttaataaaacttcttttggtcatttttcttattgagaactatttttgtgacaaaaatttaaaagggttatcttaggaaatttctctatattgtatatttatttattctaattttcttacttttatatcaaatggtaatattaccgagttaaaactcaatttgtatttaaagtttagatattgtttttttttttttgataaccgagtgttttggctccaccgtggtggtccagactagagaccgaagcgagcaaggacgctgccaggacgtcaccatgtggctcatcgtgaacggttttcgatctcgggcactgggggatccgcatgtaaattttccagtggctgggtttcgaacctaggtggcggaactcacagccgtgagtcctttacaccagagctagataccacggttaagtttagatattgtttggtatacgaatatgtcacttttcaatttatataacaaaatgtcttttgacgacaacttttattagtttattagatagttatgttttttcttttatatgaaatgtatatgtcattatcataattggtgtattagaagatggtttcttaaaagtaagaagttctgaaatttgtggtgaaacaactgtatataaaatagctgtagaattcttccttttcttttgaaagcctatttgttttcttggtaggattagatacgtaacatgatagtacaataacatgcacctaccatttacgtatgtgttaataacttaattcaaacaccatcgatggtgcaatttctagctgaatatcaaaactcatatccaaattattcatttgctgagaatgagttgctctaaaaatccaagccagggatcaactcctctcaacataaaaatttatttagccagttcaaataatgccatatattaaaaattcagtgaatcacgactacactcaccctgaaaaattatatgcgtgttaaaagcccaaatcaccttctagttataaaactattcacttataaaatatttacacatacaataattagctagaagtattataacagccataatgcatcccgtagttattgaatttatcattgcatctaccttggcgaaagcaataaattgcaaatcactcaaatacgttagatttgcagtagatatacacacgtagcacattcatattcacgtacgtatgtaccatacgtggattatccaatatcacaaaataattgcatatattattttcagtattgaatattctctctgtttcataataagtgtcattttaacatttttttgttataaaaatagtgtcattttacaatttcaaacaaattatacttatttttagttgaaaattaattgaaaactacattgattttataaataattttattatctcaaat

SEQ ID NO: 73 - DNA, 5858 bp, Brassica oleracea
  misc_feature 1..5858: note = source = /organism="Brassica oleracea"/mol_type="unassigned DNA"
  source 1..5858: mol_type = unassigned DNA
aaatcctaaaccattactccttttggtatgaaaccaattcgccgcgtgctctggttgaattgataatctatagcaagtggtttcacggtcaaactcctcatggttgcaaagatatccttctttgaatgcacctacgtcgctgctggctccaatgtagcttgtatggttgatctcatggagttggtggtccagctactgacttgaggtcctcatcatgtctccaaggaaacctccaagcttctttcctttacaaggatgtcactcttgttacttcctctatccaaaggtttgcaagggtttgtttcgttattgttacttattattaattgaaaccgtcgtaatattctccttacttatttcaaatcttatgaatggttgatacctagttacgataagctagagccacacatgcctcagattctcaacgtttcagcagttgctattgaataagatttgagcggtgcgattgcggaagagattatgaaggctatcaaagagaggaatcaagtgcggttatctcttacagattgtagagaggaacaggagaaaggtatcataagctatgaatgccatgaaaccacattgcggtttgagccaggctctgatcagattaaggcgactgcatatgggaagcaccctactcatcatcaacctttagcttatactaggctaatacattttgtgatattttcgtattatatactaccattcagttaatatattaaaaaaatacaatacaaagactacaaaaatattggttttcgggtacaatgttagaacctattccaatacgaatgtttctgactatatagaagaatcttttttcccttcaatatagatgctgaacaaatagcagcgtctctttaacgagacaaaacttgtgtccaaaaatatcacaaaacccacgacaagaagccactagagaagacctcgatgaccttagcttgcagtttcagtagtctatgtatgttcaagaaaaagatgttagtttaaaaaagcacgattgggtgtcggaagttagaaacagggacatatcattgatatcaacacatttctgcgatttcttcgtaatacaaagaactttgaaaactgaagtaagtaatttcttctttaaacccaggacatatgggcctacataatagcaccaacgaggcaacgatacaatactcagaaattgcttacttaatttttttaattcatctttttaatataattatttaacggtttttttaccaacgaatcatgctttatacttgcatatatttatttgtttattgtgatcaaactcaaaaactgtactatcacgtagctgtcatgtaaatcttgagaaagctacatagttggtcttacaaagcgttaccatgcatgtactttgttttttttgaacgaaccatgcatgttctttttcgtttgattatttacaaacgttcttttttttgttagattagttccatgtaatttttttcgtgctaaagaaatattagaaattctattcctcaattctcacgccaaatgtgtacggtatctggtttcgtcagaagtattataaactgacaatgctagcaaatgttctttgactagaaaattacatttatatcatttacaaactaaaaaatccaaataaaattttaaatatttgcatataattctcgaacccatagatctattgatctttattgtaaatcttgaatgtagatttcctatccaaactacatattgttctttattaggatattgattattgggatattgactattgacttgttttctctttttttctggtcaaatttgacttgttttttctaaggtaataaatgatctattaaatacccaaaacggaaataaatctttcatttatatattgtttatcatacacatctgtatgtgctaataaatcaaatacagattatacaaccagattcgtgcgtatataaccataccaaaaacttcttatcacccatcttttcgctctttgtatcggaaagaaaaaaacataaaagatggattgtttgctttgctcaccaattttatatgtagttcttatatttctttggtatttagttagagttttgttaactcgtagtaatccatttccacctggtccaaaaggctatccaataatcggtaacatgaaattaaagaatcagttgaatcatcgtggtttggccgagttagccaaacaatacggtggtctcttacaccttcaaatgggtagaattcatattgtggccgcttcaacagctgagatggcccgagaaattcttcaggtatgtggttttctacggtttagaaattgttttgacaatttaaatattcttctaaatattttgataggataacagtgaatttatagtttttgtaagacacattcatcttaaaacattcaattacgttaggttcaagatgtggttttcgcaaaccggccagctaacgtcgccatttcatatctcacttacaaccgggcagatatggcgtttgcgaactacggtccactctggcgtcaaatgaggaaagtttgcgttatgaaactctttagcagaaaacgggccgaatcatgggcctcggttcgcgacgaaattaatacgatggttcaaactctgactaaacaaaccggttcaccggttaacgttggcgagcttgtctttgctttgacgcggaacataacgtaccgagccgcgtttggctcgttcgctcgcgacggtcaagacgaattcgtaaagattctacaagagttctcgaaactctttggagcgtttgatatcaccgagtttttaccgtggatgaaatggtttagtaatcgcgatttcagcaagcggttggaaaacgcaaggaaatcgctggatgggttcatagatagaatcatcgatgcacatatcgaaaagaagaattcaagaaaacaagacgatgatggcttggaggacgacatggtcgatgaattaatggcgttttatagcggcgaaagcggtgagaacggcggcaaatctaatgattcactgtcttcatttaaactcactagagataacatcaaggcccttgtcatggtaaatagtttaaattaatcattcaaaaaccataagttattaatgaaatgattttttttatttaggatgtgatgtttggcgggacggaaacggtggcgtctgcgattgagtgggccatgacggagctgatgaagaaccctcacgaactcgtaaagctgcagcaagaactagctgacgtcatcggattgaaccgtgagtttcacgaatctgatctggagaatcttccttacttcagatgcgcgatgaaagagacgctgaggctgcaccctccgattcctcttctcctccacgaggcggctgcggactccgtcgtctccggctactccattccccgtgactctcgggtgatgatcaatgtgtacgcgatcgggcgagacggatcggtgtggaccgagccggatgcgttcagaccgggtcggtttatggatagcaaagctccggatttcaaaggtagcgatttcgagtttctaccgtttgggtcgggtcgaagatcgtgcccgggtatgcagttggggctttatgctatggagctcgctgtggcgcacatgcttcattcatttgattgggagttgccggaaggaggtagctctgatgatctggatatgactgacatgttcggtctcacggcgcctagagccaccaggctcatcgccgttccgagttaccggctaaaatgtccgatggtgatttgaaatgcgtgttgtttgtttgaagaagttttatatcaactgtttgccgttttttatgattccttacacaaagtgtttgttgttgtttaagattctaatctatttaaactatttggtccggatcttccgattttaaaggtaaattccagattaacaggacacgggaaaaataccgagaaagaaaggagtcattacaaaaccataaatcaattctggttctccttagaacacaagaccggtttgtaaactaagcaaaagctacttagttagtacctcaaggggtagtggtggtggtggttcactaatctcccagaagggcattcatacgcaatgtgtcctcttccaccacagtttcgacatatcatcagcctagccgtgcagtctctgctcatatgacccacttgccggcagttcctgcaaaccacctcctcctccctgtattgggctctgatccctcgtggtcttcgctcttctgctgctaacacatgggttttggggcatttgatagcaacgtgaccagccaggttgcagtgattgcacacgggatcgtctctgcagtcacgagctagatgaccagtcttcctgcagttgttgcaggctttctcgttagtgcaatcagctgagaagtgaccttgcttgtaacagttgttgcagagtctcaggtcaccaggagggaggtggcgagccgtgcagtctttagcttggtgtccggcaataccgcagctgtgacagatgccttcgttggtgcagctattagacatgtgacctggttcacggcagttccagcacacagacttcgcggagcattcagacagattgtgactgccaagttttattagaagaaaaaaagacaaggtatatgttattaagttatcagtgtcggccacagaaataaaatacaaagaattagacatacatcatcaaccaatctaaacacactacattatcaaagcagctaatctacgttcaagattagtgtatattagcactaaaaatgtagcaagaacttcactataacaatctaaatcaacttagaacgtgaagcaaagaccataagaaagaggaatagagaatggatagggtggttacccaggcaggccgcagttgtggcagatagagacattgggacactctcgagcaaaatgaccaggtcgcttacaattcttgcacgtattactttgactgcagcaacagaattttaatgttaagcatatcagtaaaaccaatttagtttctttcttctcggtatacagctaagctatgggtgaagtatactaagagaaatgccagtttcgatgagaaaaacctgaaagtccgatgtgaatctcttcggtaaggatcatcacggtaagaaaaacgatcagaagcaatctttctggccatcgggttcctactcctcctgggtgtaactggatttatcttagcaacaaaacaaacaaacaaacaaacaaaaacacaaccggaaatctgaaacggaagtaagaaaagtcacgcatttggtgtagcgaatacgagatgcaaagtgtgtcttaatagaaagaacgacggaacatcctccactttttaaaacattaaaaatcattccttaacatgcgctaaaaccctaacaactcaatctctattggactcttacaaatcttcaccgataaaattacatcactacctacctcacggcgacgacaaccaactttccgacaagtaaactgtcagcagttcgacaaatctggcttgcggcgaccgaacctgtgttctgtgcgacaccctcgaacacacacaaaataacgtcatcagttggtatcttagtaaatgggcttcaaaagcctttcgtgacccattaacaaagtgagacacgcctgacatgtattaaccaattactgttatgttcggtattcgttcactcaccacttgttcttttctgatacggtccagtctactactactatacgtacaaacatcaaatttgtaacatcatgggccaatatcccatatgcaatgtaaatttctaatgtgcaaacaagagaataaaatatgtgtattcttgtttctgctttaattcataaacatatggagccctttatatataggaaattacatgaaaagaaaaaaaaaagactacgaacatgaaaag

SEQ ID NO: 74 - DNA, 6735 bp, Brassica oleracea
  misc_feature 1..6735: note = source = /organism="Brassica oleracea"/mol_type="unassigned DNA"
  source 1..6735: mol_type = unassigned DNA
ctcttttaaatttttaccggatcctaatccgtgcggttaatatatttttttttaatttgtaatatttgattgttttacattcacttagtcatgaatttttttagcgtgttaaaccatctattgtacgacgaatattcgatatcatataaataaattcaacatatatatgtatgtaattagaaaattgtatacaatatataaaaataatgtttttaaaatatgaagaaataaagaactatgacttagtgtgccatagaaaatattataaaatagttattcatgtttaaagaaaatacaatttattaaacttctataaaaaaatttacatataaaatgatcgattaataaaatcagggtgttttaagttaaaatttaatttaggcaaagacattaactgaactggaaaaaacatgaattaaataagatagtttaatttaattctcaatggcatggaagtgtaaataactttgaaaactaagagacaatttatattggtacttctctttttttttttttgaaagaattttaaatttattcaaataaaaaaaaaaccttgatacagagattcactgcttccttactacaaatattgaaatcagaaaaacttcaaaaatctcataattcctttagcttcttccaaaccaattctccattgttttttcatgcttccctcctgctttctttctaaacgaggtgattatgttgcgaactatcttgtccaatctatcaatcagacatgaaactggttgggaaggctcacccacccttctcacatttctctcatgccaaatagcataggccaccacttgcagacaatatcgcatcaaaaaagtcatacctctcccttgacatcctttcactattaatagagatgttaacctctacttgtattttagctctaaaaaaccttttcttgtattcgtagagatttatataaagaaaaatctaaagattagtctcttaattttggagcaaattccaaggcttaaatcctatgttttgtttaatgtgatatagtaccaactatttcttttacgatcaggggcggatccaggattatatttaaatgggggcacatgaaaattatacattatattgttttaattgggggcactccttatgttttatactaataactatagaaaattaaaagaccccatacccttggctttgcgtccaccactgtttacaacatagtagaagagaagagaatgtagttaatcaatattatataaaggagataactagatctcttggtggttgaaagccaacaacaaatcattatacgtacataaaaggggcctaaatcatgccaaggaagttggtcactctgccatgatataatatataatactctactaaaaattaattatttcatttatagagcttgtggaatccttatgtatgcacccaccctctttatactagtcttctctgtttcgttttgcttagttatgccatcaatttttgaatgacgaattggattatagaagcaaaatatgatgagatagcgctgtactttctttagtaacatgtattttcaatttggtacatcctttcaaaacgttattgagtacgtaaatgcacaagtagataaccttttcacttgtaagatcaccttagatatgggaaatggaaatagttatatcaatgattattggcatgtcacatgaataataaaataacagtatatcataattaaaatttaaacgctaaaacttaatagtaatatgccgaataataacaaaatgtttcacaatgaccacaaaacaatgtctatacataatttagtgccgtattatttttgtatatatgatctaccacaaaaaagttgttcattttgcaaaagtcaactcattcttttatatgaaaagtccactctttgaaagtttttatattgttaagatgaaaaaaatatactcataaatggggccaagttatggttataaaagctagcgttggccttaataatcacatcatctcaaattagttgacaaactaacaatggagtctctgttatcacaaacactaaaccaagtaatagatcccaccccgtcagttcttcttatcaccatctctcttctagttgtagtctacctcatctcacaatggtttaaaccgctctaccctcccggtcccaaaggcctacccgtaattggaaatatgctaatgatggaccaactcacacaccatggtctagccaagctagcccataaatacggtggtttgttccatttacgaatgggatttcgtcacgtgtttgccatcacatcacctgacgtcgctcgacaagtcctccaagtccaagacatcagcttctcgaaccggcccgtgaatgtagccataaactacttaacctacgatctagccgacatggccttcgctccttacggacccttttggagagaaatgaggaaagtgtgcgtcatgaaggtgttcagccggaaacggaccgagtcatgggcctcggtccgtgaagaagtaaacaatatggtccggtctttttctagcaacgtcggtaagcccgttaacgttggagagctcattttcacattgacacggaacataacgtaccgagcggcgtttggtgcggcctgcgagacagaacaagacgagttcataaggatcttgcaagagttctcaaagctatttggagcattcaacatcgcggattttataccgttcctagggtggtttgattttcaagggataaacaagaggcttgttaaggctcgtaacgatcttgacgggttcatcgatgaagttatcgatgagcatatgaagaagagggagactgtaaacgttgatgaagatacagatatggtggatgatctacttgcgttttatagcgaggattcatcaactaatcgtaataagaacaccgtaaaactcacacgtgataatatcaaagccctcgtcatggtaagccaaaacgttaccttttttatttattgattttcctcttagtaatcatttaatccttgttcacattttctataaatatataagataatttagttgtaataatgtttaagccaaattaaaattatttcgaccaatttacaaaaaagaatatttgaagttgatagtttatacaccatccgttcacaaaaaaagtaaaatttatagagttttcacgtttattaagaatacaataaatatttacaatttaatttaaactttatttttcaatacactttctaataactatctaccaataaaatttaatcaattcaaatattcacaattaatgtttctcaaaagtatacaaaagtatcttaaaaatatagaaaatctatttatttggaacacgaataagctctagaaaatcttacctttcaggaacagatgaagtaatatattgggatgactgatagtttcgataatattaattagtagcaattatgtgttccctctgttcctgaaagtaggattttctagatttattttttattccaaaatgatagattttctaaaactttaagatatttttagtagttaatgttgaaaagttgtatatttttaagaaacattaattgaaaatatttgaattgattgaatactattggttgatatttattggaaaatgtatagtaaattaaataataaattcaattgtaaatatttattatattcttaatatgcgtgaatactctagaaaatctttctttcgggaacagatggagtattacttttcttgtttgtatataaaaataccattttggttttggaaatgatatatatctttgtgttacactgttcggaatcatacatttgatttgagatggaatataaagtagtacagaatataaattattatatcccagttgattatgaatttgaaattcgtgatttttaacggtttttgataactaaaatggtagctcgatatactaattttttttttttgaactattaatttactttattactggaaggatgttatgtttggaggaacggagacaatggcgtcagggatcgaatgggctttgacagagcttctacgtaacccagctgaactcaaacggctccaacaagagctcaccgaggtcgtgggtcttgaccgacgcgtggatgatactcacctcgagcaactaacattcctaaaatgcacactcaaagaaaccatgagactccacccaccgatcccactcatcctccacgaggctattgaggacagaaagctccaaggcttctttgttcccaaaggctcacgcttgatgatcaatgccttcgctatcgcacgtgacccgaagttgtgggttgacccggaagcgtttcgaccttctaggtttatggaaccgggtatgcctgatttcatggggactaactttgagtttatacccttcggggcaggtcggagatcgtgtccgggaatgcaacttgggctttatgcgatggaggtggctgtggctaacatcattcactgtttcacgtggaagttgcctgacgggatgaaaccaagtgagctcgacatgagcgacgtcatgggtcttaccgctcccagagccacgcgattgatcgcagtgcctgacacgcgcctcatctgtccggtgtatccttgaccatggagagagttttactgctctattcaggattaatggttcaaaagttttgtcttctctcttaagatcttgctatctttaaaaaaaatatatatatatatgtttttccacattgggcatgctttgttttttgttcacaggggattttcttcctcaaaatgtaactgtaaataaataataaatagggtaataaggtttttagtaattaaactcttcgattaaagatgaatggtaaataaacccctcaactaaaattttgacgaagaaaacccttaactttaattccgttaacgtttgtcaccctccgtcacagtattttagacggagggtgacagacattaacgaaattaaagttgaagattttttcgtcgaatttttagttgagggttattttgcgatccatctttagttgaagagttttattactaaatgccattaaatattttgaaatatttactatcatttatgcattatttaaaattaggtgttttcttgcacaatgtgcagtaataaaattttttaatttaaattatatttaaaataattttattaatattttattattttcttaccaatattttaatataaatttgatttaattatcaaagtacaactaaactgtatttctaaaatttgaagatatataaaagaagttaataatattacgtttaaaattatatatatattttttagattgtaacaattttgaaaatctttttagtaataaattgtataattataaaaaatagaaataaattattacgaaagtgattattttatcaaatttataaccaatttataaagctttataaatattttactactttataaataattaataaggtttaataataactttataagattaaaggtatggttgttctccataatacaaaaacatgaagtaagtaaataaaagaagaaaagtaaataaaaaatattgttttatttattaataaggtaggagtataatgtattggttccgaaagcaaagagaaaaaaatagtcatgattataattttttctttgatttccgaaaaaaatagtcatgattataattattaataaggtaggagtataatttattgttttatttatttacttcatgtttttatagtatggtgaacaaccatatctttaatcttataaaatcattgttaaccttattaataatttataaaagtattaaaatatttataaagctttgtaaatcggttataaaattgaaaaagtaatgtatatgaatttataaattatcttagaggcttataaatctattgtaagagttacccttttaaatagtgcataagtgacaataaagattttaaaatatttaaaggcatttagtaataaaacccctcaactaaagatggatagcaaaataacctctcaactaaaaattcaacgaaagaaaccctcaactttaatttcgttaacatctgttaccctccgtctaaaataccatgacggggggtgacagacgttaacggaattaaagttgatgagttttttcgtcgaatttttagttgaggagtttatttaccattcatctttagttcatgagtttaattacttaaattcctcataaataataaatgaaataaatttgacaatggaaggattataagtaccttgcatggaaagcaaccccgagttgctgaatttgaagctttaggcctctacaccattttgctcaatttccgatcttttttgtttctgtccagtgttgagaagcttcttctctggttgagcctttggctatatacactacctttgttcttcacaattgaagcatagcggctatcaacgagagtttattaccctacaaagtgtattctgaccatttatgtttctcatgcatcctctagaatctacatatgcaactcaaggaacacagcattaacatacttctaataaatcacacataatgtactcttatagctaatgaatcgagtagacctatcatgaaacattataagtttaaacttgaatttctcagacctagctaacatcaaagcaaactatcacatagagcttggatccctttctgtaagtgatacaaacccccattagttaatgattttgcttgatacacaactttggggatgaaagaattctcttttgcctttagtatatacgtgccttataggtcaagagtttcagagtattc

SEQ ID NO: 75 - DNA, 6472 bp, Brassica oleracea
  misc_feature 1..6472: note = source = /organism="Brassica oleracea"/mol_type="unassigned DNA"
  source 1..6472: mol_type = unassigned DNA
atcatctcttaaccgaccaggtagaacatccatacgtcctaatgaaccataaatccttcttgtaaatgatcttactctcactctataaaaatgcatgcttatttcacactccaagtcctcatatttaattaaatcagttccataacatgaaacaacatgaaacatttcatgttatgccataatatataacctgcaaatgaaaatgtaaaatgtgagtaagatttttaatagttcacacaccatcaaaatatatatttcctccttaaaacttcatattctatcatatctacaaagtttaagtctcattctatttctccataaattgattatcataaccgatacgctggctaaattttgagcaattacaatttttgggcagttacaattccctagatgaaccatatataacacgaaaagggggaagcacattttattattcggaaagagagattcataatgacttttaattagttctaacaatggagacgagagtgacacgaagatgtgaaaacagaagaagccgatgtgtgtttgatctatttgtatttgcctttaactaaataacatttcctttagataggtatgtttattaaaagattcaaaaaatgcatattatattcatataattttagagttacatacatgtttaaattatgagtgttcatatatatgtcgagtttatttagatacaatgtacttaattaacagcatatacataactttttctaacaaccaatcatgtttgctaaagataaaattcaaaattcagagagaaaaaaacaaaataaagtgaatagttttgaaaccccccttttatttaaaaaatatttattaaacatcataaattgtgttttattaaatccaatttgtgttgttctttgttgatcaatattatttcttgtttagggtattttttcttgttttctttacttttatttatttttctttactttctttacttttatttactttacttgaaattgaatgtgttttttttcgcatcaaggcagaaaccttgtcataatatcttagtacctagtttcaaaaaaaaaaaatcttagtacttgataagtttaatgtttttttttttgaacttcaatatgtttgtttgatgtttggacgtcgttcccatctgttaaaattttctctttacttttttcgtacatcaaattttctttttacttaaattaaatatttctaatcataataatattttttgcaaaagcacacgaggctaaaagattgtctcctataaaaacaatccaaattataagatgaatttaaaatcctttttttgaacggcaatgcatttaaagtccatacatataaaaatgtacacattaccaaacaaaagcaccatcgtttttcacgagtcattccacgatgtgacaaatattgacctaccaataccatcatatgattatactgtccatgtgatcataaaattggctaaacatcgagtgagatcattggttggttttgtttatgattttgttgaatatcatgttgtcccacaaaagattttacgtggatagccagataagacgtcgtcattgcgagacgaggcctaccatcaaagctgaaaaagctaaagaaaaatccaacagtaccatcatactgacaatatattttaacttagacatggttaccagactacatagaagatatgagatctcgcttgaacaaaagattctacagcttaacttcaaatcaaagttcaatcaacaacggattaaccactactaagtttgctccactctgtctcgctcactctaaatgtgctctatgtgcacctcttcaacaaaaaaggtctctctaaagaatttttgtcgtctacctaatttagtaaatatagtgagatgataatgcaaatatacatgccatacaagctacttatcgttttcaccaaaatactatataataagccctaatgatcaccctgagcgttgtgacttctttggttgttctttgcacccatgttcacaagaaacacattattaaagaccaatttcaactaaaataatagaaaacatacacaaaagaggtattagaattgaggtagttgttaagtcgatatcaatcacataatttcttaacagacataaaatagacccacgcacactaacttgtatcaaatgtatactctaatgacactcatccttcttgtccctcttcttctttttcttttccgtcacctcctttcacgacggctaaggcagcggaaaccataccctccgggacccaaaggcttacccatcattggcaatatactcatgatgaaccaattcaaccaccgtggcctagccaagctcagccgcacatacggtggactacttcacctccgcctcggaatttcccatctttttgtagtttcttctcctcaaatcgcacgtcaagtcctccaagttcaagaccacgttttctcaaaccgtccaaccacaatagcaatccgttacttgacctacgggcaatccgacctagcattctgcaattacggcccgttctggcgaaggatgaggaaactctacgttatgatgctctttagccgtaaacgggccgagtcgtgggcctctgttgacgaagaggttcacaaagcggtccgttctgtagcggccaatgtcggaaaaccactaaacgtatgcaaagtcgcattctccttaacaagagacataacgttccgagcagcgttcggctcctcgtcgtccagttctaacgaaggcagactagatgagttccttgagatcatacaagagttctctaagctctttggtgagtttaatgtagcggattacgtcccgtcttggctcagttggatagacccgcaagggataaacaagcgggtcgagaaagcccgaaaatctctagactgtttcattgagtcaatcatcaatgatcacttagacaagaagaagacagaaaagaacgttaacgttgacgaggtgaccgatatggttgaccagttacttgcgttctacaaagaagaagttaaagtcaaggactcagagaccaaaatcaatctcgataacataaagggcatcatcatggtaaaaaattaaacatactccattccatccgtttcaaattatacactattttattaattagtagaatgtgcaattttttgtttatttaactattacaattatgtagcatttattgtttaattttttaattagtgataaaagaggtaaactaataaaaatgtcattaaaatcattaaaattatatttattttaaatcaaaaattttactctatcaaaataatttttttaaataaaatgaaataaaggatgtgatgttcggaggaaccgagacggtggcattagcaatcgagtgggtactaaccgagctactccggagccccgagaacatgaaacgggtccaggacgagctagcgactgtggtcgggcttgagcggtggagcgtggaggacacgcatcttgagaagctctctttcctaaaatgtgtactcaaggatactctccggctccacccgccgttccctctcctcctccacgagacggtggaggaggccgaggtctccggttacttcattcccaagggatcacgtgtgatggtcacacctacgctcttgggcgtgacccggcttcgtggtccgacccggaaatattcaacccgagtaggttcttggatccgggtgctcctgatctgaaaggaaacagtttcgaattcattccgttcgggtcaggtaggagatcgtgcccgggtatgcaactcgggatgtatgcgtttgagcttgcggtggctcatcttctacactgcttcacgtggagattaccggacggcgtgaaattcggtgacgtggacaccattgaagggccgggtctcacggttgctaaggctaactctcttgtggcagtgccgatcaagcgcctcctctgtcccatggtcttggaaagccacaatgtttaaaaacttgttttcataattattattttacgttgatattttcttgtgtaatgtatgtatggtgttcatgtttacgttagcattattttctttaatataatatccaaataaatacataaagcgtaaaatatggttattttaaaaatatactaacatcaagaagaaaaccatgtttttttcaagactaatatagtcccaatgaacattaaactagcttatgaccattttctgagtagtagatccaatcatatctagcatcccacaagaatcgtgcgccgaattttatgaaataacgagattcttcatgtattatggcaggacataaaatatttcatgttgtgaatgttacgaaactgatttaattaaaaaaaaaaaatttgaagagtgcaataaacatgtttttttatagagtgaaaggaaaatcatttacacgaaggatttttcatccatatgtcataagatcattggacgagaaatctttcgtaaaaatgatttcatttcgttttataaaaaaaacatgcttatttctctttccaaatcctcatcttctctattaaaagagaagtaccatttttatctactatttagaagtctactaggaccatttcattaatcacataatatgatataataattaataagaatattaataataaattaattttaactatagatttgaattttattttcagttataacaaaatctgttgagaaaaatctaacaaaatcctactaaatttttaaataatttttatttaatatttgtttaattaatttcgtaattaaataatataatgctttcatttaaataaattatcatctaccactaaaacgggttcaaattttttaatcatgtcagatttgttttatacaaatgatgttattaacatatgttaaaaatttatttctacagctatatattttcaaaaatcatatgttgaagaatattttttatttgatgatttcaaattatgaaaactcgaaaacgtaataaaaaaggtaaaatgtataaaacaaacccctatattaataaagtaataagaaacttaaacaataaaattaaaaagttataaaatactaaacactaaaatataaattgtatatttaaatttatataataatattaaaattaaatatttataaaatgaaaatatacccgcactatgtgcgggataaactctagtttttaattaaattagttataacattcaacaacatgcaatatgtcacgtcttgccacaatatataatctgcaaatgaaaatataaaatgtgagtataaatttgtttatagttcacacaccatcaaaatatatattccctcctcgaatctttcgtatagtatcataaaagtttaagactcattctatttctccaaaatttgaaaatttcaaacctattaatacacgaaatgcaagtttgaaacatatatacacgttgtcaaagaaaaaaaacatatatacacacgcaaagaagaaatttcgaatgaagacaaaatagtgaagaaaaatagcgttggatgtgttcatcataatatgggattgtgaaatttttattttcatcaatttcaatggagttataagaatgaagccaacaaccgggaatgagaatgcaaaggaggaaaaaaagttttgttttttctgggtaaaaagaaacaaggtcgttttcaaacaaacattccaagcaatttactgaaatatcgaaataattttttttttaaactcttacgctaacaagaaaagtaggtcgttactccagaaatactgaaccgtacttaagaaactgcagcaaacacgaaaactggactcgttaaaagacaaagataaagttaagcaatcgtcattcaagaaacagattaagaatcgaaagaagcattaagattacctcaactcggagaattagatgcagcagctagctagcgatctgaatgtttatcgagagagagagagagatagagatagagtgaatgagaaaggaagggagtaagacgaaacacaagaagaaattctatcgttagtgaaagaaactcaagtttttagcaaaggcattagatatatgggaataagactattggtgaggaatggaacaagaagctgtggtcggttgtataggagtgaggttagatttggtatagtgtgtcaaagtaaaaggcaaatatacgattaatataagtaatcgtgttactatgtcgcaaatcataaaagcatcctcctaagtacgatatgtataaagtacgttaagtatatcgatcgctatataaagccagtctggtagtctacactctatacatatagatagggacatggatgccatgtgccattctgtattgtacattcaactgccattaatttccttgtcccagtcttcttttgaatcaattatcatgccttctcgaattattgtactctgctattgatcaatttatttgtccaaatgtactcttcttttgaattatttacgggtgtcttcttccactactatactactttgttttgttcacctttgaagtgtttggaataaacggttgaacgtgtttagggtga

SEQ ID NO: 76 - DNA, 1575 bp, Solanum melongena
  misc_feature 1..1575: note = source = /organism="Solanum melongena"/mol_type="unassigned DNA"
  source 1..1575: mol_type = unassigned DNA
atgaaagagatggtgcaaaaaaatatcaattcaattcttgaagctttacaagctaatcctatgctactcttcttgtttattatccctctcttcttcttataccttttctcgacgtctagccggagacgttatcctccaggtccaagagggtggcctctcattggtaacatgatgataatggaccagttaactcaccgtggtcttgctaaactagcagaaaaatatggtggtgttatgcacctcaaaatgggttatatccacaaaattgtaatatctggtccggaggaagctagacaagtactacaggtacaagacaacatatattcgaaccgtcccgcgaccgtagcgataagctacttaacgtacgaccgtgcagacatggcttttgctgactacggaccgttttggcgtcagatgagaaaattgtgcgtcatgaaactcttcagtcgcaaacgagcggagtcatgggactcagttcgcgacgaagtggactcactgatcaagattgttacaaccaacgctggcacctccgtcaacttaggggaacttgtttttggtctcactcgtgacattatctaccgagctgctttcgggactagttcagctgaaggacaagaagaatttattaaaattttgcaagaattttcgaagctatttggcgcgtttaacatggttgattttattccatggctaggatggattggtcagcaaggtctaaatgttaggcttgctaacgcacgggcatctcttgatgggttcattgattcaataattgatgaccatattgaaagaaaaaaggctaatgttactattgataatggtgacagagaaactgatatggtggatgagcttttagctttttacagtgaagaagctacagtaaatgagtctgaagataatttgcagaatgctataaggcttacaagggataatatcaaagctatcatcatggatgtgatgtttggagggacagagacagtagcttctgcaatagaatgggtgatggcagagcttgtgaaaaatccagaggagcttaaaaaggtacaacaagaattagctaacgttgtcggactcaatagaagagttgaagaatctgatcttgaaaaattaacctacttaaaatgttgtttaaaagaaacgctccgtctccaccctccgatccctctcctcctccacgaaaccgcggaggaatcgaccatcttcgggtaccatattccggcagggtcacatgttgtcataaattcatttgccattggacgtgacaaaaattcatgggaagatgctgatagttacaagccttctaggttccttaaacaaggtgtcccagattttaaaggaggtaattttgaatttttaccattcgggtcgggtcggagatcttgccccggtatgcaacttgggctttatgcattagaaatggctgtggcccatcttcttcattgttttacttgggaattacctgatgggatgaaaccaagtgaacttaaaatggatgatacctttgggcttactgctccattggcaaatcgactagtggttgtgcctagtccacgtttattgtgtacactttattga

SEQ ID NO: 77 - DNA, 7670 bp, Solanum melongena
  misc_feature 1..7670: note = source = /organism="Solanum melongena"/mol_type="unassigned DNA"
  source 1..7670: mol_type = unassigned DNA
ttatttcatcataataatttttaaattttttatccaagctaacataaattaaaatggatggattattaattaatcatgataattgcataaatctttctttatcttgttaaatttatgtccaatcaatagataatcataaattaaaatggcccccttatttaataaacctgccctcatattatcttcaatcgatcaaaaagatttaaggggtcgtttgagaatttacgtggagattagttatattgatactagtcatgttaaaaataattatatcaatattattattattaaaaataatatttatggcataagtttgatttatacaaagatttaagaaattgtttatttataaaaatacttttcgccttgtttagtagaaaaaaaaattcaaagaacctcaaggatatgtatttttgtaagtttgttttatttctgtaaacaactagtttgggatatattcataataaataatcaaataaggaataagacaatacaattttattttgaaaattacttggaatatttatggccaaatgggaccttagatctaggggtgtgcaaaaatcgaactgacggataaatcgaatcgataaaaatattattgatttattgttattgggttattaggttatcggatttttaatagtatttgaaaaaaaacttattggttatcggttcgatatcaatttttactattggataaactgataacccaataagattatataaatttactattttaccccatcaaaaatattaaatattaataatttaatagtaataggacacaataatagtactttacctattttaatctttagactagttacccactttaataattaattatggcttttgtaagttagcaaatgtagccttcatacgtcatttgcttttattgatgcaaattcttatgaaaattcattattgtcgggtaaatcgaaaaccaaattgttaaagatttaaaatcgataaatcaaaaactgataaaaattattttattgatttgttattagttttatatatttaaatactaaaaactaataaatcaaatcaataatatgtaaaatcgaactaaaccgatcgatgcacacccctccttaaatctatcacttcatgagcagagcaattactcgctttaattaatttatggcaaattgcactagcaagtagaagtatacataggtctatttgttttttttttatagaaaaaatcaaatcaattatattgatttgttaaagttataaatcaaatcaatctaatatttatttttcttttatagttataccgtaattaaaaaagagatagaattgaagaggttgttcttttgattgtgtgtgatttaatgcttaatggcatctcaaagacattggaataaataaagtttaaaaaaataaaatgtacaatgacatcttcaagacattgttttagaaaaacatatcaaaaactcacgaaatgacaatatatttaatttttttggtcatattacaagcctaaatatgaaatgatatatgtaaaagttaaaatttatagactagttttaaaaaatacttacaaagcatattataatagtaaataattatgtataaaaaaacttaaattataaatgtattatgtcagtttgatttaaattcgatttgatttatttttttctattgatttattttttcaacaccaaatcaatcaaatcaaaccactaattattatttttttagtcaatttcttgacgatttaatttgtacactctacttgcaagcgatatttaagagaatttatatatgatatcatgaagtacaaattgaaaattatttcttgactatagtttcttgacattatatgtagttgagctaaatatctaacacaatatccaagccaacttggtgatgacattcaaatgaaacttgtatatatagacccccatcattttgtatacactcaaaatattaaatctagcaagtacattcatctcacaaaaaattcaagtacccctcataagataatgaaagaaatcatacaaaataatacttttttcatacttactactaaagaaacaaacctcatgatgccactctacttcattttccctatattcatctttttcttcttcactatatcaagattaaacaagaaaaattttccaccaggtcctaaagggtggcctataataggcaacatgatgatgatggaccaattgacacatcatggcctagcaaaattagcaaaaaaatatggtggtattttacatctccaaatggggtatcttcagatgaccgtggtgtcaagtccagctgaagcccgtgaggtgctacagttgcaagataccatatttgctaatcgtcctacaactatagcggtggaatatttatcatatgctcgtgcggatatggcttttgctaactatggacccttttggcgtcaaatgagaaaattgtgtgtcatgaaactttttagtcgcaaaagggctgaatcttgggactcaattcgtgatgaggttgaatccatgactatggctgttgccactagggttggttcaagtgtcaatattggtgaacttgtctttggagttgcaaagaacattatctataggtaattaacgcggagaatgagcaggttgaattaaatctgagaaagttatggggggtgggggtcaaatcaatgaaagggtcaatgatccaatatgtcaaataacaagtcttaacctaaatccttcgacttgatttgaccttacttgacatgaaccccttcttttttatcttgcttgtttaccaaataaatatgaaagctaatatattttttaaaaattattactttatactcttacatatttacatgattctcgtttttccaagagtttttaattttgtatatatggatatgagattcatttgatctgtctaatcacatcttttttactagttttgattcaaaaattgaataagttattccgaattcaatttattaattcctaaaataagttgattttgtcatctctatctacagggcggcttttggaacatgctcgaataggggacaagatgagttactaaatatcatgcaagaattttcaaagctttttggtgcattcaacttggctgattttataccttggcttggatgggttgatccacaagggttaaataaaatgatcataaaagctagggcatccttagatggtttcattgatacaataattgatgatcatatacaaagaaaaaagactaataattataatgatgaaggaaataataatgatatggtggatgagctcttggcattttatggcgaaaaaacaaaattgaatgactctgatgatttgacgaatgcattgaggctaacaagggataatattaagtccattatcatggtaagttcaaaatcttgatgttcctatttctttatagtttaattatacctatctttggtttcagtatgttgatagtgtaaaagaatttttaatattattaaaatcaactattgataaaaaaaatgagatttataatctaaatcagatattaggtcagttagggagtttaaagtcatgatacatgtttgatagaattcagttactcttaatttgctcaaacaatatatttatgctacaaaatttattattattattattatatatgtatgtatgtatatattaaatttagaatttaatcataattaacaattgaaatcattattctaaccatcagaactcataaaattaaaattttgattcggtagttgccctaaaaattgcaccaataatgtaaaccatacttggcctactgtcacataattgtgagtaggcctactgtcacataattgtgagtaggccatgcattgaaattatactttttaatgtttataatattattgaacgcttattttggagttttattttctttaaaagagagagagagagaaattcaaaatatagattgaaaatataaatatttttaccatgatcaccacaaaattttacctattatacctactatcttatgtatcttaaatcattatatacgaattaaatattcataatatctttcaaatgatttgacagtaaatttaccatgacaggatatcatgtttggtgccacggaaaccgtggcatccgccatagaatgggccatggctgagctaatgaaaagcccagaggatctcaaaatggtacaacaagaactagccaatgttgttggcctccatagaaaagtagaagaaatggattttgaaaaattgatcttcttaaaatgttgtataaaagaaactcttcgtcttcgccctccgatccctctcttagtccatgagtccgttgaagataccacaatcaacaattactatataccagctaagtcgcgtgttattgtgaatgcatgggctattggacgtgacaaaaactcatgggatgatcctgagagttttaaaccttctaggtttttgaaagaaggtgtagctgattttaagggtggtgactttgagtttttaccatttggatcgggtagaagatcttgcccaggtatgttcagacagaactaaaatttaaagtttttgaaaatatatgagttcaaaacttaattacattcatagtgtattccaagaaaaatataatggcacattcttaatataaataattttaactttaaaattttctaatgtttataacaattattgtagtcgtaaactcagtccattcagtttaaaatgagaagtaaagttaaataattcattttaaaaaaatatataaaggcattattctttttttaaacagattaagaaataaagtgtgtcacatgaaatggaataaataaatattatattttaatagctatataagtatcataacatatttaagatataagtttcaaaagtattttcagttttcttgattatattttcattcaaacacccttacataaaataaaaacaggcaggcagagtacatattttagaatatatttagcatatatatttaatattatactaatatatattgaaatatgacaaattcaaaagtgtgttacatgaaatagaataaaggaaatatattttaatagctatataagtatcataacatatttaagaaataagtttcaaaagtatttttcatttttcttgattatatttaaacatcattacataaaataaaacaaacggagtacaaatatattttaaaatataattagcatatatatatatgtttaatataatatataattgaaaaatgacgagttcaaatcaatccttgtggtctaatcctttcctgggataaaactacatgcgataaaattttacaatatgattctttgttgaatctcgatcaatacagaataaaagctttagtatttcgatcttgtagtttaaccctgcccttcgataaaacaataatatacaataaaacttcatgatctaattctttattgactctcaattcatacttagcaagagttttagtgttccgactcgtgagaatttcaagcatgtgtgtttgtgtaggtatgcaaatgggattgtatgcatttgagatgacattggcacatcttcttcattgcttcaattgggaattgccaaatggaatgaaaccaagtgacattgatatgaatgatgtttttggacttactgttcctaaagctacaagacttgtggctgtgccaactccacgtttgttatgtcaactttattaattttgtgaactaataattagttaggaattcttagatattatataaaaatatacaagtatggtcatggaaataaattcttgaatgagttgatcatggaatatagagaaaaatattgtgaagttattggttttctttcatgtattgaacatgcaatatattggcaaaacaaagaaagtggatatcttatattatttaatattattaactgttaatttaaatattatatttgtgttgataaaattttattatgacatattgttatatgtgtctttaggtaatgatgaaaaaatttcaatcttgtgtgacgatcaacttgatgtgattacaaaaaacatgttttttttttaatcttagtttaacttaagtcacatttatttatttgcacttaataaaagtcttgatcttaatcatcaactctaaatttcaaccattcagatctaaatcatgaagtgcatttaattttttttcatgttacgatcacatagtacatttgaataaatttaaatgattaagatatataacaaataacataaatcaatggaacatagtattggcacaaattgcaaatctacgtaaaagtgaaaactcctgtggtagataggtcaaaaaaatgatgaaattttttactatggtcttttttagtccacctcaattattccgacgaatatatgtcgacttggcacatcccaccaacaaagataccaaacgacgtaatagataaaaagaaatctccttatgtaatacacaattctcaaactaacatgttttttggaaagaaacaggaaacagaagttagctgacctacttttcattaaaatttacaaagttgcatattcttgtttcagaatcttacatgctaaagctttacagttggtaaatatgtggtcacaactcacaacaatgagtagtctacaagaaaaagaactgtcaaatctccatttttacagcacgacgaccgaaaacaggataatacgtatccagctcaagtagttctaagatctcttgtgtagcccatagatacgacgaatggccttcgatgaattcagttacatcaacctgtaaatggagtaccaatgaaattgaacttcagttaaataaacaactgcaatacagataaagatgggaatagaacttacattctcaacaccaggaacctcaactggttgaattccagccaagccttgcgtaaggagactgtttagagaagaaattacagaagtcacaagaaagaaaggcagaaaacagcaaatcaaacacgaaagcagatagtgcggaaatccttcaaacagcggcagtacttgacatgttcaacattagtactatgcttgcgggcaatgcatacctggcacgaaaggcaactccaagcatccaatcatttgtagaataagcattcacaaatctacccgctaccatctgcaaaacaagaacagcgagcttcagatagtaatgaggtcgaatcctatacttgtaaggaaaacaaaatatgaacaaaagggcatgcggaataggggaagaccaactataattagaagtacttaaaataaagaaaaacacgagaccgataacctttctagtagcttcccagttcatatccttaattgcaatgggtgccccaagaagaacgaccctttccacaagtccagctgccaatatttaagtacttcaggatcttaaagcaaatctgaatgaccctaaaagaatattaataataataacagtatgcatattatgcattatctttcattaatgaacgtaagcacaatgctttgtcttccactaataatagtaagcatattatgccttatcttccattaatgaaagtaagcaccatgctttatcttccattaataaaagtaggcatattatgcaatatatcattcattaagaaagataatgcaatatatcttgtcttccattaacaaaagataattatgttaagatttcaagtggacgataattttatttggaatttattctccacttgggaatgtttccacttt

SEQ ID NO: 78 - DNA, 7964 bp, Solanum tuberosum
  misc_feature 1..7964: note = source = /organism="Solanum tuberosum"/mol_type="unassigned DNA"
  source 1..7964: mol_type = unassigned DNA
atgatattgtgtaccctgatttcgatttgtttgttttaattatcattttaagagtaaatgatttctttttcaaatactttctaaatatttgaaattgttaactatatatattttaacttataatagttgttttttaaatataatttttaaataaatgaattttatttttaaaaaattaaattttttataactaatttcacattaaacattaatttatgtgtgagattaggaaatatgtacaagcttgagggagtttttacaagatttgacgtttgcgtgaaagattaaatatacgaaaaagggctaaaaatatccctaaagtacaggaaaaggtctaaaaacacccttcatccatcttttggtctaaaaatacccttttactctctcttttttttcaaatacttattatgtgtcattttcttattgaatgaaataaaaatccacctctattaattttccccccataatttatccaaatcaaaacaatatatcttttcaagatccaaaaaatatattttttaaatctagcaatttctattttctatagctcttttccaaaaaaaaaaattgttttagataattaaaatatttttaaaaatattagtcatgccacaattacaaagacataatatattgatataaatcctaaaaaaaagttgttccaattggtgcaccagtccaagatttgatcactaatgacttggatgacaagattattagtcaaaaaaattaaatgctctttttttttaaaaaaaagaagaagatagaattgtacccgcatataaagttatgtttcttcaaattttatttgtgtaatttatttattttccatatgtttaaaatttgttgcaaacaaattggcatgcctagtgggaccaaatctgcgcttcatatcttctctcttaaatcaaatatgaaaatctaaagctgcaaaggtggaagaatgagttcttcaagcctcgtctttgagtttcatcgattctacactccaacaagccttgaagcaaggggcatttgtagacattgaaattttgcggactctacaagacatgttcaattcgaattgggactctagaaggtgtgttcagtttcaattagaatttttggagtttactcaaccctaaaccctactttatgcctatataaaaagtactaaactcccttaaaggaatcgcaaaatatccataaactcttgggtatttttaaagatcaagatctcgaatattccacaaaagtaatggaatattcatatagaagttaaatctaaatcattctagttcgagaaatacgtcactaacggccctcgaatcatggataaatcttaggagagtagaatcaagagagaaacagaattgtacccgcatatttatcaataaagttatgtttcttcagattttatttgtgtgatttatttattttccatatgtttaaaatttattgcaaacaacaatctctcaaggcctcaattaattcatacattacttcttcaaattttaaatcctcttaataaaatttttgactccacacgccatggtccagttcgccaaacttaggcaaaaaattttggtgaatttggccgttcagctacctgaagtacaagatgctagtgactagttcccctctcatatgattttttatttttggttcccacccggtgtccagagcccacattgaactgcatggctcatttggggggggtgctcccaacaagactttctacatagtcagggctcgaacccgagacttctggttaaggataaaaacacttcaaccagtacaccacaacccatggtggtcctctcatatgaagttgtggactagcttgggtcagtctttgacttaaaagaccaatctacaaatagtttaatataaagcttaatataataatatgtattggtatattttacttgacatataaagcatatttttttcgccaaatcaacagtatacaccaacttggtaccgactggaagtggaataaatttgtatataaagaccccatcctatccaccaataaaaggtagaaccaaaattttgagttcatcccaacaagtccctatctcctacatactccaccaccatctcccaaaatctccactgaaaaacaaaccaaacatatcaaaacaacaaagaaaaaaagagagaattgtaaacaaaatgaaagaaatggtactaaacaatatcaattcaactcttgaagctttacaagctcagcccatgctactcttcttcttcatcatccctctcttcttcttataccttttttcgacgtctcgccggaaacgttatcctccaggaccattaggctggcctctcattggtaacatgatgatgatggaccagttaactcaccgtggtcttgccaaactagcacaaaaatatggtggcgtatttcacctcaaaatgggttatgtccacaaaattgttatatctggtccagaggaagctcgtcaagtactacaggtagtattggaatatctgaccatctcatctaaaaagaatttatacggacggtataaataatttgtcagtagtataacataacatattgtagtactactaattaataagtttgggtatcttattatttttcaaatattaaattaaacagattatttaatttctaacaggtacaagacaacatatattcgaaccgtccaaagaccgtagccataagttacctaacgtacgatcgtgcggacatggcttttgctgactacggacccttttggcgtcagatgagaaaattatgtgttatgaagctattcagtcgtaaacgagctgagtcatgggactcagttcgcgacgaagtggactcaatggtaaagattgttacaaccaacacgggtacatccattaacttaggggaacttgttttctgtctcactcgtaacattatctaccgagcagctttcggaacaagttcagatgaaggacaagatgattttattaaaattttgcaagaattttcgaagctatttggtgcgtttaacatggctgattttattccatggctaaggtggattggtcagcaaggtctaaatgttagacttgctaaggcaagggcatcacttgatggatttattgattcgattattgatgatcatattgaaagaaagaaggctaatgttattaatgatgatggttatagagaaagtgatatggtcgatgagcttttagctttttacagtgaagaaacaaaagtaaatgagtctgaagatttgcagaattctataaggcttactagggataatatcaaagctataatcatggtaagtttttaacctaatgcttttccgtacattttttttgtgtggttgatacatgttccaattaacgtgtcataatttaaatcaactcgtggtttaaaaagaaaaaaaaaactaaatttgtacttgtggcatttgtggttaaaaagaaacatgtcatgtgacaacaatttttttttttttaaacttgtggcatcaaatatatcatttgatcagggaaatgagaagggtaaactttgaagtttgaattgataagagatttaagaaagattcttttttttagaaaaataactagtaatcttaaatatgtcataatatttgtgtgataataacttttttgaaacttatgtcataatatttgtatggttacagaagattcttgttaaggtaaaataggaagtacaaagttgataattttcaaattttaaaagtgacatatttttaattaacaaaaaagaaaattgttttacatcaatcgaaaggaatagtatattgggaaaatacaaacaaataaaagagagtattattttaacataaattaatttgatgttgaaaatcttgcccaaaacaagaaactttaaattttaatcagttgctactaggtaggatcactgaacacggctatctctggcagctcatgtataactatcaagaatagtggaagcttaggaaacactattatttttcgtgaaaatatgtgcaatgtataattttagtactttacttatattttaaaacttcctttgttagatgaaaattttgataattatttaattaagttgactctttatgatttgtttttagaataacaagatgcgttaaagtttctcaatcatcacaaaattgatattaagagtgaaaagaaaaaatattattgaaaattgataacctagtataataaaatttgtgctaccccatatttctatggttttagcatcacctagctagactttaattcaaaaatttgatgaaacttattactaattcaattcattatataggaaaaaatctccctttaatttccaatttttcatacaacttgatcctagtggaatgtcataaaatagaaacattaatttcctctaaatcattagcttcaaatcatttgtttttttgaatgtctcatcaatataataaacaccaaatatgattacgactttatcttgttatatacgatataattgttaatgtcctagctatggtctactatatagttttctagaattaaatagtacaaaggaattagcgtactgtacaactactagatcttataagtagatttttatttatttataggtctctctactgatgacatatatcatgagactaataagttgatttgaactttccctattaatcttgattttagcttcaatgctatccaatattttgccaagtggcacaaatggaagatttattaggtaacaaaagttttcaagtacaattgcctaatgtttttaatgtgaaaaagttactccagaaagaatttactaatgattaaataaaatcatactagccaatgaatatacttaacgtgtaaaaaatgagttagagccgtaaaaactatattctatgattaaaaattcaaactggccaataaatacctatctagaaaatttggtaccattccttatccctttgaaaaatcatttttccgaaaacgatgtacaagttatagtttatcttacttctacctaacccccttacctaactacatacctatttaacttttataagcccaccatatcaaaaaataaaataatattgttatgtattatattacttgactcgtattgatctgacgtggaagagaatcgcaggatgtaatgtttggtgggacagagacagtggcatctgcgatagaatgggcaatggcagagcttatgaaaagtccagaggatcttaaaaaggtacaacaagaattagctaacgttgttggactgaataggaaagttgacgaatccgattttgaaaaattgacctacttaaaatgttgtttaaaagaaactctccgacttcaccctcctatccctctcctcctccacgaaaccgcggaggaatcgacagtctctggctactatattccagcaaaatcacatgttatcataaattcatttgcaattggacgtgacaaaaactcatgggaagatcctgatagtttcaagcctagtaggttccttaaagaaggtgtgccagattttaaaggtggtaattttgaatttttaccatttgggtcgggtcgaaggtcttgccccggtatgcaacttgggctttatgcattagaaatggctgtggcccatttacttcattgttttacttgggaattacctgatgggatgaaaccaagtgagctaaaaatggatgatatatttgggcttactgctccattggctaatcgactagtggctgtgcctactccacgtttattatgtaattattgaaggagaaaaaagggtattttctacgtgtatacgaatagttatacacaaaatattggtttgcttggacatgtttatctttttttgttttgtctattagatgtaataatagatgcatatacataatggagaggacgaaaataattgcttgaaagggaaaagagagaggaattgattatatggtatttgtgtatagtagacactatatatctctttccttttgttttttaatattcatttggcttgcattgttagttgtaaatgtcccctcctcttgtttgtttcttctttgaaaacataattttaatcaaataaagactttatccttgttaccttattgtattataatggttcattttgattggatcagtaaggagaattcaggtaaatagtccgacaaatttactgtttaatttttctgatcgtacatagattacacattaattatacatgattatatacatacacacacaaatttatacacgattacggaggctattgtgttcttttatgagggaaaaaaaaaatgaatttccatatcctgtgacacccaatcgggttgcttctaaccaataccccctttgcttcagaaagcaggaaaaggatatttatccttttgatattcactttttacctgtttagtttagaaaatctagaaatattttattgtttattttttaatttattcttattatattaattaggaaaaatgcataagtactctcaatctatacccaatattccaaagacacacttatactatactaagatcctattactctcgaacttattttataaataattttctacctcttttcggcctacatggcactatcaaccagatagcttgaaaaaattgtcaacacgcgttgggctcagaagatagtgccacgtagaccgaaaatgggtagaaaattatttataaaataagttcgaggggtaataggaccttagtataatataagtgtgtctctgaaatttcgggcatatgttgggggtacttatgcattttccctattcattataatcctctttcaatgcattcttcaaagcattaaatttgttataactaaagggtaaagcatgttctaggttaatattaaacaagtaaaaatgaacgaaatgaataataacaaaaaaaatagacacatgaaaatgaatgaaagaagtaataacaaaataaatctgataatggagggtagggttgagtaggtccaaagctgtggaggggaccgttgagaaggtttgaatttcgacgctgaaaattaaaatagaaggacacgacactagtaaaggaagggaaaatagaggatcgaattcttttgtgaaagttcaaatagttaattaatggagttattaattagatgtttcatgtcattagttttttcttcttaaattagtgtaagacaaaggaaaatagggaatcaaactcttaagtgaaagttcaaataattaattaattgagttactaattagattcttcacgacactattttttattcttcttaaattaaggtaaggaaagagaaaatagaaaatcaaactctttccgtgaaagttcaaataattaattaattgagctactaataattatttttcatggcactaatttttcttcttcttaaattaggtaagggtaggaaaaatggagaatagaactttacatgaaagttcaaatagttaatcaattgagctactaatggatttttcatcgcacaagattttcttcgacagggaaaatagagaaccaaactcttaggtgaaaatttaaatagttaattaattgagatattgattaaatttttcacatctgtttcttgaattagggtaatgaaagagaaaatagagaatcgaaccctcacgtgaaagttcaaatagttaattaattaagctactagttattaaatttttcacggcactaatcattttttttcttcttaaattagggtcagagaagggaaaatagagaattgaacccttataaaagttcaaataataattaattatgctactaattagttttttcacggcactagtggatgcatggttccaaattaaagtaagccaatttttcaactccccaaaaatgtagttgttgtagaaagttgacactaattggaagacgtacgaaagcaaccttaattgagttgaatatgtgatgcattaatcatagggagtttaattacgttggataatcttgaaaagtttaatttagggatattaaatgacaaaatttcttttttgacatataagaata

SEQ ID NO: 79 - DNA, 6217 bp, Malus domestica
  misc_feature 1..6217: note = source = /organism="Malus domestica"/mol_type="unassigned DNA"
  source 1..6217: mol_type = unassigned DNA
agccaattagggttttacccaaaaattaaaaagaaacccacaaaataaatcaagatttgttttccaaagaagcaccatcataatttgactagtaatttaaggaaccaatatgtaactcacacatatatatatgattcaataaaaatataagagactaaaaagagtgcccaactgataactaaaacttgatttacatgatatttttttaggaaacaaatcaacaatcgcatgagattcgatcaatttggtgaatatatttatatttgggtggagaggaccaatatgcctcacatgtatgtgactagttcgacgaaagtccacaatggcagcagctcttcttgttgccttccagccatggttgaactttagaaccattaatttatttttctcaggaaaacattaataaaaaaatgtcaacacatattatccaagattcgtctgtaagttattttgtctaaattgcaagagctgaactcatttcttgtaagtttagttttatttattagtccttttattgaaggaatttcagataatcaattccatttgctgtacgaaatctctgttaattaacttaattacatgtatatctctttcgtgttaatcagtttaagatgtacttaggtttccaagacatataaattatttcaaggcgccacataaggtatatcttataaccaaaccatgttttcttgtaatcatgtatatgcataataatattttattttgacaacatgatatgatgtctataacgtaaaagttaatattgttataacaataatgatgtcattatgtgttatgtaactaaaatcttactattattgtgtaaccttagactataaatcttaacgattatggtgtaattaaccttatatttcttgtagtccagacgttgaatgttaacattattttataacaccaaatgtgaaggtaaagagtcaaaagtgaatactgataaatgtgagatttcttgacgtctatttttaataacttgcgtctataaacgctatactataacaagacattcataaccatcaaacattagtgtaacaatatcattccataacctcgatcactatttatgtaacgttgaatagtaaatgacaattataatataattgttgttatacaaatggtggcgtagaaatttgctttattaatttgggacttatttgtaaaaatatcagttgttttacttcagacaaagtcaaattgaagcaattttgtgacgttgttcctatgagtagactctatcgttcctatgaggagactctacgaactctctcatttcttgtttgttttaggtggagcgtcttactattaactaattaatactcccagtccacacttcatatatttataatcaaattcytacgtctctactatgattggatgaaagaaataaacttagaatttagataaaaatcagaatttataaattgatatacaccaattcgcttgtttgaatttataaacatagaaatttagaatttcagcgtgaaagaaaaacttggaatttaggacctccaattcccaagtttaaattccatgtaaataggtatcatttctcaatttctatgattgagagtttaaaattaacaaagttcattctcaattyyattgttcttttaggttaatcaaataagaaaattcataaattttagaaaataaaatctcgtcattttaaaatttcttaataactttaaatttctccattcaaacatagtataaacgtcgaaacggctcgaatttggataatgagaaaaaaaagggattcaatccaaaaatccccaaacttggtttcttgtcccatactatttcttttcttttccaactttcataccataaaaatactataaacaaaactaaacaatctcaattgtttttaaatagtttatttctcgatagcctgaccaaattccactgtagcactaaaaaaataaataaataatccaacagttgcacaaactttgtatataaaaggcaccataatatctcttccaacttagcaacatttggaggttgccctgctacttctgctaatcatacccacgaaaaaaggtaaaaacaaaaaacaaaagaaacacacacaatatggattatcttctgcagtccttgcaacccttacaatccatgacaccactcttgctcataatcccactcctatttctcctccctctaattttccgtttccggcgaccaccaccatacccgcccggccccaaaggcctaccccttattggcaacatgttattgatggaccaactaactcaccggggcctcgccaagctggccaagaaatacggcggcatattccacctccgcatggggttttttacacatggttgcgatttccaaccctgacgtggcacgacaagtccttcaagtccaagacaacatcttctccaaccgcccggccaccatcgccatcagctacctcacctacgaccgcgctgacatggccttcgcgcactacgggcccttctggcgccagatgcgtaagctctgcgtcatgaagctctttagtcgcaaacgcgccgagtcctgggagtctgtcagggatgaagtggactcatcggttaggaccgtcacagttcatgttggttcggctgtaaacatcggagagttggttttttcgctcacgaaaaatattatttatcgggcggcgttcgggacgagctctcaggaagggcaggatgagtttattgggatactgcaggaattttccaaattgtttggagcttttaatattgctgattttattcctagcctggggtgggttgatcctcaggggctaaacaatagactcgctaaggctcgtgagtccttggatcggttcattgacaccatcatagatgatcacatggagaagaaaaggaatggtaagggagtgagtgacggtgaaacggacatggtggatgagttgttggctttttacagtgaagaagctaaagtatatgaatctgaagataatttgcaaaacgccatcaaacttactagggataacatcaaggccatcatcatggtaaataactattacacaatttcatatcacatatttatattataaccccacaggttgaatgtctatgtaacctgttttcttggactaaacttttggctaattttttctttttatctgatcagatatattaattgcgtattgagacaatgaagatagaaagtctaacatattatattattttgaaactgattgtttaaattgacgatctgactaggtatataacatgctgtatatgcaaattaataagctaattatatatgctacgtactttcaggacgtaatgtttggcgggacggrgactgtggcgtcggcaatagagtgggccatgtcggagctgatgaagagcccggaggacctaaagagggtccaacaagaacttgctgacgtggtgggtctagaccgtcgacctgaagagaccgacttcgagaagttgacatacctaaaatgtgccctaaaagagacactacgactccacccaccaattccactactcctccacgagacctcggaggacgctgtagtagccggctaccgcattcccaaaagatcgcgtgtgatgatcaacgcgtgggccattggacgtgacaaggactcgtgggaggacgccgagtccttcaagccctcgaggtttttgaaagaaggtgtgcctgactttaaggggagtaacttcgagttcattccgttcgggtccggtcggaggtcgtgcccgggcatgcaactagggttgtacgcgctggagatggcggtggcacacttgcttcattgttttacgtgggagttgcctgatggtatgaaacctagtgagctcgacatgaacgacgtgtttggactcaccgctccgagagcgagtcgactcatcgccgtaccgagtaaaagggtggtttgtccactctgatggtgatgaaaaaacagagagagaaacagtggtggaatttgtttggagtgtaatgatactgtgaaaactgcacaggtaaattgcaagcgttttcttattgttcttttgaaaaacaaaataattacaaagagaacatgaacagttgtgagggaatttttwattcactctctttgtgtatatagacactctcttttgttttgtttaatatgtatgcaaatttatcgttaatttcctkkttttttcctttttgttttkttattattgtattgaaataaaaatataatggctgagttacaaaaattatttgtcatggtggaaaaattgatggactgcttatgttacgatttcaatttgttttctgggtcggtggcgcaatagtagaattattttgtttaaaaatgaatgaataaagattaaataaaattagctcttgaacaacggtagaataatgttaaatcatgcttacgatagaagtagctcacgctgtcctcagcagttgtgaaaattgacatttaagttgactgttgcatgtcacttatatatttatttgtggttaaaattgttacataaaaaatatgaagagtaatgctgtggaggttaaatttatagactaaaattgcaaactaaattatgtgtcactaataagaaattagcacttttatcaacgtttaagtaataattcaatcatcaatatccatatcatttaatttacaaaacttagtctacaaatttaatctccctagcattactcaaaagtgaatgattgtgaaaatgaattaaggttggtaggactttgacatataaatctaaatataattaagaaggctctagggtagtgctgttcacacaccttttttwacttctctcactcttattaattttgtttattgatcttcttcaattaattcaatccggcgaccgaaaattgaaaatagtatgtgagaagtaaaaattagtgtgtaagtagcactacccgtggctctatatatggtgcccaagtcaggttgatgcattttggtggctccatatttttgcaagacaagtgccctagctagtgacccgaagagatcttgtttatgaactagttgtatacaactgtttgttatctctcatcatgttaaaaagttaattaatatatagacaaaattatctgtaacatactagtaattaagaaagtttgtaaccaattgtgtataattatgtatctaccttaaatgtggtataagaccagaagaagtaccgtatatcatataacatacttgatggtgcattccaaccgatttatgtgtggaggaagtgtcgtaaatgccttgttgagctgcatcagcatctgtttgtgtgcgcgcgcgcgcgtgagagagagagagagagagagagagagagagagagagagagagagagagagagagagagagagagagagagagagagaatgaatatattgagtttaggtgggcagtgaaaagtgaagtgaggaggacgtgaggccttttccttgtactatgaagaggtaagtggaggagcatgccggagaaatcgagaaggggaagttgagcagagtttggtaggagaaggattgcaagaagaaattaattttgatagaaaagagattaatgttaatagaaaagaaaataatttaataaaaattatagtatttttttatttgtaatctaatactacatcaaatgcttcactaagtcaatctaattaagtctagtccttgaagctaattcagtccgaaacagtccggtgcaacaaatgccctctatgtgtatcaatgatcatgaatcaaaataatatgtataaaaaaatatataaatatgttgtgatttagggacacgtttgtgacacattttgacacacatttttatggaactcactctgtaatgtattttagggatccagccgtctatcttttatgtcttaattttttttatgtcttatcttaaagataatgtctaccaaaaaccacctaaatttgaaaccatatgattgttgattgaatttagtgtttcattgtagaacaatattcgtttattttcttccctacaaataaatgtcttaatagtcttggatttgcataattttttataagaatgatttataaatgatataataaaatatagacaattctgatcgtcgaaatacatataaattagactctacgtactataaaaatagatgtcaaaatatatgccttcgtgtattgtaactctctacatatgtgtgtgtgaaagcagattgtgtttatgtaagtaagagagagctggaaagggacgccatccgatattttccatcct

SEQ ID NO: 80 - DNA, 6188 bp, Malus domestica
  misc_feature 1..6188: note = source = /organism="Malus domestica"/mol_type="unassigned DNA"
  source 1..6188: mol_type = unassigned DNA
aacctacaaaataaatcaatcaagatttgttttccaaataagcactatcttattttgagtcataattttggtagcataataaacaatatattgaaagtataatataatttatattttaagatataatataattttattaatacgtacagcttagaaattagtttataaatattcaattgctttactttagacaaagtcaaattgaagcaattttctggggtaatgttaggattctttataaattaaattttgtaaactaaatgagatggttgttaatgattaaattattatttaagtattgattaccatacttatttcttattgatgacacttcatttaatttaaaattttagtctccctatcattatctgaatggtcgtttttgttagtaaatgcaaccaactctcttatttcttcgagtttagaatatttgtattttttacgtattaatcaatagtactattctcattacatttttcataccagattttgtaaacctctctaataaagtgagactcacatgtgttgatgggttaatacttctactaaaaaagtaatgttaggagaccaaattttttaaactgaattgcaaatcaaatgacgtgtcaccaataaaaaataaacatgttaatcgacacttaattaataatccaatcatttacaaccacatcatttaatttgcaaatttggtttaaaaaaattggtttctccaatggtggcttataacctaaatttcccctyccccccccccaccccaaaatccactccaacccaaaacataaaccagacctaaaacctaaaacttaaaatgaaggtaaatataggtcacaaatttagcctacaaaagccgctgagacccacggatgagagtgaaaagtgagcattgtcctgtagccaactgctacttttcttcatcgggtggtggtggtaattggaccgttggttaatccaacggtccacattcaattttttttatatttatatatttattgaatccaacggcttaaatcgaatataatcaaatctaacagtaaaagaaaagatctaacggtccaaatttaaatccaatggctagaataatcttaaaatttatcggaatttaaatatttttttccaacggctagaataatttttttttttttaagtttatgtggtttatcatgattttcatgtattgatttagtgatttttcaaaatttatcggaatttaaatatttttaggttaaaatgttcataaaattaatttaggatagtctacataatttttaaaaaaaaaaatagccttaattcattttttgataatctagggctaaaattttaggccaaaagggttagagtagaaaagctgtttctgggctaaaacctaaattttctgggctaaatatttttaggttttaggtcaggattggagatgatctaacattactcctactaaaaatatggtacaaatatagtatgaaaaatgctataagtgtagtatgacttggtatttataccaacaggaccatctctgaatcctttggtgaggatcttgaggatcattgaatcgtgtctgtttatcgtacatcgtgcagttaatttttgtcaagtactgtttgtgtttaattttaaatgaaaatatttaaaataatttctgaccgcatgatgtacgataaacagacaagatcgacggatccttgggatcctcacaaaaagaatctcgcgaggatccggattcgtatttatatgccaaagcggactcggacctttggataatgatcagggaaaatgacaattcaaaaaatgggtaaacttcttatctcatccatagcactattactttttctttacagacaaaatactctaactttcaaaccatacaattttaaaaacaaaaaagagaaaactccttgaattagttcaattcaagatagcatgacccaatgaaacagcaacaccaaaaaattaaaaatatataaatcaaacagtagcacaaattttgcatataaaaggcaccacaatatctcttccaacttagcaacattttgagttgcccttaaaatccacacactatggattctcttctgcaatccttgcaacccttaaaatccatgacaccactcgtgttcataatcccactcctattcctcctccctctaattttctgtttccggcgacgaccaccgtacccgccgggccccaaaggcctacccctctttggcaacatgttaatgatggaccaactaacccaccggggcctcgccaagctggccaagcaatacggcggcatattccacctccgcatggggtttttacacatggttgccatttccaaccctgacgtggcacgacaagtcctccaagtccaagacaacatcttctccaaccgcccggccaccatcgccatcagctacctcacctacgaccgcgcagacatggccttcgcgcactacgggcccttctggcgccagatgcgtaagctctgcgtcatgaagatttttagtcgcaaacgcgccgagtcgtgggartctgttagggacgaagtggacacggcggttaggaccgtcacagttcatgttggttcggctgtaaacatcggagagttggttttttcgctcacgaaaaatattatttatcgggcggcgttcggtacgagctcgcaggaggggcaggatgagtttattgggatactgcaggaattctccaagttgtttggagcttttaatattgctgattttattcccagcctagggtgggttgatcctcaggggctaaacaatagactcgctaaggctcgtgagtcgttggatcggtttattgacaccatcatagacgatcacatggagaagaagaagaagaacaataagggattgaatgatggtgagacggacatggtggatgatttgttggctttttatagtgaagaagctaaagtaaatgaatctgaagataatttgcaaaacgccatcaaacttactagggataacatcaaggccatcatcatggtatataactattacacatttttatatcagatattataaccccgcattcaggttgcatgtctctgtaacctgtattcttgaactaaacttttggttaatttattctttttatctgatcagatataataaattgtgttgattgagattatgaagatagaaagtctaacatatttaactttaaaaattaataatagttgaggttgacgatctgactacataacatgttgtatatgcaaattagtaagctaattgtatatgttatgtacatgcaggacgtaatgtttggcgggacggagactgtggcgtcggcaatagagtgggccatgtcggagctgatgaagaacccggaggatctaaagagggtccaacaagaacttttaaatgtggtgggtctagaccgtcgacctgaagaggccgacttcgagaagttgacttacctaaaatgtgccctgaaagagacactgcgactccacccgccaattcctctactcctccacgagacctcggaggacgctgtagtagctggctaccacattcccaaaaaatcgcgcgtgatgattaacgcgtgggccattgggcgtgacaaggactcgtgggaagaccctgaatccttcaagccctctaggtttctgaaagaaggtgtgcctgactttaaggggagtaatttcgagttcattccgttcgggtccggtcggaggtcgtgcccgggaatgcagctagggttgtacgctctggagatggcggtggcacacatgcttcattgttttacatgggagttgcctgatgggatgaaacctagtgagcttgacatgaacgacgtgtttggactcaccgctccgagagcgagtcgactcgtcgccgtaccgagtaaaagggtggtttgtccactctgatggcgatgaaaaaacagagttggattgaggagaaagaaacagtgtgcaacatgtttggagtgcaatgtcaaagtatacatacagtaaaaacggcaggtaaattgcacttgtttttaattgttcttttgaaaaacaaaataattacaaagagactttaaaaagtttgtgggactttttatttctctctctttgtgtatatagacagtctcttgttttgtttaatatgtaagcaaatttatctttaatttccttttttttttgttttgttattattgtattgaaataaaattagtggatgagttgcaaaattatttgtcatggtgtaagaccatctccaacctttggattaaaacctaaaatttttagtccagaaaatttagagtttaactgagaaacaacttttttgctcaaacccttgtagattaaagttttagtcccagattattaaataatgaatataggctcttttttttaaagttactttaaaaaaaaaattatgtagactatcctaatctaattttatgaacattttaacctaaaaatatttkgattccgacaaatattaaaaaatcactaaccgacaccatgaaacttgtgaaagaattttttttaattactttagccgttagctttatatttggaccattcgaatttttttattcttttaccattggatttcattaaattagatcttaaccgtttaattcaataaatttataaatataaaactaaaaaaawwatacatatatagtgagccagtctttctaactcatggggaatcttgagctaaatttagcccataaatgagttttgagttttaactcatatttgccctaagaccatctccaatggtggcttataacctaaattttccccttccccccccccaaatccactccaacccaaaacctaaaccaaacctaaaacctaaaacttaaaatgaaggtaaatataggtcacaaatttagcccacaaaagctgctgggacccacggatgagagtgaaaagtgggccttgtcctgtagccaacggctacttttcttcatcgggtggtggtggtaattggaccgttggttaatccaacaatccacattcaattatttttttgttttatatttatatatttattgaatccaacggcttaaatagaatataatcaaatccggtaaaaaaaaaagatctaacggtccaaatttaaatccaatggccagaataatcttaaaatttatcggaatttaaatatttttttccaacggctagaataattttttttttttaagtttatgtggtttatcatgattttcatgtattgatttagtaatttttcaaaatttatcggaatttaaatatttttaagatagtctacataattttttttttaaagttaatttaaaaaaattagccttaattcattttttgataatctggggctaaaattttaggccaaaagggttggagtagaaaagctgtttctgggctaaaacttaaattttatgggctaaatatttttaggttttaggtcaggattggagatggtctaagagttggagcaagttggggtaaatttgtaacttaaaatttgagctttactccaagggagtaggtctaataattgatggactgcttatgttacgatttcaatttgttttttttttttttttttttgtggtcggtaggaatggtagatttatgtacagctgtgcttacgatataagtagctcatactgtcctcagcagctctgaaaacagacactaattgactcatttcgattatacttatatagtggttgaatttcttatattaaagatgtgatattgtgaggatggtgagaatgaatgaatgttggtgaggacgttgacatataaagctaaatataatttagaaggctgtatatatggtgcccaagtcagatcaatgtcttttggtggctccatatttgcaagaaaagtgcgccttttgtaaatactatttatttgttatataagcgtgtccttgctagcgacccgaagagatcttgtttacgactagctgtgtacaactatgcgttatcttatacatttatacaactgtgcacacgtatagaattgtccatatataattagtaaaagaatttcttgtttgtaaaattagttttgtacaactatatgtgatctgtgtakatgatcagaagaagtactgcaaagcatatatataggacagtgcactagttaacctacgcgtgcaggaaatgttgcagtgactgcccttgttgagttgcatcagcatgtgtgtactttgtacaaccgagagagagagagagagagagagaga

SEQ ID NO: 81 - DNA, 5918 bp, Malus domestica
  misc_feature 1..5918: note = source = /organism="Malus domestica"/mol_type="unassigned DNA"
  source 1..5918: mol_type = unassigned DNA
acatctcaaagcccaaccccaagggtgagcccaaatttagtctccaggcctattactctaatcaatttctataaaraacaaagccttgtaaagtaataaaatcttttgggaatgaccaatgtgggacaaagaaatttctactcaaactactcaaatttccaacaataccccacatttgagttgaaatttcattcaaactycawcaattaccccacatttgaatgcaaatggatgactaggctgcytawamctgagagwgaatagacatratatgcatcgagtgaagtgtcttttggacttgaacttacacagatcatgtctgycayacgtaaattcatacgmttgtgttcattttggccctgaacagatcccggatttcgtaggagctttagagaatttagccatgtcatttctcataaatgtgggccctaaaccccaaggcccatcttttgacaataaatatatattaattaccaattaattagtataccccaaagtccaaccccaagggtgagcctaattttaatctccaggcctattactcaaatcactttttataaaagacaaagccttataaagtrataaaatcttttgggaatggccaatgtgggacaaagaaatttctactcaaactactcaaatttccaatagacagagaatcagtttgttgaggttgtttagcccccttttcttcacttaacgacacactagacaatccaccacttgcctgtttatccccattgaattaccgaaaataaaaataaaaggtcagagtttcacttacatagcaaattaatacatatattcaagtggcctggggacaatgtggacaagggatatattcaagtcgtcatcttctatgcccaaccattgcgccttcgtaagttggtgcataaaggatacacttgttcagcatagctttcattgactgatctgagaactcgtgtcggaaaattttgacatttaggggtacaaggtatgatcacagaaaactagagttctcccagtggcgagagaaactctgcaatccatcataagtgaagtagtctatctctctcatcatgtgtcaccaggggctcaaagtaagggcgactgggatttatcagtaaccatgcattgatacaatatcattattcctcttttgcctctcaaatatagcaaaagggaagcaaaacaaggtcaactgtgaatcaacattttcttgctccattcagtttactaatcgttcgttctaacatcaaattgccatgaccatagcagctaaaaccctgtaacagatctagcatcacactgccgtaaaaagaatagtgatctctgagtttctgtatctcatcgataacaaaatacctacaatcgtatgtgtcaaatatttattttattaatatattcggtcccatagttcttgggagcagcctctccataaatgggggtaaggctatccgacattcacctctcccagaccctgcgtaaagcgggagccttgtgcactggatacgacctattcggtcccatagttacattaattattaaaaaataattaaaaatcaattttaatcctattggttgtcaatgagactcatgaattaaacaattaaattaaaggcatttgactccatctttatttgcggtaaaaaatgtcatatatgacatctcctttagagaataccgtgatgtcttttaatcttagttgacgtatttgacatctcttttacatggtaagaattcaaattattcttcccttcttttgtcgaaattcaattaattccttaattaactggggtataatctgtttccagacaagtggatccaaaattcccactggcagtatacacaaaacaactcgtgacgtttgcaagataaaatacatagcagatatcaccaccaagtcaccagttattacttgccactttagggctgcagatcccttattaattatataaagaactctcatagttttatatgaacctcaccaaccccattttcccaacacaaccaaagccatggaactccttcaccaagctcttcaatccttgcaatcttcacccatgttgcttattcttcttcttcttctcctcatcatttcctttatttttctgtttaattcgcgccggaaacccccatatccgccaggtccaaaagggtggcccatcatcggtaacatgctcatgacggaccaacttacccaccgtggcctggcccatttggccaagcaatatggcggtctcctccatctccagatgggggtcatacatgttatggccgtatcaactcctgatatggcacgcgaaatcctccaatcccaagacagcttatttgccaaccggccagccaatgttgccatatcttacttgacctatgatcgggcagacatggcctttgccaactacggcccgttctggcgtcgcatgcgcaaaatttgcgtcataaatctatttagccgaaaacgggctgagtcatgggcttcggtgcgcgaggaagtcgatgagatggtccaaaccgtggcggggaaaaccggctcgccggtgaatatcgggcagttggttttcgctctaactaggaacataacatatcgggcggcttttgggtcaagctcacatgaagggcaaggcgaatttgtgcagattttgcaagaattttcaaagctttttggagcttttaacatgcaggattttctaccttggttgggttgggttcatgcacagggttttcaggacagaatggctagggcacgtaaatcactagacgttttcatcgacaagatcatcgacgaccacatggctaagaggaaagcaaacatggagaaggatgacagtgaggctgctgatacagatatggtggacgaattgatagcttactttagtgatgatgctggaaaggaaggtgacgaccccaattctggcttcaagctcaccagagacaatatcaaagcacttattatggtatgtaaatttgacaattaatattttcttaatacgttatatttgttggtgaatagtcacacatcagagaaaaacaaaaataatatataatttttatatttgggacataaacttctaatatcatcgagattttccaatgataaaattcaacacatagcaataagtggtatgcactttattaatttttaagaaacaaatctgattttaagtatttgttgttaactattttggtaaggaattaggagagaaaactattctgataaggaattcatgtacgtaactattactatatttatcatttccgattgatatcgattgatttagtactaaataatacgattatcccatatatacgtaggatgtgatgtttggtggaacagaaacggttgcatcagtgatcgaatggacaatggcagagctgatgaagagcccagaagatctcaaaagagtacaacaagagctcaccgatgtggttggattgaaccgtaggctccaggaaaccgacctcgaaaacctaacctacctcaaatgcgcagtcaaagaatccctccgtctccacccgccaatccctctcctcctacacgagaccgtggaagacacctctgtggctggctactcgttcccagctgggtcacgggtttggatcaatgcgtgggctattgctcgtgacccgactgcatgggacgagccagaaacattcaaaccctcgaggtttctggaagatagctcgcctgatttcaaagggagcaacttcgagtttattccttttgggtctggtcggaggtcgtgcccgggaatggcgttggggctgtatgggttggagatggctgtggctcatctgcttcattgttttgcatgggagttgccgggtgggatgaagcctagtgagcttgacatgaatgatgtgtttggactgactgcaccaaaagcggttcagcttgttgctgtgccaacttacaggttgaattgccccctttgaattaatagtttcaagtagacgaggagacgatgatgatgaaatattacttggttttaaatcagtgatcgatgagtcaatttctctacgtaaaggtaccttaactaataagggtttgttatgttcttggtctctctttactaattggataatactattgctttcccccatgggggatgagcttattattcctctattgcaaataacaatatcttcaacatagaaatttcataatcggaacagtccaatatcttaatcttcatttgaagatcatccttacaaaaaataatttgattttgtgatcggttcgtcatccaaatgtatcagtaaatcaacggttcatcatgaatatgctacttgatacctacacttatgatttatttgatacatttgaatggctaaacaatcacgtcaaattatttttttggagggatgatcttaaaatgaagattaagatattgaatggttctaattataaaaaaatctacgttaaagatatgttatttgcaactggagaaatgacctcatctcccatggagagtatgtaagcagtatccattattgtaataatcatttttaattttgtgatcgatataatatcactatgttgttaatagttaacataaatatatgcgtagttgtttgttaacctttgttggctttctgtcctttgtggctcggccgtgtatcttttagtcagttttccgaaagacaaaaagggaagagatcctatccggatctctcccaccaaattctagggattcgaagatccggactcttaaaattgaattcaatggccacaaacagaggatctctctaaaagttataataattacaatagttggatcaaatttcaaaggtttggatcttcggatccctaggacttggtaggagagatccggaagggatctcttccctacaaaacaagtggggatgtacttttaatgttggactgttaaagtcctggaaaaacatcttttgtcaaactatatagcaaaaatagtagtaagctaagatgcttatggtgttagattatggatcgaggttattgatcaagatgaatctttcctaaatttataccaaatttataatctcacaacataatttgatgaactcgatatgcaatattgatcacacaaagagttcatatagaatattggtttatatcataaaaaattaaatttgtagggttcgtaaactgttctattaggtagtagtaacatattaacttgatattgtgatgatacaaggctaaaattaatagtattgttatcaactaactattgatctagtaattttttttcttccctaatgttgaaataaacgtcttgagctcgattcctctcttttatacaaaagataatataaacagtagcatagatttcaacaaatggccgttgaaataaacattttgagttcggttcctcccttctacacgaaagctaatataaatcaatagcatagctttcaacaaatgaccgttgcatttagatgaaagcccttaaagtttgccgaccctttgacatgtggataagtaaataatagaactaggtaattgaagtggttgagaaatggacattgtgagattggaatgcgttagcaattgcgcaatgcaaactatgctgtcggtagaaggtgaaaggataaacctcagccaatgacattctatgcagctgtttagctatagctttttgaacctgtacaaacgatttttattttctttttaatatttaaaccaagaaatataaattttgagtccaacttcaaggtagatgatcgattactcatcaaataaacttatttattccaatcatctttaaaataagaggaaaaagaaataagcaaagttccgttgttgagcaagaaatgaaaaattcatagaaaggtataatctccaccacgtaatgtgcacctacacgccttcattgacgtcgtttctcaattggatagataaaagttttcttcaagtctaaaccccctttcatcaagtggtgaattgacac

SEQ ID NO: 82 - DNA, 6115 bp, Pyrus x bretschneideri
  misc_feature 1..6115: note = source = /organism="Pyrus x bretschneideri"/mol_type="unassigned DNA"
  source 1..6115: mol_type = unassigned DNA
gctcttcttgttgccttccagccatggttgaactttagaaccattaatttatttttctcaggaaaacattaataaaaaatgtcaacaaatattatccaagattcgtctgtaagttattttgtctaaattgcaagagctgaactcatttcttgtaagtttagttttgttcattagtccttttattgaaggagtttcagataatcaattccatttgctgtacgaaaactctgttaattaacttaattacatgtatctctctttcgtgttaatctgtttgagatgtacttaggtttccaagacatataaattatttcaaggccccacataagatatatcttttaaccaaaccatgttttcttgtaatcatgtatatgcataataatattttatcttgacaacatgatatgacgtctataacgtaaaaattaatattgttataacaataataatgtcattatgtgttatttaactaaaatcttaccgttattatgtaatcttagactataaatcttgacgattacgctgtaattaatcttacatttcttgtagttcagacgttgaatgttaacattattttataggggcttttagatccaggtccaaaaatgtagagtgtttttaggagtgagtccaattatctaattatatgtttttatttcttttatactcattttatattttctaaaaatattaaaaatcaattaaaatcttctaatattatgcattttccaactccaaaagaatatatttaatatcttataacaaaaaaaactaatatactaaaataaatttatctgcaaaacattctaccctaactcaagtaacaaatatatgaatgtatataatatacctatacgtgagatatgaaacctaattagagggtaaaaaaaaaataacatacctacaagtaagacacattaatctgtgacaggttgattataaaaaaagaatctgtcatataggtagataaacataattaacctgtgatataggtcgattataaaaattaaaaataaaatctataaatggggtttaaagtaggaggaagaacgagaaataacaaaaagataaataaaaagaaataagcaaagagataattgggaagaaatttgatttttataataaattttatcattgaacaaataactattgataattaaataattaaatataacaaattggacttattttaataaactggactattcctactattggctcaaaaaattggacttatatcaagtttcccctatttttttttataacaccaaatgtgaaggtaaagacccaaaagtgaatactgatgaatgtgaaatttcataacgtctatttttaataaattgcgtctatagacgctatactataacaagacattcataaccatcaaacattagtgtaacaatatcattccataacctcgatcactatttatgtaacgtttaatagtaaatgacaattacaatataattgttgttatacgaatgatagcgtagaaatttgctttattaatttgggacttagttgtaaaaatatcagttgttttatttcagacaaagtcaaattgaagcaattttgtaacgtggttcctatgagtagactcaaccaactctctcatttttgtttgttttaggtggcttcttaaaactaactaattaatactcccatgtccacacttcatatatttataatcaaattcctacgtctctaaacgtcaaaacggctcgaatttggataatgagaaaaaaaaggattcaatccaaaaatccgcaaaacttggtttcttgtcccatactatttcttttcttttccaactttcattaccataaaaatactataaacaaaactaaacaatctcaattgtttttaaatagtttattactggatagccgaccaaattccactgtagcactaaataaataaataaataatccaacagttgcacaaacttcgtatataaaaggccctactacttctgctaatcatacccacgaaaaaagaaaacaaaaaaaaacaaaaaaacaaaaaaacaaaaaaaacaaaagaaacacacacaatatggattctcttctgcaatccttgcaagccttacaatccatgacaccactcttgctcataatcccactcctatttctcctccctctaattttccgtttccggcgaccaccaccatacccacccggccccaaaggcctaccccttattggaaacatgttattgatggaccaactaactcaccggggcctcgccaagctggccaagaaatacggcggcatattccacctccgcatggggtttttacacatggttgcgatttccaaccctgacgtggcacgacaagtccttcaagtccaagacaacatcttctccaaccgcccggccaccatcgccatcagctacctcacctacgaccgcgctgacatggccttcgcgcactacgggcccttctggcgccagatgcgtaagctctgcgtcatgaagctctttagtcgcaaacgcgccgagtcctgggagtctgtcagggacgaagtggactcggcggttaggaccgtcacagttcatgttggttcggctgtaaacataggagagttggttttttcgctcacgaaaaatattatttatcgggcggcgttcgggacgagctctcaggaaggacaggatgagtttattgggatactgcaggaattttccaaattgtttggagcttttaatattgctgattttattcctagcctggggtgggttgatcctcaggggctaaacaatagactcgctaaggctcgtgagtccttggatcggttcattgacaccatcatagatgatcacatggagaagaagaggaatggtaagggagtgagtgacagtgaaacggacatggtggatgagttgttggctttttacagtgaagaagctaaagtaaatgaatctgaagataatttgcaaagcgccataaaacttactagggataacatcaaggccatcatcatggtaaataactattacacaatttcatatcacatatttatattataaccccacaggttgcatgtttatgtaacctgttttcttggactaaacttttggctaattttttctttttatctgatcagatatattaattgcgtatcgagactatgaagatagaaagtatatattatattattttgaaaactgattgtttaaattgacgatctgactaggtatacaacatgctgtatatgcaaattaataagctaattatatatgctacgtactttcaggacgtaatgtttggcgggacggagactgtggcgtcggcaatagagtgggccatgtcggagctgatgaagagcccggaggacctaaagagggtccaacaagaacttgctgacgtggtgggtctagaccgtcgacctgaagagaccgacttcgagaagttgacatacctaaaatgtgccctaaaagagacactacgactccacccaccaattccactactcctccacgagacctcggaggacgctgtagtagccggctaccgcattcccaaaagatcgcgtgtgatgatcaacgcgtgggccattggacgtgacaaggactcgtgggaggacgccgagtccttcaagccctcgaggtttttgaaagaaggtgtgcctgactttaaggggagtaacttcgagttcattccgttcgggtccggtcggaggtcgtgcccgggcatgcaactagggttgtacgcgctggagatggcggtggcacacttgcttcattgttttacgtgggagttgcctgatggtatgaaacctagtgagctggacatgaacgacgtgtttggactcaccgctccgagagcgagtcgactcatcgccgtaccgagtaaaagggtggtttgtccactctgatggtgatgaaaaaacagcgttcgacgaggagagataaacagtggtgcaatttgtttggagtgtaatgatactgtgaaaactgcacaggtaaattgcaagcgtttttttattgttcttttgcaaaacaaaataattacaaagagatcatgaacagttgtgggggaattttgaattctctctctttgtgtatatacactctcttttgttttgtttaacatgtatgcaaatttatctttaatttcctctcttttttcctttttgttttgttttgttattattgtattgaaataaaaatagaatggctgagttacaaaaattatttgtcatggtggaataattgatggactgcttatgttacgatttcaatttgttttctgggtcggtggcaatagtagaattattttgtttaaaaatgattgaagaaagattaagtaaaattagctcttgaacaacggtagaataatgttaaatcatgcttacgacagaagtagctcacactgtcctcagcagttgtgacaattgacacttaatttgactgatgcatgtcaattatatatttattttgtggttaaaattgttacataaaaaataagaaggataaggttaaggagattaaatttatagactaaaattgtaaactaaataatatgttatcaataaaaaatgaacacgtttatcaacgtttaagtaataatacaatcatcaactttcatgtcatttaatttacaaaatttagtcttcctagcattactcaaatttgaatgattgtgaaaataaattaaggttggtgaggactttgacataaatctaaatataattaagaaggctctaaggtagttctattcacacactcgtttttacttctttcacactcttattaatttttgtctattgatcttcttcaattaatttaatctgacgatcagaaatcgaaaatagtatgtgagaagtaaaaattaatgtgtaagtagcactacccgtggctttatatatggtgcccatgtcaaatcgacgcattttggtggctccatatatttgcacgacaagtgccctggctagtgaccatgaactagttgtgtacagttgtgtgttatctctcatcatgtggaaaagttaattaattaaaaaaagtttataacgacatgtgtacaactatgtatcgaccgtggtataagaccagaagaagtactgtatatcatataacataggatagttggcttgatggtgcattccaaccgatacatgtgtggaggaagtgttgtaaatgccttgttgagctgcatcagcatctgtgtgcgagagagagagtgaatgaatatattgagttttggtgggcagtgaaaagtgaagtgagaaggacgtgaggccttttccttgtaccatgaggatgcgtttgttacaccttcttaagagattggcttaaactgacttaactagaactataattctacgtttggtatgtttctggactagattaaatggaactcgctccaactaacaatccctccacgtttcgttaaagctcgttatttagttcggagctttcgcttggtattaagcatgactaaaatggaaaacgaggccgacgcttggtcgtttgctacacccgacatccactctgccttctttctgcttgcaccgttactatggccaggtcgtcggcggtgccttctcttatgtggcccaaccttaggtgatagctgcagctcacagctgaagaggcgctgctttaggccaaaatcgcgagtcggtggaggacctggcaatgctaccgtcggtgggcgttgctgacgagaagcagatcaagagcttgacgatgaaggttgggaagatctgtacaatattatgcgatttgattatttggttgcaggattgggagatctgtacattttttatgcgatttggttgctgacatgattgaatgagaaaattaattagcttttaaaagttaagataactataaaataagtgagagagtggtgaaattgtggagataaaaaggtaagtggaggagcatgccggagaaatcgagaaggggaagttgagcagagtttgatgggagaaggattgcagtaagaaattaattttgatagaaaagaaattaattttaatagaaaataaaataattaaataaaaattattgtacttttttatttgtaatccaacactacattaaacgtttcactaaatcaatctaattaaatttagtccttgaagctagtccagtccgaaatagt

SEQ ID NO: 83 - DNA, 6148 bp, Pyrus x bretschneideri
  misc_feature 438..586: note = /note="n = a or t or c or g"
  misc_feature 1..6148: note = source = /organism="Pyrus x bretschneideri"/mol_type="unassigned DNA"
  source 1..6148: mol_type = unassigned DNA
aaactaatgaatgatattgtggtgatgctacaacacgcaatctaatctaatcttgtaatcttatcttaggtttgaagtacaaattaaccccatttatcatatattgcagtgaggtgaaaaaggtttgaaatggtgaaatgaaaccgtcaatggctggaaagtctaaaccctgacagctacttggtatgtctaataaggttgagcatcaaatctatcacatttcagatacaatacttactaataaattcatcatgcatgattcacattgaagagatcgggggttaggtgtagccaaatagggttttacccaaaaattaaaaagaaacccacaaaataaatcaagattgattttccaaagaagcaccatcttaatttgacttgtaatttaaggaaccaatatgtaactcacacacacacatatatacatatatatatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttaaggaaacaaatcaacaatcgtatgagattggatcagtttggtgaatatatttatatttgggtggagaggaccaatatgcctcacatgtatgtgactagttcgacgaaagtccacaatggcagcagctcttcttgttgccttccagccatggttgaactttagaaccattaatttatttttctcaggaaaacattaataaaaaatgtcaacaaatattatccaagattcgtctgtaagttagtttgtctaaattgcaagagctgaactcatttcttgtaagtttagttttgttcattagtccttttattgaaggagtttcagataatcaattccatttgctgtacgaaaactctgttaattaacttaattacacgtatctctctttcgtgttaatctgtttgagaagtacttaggtttccaagacatataaattatttcaaggccccacataagatatatcttttaaccaaaccatgttttcttgtaatcatgtatatgcataataatattttattttgacaacatgatatgacgtctataacgtaaaaattaatattgttataacaataataatgtcattatgtgttatttaactaaaatcttaccattattatgtaaccttagactataaatcttgacgattacggtataattaaccttacatttcttgtagttcagacgttgaatgttaacattattttataacatcaaatgtgaaggtaaagacccaaaagtgaatactgatgaatgtgagatttcttgacgtctatttttattaacttgcatctatagacactataatataacaagacatccataaccatcaaacattagtgtaacaatatcattccataacctcgatcactatttctgtaacgtttaatagtaaatgacaattataatataattgttgttagacgaatggtagcgtagaaatttgctttattaatttgggacttagttgtaaaaatatcagttgttttatttcagacaaagtcaaattgaagcaattttgtaacgtcgttcctatgagtagactcaaccaactctctcattttcgtttgttttaggtggcgtcttactactaactaattaatactcccatgtccacacttcatatatctataatcaaattcctacgtctctagacgtcaaaacggctcgtatttggataatgagaaaaaaagaattcaatccaaaatccgcaaaacttggtttcttgtcccatactatttcttttcttttccgactttcattaccataaaaatactataaacaaaactaaacaatctcaattgtttttaaataatttatttctggatagccgaccaaattccactgtagcactaaccctaaataaataaataaataatccaacagttgcacaaaccttgtatataaaaggccctactacttctgctaatcattcccacgaaaaaaagacaaaaaaaaaaacaaataacaaaataacaaaataacaaaagaaacacacacaatatggattctcttctgcaatccttgcaacccttacaatccatgacaccactcttgctcataatcccactcctatttctcctccctctaattttccgtttccggcgaccaccaccatacccgccgggccccaaaggcctaccccttattggaaacatgttattgatggaccaactaactcaccggggcctcgccaagctggccaagaaatacggcggcatattccacctccgcatggggtttttacacatggttgcgatttccagccctgacgtggcacgacaagtccttcaagtccaagacaacatcttctccaaccgcccggccaccatcgccatcagctacctcacctacgaccgcgctgacatggccttcgcgcactacgggcccttctggcgccagatgcgtaagctctgcgtcatgaagctctttagtcgcaaacgcgccgagtcctgggagtctgtcagggacgaagtggactcggcggttaggaccgtcacagttcatgttggttcggctgtaaacatcggagagttagttttttcgctcacgaaaaatattatttatcgggcggcgttcgggacgagctctcaggaagggcaggatgagtttattgggatactgcaggaattttccaaattgtttggagcttttaatattgctgattttattcctagcctggggtgggttgatcctcaggggctaaacaatagactcgctaaggctcgtgagtccttggatcggtttattgacaccatcatagatgatcacatggagaagaataggaatggtaagggagtgagtgacagtgaaacggacatggtggatgagttgttggctttttacagtgaagaagctaaagtaaatgaatctgaagataatttgcaaagcgccatcaaacttactagggataacatcaaggccatcatcatggtaaataactattacacaatttcatgttacataattatattataaccccacaggttgcatgtttatgtaacctgttttcttggactaaacttttggctaattttttctttttatctgatcagatatattaattgcgtattgagactatgaagatagaaagttcatagtatattattttgaaaactgattgtttaaattgacgatctgactaggtatacaacatgctgtatatgcaaattaataagctaattatatatgctacgtattttcaggacgtaatgtttggcgggacggagactgtggcgtcggcaatagagtgggccatgtcggagctgatgaagagcccggaggacctaaagagggtccaacaagaacttgctgacgtggtgggtctagaccgtcgacctgaagagaccgacttcgagaagttgacatacctaaaatgtgccctaaaagagacactacgactccacccaccaattccactactcctccacgagacctcggaggacgctgtagtagccggctaccgcattcccaaaagatcgcgtgtgatgatcaacgcgtgggccattggacgtgacaaggactcgtgggaggacgccgagtccttcaagccctcgaggtttttgaaagaaggtgtgcctgactttaaggggagtaacttcgagttcattccgttcgggtccggtcggaggtcgtgcccgggcatgcaactagggttgtacgcgctggagatggcggtggcacacttgcttcattgttttacgtgggagttgcctgatggtatgaaacctagtgagctcgacatgaacgacgtgtttggactcacggctccgagagcgagtcgactcatcgccgtaccaagtaaaagggtggtttgtccactctgatggtgatgaaaaaacagcgttcgacgaggagagataaacagtggtgcaatttgtttggagtgtaatgatactgtgaaaactgcacaggtagattgcaagcgtttttttattgttcttttgaaaaactaaataattacaaagagaccatgaacagttgtgcgggaatttttagttctctctctttgtgtatatacactctcttttgttttgtttaatatgtatgcaaatttatctttaatttcctctcttttttcctttttgttttgttttgttattattgtattgaaataaaaatataatggctaagttacaaaaattatttgtcatggtggaaaaattgatggactgcttatgttacgatttcaatttgttttctgggtcggcggcaatagtaaaattattttgtttaaaaatgaatgaagaaagattaagtaaaattaactcttgaacaacggtagaataatgttaaatcatgcttacgacagaagtagctcacactgtcctcagcagttgtgaagattgacacttaatttgactgatgcaggtcaattatacatttattttgtggttaaaattattacataaaaaatatgaagagtaaggctatggagactaaatttatgagctaaaattgcaaactaaatgatatgtcatcaataaaaaaatgagtatgtttatcaacgtttaagtaataatacaataatcaacttcaatgtcatttaatttacaaaacttaatctacaaatttagtctccctatcattactcagatgtgaatgattgtgaaaatgaattaaggttggtgaggactttgacataaatctaaatataattaagaaggctctagggtagttctattcacacactcatttttacttctttcacattcttattaatttttgtctattgattttcttgaattaatttaatctggtagccggaaatcaaaaatagtatatgagaaataaaaattagtgtgtaagtagcactaccctggctctatatatggtgcccaagtcagatggatgcattttggtggctccatatatttgcaagacaagtgccctagctagtgacccgaagagatcttatttatgaactagttgtatacaactgtgtgttatctctcatcatgtggaaaagttaattagtatatagacaaaattatctgtaactagtaattaaaaaagtttataaccaaatgtgtacaactatgtatctaccgtggtataagaccagaagtactgtatatcatataacataggatagttggcttgatggtgcattccaaccgatatatgtgtggaggaagtgttgtaaatgccttgttgagctgcatcagcatctgtgtgtgagagagaaggtgaatgaatatattgagtttaggtgggcagtgaaaagtgaagtgaggaggacgtgaggccttttccttgtactataaggatgcgtttgttacaccttcttaagggattggcttaaactgacttaactagaactataattctacgtttggtatgtttctggactagattagatggaactcactccgactaacaatccctccacgtttcgttaaagctcgttatttagttcggagctttcgcttggtattaagcatgactaaaatggaaaacgaggccgacgcttggtcgtttgctacacccggacatccactctgccttctttctgctgcaccgtcacaaggccaggtcgtcggcggtgcctgtcttatgtggcccaaccttaggtgatagctgcagctcacagctgaagaggccctgctttaggccaaaatcgcgagtcggtggaggacctggcaatgctaccgtcggtgggcgttgctgacgagaagcagatcaagagcttgacgatgaaggttgggaagatctgtacaatatcattcgatttgattatttggttgcaggatggggagatctgtacatttttttatacgatttggttgctgacatgattgaatgagaaaattaattagtttttaaaagttaagataactataaaataagtgagagagatggtgagattgtggagataaaaaggtaagtggaggagcatgccggagaaatcgagaaggggaagttgagcagagtttgctgggagaaggattgcagtaagaaattaattttgatagaaaagaaattaattttaatagaaaataaaataattaaataaaaattattgtacttttttatttgtaatccaagctacgttaaacgtttca

SEQ ID NO: 84 - DNA, 1551 bp, Pyrus x bretschneideri
  misc_feature 1..1551: note = source = /organism="Pyrus x bretschneideri"/mol_type="unassigned DNA"
  source 1..1551: mol_type = unassigned DNA
atggattctcttctgcaatccttgcaacccttaaaatccatgacaccactcgtgttcataatcccactcctattcctcctccctctaattttccgtttccggcgactaccaccgtacccgcccggccccaaaggcctacccctcattggcaacatgttaatgatggaccaactaacccaccggggcctcgccaagctggccaagcaatacggcggcatattccacctccgcatggggtttttacacatggttgccgtttccaacccggacgtggcacgacaagtcctccaagtccaagacaacatcttctccaaccgcccggccaccatcgccatcagctacctcacctacgaccgcgctgacatggccttcgcgtactacgggcccttctggcgccagatgcgtaagctctgcgtcatgaagctctttagtcgcaaacgcgctgagtcgtgggagtctgttagggacgaagtggactcggcggttaggaccgtcacagttcatgttggttcggctgtaaacatcggagagttagttttttcgctcacgaaaaatattatttatcgggcggcgttcggtacgagctcgcaggaggggcaggatgagtttattgcgatactgcaggaattctccaagttgtttggagcttttaatattgctgattttattcccagcctagggtgggttgatcctcaggggctaaacaatagactcgctaaggctcgtgagtctttggatcggtttattgacaccatcatagacgatcacatggagaagaagaagaacaataagggattgaatgatggtgagacggacatggtggatgagttgttggctttttacagtgaagaagctaaagtaaatgaatctgaagataatttgcaaagcgccataaaacttactagggataacatcaaggccatcatcatggacgtaatgtttggcgggacggagactgtggcgtcggcaatagagtgggccatgtcggagctgatgaagagcccggaggacctaaagagggtccaacaagaacttgctgacgtggtgggtctagaccgtcgacctgaagagaccgacttcgagaagttgacatacctaaaatgtgccctaaaagagacactacgactccgcccaccaattccactactcctccacgagacctcggaggacgctgtagtagccggctaccgcattcccaaaagatcgcgtgtgatgatcaacgcgtgggccattggacgtgacaaggactcgtgggaggacgccgagtccttcaagccctcgaggtttttgaaagaaggtgtgcctgactttaaggggagtaacttcgagttcattccgttcgggtccggtcggaggtcgtgcccgggcatgcaactagggttgtacgcgctggagatggcggtggcacacttgcttcattgttttacgtgggagttgcctgatggtatgaaacctagtgagctggacatgaacgacgtgtttggactcaccgctccgagagcgagtcgactcatcgccgtaccgagtaaaagggtggtttgtccactctga

SEQ ID NO: 85 - DNA, 6142 bp, Pyrus x bretschneideri
  misc_feature 1..6142: note = source = /organism="Pyrus x bretschneideri"/mol_type="unassigned DNA"
  source 1..6142: mol_type = unassigned DNA
acaaaaccccttgaattagtttaattaggagcgcagttgctattagcttatttatttacttgacgcataggcataggtgggatagggctttgctttagcataggccaaaaagaagctcaataagccgaaatatagtacgtgtaaaccccttagttcctcttctttaccatagggtagggggtgtgaagctatagaaacaaaaaggctacttaacagctactaaagccaatggcgtagccacccacaggttaaggtgggatgtagcccagtgaggtttttttgtaagagtagtgttatttatgcactcatttttacttttcacacatctttcttaattttcagttgtttgaatcaaatgagatggagataatcactgacagaaaattaataaggatgtgttggaagtaaaaatgggcttgtaaatagaactattcttttgtaaaatcatgtttacagtttactcaatttcaatagagattattatttaatgtatattttatagaagataggtaataaataatacattttatgctaaaaattataaattagttctatctacaatcctttttttatcatattctttaaagaaaaagaccatttaccctatgtaatttagcctttgtgcttcttactcaagaaaagaaaaataatttctttaaatccacaattaaatccttgtctccacaatattccttaatttccgtagcgattttccaataaatgtcacctttgtactttcttaatctctatattattcttgatttaattagtaggttaattatttatatggtccatgaatttatctgttaaggttatgattttgagaataatttatattatattgttgttcttttatattgattattatataagtttatacaagcaaaaaataaacatatgattttatagtactttttttcaattagcccacccaaagaaaaaatcttggctccgcccttgactaaaggaacgctattttgctattttacagcttgaaaatggctcctaaagctaactactagcactagcaagagctaatagcaaaaacagagctagtagctagccgcttctatctcttcctatatagcttagcttcaaaatagcccaaagcaattgtcctatagagcttccaccttccctacgatattaatctttaaaaaagatttagtttagaaaaaaaccgtatgcaagattaataattaaattaagtagctgacccattagttatcatttatttctttgttattcttccaattatacccttttgtgattgtttgtaatgaagacttttttttttggttaacaatcataggacgtcatttattatagcttatctttaggatgtatatgatattttgtagtagtttttttttttattttcattcaaatttcaaactgattttagttcatattcgagaaattagtaattcatattctaaaattaagagggctatacatatagtatattgtaatgatatcaccttttttatatacatattattgtggtaggtaccatgatttaagtgcatttgacttggtactatgatttatgtgcatttgattatcactaatatttttttggacgtctagatgaggtatgtttatcatttgagaaagtcaaataataatcaatgccaaaaaatatggagcaaaataaaaccaaatatttaatatgggcattatgggaacaaaaaaaaaacattaaatatttaaaagctagaaaatatgtattttaatatttatgatgacatggaaatttagtcaaaagactataatgaatataaaatctaacataaagttttaattgaatttcaatatttatcaaggattaaaactgagaaacccctagtttaattcaagatagcatgacccaatgtaacagcaacaccataaaattaaaaatataaaaatcaaacggtagcacaaattttgcatataaaaggcaccacaatatctcttccaacttaattagctacattttgagttgccctacaacttctactaggcatacccacagaaaaaagtaaaaaaaaaaaaacaagaacaaaagaaacacacacaatatggattctcttctgcaatccttgcaacccttcacatccatgacaccactcgtgttcataatcccactcctattcctcctccctctaattttccgtttccggcgactaccaccgtacccgcccggccccaaaggcctacccctcattggcaacatgttaatgatggaccaactaacccaccggggcctcgccaagctggccaagcaatacggcggcatatttcacctccgcatggggtttttacacatggttgccgtttccaaccctgacgtggcacgacaagtcctccaagtccaagacaacatcttctccaaccgcccggccaccatcgccatcagctacctcacctacgaccgcgctgacatggccttcgcgcactacgggcccttctggcgccagatgcgtaagctctgcgtcatgaagctctttagtcgcaaacgcgccgagtcgtgggagtctgttagggacgaagtggacacggcggttaggaccgtcacagttcatgttggttcggctgtaaacatcggagagttagttttttcgctcacgaaaaatattatttatcgggcggcgttcggtacgagctcgcaggaggggcaggatgagtttattgcgatactgcaggaattctccaagttgtttggagcttttaatattgctgattttattcccagcctagggtgggttgatcctcaggggctaaacaatagactcgctaaggctcgtgagtcgttggatcggtttattgacaccatcatagacgatcacatggagaagaagaagaacaacaataagggattgaatgatggtgagacggacatggtggatgatttgttggctttttacagtgaagaagctaaagtaaatgaatctgaagacaatttgcaaaacgccatcaaacttactagggataacatcaaggccatcatcatggtaaataactattacacatttttatatcagagattataaccccacattcaggttgcatattgtctctgtaacctgtattctttggactaaacttttggttaatttattctttttatctgatcagatataatatattgtgttgattgagattatgaagatagaaagtctaacatatttaactttaaaaataaataatagtttaggttgatgatctgaatacataacatgttgtatatgcaaattagtaagctaattgtatatgctatgtacatgcaggacgtaatgtttggcgggacggagactgtggcgtcggcaatagagtgggccatgtcggagctgatgaagagcccggaggatctaaagagggtccaacaagaactttttaacgtggtgggtctggaccgtcgacctgaagaggccgacttcgagaagttgacttacctaaaatgtgccctaaaagagacactgagactccacccgccaattccactactcctgcacgagacgtcggaggacgctgtagtatctggctaccacattccaaaacaatcgcgcgtgatgattaatgcgtgggcaattgggcgtgacaaggactcgtgggaagaccctgagtccttcaagccctctaggtttctgaaagatggtgtgcctgactttaaggggagtaacttcgagttcattccgttcgggtccggtcggaggtcgtgcccgggaatgcagctagggttgtacgcactggagatggcggtggcgcacatgcttcattgtttcacatgggagttgcctgatgggatgaaacctagtgagcttgacatgaacgacgtgtttggactcaccgctccgagagcgagtcgactcgtcgccgtaccgagtaaaagggtggtttgtccactctgatggcgatgaaaaaacagagttggattgaggagagagaaacagtgtgcaacatgtttggggtgcaatgtcaaagtatacatacagtaaaaatggcaggtaaattgcacgcgtttttaattgttcttttgaaaaacaaaataattacaaagagactttaaaaagttttgtgggactttttatttctctctgtgtgtatatagacagtctcttgttttgtttaatatgtaagcaaatttatctttaatttcctttttgttttgttttgttattattgtattgaaataaaattagtggatgagttgcaaaattatttgtcatggtgtaataattgatggactgattatgttacgattttaattttaatttttttttttggtcggtaggaatggtagatttatgtacagttgtgcttgcgatataagtagctcatactgtcctcagcagctctgaaaactgacactgattgacttatttcgattgtacttatttggtggttgaatttgttatattaaagatgtgatattgtgcggatggtgagaatgaatgaatgttggtgaggacgttgacatctaaagctaaatataattaaggcggctctatatatggtgcccgaatcagatcaatgtctttggtggctccatatttgcaagaaaagtgcgccttttgtaaatactatttatttggtatataagcgtgtcctagctagcgacccgaagagatcttgtttacgactagctgtgtacaactatgcgttatcttatacatttatacaactgtgcacacgtatagaattgtccatatgtatatgtaattagtaaaagaatttcttgtttgtataattagttttgtacaactatatgtctatctgatctatggtatatgaccagaagaagtactgcaaagcatatatataggacagtgcactagttaacctatgtgtgtaggaaatgttgcagtgaatgcccttgttgagctgcatcagcatgtgtgtactttgtacaaccgagagagagagagagagagagagagagagatggaatattgaggttaggtgggaagtgaggactcaaagtgagaaagacgcgaggccttttccttgttttctttaagtatataaaaatatatcgatgatcatgagtcacagtgatatatttatttagagtaatgctagggagattaaatttacaaactaaatgatgtgtcaccaataagaaatgagcacgtttatcaacatataaataataatccaatcattaacttccatgttatttagtttacaaatttagtctccataacattattcatttgtttgtgtgtgaaagaagattgtgcttatcagtttatgtatgtataactgcgagagctggaaggggatccatcgaagattttccgccctcaagtaacacctggggttttctaaagtttggtggcttggtgctcaagaaataggacgcactgtgctcggttatttaacttgagttggcaaatggactgggattgagtacctatccagatgattaaatatatatctttgcatttgccaatatattatcacctttttgtttgttctttttaatatggctaaatatccaaaatggtgtctgagatttgcataatcaatagaaatggtccttgagattcaaaatcaatagaaatggtccatgagattgtctaccatccatgattttgttcattccattaaaaactctttaggcaattttcaaaacttcgtaactcaatcaattcttaaccaaattcgacccataatatatcaaaatgaagataggaaagtgtagaacaagattatacctatttggaagcccaatggttgccggagatagctggaaaacagcctgaaaggtgactggttcgcgggaaaattggaaaactcgccggaaactgggtaaactttaaatgttcataacttcttcaatactcaacgaaatcaagtgattcaaaaacgaaaatcatacttctcaatgagatgaagataatggtacctttctcgatggctaactcctctacttcgtggacgtagccaaacttaggatgaactacgtacatattgtgtttgcttccctatctctatctctttacatacttatcctcactagtgaccggagcaaataagcaaaggtcacaaacttgacactttacattgttccaaagtcttcaccgattttatgcatcaacaagtaccaatatcatcgttacagtttatccatcaagatcatgacctagaattggtc

SEQ ID NO: 86 - DNA, 5937 bp, Pyrus x bretschneideri
  misc_feature 1..5937: note = source = /organism="Pyrus x bretschneideri"/mol_type="unassigned DNA"
  source 1..5937: mol_type = unassigned DNA
catgaccatagcagctaaagccctgtaacagatctagcatcacactgctgtaaaaagaatagtgatctctgagtttctgtatctcatcgatgacaaaatacctacaatcgtatgtgtcaaatatttattttattattaatatatttggtcccataattacattaattattaaacaataattaaaaatcaattttaatcctattggttgtcaatgagactcatgaattaaacaattaaattaaggcatttgactccatctttgtttgtggtaaaaaatgtcatatatgacatctcctttagagaataccatgatatcttttaatcctagttaacatatttgacatctcctttagacatggtaagaattcaaattattcttcccttcttttgtcgaaattcaattaattccttaattaactggggtataatctgtttccagacaagtggatccaaaattcccactgccacattagtgcattcctcattaaaggctgcagatcccttattaattatatcaagaactctcatagttttatatgaacctgaccaaccccattttcccaacacaaccaaagccatggagctccttcaccaagctcttcaatccctgcaatcttcacccatgttgcttattcttcttcttctcctcctcatcatttcctttatttttttgtttaattcgcgctggaaactcccatatctgccaggtccaaaaggatggcccatcatcgctaacatgctcatgacggaccaacttacccaccgtggcctaacccatttgaccaagcaatatggcggtctcctccatctccagatgggggtcatacatgttatggccgtatcaattcctgatatggcacgcgaaatcctccaagcccaagacagctgatttgccaaccggccagccaatgttgccatatcttacttgatctatgatcgggcagacatggcctttgccaactacggcctgttctggcgtcgcgtgcgcaaaatttgcgttcgtgaatctatttagccgaaaacgggctcatgggcttcggtgcgcgaggaagtcgagatggtccaaaccgtggcagggaaaaccggctccccggtgaatatcgggcagttggttttcgctctgacgaggaacataacatatcgggtggcttttgggtcaagctcacatgaagggcaaggcgaatttgtgaaagattttgcaagaattttcaaagctttttggagcttttaacatgcaggattttttaccatggttgggttgggttcatgcacagggttttcaggacagaatggctagggcatgtaagtcactagatgttttcatcgacaagatcatcgacgaccatagcagctataaccctgtaacagatctagcatcacactgctttaaaaagaatagtgacctctgcgtttctatatataattgatgacctaagtacctacaatcttatgtgtcaaatatttattttataaatatattggtcctaaccatcctatagttactttaattattaaaaaaaaaatttaaattgatattagttgtcaacaacactcatgcattataaaattaaattcaaaccatataagtctatctttatttgcggtgaaaaaagacaactagaaagcaacgtcatatatgacatttcccttagagagcatctaatgtcttttagcctagttagcatatttgacaaaaatggtaagaattcaaattattcttccttcttttgtaaaaagtcaattatttccttaattaactatagggtataatctgtttccagacaagtggatccaaaattcccattggcaatatacacaaaacaacgcgtgacgtttgcaagataaaatacatggcagatatcaccaccaaagtcgccagttattacttcccacattagtgcattcctcattaaaggctgcagatcccttatcaattatataagaactctcattgtttatacgaacctcaccaaccccatttacccaacacaaccaacgccatggaactccttcgccaagctcttcaatccctgcaatcttcacccatgttgcttattcttcttcttctcctcgtcatttccttgattttcctgtttaattcccgccggaaactcccatatccgccaggcccaaaagggtggcccatcatcggcaacatgcttatgacggaccaacttacccaccgtggcttggcccatttggccaagcaatatggcggtctcctccatctccagatgggggctatacatgttattgcagtatcaactcctgatacggcccgcgaaatcctccaagtccaagacagctcattcgccaaccggcctgccaacattgccctatcttacttgacctacgatcgggcggacatggcctttgccaactacggcccgttctggcgtcgcatgcgcaaaatttgcgtcattaatctatttagccgaaaacgggctgagtcatgggcttcggtgcgcgaggaagtcaatgagatggtccaaaccgtggcgggaaaaaccggctccccggtgaatatcgggcagttggttttcgctctaacgaggaacataacatatcgggcggcttttgggtcaagatcacatgaagggcagggcgaatttgtgaagattttgcaagaattttcaaagctttttggagcttttaacatgcaggattttctgccatggttgggttgggttcatgcacagggttttcaggacagaatggctagggcacgtaaatcactagatgttttcatcgacaagatcatcgacgaccacatggctaagaggaaagcaaacacggagaataaggatgacagtgaggctgctgatacagatatggtggacgaattgatagcttactttagtgatgatgctggaaaggaaggtgacgaccccaattctggcttcaagctcaccagagacaatatcaaagcacttattatggtatgtatatttgacaattaaaattttcttaatacgttatatttgttggtgaatagtcacacatcggagaaaaacaaaaataatatacaattttatgtgtgagagatatacttctaatatcatcgagattttttaatgataaaattcaatacatagcaataagtggttaagttagaacaatagatacactttattaatttttaagaaacaaatctgattttaagtatttgttgttaactatttttgtaaggaattaggagagaaaactattctgataaggaattcatgtacttaattattactatatttatcatttccgattgatttagtactaaataatacgattatcccatatatacgtaggatgtgatgtttggtggaacagaaacggttgcatcagtgatcgaatggacaatggcagagctgatgaagagcccagaagatctcaaaagagtacaacaagagctcactgatgttgttggattgaaccgtaggctccaggaaaccgacctcgaaaacctaacctacctcaaatgcgcagtcaaagaatcccttcgtcttcacccgccaatccctctcctcctacacgagaccgtggaagacacctctgtggctggctactcgttcccagccgggtcacgggtttggatcaatgcatgggctattgctcgtgacccgactgcatgggacgagccagaaacattcaaaccctcgaggtttctggacgatggctcgcctgatttcaaagggagtaacttcgagtttattccttttgggtctggtcggaggtcgtgcccgggaatggcgttggggctgtatgggttggagatggctgtggctcatctgcttcattgttttgcatgggagttgcccggtgggatgaagcctagtgagcttgacatgaatgatgtgtttggactgactgcaccaaaagcggttcaacttgttgctgtgccaacttataggttgaattgccccctttgaattaatagtttcaagtagacgaggagacgatgatgatgaaatattacttggttttaaatcagtgatcgatgagtaaatttctctatgtaaaggtactttaacgaataagggtttgttacgttcttggtctctctttaccaattgaataatactattgctttccccatgagggacgagctcattattcctctattgcaaataacaatatcttcaacgtagaaatttcataattggatcagttcaatatcttcatcttcatttgaagatcatccttacaaaaaataatttgaattttttattgtttcatcatccaaatgtataaataaatcaacggtttatcatgaatatgttacttgatacctacacttatgatttatttgatacatttggatggctaaacgatcacctcaaatgattttttggaggaatgatcttaaaatgaagattaagagattgaacggttctaattattaaaaaaaaaaaaaaaaaatctatgttaaagatacgttgcttgcaactggaggaatgacctcgtctcccaatcccaaggagagtatgtaagcattatccattattgtaataatcatttttaattttgtaatatcgctatgtcgttaatatttaatataaatatatgcgtggttgtttgttaacctttgttggctttctgtcctttgtggctcagccttgtatcttttagtcagtttttcgaagacaaaacgggaaaagattccatccagatctctcccaccaaatcctaaggatccgaagatccggatccttaaaatttgattcaacggccacaaacaagaggttcttctaaaaattataataattatagtagttagatcaaatttcaaaggtccggatctctaggacttggtgggagagatccgaaggggatctcttccctgacaaacaagtggggatgtacttttaatgttggactgttgaagtcctggaaaaacatctttcgtcaaactatatagcaaaatagtagtaagctaagacgcttatggtgttagataatggattgaggctattgatcaagatgaatctttcgtaaatttataccaaatttatgatctcacaacataatttgatgaacttgatatgcaatattgatcacacaaagacttcatcttatagaatattggtttatatcctaaaaaattaaatttgtagggttcataaactgttccattaggtggtaacatattaatttgatattgtgatgatacaaagctcaaattaacagtattgttatcaactaactattggtctggtaattttttttttcttccctaatgttggaataaatatcttgatttcgattccactcttctatacaaaagataatataaataggagcatagctttcaacaaatgaccgttgaaataaaaattttgagttcggttccttccttccacacgaaagataatataaatcaatagcatagctttcaacaaatgacggttgcatttagacgaaaacccttaaaagtttgccgaccctttgacacgtagataagcaaataatataactagctaattgaagtggttgagaaatggacattgtgagattagaatgcgttagcaattgcgcaatgcaaactatgatgtcggtagaaggtgaaaggataaacctcagccaatgacattctatgcagctgtttagctatagctttttgagtccaacttcaaggtagaagaacgattactcatcaaataaacttatcattccaatcatctttaaaataagaggaaaaagaaataagtaaagttgcgtcgttgagtgagaaatgaaaaattcatagaaagttacaacccctaccacgtaatagtgcacctacatgccttcatcgacgttgtttctcaattggatagataaacgttttcttctaagtctaaaccccctttcatcgatgttgtttctcaattggatagataaaagttttcttcaagtctaaaccccctttcatcaagtggtgaattgacacaatgactaggacat

SEQ ID NO: 87 - DNA, 6246 bp, Prunus persica
  misc_feature 1..6246: note = source = /organism="Prunus persica"/mol_type="unassigned DNA"
  source 1..6246: mol_type = unassigned DNA
accgagatcttttgtgataaaacttcatacttattaagctttgtagataattaaattgtaaataatatcgatgttgttagttgtgtactactagtctttctgaatttaacatctacatcataagcagtccacatcacatattatacatctgaccttttatacagataatcttttgtataccaaacaatatagtaagtccccagtaatgacttgccacgttagcccaatcttcataaaggctgcagatcacattttaattatataagaacctcatccttttcttttcaaccttatcaaattcataatttatattaacctcctcaccaaccccattttcaagaacacaaaccaagccacggatgatctcctccttcaccaagcacttgaatccctgcaatcttcttccatgttgctcgttcttcttctactcatctttttctttattgttttgttcatgtcacgcccaaaactcccatacccatcagatttgaaaagggtccaatttcattcaattactgctaacacacctccaacccccaactttcttcacatatgatggcaatttgaaaattagaagttaacaaattagtgagaaaaaggagacaaaatcgctagtcttggcgtagccaccgaaagggtgagaagaagattgagcaatgattaagctgttgaagagagagagagagagagagagagagagagagagagagggagagagaggccgagaaggcagcgcgaggttgacgatgggttggggtgggtggtggctattttcttttttcattgagtaatgctactcttactacattcttgtaccaccttacttagcagataaagtgtacagctacatcaattagaaactgttaataaatcataaaagaaaaaaaaatattcaattaattttaattgatgtggattgtccaccttatctgtcacttaaagtggtatacaaatatggtaagagtagtattattattttttttttcttttttgattttcttagttttatttaaaaggataataataattagattccaagtccacctataaaaaaatataaatttttattcttaaatgacacatggcaaattttcaattggagtatcacaagttgagatggtagtggagtaccactaggggtgtacgtcgatttgaaactaatacaatttaatccatattcgatccaatataaattcgattgtgatattttcaatcctatccaatcatattagatgtttaaattcaatcggatatgatccaattgatattggactggattgggttggatgattggttttgaacttttgataatgaaaaaaatataatattgaaatattaaacatttattcttaggtttaatctaatcaaacatattaagacgtaaatgtaaaatataaatgaaatatcagagaaaattgttttgattatataatatattaaatataattccattaaataacttaaaatattcataacattttcaactacaactacctataagagcaaaataagcattactataatattagaccaaattttagctagctaattagtatgtttgaattaatttccttataaaaattatacgatatgtaaataatatcgacacgaatcccgagtttctagatttaaagacattccacttctacataagcagcccagatcacatattatacatgtgaccttttatatagatccttattaatcttttgtatacaaaacaaaacgaattaaaagaaatcccaatacaatttcaaatatttccttagtgcagacaagtggagccaaattcccacagggaatggagttggtgagattagatcatcctctattgtaagtccccagtaattacttgccacattagttcaatcctcataaaggctgcagatcccattttatatatataagcaactcatccttttcttttcaccttatcaaattcataatatatatatatcaacctcctcaacaaccccattttcaagaacacaaaccaagccatggatgatctcctccttcaccaagcacttgaatccctgcaatcttctcccatgttgttcattcttctttttctactcatcatttcctggtttgttttgttcatgtcacgcagaaaactcccatacccaccagggccaagagggtggcccattattgggaacatgctcatgatggaccaactcacccaccgcggtttagctcaactggctaaacaatatggtgggcttctccatcttcagatgggggtcttacatatcatggtcgtatcatccccgaaagtggcccgagaaatcctccaagtccaggacagttccttcgccaatcggcctgcaaatgctgccatatcatacttgacgtatgatcgggccgatatggctttcgccaactacggcccgttttggcgtcgcatgcgtaaaatttgcgtcataaatctgtttagtcggaaacgggccgagtcatgggcctcagtacgtgaggaagttgaagagatggttcgacatgtggcaacaaaaaccagctcaccggtgaatatcggtcaattggttttcactctaacgaagaacataacttatcgggcagcgttcgggtcaagctcccatgaagggcaaggagagtttgtgaagattttgcaggaattctcaaagctttttggggcttttaacatgcaagattttcttccatggttgggttgggttcatgcacaggcttttaaggacagaatggctaaggctcgtaggtcactagacgtgtttattgataagatcatcgatgatcacatggctaagaggaatacaaataaggccaagaaagatgacaatgaagctgaaacagatatggtggatgaattaatagcttttttcagtgatgatgctgcaaaggaaagtgacgaccccaattctacctttaggctcaccagagacaatatcaaagctattatcatggtaagtcttacatgaaaatataatgaggattcataatatctccatattattatcgtcagcaaaatataccgacatgttgttgttgcatttttataatatttatgtgcgtgtccattctttaaataatatatcctgcatagagaatttttgatatatataacagtccatacatagatatttattaataattattttttcaattttaagaaaacctcaacggaatgagggaaataattattcttttaataaacattttaatacattactataattttctttccaatattattaactaccaaacccccgttctactgatcacatcgttctccaatattgttgccctttatttgaggtgccaactcggtctaggggttttcctttttatttattttaattatctaaaattgaaattttttttatttatttaggtaacttcacaagcggattacattgtaattaatattttgtaataggtgggtcaatgcttttttccttttctaaatcaaaagaaacaaatctgatttcttgtgttttttactatatttactcagaatttatggtaaaataaagcacattctcatttctcactcagaagatcaatattatgtgttcaattttggtaatggaaattatgtgtttaattatcactctgtcacttctgatttaccactttgcattcataggatgtaatgtttggaggaactgaaacggtggcgtcagtgattgaatggacaatggcagagctgatgaagagcccagaagatctccaaaaagtgcagcaagagctcatcaatgttgttggcttgaaccgtagggtccaagaaactgacctcgaaaacctaacctacctcaaatgtgcagtcaaggaatccctccgtcttcacccaccaatccctcttctcctccacgagactgcagaggagacttccgtggccggttactcatttccagttgggtcacgggtttatattaatgcatgggctatcgcccgtgacccgactgcatgggatgagccagaaacattcaaaccctcgaggtttttgaaagatggctcgcccgattttaaagggagcgacttcgagttcctcccattcgggtcgggccgtaggtcgtgcccgggtatgcaattggggctgtatgggctggagatggctgtggcccatcttcttcattgttttgcatgggagttgcctgaaggaatgaagcctaatgagcttgacatgaatgatgtttttggcctgactgcacccaaagcagttcagcttgttgctgtgccgagttacaggttaaattgcccgctttgaatagtttgaggagattgtgaaatacctttggttttaaaacagtgatgagcctatttctttacgtattggtactggccgccggtcctgaatttttcaggccccagggcaaaagtaaaaattatgcccttatatatattttaaatttagtgatgaactcactttcatttttcaaccaagatttaacctgttcacattaactaaacatttagcaaataaattatttcaattcaaacttcaaaataaaaggctacaatgtataaacaagataaatacttgtaacaattcagtaagttctttttcatccggcaacaagcaaccaacaaatcaaaaatttatatgcatgctcaatagccaaagcaaaacaacatcaatctagttgaaatccctttgcatcctcgtccaccagcaaaataaaaaatgtctttgcatcctcaacaacacaaagaacaacataacctggaatttgagaaatccccctccacaacaacaaacttgatatttccaaatttgcaatcagttagatcttaaaagaattcttgcaaagagaatgtgtaactcatgtgagttcacaaatctacaaaaaaaataaaaatgaaaaaataatattttccttttcatttgatatcaaatcatgaaaaattactttcaattgatttttacaaggactgattttgtactgaacaaaagtccacactttgaattttgtaatctccctaatctaccaatagaaaaattaatttagggattcacaactctgtaactagtaggagaggcttaaatgagatgagaagaagaagaggaagaacgagaaaaagaagaagaagaaaaattttaaatagataaatctaacgttaacctggaggagtggagcgggttgcgcaaaagggttcttcttgagaagcgaaaaggatgcacgttcttccaaaacaccatcttttttttttttttttttctcaaaaatattaaatcagtttataataatataatataacacttttttttaaaaaaaaattgagcccttgaataataggcttagggcggtcgcactaacggcccttgttcaggccgggcctgttttagtcttctatttgttcaggccctatcactgactcgttagtacagtttgtttgtatggatcgatcaaataaacaaaccaaaatattcccaataagaatcaaatgattaaacaatgttggctaatttttataaggggtgatctttgaatgataatctgaaaaacgaatgtccaaattatgagattatattgctaagtggaccaaaaaagtgatcaatgccgtattaacttactaatcgattaatgcttgatcactcctgatcaaacaactaagccacataattatttatgtaaaaatttggccgcaagtaacaaaaattacttgtagacgaaaacccatatgttagcaattattttcaaacgaaaatatacaaaccgctcccttcgtgtgatgattaatttgttcatacaaggaggagggagggaggaacatttcaatttaatcatacaactgggatcatgccatttcaggcattgggcttcttttatgctataaattactcatacaatcaaaattgttacttaggccttctacggtaagggtatagaatgtcatccacactctgtttgatatagcttcccactctattcacattcttagcaacacctgatgctactagtattgcattcctcttaaacctgcatatatacaaatactcagagttttaaagtctaacaagaacaaactgaactggaaaactcaaacacaaaaccatgtgaaaaaaagaaagtcatggataacttcttttctttgctaactcaaagttctaccatacctgttgagatattcatctcctgttgaatgtggtggtctgtagctttggggttgaggaatcggccgaacattttctggagacacaccaagaatttataaacaaggttgctgtgaatgttataagaaaattcagtcattttgcaacactaa

SEQ ID NO: 88 - DNA, 5782 bp, Prunus persica
  misc_feature 1..5782: note = source = /organism="Prunus persica"/mol_type="unassigned DNA"
  source 1..5782: mol_type = unassigned DNA
agcacatctaaacttatcatttttgggggatttaagtcataatgatttagacacctcaaaatgggccatgatttatcaactaatgaggcatggctttgggtgctctgaggttttccacagcgcgattcacactgctaatggatgaatgaaatgaatattatttgatgaaaatgaacgagtgggaaaatatcaaatcaaatcccacagggcacagggcacaggccacaggcattgaattaattattaaaataatgatgatgttatcatgatgctaaaacacgcaatctagctacttatctcctaattctgtaatcttatcttagttttgaagtatattaactaaacacaacaccatttaacacattgcatttccataaaatatttggaagagaaaaaagaggttgaaatggtgagatgaaacagtcaattgctcgaatgtctaaacctcgacagctacctgaaatgtataaggtcctgcatcaaatctttcacctttcacatggacagactgaccaattgttatgtgaagcctcaaacatacactttgctatacactaatgaatcatcattcacgttgaataaagaattaggtggccagctagctgtgctagctttaaccctaaagacaaatagaaactcccaaaacaagctcggtttgttttctaagcaccatcaatttgaattaaaattcaaggaacaaaaattcaataaattatttagtgaaatatgtacctatccgggtcactattttttggcttaaaaatgttaaatgataagatacggttaagatggatggttttttaagtaccgtttataacaaaatttctgccaacaaaatctagcacattagaaagggacttagtactactaaaaaaaagaagatgaatgaggcatgccatcatgaagtgtttttcctgattaattttgtgaattaaacaaagtatactggacccgaatgttaaatgtgactagttcgaggaaagtccacaattgcagcatatgttttttttgtcttgcagccatggtggaacttttcggaacattcatttaacatttgttgaaattcgtgcacaccatttattattgagcttgcttgttcttttattagtgatacatatttgcgaaagttgagttcatttcttgcaagttacaatatagttactcaattgcgggaaattgagataaccaatttaaattctgctaactaacttcttgcaattaacttaattatacatctctcactctctaaaagtcttgacttcaagtcctatcatttgtataatgcgtgtaattttaatatattatcgccactttcaataaaaaatattcttaaaacaatgttatttcaccttcttattctttaaataaaaaagaaaaatgatacttttcaatcatatacggaagcttcataatatttttttttctctgttaatttcattaattctagaagtattcaattgctttacttcagacaaagtcaaattaaagcaattttcacaaaaataaagtaaaagctacctactctcttttttttttgttaattttatattttattttatatcgaatcgaagtcgactgcaaagaggcgaagaatgcctttataccgctttgactaattgatctaaactcatgtacatattgcagataaatatccctcatcaccgtttataaagaaggaatgttagtacgtatctatacgtcaaagcagtttggaaatccatatctcccagtcttttcccagagtcttctagttttacgcttactcattcagagacaatagtccaacttccatccataccacaaaacataaaataatattaaaacaaaaataaaagacaatttattcaattcatttaaattctaaatcgcgtgactaaattcaacagcagcacacaaactttgtatataaaaggccccataatatctcatccaacttagcaacattttgagttgcacacaacaccaaactactctacgcacccaaaaaaaacacaaaccacatggattctcttctccaagccttgcaacccctacaacccatgacactcttcttcatcatcccattcctcttcctctccggcctagtcttcctataccggtcccgaagacgctccccctacccaccgggccccacaggcctccccatcatcggcaacatgctgatgatggaccaactaacccaccgaggccttgccaaactggccaagcaatatggcggcatcttccacctccgcatggggttcctccacatggtcggcatttcaaaccctgacgtggctcgccaggtcctccaagtccaggacaacatcttctccaaccgccccgccaccatcgccatcagctacctcacctacgaccgggccgacatggctttcgcccactacggtcccttttggcgccagatgcgtaagctctgcgtgatgaagctcttcagccggaagcgcgcagagtcctgggacgccgtgagggacgaggtggacacggctgtccgtaccgtcgctgttcaggccggttcggctgtgaatataggggagctggttttttcgttgacgaagaatattatttatcgggcggccttcgggacgagctcggaggaagggcaggatgagtttattgggatattgcaggaattttcgaagttgttcggggcttttaatattgcggattttattccttgccttggttgggttgaccctcaggggctcaacaacaggctggctagggctcgtcagtcgttggatagatttatcgacagcatcatagacgaccacatacagaagaagaagaagaagaagagtgagggatcgaatggcggtgaaactgatatggtcgatgaattgttggctttttacagtgatgaagccaaagtaaatgagtctgacgacaatttgcaaaacgccatcaaccttactagagataacatcaaggccatcatcatggtaaattattgcgctttctataccatataaaaccacaattaattcggaaaaatctgaaaatgcttacgtattatgttgttagactgtaagtttctatcggataatattattttgaattgattacgaatcaaattcacattccaacaacgtaacatatcagtcatttagtaaactaaataaatgaactaattaattgacatgtgttgcatgcaggatgttatgtttggcgggacagagacggtggcatcggcgatagagtgggccatggcagagctaatgaggaacccagaggagctaaagagggtccaacaagaacttgctgacgtggtgggtcttgatcgccgaccagaagagggagactttgagaagttgacttacctaaaatgtgcacttaaagagacactgcggctccaccctccgattccactgctcctccacgagacggcggaggacgcggaggtggccggctaccacatccctaaaaagtcacgcgttatgatcaactcgtgggccatcgggcgtgacaaggactcgtgggaggacgccgagtccttcaagccctctaggtttttgaaagaaggtgtgccggactttaaggggagcaacttcgagttccttccgttcgggtccggtcggaggtcgtgcccgggcatgcagctcgggttgtactcgctggagttggcggtggggcatttgctccactgttttacgtgggagttgcctgatggaatgaaacccagtgagctcgacatgaacgacgtgtttggactcaccgctcccagagcgagtcgactcattgccgtaccgagtaaaagggtggtttgtccactctgaatctgatgatctcatgacgaggatgagaaaaacagagtttgcatgtgttaattggtgtcccaaaatttcaagtatacataacaatgaaacggcgacgcacaggtaaattgcagggttttttttttttttttcttccttttttttatatttaaaatatttgggaagaacccagaaaacaaataattgaaaagagactttggaaaaacgaaatgagggaatttgtattctctttctttgtgtataaacactctctttgatttgtttcatatgtatggaaatttgtcttggattttctctctttttcctcttttttttttcccttactattgtattcaaataatggatcaatttgaaaaatatatatatttgtcacagtggaaaaaaaataatattattattgcaaacacttggtatataattttgtgtaatttgcaattctttctcgtgactttagggaagatatgtggctggatgtgtgaatatttatattaagtttgtatatgcttgcgactgaaggtgttcatactgtcatcagcaactccaagaattgacacttttgactatttcacttgctatatagtgctaaatgtgaatctcaatataaggctctatggtgcatgaatgagttagagcaaagtattttggtgggtccattgacgaagtttctcaataaatcaatcatgtgatgaaagagatagacacaattatacaatacggagtatcagggtagccgccctgcccgtggtgcaatatgcatcaaacatgtgcatgcaaaagagtgttgtaatgcgcttggtgtaatatactaggtactcacgaaaattatactagtactgagcactggagtggtgcaatatgcatcaaacatttgcgtgcaaaaaagtgttgtaatgggcctggtggcaacaaaacatgtttgtacttggtacaattgatacagagaaggagagagggatatataaaatatattgagtttaggtaggcagtgaggacaagtgaggaggacgtgaggcctttcttgtgttttctttgggtagaataatgagactgggcatgaggttgagaggtgaggtgaggtggagggaggttccatctgagatttcccatcttcaacaaacgtgggatatggacaatagaggacataagggccaccaatatgtaatccaatcagattcagtaggatatttgtatattatatatccatccttgtccctttttgtaaagttggtggcctggtgcttgaataagaccccactgtgcttagaagtttagcttgcgtttgggaaatggagtggggttgaatactatcctatccaatccagttcaaacacaccaacctatttccatcccactccattttatttttaaggcctctgtattgcatttgtgtatatgccgcactcaaattagactgatgagttttgagatcaaactttggaacaccccaggatgtagatgtgtacagagaagacctagagatagattgatgaatgcgtttttatttgttagattggaggaaaccgaactcttaactactcgtcataacctattactccaatttcattccttaccccatttgagttaactcccaaagattttcagtcacaaaataagggttacaacttgaactgttcatctagtgttgaataattaggagttataatgagaggatgcttaatagtcactgattaattgcacagctattagtggaaggtttttttggttttttgttttaaaaagaaacaaaattattaatagatgctatcagacccagccaaacccagcccaacaaacttattgacgttggacttgcatcatattggcacatgagagagagagagagagagagagagagagagagagagagagagagagagagagagagagagagagaggacttaattgaattctaacaagatgaatatttggattgcgtaattgaattcttcctcgggctaggccccacttacaatgtcctaatctttcacatctttatcatatttcttaaa

SEQ ID NO: 89 - DNA, 6020 bp, Musa acuminata
  misc_feature 5523..6020: note = /note="n = a or t or c or g"
  misc_feature 1..6020: note = source = /organism="Musa acuminata"/mol_type="unassigned DNA"
  source 1..6020: mol_type = unassigned DNA
ttaattagactaattatatattattttcataattaattattttagtatctcaatcgttatatcttcaaaaattatattgaaatccttatacttattttttctcacctgcctgtgatctcattatgtgagaagacgccaagccaaaattgattgattgatacattgttcgtggtgggagctgtaagcacatcattcggtcactccctttgttcgaagtcccttcagctgaagcatcagaatccacagtgtatttgctcaccggcaaatgaaagtactctgtgaaggtaggttaaggttatggaagacaatgacctctctctctctctctctctctctcttcaccaccatcacccatttctaagtgagcataaatcccagagacttgaatgacaagctcccttcattacgacgtttgcacgcagaaaaaaaccattttttatgtttggaataaaagttaaacaccggaatgaaagattcaaacttatgctctattctgatatatataatataaaagattcaattgggttgctctaatcccaacatcctaaactaataaaagaaaaggacccaattatattttttagataattcaaaaggacgccatgcaaactaaaaaatcatcacattatagaaaggtaatagtttaaataaatagtaagaaacctctcaatcattcacttaacatttaaagatcgattcatgtttaaattcttggggagttaaagtgatgtgatataaagagaaataaacttttatatatgaacaaatattagcgggtcataacacacaacataattaaatggaatcataataaaatagaataccaaaatatacgtggaaaatcccttcaatatgaggataaaaatcacaggacaaactagagataatccactataataataataaatatacaaatctcaatctcttacctaaaaccttagcaataatcacaaaagaataactgggatacaaggatcacatcactgtgcacaatatctaaatccttcataattctcccgagtaattatagtaagaatctattatagatctgatctaatctgagatgaaagcactactagatgattgagaatagtctctctgtattcactttgttttcttttctttctttccctttctttccacgacccccttgatataaatatgtgttgtccttgttcttgtctctctgtgagcttgtgagcttgtgagctgtggattgaatataggctctaagcccaacataaagaactgatggttgcgtgacgcattgacgacatcatctgcatctccgaaatctgaggtctatcttctccacgtacgatgccatcacaatcagatgttagagttccttacgaacatacaataaagatcgagttctttcaaatgcaaacactaaaccacaactctctttttctcacgtctgaattatccgcctaatttaagcataacgtatgaagaaaattgtgcccagcataaaaataatgatttaaaagatttcttggcacatttagattctacaaagtatacgatatcatgtaactaatgagaatctgccttcgacacggaaggcagcagcagctgagtttggtgggagaaaattgccgggtgcctaaaattgtattggctgggagccttctcttcacaccacacgctgtgttctgcgttccaggcgtggggtgtcgtcacaacctcgtcagactaactcacctacttctctccaccccgactgcaatgttgattatggtgaacaatcatgcgtcatcatcatttaggttctctattacactcatcttcttcgcaaaccataaataagtgaataggacatgacgaaaggacgtttgacggattggaattgatgtcacgggaggacgaatacaaacacctgtagagatcaaagatacaaaataaatacgcggcggggatggctattatggaatgagataagcctgtggatcttttgttagtggctatttatatgtggccttctccactcttcttctctcaggagaagatatctcagagagagagagagagagagagagggtataacatatagcggcggcagctatggaatggtctgaagaggtcacgcccctgcacttcatggtatgcttcgctttacctttggtgctcctgtatgttgtcgctacgagaagacgagggaagctgccctttccgccggggccaccgcagctgcccgtcatcggcaacatgctgatgatggaccagcttacacaccgcggcctggcgaagctcggggagcactacggcggcctctgccatctccgtcttggcttcctccacgccttcgcggtgtccacgccggagatcgctcgacaagtgcttcaggtgcaagacaacgtcttctccaatcgcccggccaccatcgccatctcttacctcacctacaaccgcgccgacatggccttcgcccactacgggcccttctggcgccagatgcgcaagctctgcgtgatgaagctcttcagcaagaagcgcgccgagtcgtgggcctccgtccgtgaggaggtcgatgtggccgtccgctccctcgccgaccgcgctggctctgctgtcaacgtcggcgagctcttgttcaacctcaccaagaacatcatcttccgggcggcgttcgggacgcagagccacgagaaccagaacgagttcatctccatacttcaggagttctccaagctattcggctcgttcaacatcggggacttcatcccatggctcagctggatggatccgcagggcatcaacaagcggctcaaggtggcacgagcgtcgctcgacagattcatcgacaagatcatcgacgagcacatggcgaaccgcaaggaggctgatgcatccgatgctgacatggtggacgacatgctcgccttccttgatgagtccggctaccgctgccaagcaggggagagagatgatctccagggaaccctcaagttgaccaggaacaatattaaggccatcatcatggtgtgctccaatatcctatcgatgtctccctacttggttcactttctttgctcctcttcacgcaatacaattagataatctcacatcagaatgagctctctctctctctctctctctctctctctctctctctctctctctctatctatctacacacacacacatacccagagcatgatggtgatgcacgacatcatcaaaagatagcaattttttcctacccaaccacactttagagttattaaaagcatgatattttaattcaaagtagtaattattatttattatgttcttctgaattcttatatgatggttgtttgaatttgcctcaataaactcaattgcttgacgacgcaggatgtgatgttcggcggcacggagacggtggcctcggccatcgaatgggccatggccgagttgatgaagagcccagaggacatgaaacgggtgcaacaagagctggctcatgtggtcggactcgaccggaaggtgcacgagagcgacctcgacaagctctccttcctcaagtgcgtcaccaaggaaacgctccgcctccacccccccatccctctcctcctccacgagaccgccgaggactgcgaagtcgccggctacaccgtccccgctcgctcccgcgtcatgatcaacgtgtgggccataggccgggacaagtcctcatgggaggacgccgacgcgttccggccgtcgaggttcacccccggcggctgcgccgcgtctctcgacttcaagggcaactacttcgagttcctacccttcgggtcgggccggcgctcgtgcccgggcatgcagctgggcctgcacgcgctggagctcgcggtggcgcagctgatccactgcttcacgtggacgctgccggacggaatgaagcccagcgagctcgacatgggggacgtgttcgggctgacggcgccgagggcggtgcggcttgctgccgtccctgcaccacgtctcagctgtccactatactgatcactcttccacaaagaatgagagcgacgcgttggtgggtgtgtttatcttctgctgttcagacaataataatgctttattctatatgaaaacatatatatatatatatatatatgcatgaaatattttgtatcggagattgcttcgatgtacaccacagaataagatcactgattgttgattgcagctcattgcggtgttttccattgtctcatggtggactaatacatcacaagtataagaaatcggccacattggtcggaggtcaaatcgcatccgcatatgcgtccttatttaaagtctttggtttcgcgtcaactccactagcatatgtgattaattatatataccttcaaaacctgtgataatttatttacatcaatacatcccttcaaaacctaaaacttaattctgtatcactataattgatgtctatcagccgctcttagttaaatttataatttctagtgttatcttttagatacttattatgatgttacaaaatattaaaagtattttaaatcatatatttgtttttatttaatcaaaaaattaatatgaaacataatttttaccttaaaaaaatatttcatttaggtcatgaaatttacataagacctataattaattgatgattgatagaaaatacattatcgatcaatctaacatgtgacaaccatggtctaaggcagatgattatgtattgtctctcccttatctcttgcatcgataagagatcaaggggaggctattggaggtaattaaaggttacttcccaacagaatattctgtctatttataattatgtataaaagctcatattcaacataatgaaaaaagatgaaggataacctttttgaccacttgcattcgtactcatatatctcgggttactaacttgagtgttaaaaagatcgggttataaaacctctctcaacctcgatctttgtataggtggcacctcgagagtttaatgtttttacctcagaggcacatcggacaaccgtacgcatacgagcctagactatattggattaaccaagatgtcaataacttcttcctgtaatattttgatactagaagaggacttgatgtcgtagaaatgcctatccatcgattctcttaataaatgctcgagtaaagagactttgccctcaacttatagtactttcacctagggggacaatagacaccctttctgcacatggacatattcacctcagtgtgaatgctaatacgatactagcgtctattcaatgaccagggtctctcacccctagggattgttaggatcgagagcactaagagggggggggtgaattagtgcagcggatatttttcagcgattaaaaaccgaaagctgcgttcgttcgataaagactattttgatgcaaaaagccgattctaagattacttatgattaagtgcagtttacgtttaaacacagttagcgtctaaacgcagtttgcgtctaaatgcagattgcgtctaaactcagattgcgtctaagcgcagtttgcgtcttagcgcagattgcgtctaagtgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 90 - DNA, 6058 bp, Musa acuminata
  misc_feature 5733..6058: note = /note="n = a or t or c or g"
  misc_feature 1..6058: note = source = /organism="Musa acuminata"/mol_type="unassigned DNA"
  source 1..6058: mol_type = unassigned DNA
ctatcataagttaaaatccatctatcagcgatcttaacttcgaatctgtcataaccttcaatgtggtgggctaatcgatcaatagtctcattgtccataaaattgttagtgctactattatcaatcaaaactataacatgctgatgttctagagttccgccaactttcatagtttgcgggttagaataatcggttaatacatgcattgtattatggtatgcccaacatcttcgttagaatctataccttcatgatcagagtccacattcttagcttttggttcctctccaattggttcaatcatcaaaagttgcccttatttacattgttactccatacttcacttttcatcataatacaaacccatttggaatctttcattgagttctttttgggttagtcttcgggtgtcagagttttggttagccgtagcggaagggaacggacgttggcgcagagtggcaacaccaatgatgaattgacagcgagaagagagcttgctctaggcaagcggctctgataccatgatagaaataatagaagataagaacaataattgaagaagctttattgtagaaataaaatagcattagcttgaatctattaacatgttccctctcctatttatacaaattaggaggaaggatttccctaacagactaaacagaatagagagatttcctcatataattggggagatcttatcctttatcaagttggggaaatctttatcttctatcagttacttgcactcgagggttctaattcgaagtgattggttaaatccaactcatgcgacattttttttcatctcagaaagagaaaacaacattaattattggtggtgaggtcttggattgattccaacttacttcaaccacaaaaagaatgaaaaggaagagagaaagaatcccaaattaggttcatgaagagaagaaagagactctacctctccccctgtatgtcattttattaggttatgaaccaaatccttggttttcagtcccaatcagtgaaggaaaataagaagaatgaaagaagcagataatgatcaacttgcatcttccaagctaagataagaactagcaaccagaattcatttaaaagcggatatattttgtttatgcatctaccatcgatattgatgtaacagctattaaataagaacaggcaaaacttcatcttcgatggtattctttgacatcaaaacattatttttacccttctgatcgtgtaaatagcttagagaacatgactgttcagctacaggtttttttagcatcaaatcatttgccaggattcaaacaacattttcttcaaagatatacgttctttaatgattctttcttgtttcagaggacaccagcatattggactacaagcacaagtgaacttgtacttgatgcttaatgtatcgactcttgtggtcaagcactaataataccacgatccaaacatgtatcctttctcatcaaccgagtccacatctgaatcaagattaattttgcgtggtaagagatatgatccttgtcttgactccttgataccatcaatcagattcccaaaggcagtggaatgactacgtgagctgccatttcaatccctgaacaccagaaactaaaggacaaggcaggaaagaagtagatcgatcctctgtgttacttctcatgtgctgtgctcactgataaggtaggtgacatgcgaagtcttgcctgcactgctcaggtgatgctactcgatccaaattgggtgaagaaaacagcaaggagggtggaatgagatggagcagttgctaaggcctgttcatggatgcgcagatggagtttggtgggaggaggtaactccattcctacgtcgtcttcaacctaccccatttaaatactctccccttctacatctatggcgtccctacgtccccccaccctcaccaagatgtacttagtgatacctcattgaattgattgcagtgagagtcgcgtagtcttctgcgtcagtggctatatatttggccttcccctccgttgtactcttcggggtaagagacaagtgatcgctttagacgatggaatggctcgaagaagtcacctccatgcgcttcgtggtatgtgttgtggtacctgtgacgttgctgcttgctgcttcaacgaggtggcggcggaagctgccgtttccgccggggccgacgccgctgcccatcgtcggtaacatgttgatgatgggccagctcacgcaccgcggcctcgctaagctgtcggagcgcttcggtggactctgccatctgcgccttggcttcgtccacgtcttcgcggtgtcgacgtcggagatagcccggcaagtcctccaggtgcaagacgccgtcttctccaaccgcttcgccaccatcgccatcacctacctcacctacgaccgcgccgacatggccttcgcccactacggccccttctggcgccagatgcggaagctgtgcgtgatgaagctcttcagcaagaagcgcgcggtgtcgtgggcctccgtgcgcgaggaagtcgacgcggccgtccgcgccgtcacggacggcgccggcgccgccgtcaacctcggcgaactcatgttcaacctcaccaagaacatcaccttccgggcggcgttcgggacgcagagccacgagaaccaggaggagttcatcgccatacttcaggagttctccttgctgtttggggcgttcaacatcggcgacttcatcccgtgggtgagctggatggacctgcaaggcatcaacaagaggttcaaggtggcacgagaagcactcgacggcttcatcgacaagatcatcgaggagcacatggccaaccccaaggaagctgacgcagaggattccgacatggttgacgagatgctcgccttcttcgaggagtcccgcgaccgcacgaaggaaaacgaggcagatgagctccagagaacgctcaggttgaccaggaacaacatcaaggccataatcatggtatgtgtccatgcataaatatgaacaacactacatctaatttatgggaggaacatttcatgaacttatccgtatcaaacttgtgatcggttcatcctttgcttcaatcactcacttctcaaccaagaacacatgcggctgttcgacttggaatcgatagactgattgctgaagtgcacaggatgtgatgtttggcggcacggagacggtggcgtccgccatcgagtgggccatggcggagctcatgaagaacccagaggacatgaggcgagtgcaagaggagctggccagcgtcgtcgggctgcaccggaaggtgcgcgagagcgacctcgacaagctcccccacctcaagtgcgccgtgaaggagacgctccgcctccacccccccatccccatcctcctccacgagaccgccgaggactgccaactcaccggctacgccgtccccgctcgctcccgcatcatgatcaacgtgtgggccatcggccgcgacaaatcagcatgggaggacgcggaagtgttccggccgtcgaggttcgcccccggcggcgaggcggccgcgctcgacttcaagggcggctgcttcgagttcctgcccttcgggtcggggcggcggtcgtgcccgggcatgcagctggggcttcacgcgctggagctcgcggtggctcagctgactcactgcttcagctgggagctgcccgacggaatgaagcccggcgagctcgacatgggggacatgttcgggctcacagcgccgagggcggtgcggctggtggccgtccctacgccacgactcacctgtccactgtactgatggatcaccccgtccatccatgttcatcatttccagtgtttccggaagaaccaagacaggattcgtacgcatgccatgtctgtgtgggagattgcttcttggtcaacatggaatccatgttgcaggcagcagaaataaagatgtatgattgtgtctcatgtgttttggtggaacaagcagtgtgtctcatatatgttatctgtctcatgtcaaataaatagactttaaggcactattattcttaagtcaagcttttttttgttatattttgttgaatatttaatttatagattattttaccaacaaaatataaaacatatgtagtgctacatatccgattacatttcacatctctcagtccaaaaggagaaaagcttagacatctttcgaagaagatttgcactgtaatcgctgctgtgtgattgtgatggacatgtacaagcgtgccatcagcaacagaggaggagcaggcggatgtcaaagaatggccaagttttgggaatggcttaggacagtacgaatctgcttctgctctttcggcaaaggtgggcgccggcggcggcgacccgggttcggacgcgctcgaactctgcggcggggcaggggatggtgatgccgccggggtggtggaatccgaactcctcctccgcctcctgcagcagctccccgaacaaggggtggttgaagtagatcaccggaaccacgtacctccgcccacgtacaccgcgatgtgccccttgggcggccgcctctgctcctccaggagcggagcccggtcgccgaggtggggtctgcgtcgtaggaggcggcagatgctccggccccagtgggcgagcctcgtggtagccgtggagctctccatcaagggccgggtgtggtcacgacgtcggccgagccgcgagacgcatcgccatatcctcgagagacggcggcccaacctgaaccctcctttgtgtctcttcataatatggagaaggtgcatcaaaacacacccattccttctctttatgcactgcacgagagatccaagaagagagagtccgatgaagaggtcagagagaggaacagtgaagggagaaagagagatggcggaatgaatataaagagggctggtcaacggtattatatatgtatatatatacatatcaattgggaattgagtacggaagaaacagaagaaggcggcggcaagaggagacgtgagagtatggagatgtgaaccgtgagttggctgcaccgtagaacacacggcctgatgagctacacaagcgtcgcgtggcggagaagagtgtggtgtaaacgcaaacgcaagtggtggtgagaatcccatgacaaagacttcacatttgttctaccaattgtctccgttctccatcgcacgctgatttgaatggggaattggagtgggaagaagcatacttttcttgtgtgcatgtataacatgttcactcggtgaaagggaagcgaagaagaagaagaagaatcaaagaagttgctcgttggactttaaaaccatgtcgactgtcagtaatgttgattattatgtatgcgtcgacgactcattccccacttaaatgtatgcaaccatcggtagctgagaaaggagaaaggataagtgcactgcttgtggcagtggcgaagggatcgatggtgagatgagcacctcctctcatctaattcggaattagaagagtactgcaccgggatggccggagctcgttactcttttgtggtcaatcaactaattgttgatgatgtgtacttacttccatagatttgagtttaaagtctatacacatacttcgataataattagtattatttacatagtgtagttaatcaagtagaacattatatcacaagatcgatattttaggtgcaaattttatagagtgagattttgatgactcaatataaaataagtgagtcaaagataccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 91 - DNA, 6104 bp, Musa acuminata
  misc_feature 1..6104: note = source = /organism="Musa acuminata"/mol_type="unassigned DNA"
  source 1..6104: mol_type = unassigned DNA
aaccaaactacatacacattgttgacacttttttttatcattgatagatcactcatcaggtgcagtatgccttgtagatataatatttctactttgtcagcattgtttattctgatatagctagtgatatggtcatattcacttccatattgattggaaagttggaaaaccaagtcctagtaagagtgagcccaaaatatgattcctctcttagagacgcccgtgcaggacactgtcgatgcatgctagttttctgtccatataaaattaaagtcctgtggatgaatcataaaggtcacctgtcatatggccatgggttttcagtttctgtttctagaatttgtagtacaaaacaaaaacctgtaactttcgagcatgtttcctatcatcattcttttagtttttttttttccttttcaacaacaaaaggtaagagtctggtgcttgttagaaagtacggcatgcgcatctgccctctttcatatttttgaagtttaaatttaaatgctgtcgagcgatgtcccgtcatttattaccatttgatctgatttttgacttacaggtgtcagataatggctgggctgaaggtgaatgcaaaggaaaagcaggttggttcccttgcgcatacattgagaagagggagcgtgtgcttgcaagtaaaattgcttcaatgctatagaagtgttgctttgcacgaagtgtgcatgttgcagtgttgggcttccataagccgaaatatcgtgtttgtgtctatggtgtttgtatcttcaacttatttcttgcattttcccccatcttacggtttgtttaaatggccaatgctgtctgatatctttgtcaaaggttaaagtagattttgcaggaaaacaaagttatttagtaatccaccacttaaattactggctttgattaatataactactgtggaatatgctgctggctgcagccaaataagatatgccacgttgcaagctattggagctctaaattgagtgctaccggtgccaatggtcctcttggtcggatgcagtggacatataaccagagataatttgtagttttctgagccaggtttttctttatgttctttacattttttgcttgctttttcgtctctctctctctctctctctctctctctctctctctctctctctcatgatatatgtgtgactcgtttccaaatgtattttgagttacagtcacagtcccgtattattctagttacattgcatacaagatgatggtttaaccttgaaagtctaactcattaatcttcatttgaatccaatgacacgtaggttttgccaggatctcagtctcttccccttggaggaagtcttgcagcaagaggcagaacgaaggaatagttccaagaggtaatgttgtcgaagattctttcctttttgatttgcgatggcaaatatatcatttcatttcactaccactgagctgttgctgcctgcgacaactactaggctccgttatttattgaggtgcgtgcgccagtgggctcattaatagtataaggatatccatgctttcagaattgcttcgtgtcgtggctcagttatcattatctttcttcattttttattgtctttgtccatgctttcagaattgcttcacgtggtgatggctcagatatcattttctttctttatctttttttttcttttcatttttttattgtctttgtccatgctttcagaattgcttcacgtggtgatggctcagatatcattttctttctttatctttttttttcttttcatttttttattgtctttgtccatgctttcagagttgcttcatgtgatgatggctcagatatcattttctttcttttttttctttttttttattgtctttgtccatgctttaagaattgccatatcattttcttcctttttttttcttttcattttttttaaattgtttttgtccattctttgagaatcgattcatgtcgtcgttgctccgatatcattatctcaatacatttatcgaagtaaaaaaagaagctatgagctgattgaatcaccttggcgttttcccaagggttgaggtgggcgtgtcactaacctgatttgtggaacccgttcgttgggttccatcacgtgcctccaatgtcatgtcggtgaaagcccacatcgaccatcattctataaatctgcactcactccctctaacggtgacacaactttcatcagcacagtatggtatgggtcgaagaagtcacctccatgcacttgattctactatgtttgatgttacctttgacactcctacttattgtcaacattgccgcaaggaggcggcggaggcttcccttaccgccggggccaactccactacccatcataggtaacatgttactgatgaaccaactcacccaccgcggcctcgccaggctcgccaaactctatggcggcctcctccaccttcgccttggctttgtccaccacttcgtggtgtccacgccggacgtcgcccgacaagtcctccaagtgcaagacagcgtcttctccgaccgcccggccaccaccgccatcgtctacctcacctacaaccgctccgacctggccttcgcccagtgcggcccctactggcgccagatgcgcaagctctgcgtcacgaagctcttcagccggaaacacgcggagtcgtggctctccatccctgaggaggtcgatgcggccgtctgcaccgtcgccaagcacgccggctctgctttcaacgtccgcgacctcgcgttcactctcaccaagaacatcgtcttccggtcggcgtttgggaagcggagcgacgagaaccaggaggagtacatcgccgtcgtccaggagatcgccacactcttgggagccttcagcgtgggcgacttcatcccatggcttagctggatggatccgcagggcatcaacaagaggctgagggtggcacgagcgacactcgacctcttcatcgacaggatcatcgatgagcacatggcgaccgacgccgccaacgcagacatggtgggcgtcatgctcgcattcctcgaggagtcttctcaccaccaccgacaagaagagggagatgatctcaagggaacgctcaggctgtcgagggccaacattagggctgtaatgatggtacgtatacatacgctctaaaaagtaatactatttcctcattctctgttactgtaacctgctaagctatatatttatctccacattgatatggtttccatgtgcttcgttaatatctcgttgcgtaattaagacgattaaattctcatttttctaacctgtaaaccgtatcatctctaattacgcaggacgtgatgtttggcggcacggagacggtggccatcgccatcgagtgggccttggccgatctacttaccagccctgatgacctgaaacgagtgcaagaagaactagccatggttgtaggactcgaccggaaggtccacgagagccatctcgacaagctctccttcctcaaatgcgccataaaggagacgctacgtctccatcccccgttccctctcctcctccaccagactgccgaccactgcgaggtcgccggctactccattcctgctcggtcgccagtcatgatcaacgtatgggccatcggccgcgacgagtcggcatggaaggacgccgacgcatatcgaccgtcacgattcgctcccggcggcgacgcggccgctctggacttcaaaggcaactgcttcgagttcttgcccttcgggtccggccggcggtcctgcccgggcatgcagctgggcatgcacgagctggaactcgcggtggcgcagctgctgcactgcttcacctgggcgctgcccgacggcatgaagccaaccgagctcgacatgggggacgtgttcgggctgtcggcaccgaaggcggtgccgctcgtggccgtccccacaccccgactcagttgcccactgaattgatcgtctctttcttggatttggtttccacgaaataatgaaataaagaagcgggatttcggtagctttccactgtacacaccattaagcttccctctgttcgcagtgtttcatatatgcaataaatgaagagcttcagctctttcctgtaataccctcttcatcagctgccaagagttacataatggtgatcgccattagagatataactcatacagacatttatcgttatcatataatggtgagatcagaaatgttgtcttcgctcctgttgtggggaacaaccttaagccatggcctcggggccaacgtggctgggttcggatccgaatgatcggggatcttgctgagcgacccttgagactagtgaggtgaccgactgcagtggtccgattacgacaggacggtcgtttcctctggaaggaaactcctcgcctgggtacgcgatgggaggtgcttcgtcttcgtccctgcacacaggtcgggtcgggaggctcgacttgaccctccgacgatcaagtcagtggatagtcttagggggtttttctcatcttttttcccccttccttgagctgaatgcgagggtttttatagggaagcttattgtctcatgatgtgcccacacttgcagggggcaggcccgtactttcgatagcgtctgacaccaatgtaggcgttgcatgaagaaccaggcttgcgcaggacgtggcgtgccctgatcgtcatcctggtctgcctcggtcaggcgcgtcaagtcaaatcgaggcatggatcattatcttggaggagctgatgtcaacgcacgtcccatcgttattaccactatcaactcctatccaacaaaataattaaaattatatattcattcgggatctttggttattattataaattaagctttaaattcatgaaaagacggatgtaattttggtcaaccacaaatgcaatcatataatcttgttttgtctatatttctccactattaaccgtcatgatcttattatttcgtatttgcatattaattaattaattaatttatttattttcttgacacccaaaaataaacgctaagaggcgacaaagctgacgtgtgatgcgatcggagaacggtataagcaaggggggcaagaagtggtgcgttatggtccggcatctattgccacccagtctccttccgagaccgcgccgccccggttgatcgccatcgccaccagcgagatcaagaacttgcacggcggggccccggccgcgatggccccgtcgttgctgaacccgacgcatcggtacgcggcgggcgctctgctggccctcgccctccgccaggcccagatccaccagacccgccccctcggctacgacgaccccgaccccgacgcacagggccgctgcagcaccggaagcgccaccagcagcgagggcggtggcgacgaccccgagctctggacccacgagtcgtgcggccttctccgccccgtcttcaggtccccctctcgttctctcctctccttcctttcgaaaaaccgtttccgagtcttcctttattctttcttctttggggaaataggttcttggatatagaccccaaagcctggtcgggcttggaggagactgcggcgtcctccatggccaagcatcacataggagcggtacactcgatttccttttgactccgttgattgttctcttggtcgaattgctttctttatgggcgatcgtcgtgataatcgtagaaactgcttattagcttgcactacttgtgagttgtgatggtaattctagcaagaatggtcggacatgctttctctgtgggtgatctagtgattatgggagaatctgcttaatcttgccctagtttctggttcttgtgagtcgttaaggtaatgatcgcccgggaggatccaacttgtttgcactgtaggtggtcatggtgataatggaagaatatgttagcttgcctgcccccatctttggtacttgtgagttgtgatggtaatgatagccaagaattgtccgacttcgtttctctctgggctgtcatggtgataatgtaagaatctgtgtcaagacctgtcatggtagccaagaagggtctgacttgctttctttataggctatcctaaccatattgtaagaatacgggtctgaccgttcg

SEQ ID NO: 92 - DNA, 9140 bp, Musa acuminata
  misc_feature 1622..1711: note = /note="n = a or t or c or g"
  misc_feature 3688..4043: note = /note="n = a or t or c or g"
  misc_feature 5860..5879: note = /note="n = a or t or c or g"
  misc_feature 1..9140: note = source = /organism="Musa acuminata"/mol_type="unassigned DNA"
  source 1..9140: mol_type = unassigned DNA
acgagggggaggggcggtactacgtggtgatcggaggagttgccgggaacccgggaaaagagacgctgagcaactggttcaagatagagagctaccaaggagtctataagctggtgttttgcccgacggtgtgtgattattgcagacctgtgtgtggatctctgggtgtctacgaacaaggtggaagacagtggctcggtatcagggatgacacgccattcccgttcgagttcaagagggcatgagcttcggtacgtgaatacatctactgtcatacatctatatatcaataaaggaggcattgtcttcggttgatggtggaggcttgcatgctttgtttgagtccccgcgtgtgtgggtatgggtgtggtggattggcattctcaaactctttgtgccacactcccatgaatcataggttaccctcaaacataggagaatgaatctctctatcaacatgagtgactcttttcatgctgtgcaaaaggataataagttatagattctgtctcattaggatatatatatttctattattatgatatttaacttttttataatgatgaaattattaaataaaattaaattagctaatggtgggttaccaccagactttttcttacaatgtattcctgacaaaatgaatttttgtccattttctttttagaggtctaatagaaaacaaaaaaaaaaggtgtggaagaaaataaaaaataaacatgggttgagttgtgcggaagagatagatctcgcggagagttaattacatattacctctaactatcttatctttagcttctcgatctatattcttataagaattatattagggtttctatatctacaaaagtaaattattgagatccttatattgggttagaaataaaagaattttattgaatcaacatgattaatataatatatcaatgtaatttttgaaagtataaggatcaaaatactaggataggatgacaatggatagagaaacattagacagggttaccgatcaatagggagtaaaatgattgaaatgggactcaacaattgaaatatttcatgggtcccacccaccacgcaaatactgcacgtgtagagagcatacttgtctcccgccgatgtgatggtgttcaccgtctcacatcgaatcaaaggatgattctaacaaagttgttcgttttgtgcttaaaaaaaaaaaaacaaatcagttctttgttaataaaattaaggtaacaaagatatgaaatcaaaataaaaatattattatggacactgtcacatctgattaatggaaaaatcttatgttatggtggtatgagattcgcatatcatgtagcttttttatttccaagtgataaattctctcagcttttattatttattctttttggaggtttccttgttaattttattattttacatatcaaagacgatcaactctttggaatgtaaaataataatgagttagttagggcattttagtatattatatatatatatatatgtatatatgtatatatgtatatatgtatatatatatacatatatatacatatatacatatatatacatatatatatatatgtatatatatatatatgtatatgtatatatatatatatacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatatatatatatatatatatatatatatatatatatataactgtgcgttttcgaaattacttaacgtatatatacttgtagtgaacaaaaatctgatcacccttatacgagagccgttcattctagttaccttttaatctcgatcgttcaggttttcccagaaaaaataaatataaaaagaatatgactcgatcatatcagctggacggccccccctttgggtcactataaggagcgccctccggccactccgggtcctgccgacaccgctgtctgtcggtcttcggtgtcctcagctagaagaggagccatggattggtttcaccaactctcgttcatggttgcttccgttttcatcccactcgctctcttatcgttcttctgcatgagaagcggacgaaagcttctgctgccgccagggccgcagccgctaccgattatcggcaacatgctcatgatggaccagctgactcaccgcggcctcgcgaggcttgccgagagatacggcggcctcttccacctccgcctcggctcccttcacgccgtcgtcgtgtccacgccggagatggcccggctggtgctgcaggtgcaggacgcgtcgttctgcaaccgccccgtcaccgccgccatcgcgtacctcacctacgaccgtgccgacatggccttcgccaactacggtcccttctggcgccagacgcgcaagctctgcgtcatgaagctcttcagccgcaggcgcctccagtcgtgggcgtcggtccgccaggaggtcgactctgccgtccgcttcgccgctagacggagcgggtcgtccgtcgatgtcggcgatctggccttcaccctcgccaagaacgtcaccttcatggcagctttcggggcgcagagtcacgggaaccagggcgagttcgccggcatactgcaggagtactcgaagctttttggggagttcaacatcagcgacttcctcccatggctgcggtggatggacttgcagggcatcgacaagaggctgaaagtggcaaggcaagcgatcgatcgctacatcgacgtgatcatcgacgaccatttggccaacccgaaggaggcggatgcgcaggacgccgacatggttgacggcatgcttgcatttcttggggattctggtgacactaacgagggtggcgatctccatggtgatctaagcctcacaagatctaacatcaaggcgataataatggtacctctctctctctctctctctctctctctctctgacagctctgccgtcaattataatgcctttgctagtattagaatctcaacatagattgaggatatatatgtagacgaatacacgtttgaactcagagatcagattttagtttgagtaattctgtggcatgtcagtcgcaatcatgtcttacatgtcgtcttcagagatggagacatcctcttcggctggatgttggcattatcttatgcaagcaggcaacaaagccagcttgaaaaagtcattgaaattgttgtatttgttggggtaaacaactttatgccgtggtctcgggaccgacgtggcttggtttgggtcggaatgcgcggggatagccggggtggccagttgcgatggtccaggcgggacgttgcgtgtggagtgaagcttttccgcagtgccgggaagatgcaaccccgcccttgtacctgcacacaggtcgggtcggaagctcggctcgacccctccgacgatcaagttagatgatgtggagggagttttttagtgaagaagtgtccccccctcctcgtttagaactcgggggtatttatagggcagtttagtattacctgatgtgcctgcctgcacgagacaggatcgtacctctgatggcgtctgacnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtgggcaagccgtcctgaggtttgctcggcagtctgtggccgaggagctcggcgcaggcgggctgcggccaagggatgcaactcaggtgggcaagccgccctgaggggggctcggctgtccatggccgagccgtgtagattaagaagattttaagccacgagccgagagggggtcaggtagatactggaccttggctcgaaaagggctgaggcagggcttggatttcttttcatgaagactacatggggtagccaccgcgggtgctctaactctgttacggagcccgcggtcgggggccgttgcttctcctcatggccgcccaggcttccgccgcgatgtgccggttagcgcgctggagcatctctggcaccgtcgtgggaggttgctccgcgagtgaacagaggaatctggaaggtcgcaggccgatcatgaacgcctgcactaatagagaggagtgagtgtccggcaggccccggatttgcgtggcaaagcaatccacaaaatgggagaggggctcatcctccccttgtttgagtccgaggagtggtgccgcggaaggcttcggccgtacataggccacaaagtggagctcgaagttcttggcgagccgatcgaaggaagcgatggtcccggtcttcaggccactgtaccacgcgcgggctggaccccacaaagttgttgggaacgctctgcacatcaaggcgtcagaagtcccatatagcgccatctgggcgcgaaaagtggctacatgattcgctgggtcagcggtgccgtcgtaggcgtccagcgaagggagtcggaaatgcgatggaatcacttgatcttgtatttcgggcgcgaacggggacccttggtgtccgtccgctccgagctctccttttgacctgcgaacctcctgctgtacttcgtcgaggcgtcgactaacgaggcgcaactgggctcgcagggcgttcgtcgagtccgcggatagcgcctcaagctctgggcgactcgacgtgtcttccgcctctcggttcccgggctgggctgttcggttccgaggtgaagcagggagctcgggaagtggtgcatgagtctgggtggggggctcccgctgctgcagaggctgggttatatgcggggtcgtcggttgggagacgagcgggatgatagtttgtaccacgcccgccagtgctcggacttgatgagtgaggtcgtggaaggcttcgggcgatacaggcgacgggccgacggggtcgtcgggtggcgacaaacccgggtcgttgaacaaccgccagtagcgctccgatgtcgtggcagggcgttcgtctcggggttcatcacgcatgggatgttcttctagggtgttgatcggacgtgaccccatggctgcgggttcgccagagtggatctactgatcgctcgacattcggggccctccttctagcgccaaaatgttggggtaaacaactttatgtcatggccttggggccgacgcggcttggttgggtccgaatgtgcggggatcttgcgcggcgttcctcgggacagccggggtggccggttgcgatggtccgggcgggacgttgcatgtggagtgaagcttttccgtagcaccgggaagatgcaaccccgcccttgtacctgcacacaggtcgggtcggaagctcggttcgacccctccgacgattaagttagatgatgtggagggagttttctagtgaagaagtgtccccccctcctcgtttagaactcgggggtatttatagggcagtttagtattacctgatgtgcctgcctgcaggagacaagatcgtacctctgatggcgtctgatnnnnnnnnnnnnnnnnnnnnatcgcccttgtcgttgcgtggagaaccgagctgccgtagggtatgggcgtgcctcggtggtcgttcttctctgcctcggtcaagcgcgttgggtcaggcaacgatgaagtcttcatctgagagcgggtgacatcagcgcccatcgtgtcggcattattgccctctttcgggcagagtgtcgtccagccatgactgacgtcgtggcgcgtcatgtcgctattattaccctcgtcagtattgatctatacgtccaaacacatggaaatataaactgttggaggtcgcactctaacgtggccttcgttggcatatatggctgcatgcctacgtatcttttttctttcttcctcgcagcgttttaaacgacaaagaacgataaaatagtcgaattcccttcaaattacatgaaattgaaatagtatcgttgtgttcgcatatattttttgcacggatatcatttttgttgcacacatacaaagcatctgaggattatactaaggtcgtactgcttctatatgcattcatatgaggattgcaatctgtacgtgacacacacacaggatgtgatgtttggtgggacggagacggtggcgttgggcatagagtgggccatggcggagctactcaagagccccgaagagctgaaacgaacgcaacaggaactggcgagcgtcgtcggcctccaccggaaagtggacgacagtgaccttgacaagctcccctacctcaagtgtgccgtgaaggagatgctccgtctccaccctcccctccccctcctccaacaccaggctactcaggactgcgagctcgccgggtacttcatccccgtcgggacaagggtgttcgtcaacgcgtggggcatcggccgggaccgggacgcgtggaagagccccaacgcgttccggccgtcacggttcgccctgggaggcgacgccgcggcgttcgacttccgagggagctgcttcgagcttctgccgttcgggtcggggcggcggtcctgcccgggcatgcagctggggctctacgtgctggagctggcggtggcacagctgctgcattgcttcgactggagcctgcctgccggaacgaagcccggtgacttggacatgggcgatgtgtttgggctcactgcgcccaaagcagtaaggctgatggctgtcccaactccgcgcctcacttgcccgctcctataagaagcttgcttctctcccgcgcggcactctcgttataattgagatattgttcctcgttaattggcctatgaacatgcagctgaaacatctcaattgtgcctttgttatgcaactattaagcttttactccttgatatataaatccaaccgcttacaataacgaaacatttgccagagaacaacatgaggagcaagtgcagaacctgtatattcccatgtttgcaccgaacttaccataagagttgttgaaatcacaaaagattaatgaccctggcgcaaactctatattaacacaaaagatctggtacatgcatgccaccagtatgaagatgacaagatttccggggagtcaagtctgggccaatgggcataattggttaatttgttttgcagcagaccagagtcttggatgttgttggtcatctttaaacataatcaaattcgcatcggttcccagtatcccctggctgatcaacgttgctttttcaacatcaaacagtgatatcttcctcgtcccctgtccaggtgtagcgattgtcttcccacctacaaaacaaggtctatatatacggtaacaagttccagtgttggattctacatgaccttgaagttggctgtgaactggtccagcagcgtttctgatgtcgtagagatgcaatatcgggtcactaccggctgataggatcaggaaattgttccatttgttccattcaaccgtgttgacaccagtagagtgcagtgatttgatggtaagagcacaaggctccggcgaccttggatcgaggacacatatacgtccatcgactccacagtcggcaagtcgttcttttccatccctgcaaaaatattcaggaacaacatatcggtactctgaagcactgaaaaatacatggcaattcattttcgtaaagcaaatcattgtgttgaaaattataatgctctagagttgaaaatattgttatgatattctatttagattattccgactctcaatttaaagtttgtttgcagctacactcaatgcatagttaaattcagaaatacatgtgcacaagatgggtcaacagaacattgcttctatatttgcatgttctataaaccattcctccgtaaatttcgccagtcactagcagcaaacttcatcctcaagaaagttccagtcattaaaagatgaccagtttggtatataagtcggcgacgttatgaaccaagaggtcgttgtcttctattgacaagtcacttggtatgtccttgtagaactacacaagaaccttgatgctattgagtgaattccaagttcatgaacaatagcaagattacaacagttgagctggaaactcatcagctaccggctttcagaaatgtcatgtagaatacgctacaacccaccttgactgctacacacattattcatccattgcttattccgttatccccttctatgcaattaaggagtgaatttgaagatgagtgtcattgactcagaaagaaaagacaagaaaatttaagagtcggtcttcgaaaataacagtgctacaacataagatagtatcctctacatttgaatttgtgagagcatcctctttttgtaatgcaagttttgggtttcgggagccgcctcatagatcataacaagttgataactagtttatttttctttaatgaaccgattgagttgatgatatgcgatacttatgataaacacattatcgatctatattttctaccacaaattgtaaataaagaatcaggatttcaagagttcactttcatactatcaggaaaaaaaaactaattctaagatttaaatatctaaatcatgaattaagaccggaaatctccttcagagaaggtggcatggaaaccaattcaccaacgaaaaccagacatatcttaaattgaagaatgatgactattgtgcatcaaagagttaagtaatagaagggttacccaaaacaaacgcccctgatgactcctgaatggtggccggtaatgttccgctctgccaccagctcccctgccggcgtcagcctcgaaacaccaacagaggagtcctttgatgccgtgg

SEQ ID NO: 93 - DNA, 5925 bp, Triticum aestivum
  misc_feature 460..475: note = /note="n = a or t or c or g"
  misc_feature 4207..4216: note = /note="n = a or t or c or g"
  misc_feature 1..5925: note = source = /organism="Triticum aestivum"/mol_type="unassigned DNA"
  source 1..5925: mol_type = unassigned DNA
cctggttgtgtgggggtcacactgcctcataaccaagcaaccagtggttgcttctcagaaatggattattttatcgatgtgtagagttgatagaatggtataagatccgtccactacatcaacacattgaaagtacatagaggatgcacacaaccattatactcacaaagctaaattgagacaagaaagggccatgaaggaaaaggacaaaccattgacctcatggttacaaccacccacccatgttcatgtcatgtctaaactattcgatccacaaccaaagaaagatgaaatgcaagtcgagctgatgaaaatgtccgcactcaaaacctttactcatcaccaatgaagacatgaacgtctaaaactcggaaatgattactccaaggccatagcctgaaccagagatggaacccggtaccgagataatacccgaaagcacggggtggtgtgggggggnnnnnnnnnnnnnnnngggggggggggggaggggggttcgcacctccatttttctgttggtatgggtttcgtgggaagaataaatgaactttacaatccactcaatgaaacctcttttaaagatttctttcacgcagaataaggcaaaaaccataagaagtttaaatatcaacctaatcttacctgtgttttattgttaattttttttgaacagttttaatgttaatttgagtatgtatttttttattcataagttttcctttaatataaatcaaacaaacaattctgcagaactcactatgtccacaattttgttgcccatacaatcctatccgtgttttaaaaattccgcaaattatagaggccctttaaaggggtatgtgcgacaattccctgctgccaccgaatgctagaatgatccagagaatcccagcaaacacagtagatgccacaaacaccaccatcccctacaacgtcaccccatgaggttgttgagatctgatttccgcgtgcagccgtggcatcaatccaatggcacagagccccgttgaaacctaagtttaatgtccacatgttcacgtgctataaaacaattaacatacactgcttgtgacagcttactaattgaaaggtgcttagctagaaatcctcaatacactgcttgcgttatagcttactaatttaagtcgcttagcttgaaatccccaattattgggagcactacacaagctatatatgctgactggacatatatataccctcccccgtaccaccaactccatttaaaaaaaaatgctaacaactccacccaagaaactaaaaaaaacttaaaagaaccctcaactactcatctaaggcatcaaagaatatatcatgcacaccttcttttatgaattataaattgcattattattttgttttaaaaagtcatagatttagatgttttaacatatctcacttaggttatttttatgaaatcaacaaaagcatctgttgattattcttggtccaggctcatgtgagggtaaggctcccaagcacggaaatagccaatttaaggttcttaaggtttatcaagaaaaatattattttcaataggtactttgcgtaaatacagcaggagaatactactttatgttggatctagtgatattgatttgatagcgtgcatgttgatatttttcttgataaccagagtcaaacttaaaaatcaagtaagccttgtataaatccatctggagggagtattaattatttgatgcaaaaacagaaacactagtgatagttcacgtgtgatgcgcagggcgtgcaagtggaataaaagaaaacacctcacccacaagaaaaaagaaaagaaatccttttctaaaaaaataaaaaagaaaaggaaatagtcggcctcgtgtgcaattgctatcatgcggtgtgacgctactgtttttcaactacagtagcacagtatatataaaggccccatgcatccacagggattacagcggagtttcttgtttgaagctagtgctcgatcgttcttattgccaatggcgacctttgcaaagatcgccatggagctcctcgcggatccactgatgtggctgttcctcgcctcgctggccttggtagccatgcagaggcggcggctgggcagcgcgccgtttcctccgggccccaagccgctgccggtcatcggcaacatgacgctggtggaccagctgacccaccgcggcctggctgcgttagcgaaacagttcggcgggctgctgcaccttcgcttcggctggctccacgtcttggcggtgtcgacgcccgagtacgcgcgcgaggtgctgcacgcgcaggacggcgtcttctcgaaccgcccggcgaccatcgccgtggtctacctcacctacggccgctccgacatggcgttcgcgcacaatggcgcctactggcggcagatgcgcaagctctgcgtcacaaagatcttcagccgccgccgcgcggagacgtggctcgccgtgcgcgaagggtacggggcgctagcccgcgaagtcggccggcgcagcggcgaggccgtcaacctcggcgagctcatcttcaacctcaccgtcagtgtcatcttccgcgccgccttcagcacctgcgacgaggacgggctcatcgagttcatcgccatcctccaggagttctccaggctcctaggattgttccacatcggtgacttcttcccgtggctcgcctgggtgggccgtcgtggcttcaaccgccggcttagcacggcgcgcggcgctctcgacaggtttatcgacaagatcgtcgacgagcacatgaggaggggcaaggacccggcagaccccgacgccgacttggtagatggcctgctcggcttcctcgctgacgcaaacccagtcagcggcaagcgcagagaggacgctctccgcttcacgcgggataacgtcaaagctatgatcatggtacgcgagtaatttgctgtactggtatactctccctgttcacacatctcaagaacttcacttggtacattttttttattactgtgtactactaatacatttttttttgcgagggagtactactaatactaatgaactaaaagcatggcaagtattttggaaccaagggagtacaatttttttaagatagctaacgtctatccatcatatgaaatgttaaaaagaaattccacacccacaaaaaaaatccacacatattaaaaaaagttccatttgttcagtttatcattctactggtatatgacttcctgaaagagaaggaatattatttcctacaatatattgtggtgacgtggctatcttcatacggcatataggacatgttatttggtggaccggagacggtggggtccaccaccgagtgggcgatggcagagatgatgcgtagcccagatgagctcgagcggctgcagcaggagctcgccgatgtggtggggctcgaccgggctgttgaagaatccgacctcgacaagctccctttccttaggtgcgtcgtcaaggaggcgcttcgcatgcacccgcccatcccggtgctcctccacgaggccgccaaggattgtgttgttggcggctactccataccgagaggctcccgcgtgttggtcattgcttgggcgatcaaccgcgactgcggggcctggaaggatggcgacacatttcgaccagcacggtttatacccggcgagggggaggccgccgggctagacctcaagggaagctgctacgagttcttgccgtttgggtctggcaggcgctcatgccctgcgcaggggctcggccaacatgcggttgagttcgcagtcgcgcagctggcgcatggtttcaattggaagctgcctgacggcatgaagccagcagagcttgacatgggcgacatttttggcctcaccgcgtcacgctccacgcggctctacgccgtgcccacgccccggctcacctgccccgtgtaatgtgataattattggacaaggcgtgcagtgcccacagagacgtaatatcatattgccaataatgctacacctacaaacaacctataaatgtttacttaacatttcaaactataattctctgccccctgaatttagcaaagtttgccccgttatcacgttttttgtatgtgacaagcataacatatccgactattgtcattgtgaaccatcatcggagcttgagagagagagagagagagagagagagagagagagagagagagagagagagagagagagagnnnnnnnnnngagagagagagagagagagagagagagagagagagaatcatatttggtcttacaataaatgctggtcttcttgtttattattttgcaatgtttgacttcggctaaaagatataatctttacgtatcaagacaaaatataccattcattagtgtagatatgcctttctcatttagcatgtaagtggcattgcgcacaagtataagagctggctaaagttgggaatgtgatatggaggagttcaagtcataccggaacctaaaatctcctcgacatagattctcaaagaaaaatcttctccacagacaaaattaacatgtgagcgagctataaacctgctattgtatttactctgatacgctgatgacttcgacttggggagcatgaccgcgaccaccacacaatgcccaagacaaaactggtcgattagccaactgggcgtccacatctcctaggcgtgccacatgagcgagctcgtcaggtatatatacatataccataccacactgtcattttggagcatcccccaccacccactggtccagtaaatccagccagttaagcctcctcttcctcctcctcctcctcctcctcctcagtgcactgagtgctggtccagaaaaagaaaatgtgcttcgagttcagcttgtgcttcggcggctttagagatgactacaacggcgatcggtccaaccacaacaagtccggggactgtcagcgacgccgtagagggaacaacaataggaacgactaccatgcatgtagtaatttttttcacgaatacatgtagagcgtatcaacgcatgtgcatggctgtcgttagctcgtgttgtaaggtgttgggttaagtctcgcaacgaacacatccctcgtgtctagttgccactgtaagtttggaaccctgaacagaccgctggtgttaagccggaggaaggagagaatgaggccaagtcatcatgccccttatgccatgggcgacacacgtgctataatgggcgggacaaagggtcgcgatctcgcgattgtgagctaactccaaaaacccgtcctcggttcggattgcaggctgcaactcgcctgcatgaagcaggaatcgctagtaattgccggtcggccatacggcgatgaatccgttcccgggccttgtacacgccgcccgtcacactatatgagctggtcatgtttgaagccattacccttaactgtagggaagggatgcctagtttgcttccttctattaatgcattgatatgctctgcgtattcgcaaaaaaaaaaaactaaaaaaaaactactatgccgacggcgatagaaggcatcctaccacaacaaacagcccaccgccgtagacgaggccgggcacaaggcctatcatgacggcgctactggccacccggccgtttcgccgccttcaagacaccaaagcttcccagcgttcccgggccgcacaacacggcgcacctccaagaaccagctccaacactgcccaactgccactaccacgaccgctcttggcaggtagtccagattagctatgcggctatacaccactgtaagtaggattcgctagcatgggcatggcagtccagttcatcaatcgctccctctccctaaatactgcatatgcttctgttctatccgtagctctgtttttctcctctgttttttctctcttttcgaattttgatcaagtggaagtgcagtgatgtatg

SEQ ID NO: 94 - DNA, 9466 bp, Triticum aestivum
  misc_feature 356..456: note = /note="n = a or t or c or g"
  misc_feature 1623..1635: note = /note="n = a or t or c or g"
  misc_feature 5116..5148: note = /note="n = a or t or c or g"
  misc_feature 1..9466: note = source = /organism="Triticum aestivum"/mol_type="unassigned DNA"
  source 1..9466: mol_type = unassigned DNA
cgggataggtggccccacccggtggacccgcgggacccttctggtggtctcggtacaataccgatgacccccgaaactttctcggtggccgaaactggacttcctatatataaatatttacctccggaccactccggaactcctcgtgacatccgggatctcatccgggactccgaagaacattcgggttaccgcatactattatctctacaaccctagcgtcaccgaaccttaagtgtgtagaccctatggtttcgggaaccatgcagacatgaccgagacgactctccggccaataaccaacaacgggatctggatacccatgttggctcccacatgttccacgatgatctcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnactagtgcacctatacaatttaccattgtattgggtgtgttggggacacaagagactctttgttatttggccgcagggttgtttgagagagaccatcttcatcctacgcctcccacggattgataaaccttgggtcatccacttgagggaaattttctattgtcataaaaaaactctatgcttggaggcccaacacgagtctacaagaagaaggttgtgtagtagacatcaggaggcgcgatgatagaacgtctctactgtgtctgagccaccccgacgatgagtgcttgaccgtcgcgcgtcaacatcatcctcggaccgactatgatttgcatgcatgtagcctatgcggcacctgtggtggctacaatgaggccgagacacgccatgcctgtcgcgagaaggcgaaggcggtgttagtgacgtcccgatccaacgacaatccggtcatccgcgtcgtccagcagcgggccctcacctccaaggaatcaaatgcgcgctgccgccgtcaagtcaacaccaaggtcctcgcggatggcatcaatgatttggtccgcgccaccgagaaggtcgcgcatgatgctcagctcacctatgtcgtcccaggccacccccgaactccggcaaccccacgcagggcacctccacagattcctcgtcctccgactatgagatctgtggcgttccagccatccctacattttctttagccgctactaccgtgacaaagacgacaagggcaagggcagtcgtcggtgtgaaactttcattgtcctagtctagtttattgttgtgttttgttgtcatgtgttccagtgttgaacaatgtcctatgttaaactttgttcctatgtgcatgctatgtacatatatgatgatcgaggttgcatttatttgtatgaatttgaggattgaaaaatgagggattcgactacccgaacaaatgaaggttgagtccatgctaccggggacacgtctggacaaatgaggcgccaagatagcccgtcccattgtggttgaagatgatcttacaatccgtaacttctctccttatgtttagcaattttgaaaaagtttacacttttgattatcgaaagtcctacatccacgggctcttatgaagcccctacataaacttccttattaactctatctcccccccccccnnnnnnnnnnnnncaccctaatttaacgagggtgggccctagcccctgatccgggtccaattaattactttcacgtcaatttttatgtaaaattctcacgtaaatttttgtaggtgtaacattattctttgactattcacttgtgagacctagggggaggggctcgaaagttgaacaaatatatagtatcaagtgacataggattaaaaactaaaagaaaaaactaaaaaaatattacgccagtctccatgcaagatctcacgaatataatatcgactaagacataacaaagtcttagtcgactgaaatttagcaacactgatgaatatataaaccatgcatccacacggatagctgtggagggctcttgcagtattactattatcgctatccagctcgtgagtcgtggctatcgtcaatggcggcctatgcaaaggtcggcacggagtttctcaaggatccactgatctggttgttcctcgcctcgctggctttcgtcattctgcagaggcggcggctgggcagcgcgccatttcctccgggcccgaagccgctgccggtcatcggcaacatggcgttggtggaccagctgacacaccgcggcctagcggcgctagcgaagcagtacggcgggctgctgcaccttcgcctcggcaggctccacgtctacgcggtgtcgacgccggagtacgcgcgcgaggtgctgcacgtgcaggacgccgccctctcgaaccgcccggccaccatcgccgtcgtctacctcacctacggccgctccgacatggcgttcgcgcacaacggcgcctactggcggcagatgcgcaagctctgcgtcaccaagatcttcagccgacgccgcgccgagacgtggctcgctgtgcgcgaggggtacggggcgctggcctgcgccgttagcaggcgctgtggcgaggccgtcaacctcggcgagctcatcttcaacctcaccgtcagtgtcatcttccgcgccgccttcagcacccgcgacgaggatgggctggatgagttcatcgccattctccaggagttctcaagcctcctaggattgttccacatcggtgacttcttcccgtggctcggctgggtgggccggcggggcttcaaccgccgcctccgcacggcgcgcggcgctctcgacaagtttatcgacaggatcatcgacgagcacatgaagaggggaaagaacgccgccgaccccgaagctgacttggtagacggcctgctcgcgtttctcgccgaggcaaacccaatcagcgggaagcacagagaggacgccctccgcttcacccgcgataacgccaaggccatgattatggtacgtaactggtactgtatttaccatcttacagtctttactctttactgtatatccatggcggtgtgctcctgctccacactagttctactgacggacggcttgatctgcagagtagtatgaccaatatcctacgacagaaacctcgtacatgtaggctagcacctctctttctttttgacgtgtttgctatttgtaattgtaagttgcttgataatgacaaaaacgagatcggtcgattttccttaatgaagaaaggcgaggcggcgccgagctccaaggcggagaggggggcgacgaggccagtgtcctgagtaagagtaagagcatctacagccgtacgcctcaaatcatcccacataagcccgcggacacgcccggccagtgaccggaagggagagagaagaaaaaacgtgacccaatcggatccctcgtaccatccctatatgcccgggctgtccgcgaaccctcatatccatctcaaatatggggaggatatgagggctcgcggacgcgcccgagcgcgttcgggcattctgccacgtaggactcggccccacaccagaccacctttttttctttattcattcttttatttctctctcttcatctccaccgatcacgtgcaagtgaccggagaagaaaaataaggagtgtggctgcgcggacgaataaatgggggctcaaaacggacacgcccggccactggccggacatgtccacgggtgtttaaggggtcatatttgctatgtccagctgtagatactctcagaaaaagccattaactagcgggagacggggatgcagcgagtgggagccttgacggttggtggtgtggagttggcacccgggatcagagaagaagggtggtggagggttagaggggtagcaggggtggcttgggcacggaggcgaggcgcgtgaggcagtggcgccagctgtaggtgacgagggctgatggtctggcggatgtcagacgggcggctcggcgcaaggaggaagggaagggagtgctaaaaaatgcacgatcccttttttcaactcactggtccttttctttctatcctcgttatttttagtttctttttttacttttgtatttcggtcaattgactttctactacgtggttttgatacctaattattatctattatttaggtaatatactctggcttggatggtggttttttgcttgttgctgatttcttgttttattataattattttccctcttattgtttttgtttttatttttggtttgttgctagtttcatcaagtgcatatacgtatttagatgtccttttgtgtcttttcttcacaaaaaagtgtatatacgtattttagtgttttttacatttcaaagtaacggagtcgcagtagacatgtacctttgatctttagagtataagtattaatagtattacgacaaatacaaatacacttgttcactgaatggtttatggtgaaaggtgagattcatgtaggttgtattttcattgtgtttttggcgcaagatctttgtctagaataagtaaccgtgcattacaattgttttcacatgctatgaaacatgaaaaacatagcaagcaatgctgtaactgaggtttgaaatcacataaatttggtttttatttgatagagacgtgtgtcattgtttgtatggtgcatttttttatttatactgagttggaaatcggtcaacatgatagtgccattcgtcgctgtttgtatggtgcaaaattttgatttgttttttgtagcaaaaatgttgtacggaaccatggttcagccgaagctacgctcgacaccattgatttttaggcactattcataaggatccatgcatggttgattaaaaaatacagctgacatgcatgtctactcatgaggatccatgcatggttgattaaaaaatgcagttgacatgcatgtcacagatccacatgtagtaggatgttaaatcgtatgacatagaccataggttcgattgaaccatggtcctgaatcatgtctctctttttgtagcttgtccttcgtggtacctgctatcgtagccattcatatatgctcagttccatgtggatgtgtgcatcgatggaaaattagcaactggcgttagctctcaaatcttaatctcctgccgctaggactagtagggnnnnnnnnnnnnnnnnnnnnnnnnnnnnnnnnnccccccccacccacagactgagacgtagtgcagacctctctctctttttgcatgcagcgcatctttctctcataaactttacatgcaactctctctttttttctctctttcggtaaagtctgctctttaaaaatgtcaaataaaaatcctaaaaatcacttccttgaaaaagaaatcactattactcaatgcacatgggacagaggggtggagataaaagattctccatgcaaacccctctctttctcccggttggacaaagattccttcatggacccgttctcttctctcgcctagcacatttagtgagatggagctaggaaggaactttgcgaattttgattggtaccaattgaaaaccaatttaatggtaaaaaatagtcactctctacataagtatgtataatcaaaggctaatataggttaatatgcacaactccttgccactgaagacatgaataattttgtaatggaaataaaattcttcaaggtgccctttagaataaaggaaccgtggagaataagaaagtgaggcttgcaatcataggatacacaaatatgggatttgtacattttttattattagattgcattaaagaaaatttatgtatgacttgaacttttgctacaaatttgggaggaaattaagtctgagttccagtctcgtgtcaaacttctttagaaagttcaaatgtgtcttctttgtttttgtctcttaaatataacacctaatctcaaagtccatgtatcttgtgcattttttgaaaagatgtatcttttgtgcgcgcatctaaatatgtcttctcaacaactcgtatttctcagatgaaaaaatagccaagtaaatcattgtgtgtttttttgttcttaccttgtcagattcatgtgtttaagggaaagaacccaaaacattaacaatagttaaaataacttatcgagtatgttaaatccaaacgcggttttgggtgggagagagagagagactacctaccatcaacatcaccgaattcctttagcaataaagaattattcctatatatagtactccctctgttttaaaatatagtgcgtccgcgctttctgatgttcaactttaatcataaatttaatcaatgagaccgactatggcgggagcaaaaaatatatactccctccgtccgaaaaagcttgtcccaagcttatctctcaaatggatgtatctagcactaatttgatgctagatacatccatttgagggacaagttttttcggacggagggagtaattgaaaatttcttttgaatacgaattcattggtataacttttgctcccaacacagttggtctcgttggttaaatttataatcaaagttgaagcacaggaaccgaggacgcactatattttggaacggagaaagtatgtggtaacttgcatgtggctatctacacaggacatgttgtttggtgggccagagacagtggggtccatgaccgagtgggcgatggcggagatgatgcgtagcccagacgacctacggcgactgcagcgggagctcgccaatgtggtggggctccaccgtaccgtggatgagaccgacctcgacaagctccctttcctcagatgcgtcgtcaaggaggcgctccgcatgcacccacccatcccactactcctccacgaggctgctaaagattgctttgtcggtggctactctgtgcccaaaggctcccgcgtgttggtgaatgcttgggcgatcaaccgtgaccccggggcctggaaggacggcgacacgttccgaccttcgcggttcatgcccggcgagggagaggccgccgagctggacctcaacggcggctgctacgagttcttgccgtttgggtccggccggcgctcgtgccctgcgcaggggcttggccaacatgcggtggagttcgctgtcgcgcagctcgcgcacggattcaactgggagctgcccgatggcatgaagcccgcggagcttgacatgggcgacatctttggcctcaccgcattacgtgccacgcggctctacgccgtgcccacaccacgcctcacttgccccatgtaagtatcgcaccagacttgctcatggagtgccttgtaaccgttggtccgttcattgtgtaccccgaccggcttggtttgttttggacttgatctccgagtcctaattcgcaagaagagacttcctatcaataaagatcatggtcatagatgtgcgttcatgagctatctcaatattcaaattgctcatgatattcgcgggggcgtgcgtgtgcgtgtgcgtgtgcgtgtgtgtgtgtgagagagagagagagagagagattttacatcatatcaaaaggatgtgcttttcagaaccatattggtatattatagttgttgatattctgatctatgatatggaattttataatgtttgaattataatagaaaaaaaacatgctttatgtttagaaaccggcgagtgccatttagggccagttattttggaataataccattttttgggtaaaaaacaaattaccgaattcctccccaatcctagcccaatagactccgcaccatgcccatcctaagattcgctacgctccgtgatcattactccttccaactccaaaatgaagtcttataagttttgtctaaagtcaaatggtgcaaagtttgatcgattttttaacataagttataggtgttctttttggagccgaaaggagttcaccacaacccattatgctcttgagcatactctcttccccctaggttcttaaatagtttacatgcatctacttgtataggcctaagtccgtaaaaaaagtacttgtatgcaactccaatgatgtacatcaaaatgaacacctcaaatgtccgcggtcagcggagttggtccttcaaccgtatgcatcaaatccggacagcggagtgagccagacgaacctaaaggacaaaatacatgagttgggaaggacatgtggtccaactgatagtggttcaaatggattatccgaactctcctaaagctccctcaggtttggttcaggtttgtaggaattcagatgtccaacctagtccgcagacgtttgctgaactgcattggagggcaaaaagtgtctgtaccgcgtggttcaaacgttcaagggtgatttggtgcacatcgtttccaaactaggttcagagccagctctttttagtttactttgcaacttatggtcaaaataagctaaagctagacataacttgaaatttctctccagcttaatttctaagcccgtccaaataatagttggtagaaaaaaactacaagagaagtgcttctagagcttctccgttagggctggaattcgagccgagtcgagccagctcggctcgacttgctaaagctcgtttcgttaacgagctagctcgactcgactcgttactataacgagctcaaatccaagattggctcgactcatataactcgcgagctggctcgtttagcttgttaagctcgttaaggatatgaacataaaaccttacgaatatgaaaaaaaatattacttgagaatgtataatgtacatattaaatatgtacaatgttcacaaaaaattgtaagcatgtatgagcatcacttacaattgttagtatcgtgcaggcgcagcgccgacgccgtcacaatttataggataaagaaacaaggaagacatgagggaaatcgaccgcctataacttcgctaggaatagaaatggaatgagagaaatcttcgtgtgcgatgtgtgagtcttgtgggcagttgggccttgtgttgtgcgcctgggacaccgagagcttggctgatcttttgtaccgagcctaaacgagttacacgagtactcgtgagatcgtctcgtttaacttggtctgtaaatgatcttaaactagagctcggctcgactcgttattcttcgagttcgagtcgattcgagccgagtcatgagctactcgtttagctcgcgagcttcgagcttttcttccagccctattctccgtaattcaaacccttccataaaaaaaagataatgtttccctctttttaggaaaaaaatgctaccgcataactcttcgtgctcgaatagaaaaccacatgcgcaattgcttcccctttgctccaacaccaactactactacgcctaactccacttaattaaccccaagggacaattgaagtactttctagtttgatgtggaagtattgagtgttcaagccctaaggagcaaatggataagcctcatattctatcaagttatatatggttgtgtgaatttctttggtactctactcgttttaataaatggttgtcta

SEQ ID NO: 95 - DNA, 5861 bp, Triticum aestivum
  misc_feature 1..5861: note = source = /organism="Triticum aestivum"/mol_type="unassigned DNA"
  source 1..5861: mol_type = unassigned DNA
ttaaattatcctttactaacgaacatatacgtgatagcattgaatgatgtctttgaaatcagagtttgcggacgggtcatttgtccgcggacagatgccgatggaaattttacgggtcagccttggagatgcttttatactactactccgatacacggaaatctcaggatttgttacattatcagattattaatttttttgcgaatgtcattatctggaagttgaacaatctgttggaggttgacggaagaacgtgaagaatccacgcagctcgggcgccgtgtatatgcgacgaagacacacgaattccaatgactttgttaatttcaacatcactggttcgccttgtctgatacgttctcacaggtcggtttgctgtcacggaccgggggagtctgaacgagtcctgtgtacgtggtacaaccagtaactgtcttccgccaacattcaaggattggagcgaagctagaaactacaagaggttccgttgactaactcagtgatctcctgaaaagatcactaataaggttttactgtcaccttacaattctatcgaacccatgtatttgtaacagattttcttttttttgcaagggatattgtgacagtttctttactggtaatcgacgggggaagcccgttgttttccctcctcattttctcattgtgctttcgaaatcacgtgaatcaaataagatgcctatgggtatacttttcttttcttttcttcagattttcttatcgaggtgcagaattgtaaggtgacggtaaaaccttattgttattttctcaggagattactgagtgatattgatggaaaaaaacaacaacatgctcctgtagccggtccaccgaaagcacttttcacccttaattttgtttcccaccctcaagcaaacagtgagatttagaaaactcgacttttttatttatttgtggaaaaaagagagcatcccgtacggtttcaagtaactatataatgtctttcttacaaggaaaatgattgataccaaccagctgaaaaacaatttggcgccggtcgtctccttcagttgccacgcgtccgcttcgcttaccgcaaccttatctctttcttcttgcctcttcttccccgctggcatgcacaacctcctcttgctccaaccgcgatttgtggctcccatcaccggtcatcttccctgatgtatgctccctcacccggattcgtgagtcagattcacggcctctattgccggccatctccctcaccatgagcacccgccccgccaatgttgcaggtgtgtcacttgccgccagaccaaatccacaagctacatcctctctctctctctctctctctctctctctctccacatgatttttcctaccgacggatttcttcttcttctaaaaaagccaaatatgctttttccaacaagtgtcttgttgcaatgacaattatgtcatcgagcaggcgcgatgagattttctacaacaagatatctgttacaaaggtttctacaacatgaattctgttgtaaaggattctgcaacattaactgtgttgcaaccgtgaagggcgccgcttgaccgcatcagatctgacagctcgcgacctgaccgatcttttcggaatatcaatctgccgacgcatggcacgccccttcatacctctcctgcactagaaggggtaaaacagaatagtacacgcatatcatcccagtccggtgccaggccaacctctctcggagggaaggccttattcgtctcggaggggggctaacaaagaccggggcaacggttgttcccattaccatttgaattttcttgtgccatgcatcctcgagcctactcctttccctacttcattccagttagcaaatctcactatataaatccggctccttccactttcctcctcagctcactccacgagcgttatcaacatataggttcccccacaattgaataacccaacaaaacaaaaaagctagagcttccagcagtttgagtatccgtcaatggtgggcttggccaagatcgccatggactggctccaagagccactgagctggctcttcgtagcctcgttcgtcttcgtggtgctgcagcggcatcggcagcggctgcgtggcaaggcgccaccgctgcctccggggccgtctccgctgccgatcgtcggcaacatgtttatgatggatcagctgacccaccggggcctggcggctctggctaggcagtacggcggcattctccacctccgcctcggccaggtccacgccgtcgtcctgtcgacgccggagtacgcccaggaggtgctgcaggcgcaggacgtcgccttctcgaaccggccagctaccgtcgccgccatctacctcacctacgaccgcgccgacatggcgttcgcgcactacgggcccttctggcgccagatgcgcaagctgtgcgtgatgaagctcttcagccggcgccgtgcggggacctggctcgccgtgcgcgacgagtccgccgcgctcgtccgcgccgtggcccggcggagcggcgagtccgtgaacctcggagagctcatcttcaacctcaccaagaacgtcaccttccgcgccgcgttcggcgctgacgccgccggcgacgccgggaaacgggacgagttcatcgccatcatgcaggagttctcccagcttttcggcgggtccagcatcggcgactttatcccgtggctcggctgggtggaccagggcctcaacgtgcgcgcccgcaccgcgcgcgccgccctggacgagttcatcgacaagatcatcgacgagcacatgaggagaggcaagaaccccgacgacgtggacgccgacatggtggacgacatgctcgcgttcctccctgaggcgaagccgaagaaggacgctggcgacgacctgcagaactcgctccacctcacccgtgacaacatcaaggccatgatcatggtatggataccggaactcccatttctaattcacccgcaaaaagaaaaaaaggaacttccatttttaatttgtttgattcatggtatggtgcggtaaactgaagtgtgtatgtgacaggacgtgatgtttggtggcacggagacggtggcatcggggatcgagtgggcaatgacggagatgatgcacagccccgacgacctccgtcggctgcagcaggagctcgccgacgtggtgggtctcgaccggaacgtggacgagtcggacctcgacaagctccccttcctcaagtgcgtaatcaaagagacgctccggctacacccgcccatcccaattctgcaccatgagaacgccgaggactgtgtcgtcggcggctattcagtgccccggggatccagtgtcatgatcaacgtattcgccattggccgcgacgccaaggtgtggaaggacgccgacacattccgaccgtcgaggtttatgacgggggaaggggaggccgcaagggtcgacttcaagggcagctgttttgagttcctgcccttcgggtccggccgccgctcatgcccggggatggcgcttggcatctactcactggaatttgctgtcgcgcagctcgcccacgggttcagctgggcgctgcccgacggcatgaagccgtcggagctcgacatgaccgacatcttcggcctcactgcgccgcgcgccaccaggctctgcgccgtgcccacgccccggcttacttaccctttgatctctgatgttgatgccacgcacaaggcgtgacgtcaagtcgtatgcgcgcacaaaagtatattcattatgtagcacaataaagatgtttcactcgattcagggtaacactatatgagcttatattagacgacgggtccatggattcaaaatcgagtcaaactttgtaaacattgaccaagcgataaatcacgagtggacatgcggccacgcaaggaggaaatcagctccgtccactcccgcgtgattcgtgcacttattatacaaccagcttgtataataagcattgtatagtctttgtattaaatcagagagggcagctgtaagtcccacacgtcagttgcatggaacggcccgacatgcaattaatcagtcacccattacaacggctagctgcagcctgcagcatactcctctcgttgcctgcaccacgctcctccctctttaaatcactcaggccttcttcttcctctgcccaatttgcttctactatttgctctttctctctcacacacactttctagcatcgaatcgacccctttcacaaccatgtgttcatccatgtcccaagctatctctccaccaccttcatctcacgtcatttcccaatcaaattgcatctcttcctctcatatgctgcctcgagtgccaccctatctacgtgctctggttcattctagaagtgggagaaacaactacatacagttctacagcgccacatgaggtcaactcatatcgcactcaacagtctactgccatgtcccacttttcgaagataatcaagctttgctgaagaagataaccagccatgcatcagccattgccgtgctcttcctccacaaacacgaaaacttagtcccaggctgcaatcgtcgtctacttcatttacttttcagtttagaccttatctcaatccaaaccagccatgcatcagccatcaatttttttttctcaccaaggtcgtcaatgtaatagctagtaatttcagtttggaccaagctactcaatgtatgatccagtattggctgcttgttataactacccactcacattgtaaatgcctgccagcaatcaaggcacccaggaataattaatggcttcatttatccagtgtactacctgctcagcctgtattcatttattaggagcaggtcgtgattcgttatccttggccataatcacagtttggtattttaattttgtcacatgttttggactactgatttacaactgaaatctttacatgtatcattttctcacaaaaaaaatcagttagcttcattcttcattgttagctcgaatcatgaaaattacaacttgaatgtaaaacacaacatcaataaaaaatcagtacacagattctacataaaagttaaaatatttttacatggcttatagaatatttttgcggctttgattttgatctcagcttcacaatcatacttgtttaccaaaaattataattctcattgacaaattagagacatcaaaatcagctccacaaattcctaagtgaactacaagcagcatttaattaccaaaatgtatattgaaagtttgtcatggttgcagctaaatttgtgtgcttagtcaaaatggtagattaactgtggcacaacgaaccttagccgaagccaatcacacttgcttgctctccacagggacgcaacttgaacaagagctacaaggcatgccatgagcaaggccgatgcttctggacggctgccccacgtccacagcaattatgactgacccaacatgcaaggggcgtcgctattagatgagccgatattcatacgaaaactaaatgagatctggcccacatgtgcaaggtataacattgtgtatttatgctggcctgtgaattacctgctccgtattacgcagaggcctatattaagtagcaatcgcgatgcacatgcagacgcttgagatttcggcatcacgtgccaccatgtcccaaaaatccgcgtcgttgaccaagtccatagatacttttagattccccatgaaacatatcgtgcatttgtgggagcacttgctacagccaagcatgcatttttattccctccgttcctgaatagtctttttagagatttcaataagaggctatatccaaagcaaaatgagtgaatccatactctaaactatgtctacatacatccgtatatagct

SEQ ID NO: 96 - DNA, 6651 bp, Triticum aestivum
  misc_feature 1..6651: note = source = /organism="Triticum aestivum"/mol_type="unassigned DNA"
  source 1..6651: mol_type = unassigned DNA
ctgcatttgaatagtgtcatttctattgtttgtcttcgaaactcccatgttttgaagacttccataaaaatattcacccccccccctctagtcgataactatccctttcaccttctctggtactctgccggagggggaatcaatcacggggggcttctacatcatccttgctgcccttccgatgatgtgtgaatagtttaccacagacctacgggtccatacctagtagctagatggcttcttctctatctttgatcttcaatacaatgctctcctcgatgttcttgaagttctattcgatgtaatcttctttcacggtgtgtttgttggatccgatgaattatgggtttattatcatattattcatgaatgtaatttgagtcttctctgaactcttttatgcatgattgttatagctttgtatttctctctgatctatccgtttggtttggccaactagattgatttatcttgcaatgggagaggtgctttgtgatgggttcgatcttgcggtgctcaatctcgatgacagaaagggacatgacacatatttgtattgttgccattaaggctaaaaagatggtgtttattcatattgattgtgtttacttttgtctacatcatgtcatcttgcataagacgttactctgtttgtattaacttaataccctatatgcatgctggatagcggtcgatgggcgaagtaatagtagtagatgcaagcaggagtcagtctacttgtctcgcacgtgatgtccgtatacatgatcattgccctgaatattgtcataactatgtgtctttctatcagttgcccaacagtaatcgttttacccaccgtatgcttttgttcaagagagaaacctctagtgaaaactatggccccgggtctacatttatcatatataaattccaaaaataccttgctgcaatttatttaccattatttttttgtgttttattttatctatctatcactacgagatttgatccttgcaaataatcgtcgaggggattgacaaccccttgattgcgttgggtgcaagtatttgcttctgtgtgtgtaggtactgtggccgatgttacggtactcgatcccgaggcacactccggcgttgaatcgcaaccgagagagcgggcatttcctactcttgaaggacttcttcgaacaccaaacccgctcttcaaacatcacctatttccattgtcgtttccgtatggctaggtatgttttcaaccgtatccgagagggagtggtgtcatacggtaagtatttcgagtgcaagaaatatgccctgggaaagattggcttctcctcttatcagaaatgcaatacagctattcggatgcttgcatatggagctcccggtctcattgatgagtatgtccatatgagcgagtccacgtgcctagctgacggtatctatcctcagtggactactcttgtgaagacaacctccaatcctgtagaagagaagaggaagagatttgcccaagagcagaaacttggagcactaagaagttgtggaaggtgatgactgcttgtgtgagcatgcataatatgatcgtagaggatgagcatccgaaacgtatctatgaccaagggtttcagtttcagggtgacaatgttgttcctgagcatggagggataacgttttgcacagttttccaatttcatcattaaatgcgtgattgaaaattttgcattcagctgcaaaatactatcctcattggatttgaaatatgtggatttcaggagcaaaatatgagggcccgtccggatgcgtccacgtccacggacatagagaagtcggatttgcacgtccggttatagatgctcttagatctcactcgtgagctttgctgatttttgggccggcaccctcgaacttttctcctcggctctactcgaaaagcgttactccagtctaaaacccacacagtagaataccccaagaagaaaacaataagaactagagcttctagttcgagtaccagcaatggtgggcttgaccaagatcgccatggaatggctccaagatccgctgagctggctgttcgttgcctcggtggtcttcgtgatgctgcagcggcggcggcggcggggcagggcgccgccgctgcctccggggccgaacccgcttcctatcgtcggcaacatgtcaatgatggaccagctgacgcaccgtggcctcacggcgctggcgaagaagtacggtggctttctccacctccgcctgggcaaggtccacgcctttgccgtgtcgacgccggagtacgctcaagaggtgctgcaggtgcaggacgccgccttctccaaccggccggctagcctcgccgccacctacctcacctacgaccgcgccgacatggcgttcgcgcaccacgggcccttctggcgccagatgcgcaagctgtgcgtgatgaagctcttcagccggcgccgtccggagacgtggctcgccgtgcgcaacgagtccgcggcgctcgtccgcgccgtggcccggcggagcggcgagaccgtgaaccttggcgagctcatcttcaacctcgccaagaacgtcaccttccgcgccgcgttcggcgccggtgccgccggcgacgccgggaagcaggaggagttcatcgccatcctccaggagttctccaagctcttcgtcgagttctgcatcggcgacttcatcccgtggctcagctgggcggacccgcagggcatcaacgtgcgcctccgcgccgcacgcgccgcgctcgaccagttcatcgacaagatcatcgacgagcacatgaagaggggcaggaaccccgacgacgtcgacgccgacatggtggatgacatgctcgcgttccttccggaggcgagaaccaagaaggccgccggcgaccgcggggacgacctccagaacacgctccgcctaacccgtgacaacatcaaggccatgatcatggtacgtggtagatccaaacttccgtcaattatttactgccatatgcgccaaaaatagattttttctaagaattaggtaagcttcagtcaactctctagctcccagatctagacatcaagagccgtgtagaatttctgtattagataaaaaaatgtttgtttattgacactaagaaccggaccgactccgtcaacttttaaatgcttattatacgaaaagtagccgctacactcggggtgtactacacccgactttgcaaaaacaactgaaaaaacgttatcaaaccaaatctgaaaatctgagacatcaaaccttatcaaatgttcgagcttcttgtaaactttcagcaacattgattttgtctttttattcggtgagaactcctcggatgttatttttggctgaaattttacaagatcctcaaacatttttataaagtttatatgttaaattttcagatttttcaaatttggtttgatcatgttttttcagtttttcacaaacttggttgtactaaacccacgagtagaacatctactctgacgcttataaggtactccctctggtccataatgtaagacgttttttaacactagtgtaatgttaaaaaacgtcttacattatgggatggagggagtagatctatcgttcaaatgtagtggcggtgcacaagccactaacttcagatcaactcgattcttagctctaacttcagatcaacccaaaagaaaatgagttactattagcctccttttgcagctcggctacaaaatgagctgagtttggtcgcaagtcttacctcgctcgattttacctcgacatagctcagaatactcccttcgattcatattacctgtcactcaaactgatgtatttagtcatccagatacatccacttaaacggcaaataagtaatatgcgtcgaagagagtatcataatgtgttttttctttaataatacagggaagctaaaatgtgtttgttaccagtactatgttttttaaactaaaatgtgcatgcatatatgtatgtatgtacgacaggacgtgatgtttggcgggacagagacggtggcgtcggcgatcgagtgggcaatgtcggagatgatgcactgccccgacgacctccgacgtctgcagcaggagcttgccgacacagtgggtctcgaccagaacgtggacgagtcggacctagacaaacttcccttcctcaagtgcgtcatcaaggagacgctccggttgcacccacccatcccattgctcaaccatgagaatgccgaggactgtgtcgtcggcggctactccgtgccccggggctcccgcgtcatgatcaacgtctttgccatcggtcgtgacgccagcacgtggaaggacgccgacgcgtttcgaccttcaaggttcatggagggggaaggggaggccgctggggtcgacttcaagggtgggtgctttgagttcctgccgtttgggtccggccgccgctcgtgccctggcatggcgctcggcctgtactcgttggagctcattgtcgcgcagctcgctcatgggttcaacttggcactccctgacggcatggcgccgtcggagctcgacatgcgtgatgtcttcggcctcactgttccacgtgccaccaggctctgcgtcgtgcccacacctcgactcacctgctctttggtcgctgatgatgatgccgcgcaccaggcatgatgtcatgcgtgcacctattgtctttgtgtgccccaccgcctgagtttggtgggtttctctttgcctttttcttcttctattttctgatactacagaacacaataattggatcttctatccttagcttacagattgtgatgcgtggctatttttcatgtatatttttttgggagggggtaatgacacatgaagcaggctctaccatgaagatccatatttcggtggtagtttgagcattgcacgtttcatgtcatcatcctcgcttgttgcgacgagttgttgagcaattatacaacaacgattgggataacccactacagcaatagagacatgttttttttttgcttgatttttgactttctagttggctttctcccaagaacatggagtacaataggaagttagtaaaaatgtaggatttaaagatgtactaaatttcaaaaatgtgcacgaaagtgaaaaatgttcagaaattttaaaaatagtcatgttctaaaaacacgaattggaaaaatatacactaatatcaaaaaatgttcacgcatacaaaaatgtttgtgacctttaaatttttttgtcaattaaaaaaatcaaatgttttgaaaataaggtaaaagaaaaccgaaagaaagaaccgaccctgctagccactccgcccgcttctcgttcatggtagtagtagcggggcgttctcttgaggcaattcgagccggttctctcagtttttttttctttttcactctttttgcatttgtttctttggttttttttatttatttttctttgatttattttgtttcagtttttgtttttaattttttagttttctctgtttcctttggttttaattctacattttttgtgtatgttaacaacatttttcttcacatttaacattttttcaatacaaaaactattttttaatatatggttaagatttccctatcacatttttaaatgcttgattaaaaaaattgcatacaagattaaaaaatttaatacatggtcaacatttttctatacacaactaacattttccatacccttgattaacattattcaaatacttgttcaatatgttttttcaaatgcttggttaaaaaattatatacatgataaaaaaattcattattttttaatacatagtcaatattttttatacatatttaaaaaaattcaaatacttgctcaatgtttttcaattgattaacatttttatatacatgatcaaaaggattcatcatttcttaatacatgatcaacaatttttttatacacgttcaatattgtacaaattcttcattcacatttttcaaatacttgttcaacatttttcaaatgttttttaaaagtataaaaaagtacaaaaataaagcaaaaatgaagaaaaaaataggctgtggctagtcaacaaggcccaaagtggtcaacaaaagtcaagaaagctcagggaaatcaacacaagtccaagacagacaacaaaaatccaagcaagttgtcgttgtcaataagtcacaagagaaggcaaaataaaaggcccaactagccaaaggcccattcatgcatggatgaagctgagtggtgcaccgtaaactatggagaccttcaactttggccaattttcatggactcatgggcctagttttatcgcactacacctcctacatacccaccaccatgggcagcattaaatcatttgtcaaggcacaccccctgtataaggcctccataggcatgtaacaggttcactcacgtcaatagactaaggggagggcactagagatcttcgagttttgctcctaaaatgctctaggatcgagtccgaggagccgcatcgcgactcagatcgacgctagagatctagaagcctgctctcaagacgttgtaggggcaagtccttggagccgtatcgcctctcggatcgacctcggtcgagagttagggagagggtttcaaggagtcgcgtcatgacttcgagcctacgacagtccatcctaccc

SEQ ID NO: 97 - DNA, 6761 bp, Triticum aestivum
  misc_feature 1..6761: note = source = /organism="Triticum aestivum"/mol_type="unassigned DNA"
  source 1..6761: mol_type = unassigned DNA
agtcataataggttaagtcttaatccgacttggactctacatgtaagccactccttgaacttatatatggaggggcaggtgaccctaagagggtcaagtttttagagggtcacagtagacaggttaagatagtcagggctttgacagacaagtctaaaacaagatcaaagtagatgctctcgagatagaggtagtgaaacacgatcatcatcatcaatatcaatgaagtaggatgtaggcttttacctccaccgcgagggcccgagcctgcgtaaaatcttgcgtctctcgtcccactcaaccccgctcaagctaccatttagatgagttggcctcacgactaagtcctcacactaagatatctgccgtgacaaaatcacgacaacgagtgttgtcatccgtggtggttgttgttgggtggtcgagttcgcttcgacaggaatctagtggttgcattttatttttcgtgttcgtattggtgttggttgtgtgcctcccaatcgtgcaaaaatcgagcatgcgaccccttcggtaaataaaattcgccgttcaccaaatgactgcaatttaaacaccaagagacaggacgcatcttgctgtaaacgagaacttgggagctgtggctcgctgactcccattcattagaaaactgacatatcttatctactactactatatatagtctagcactgagatcgatctggaatcgtttcggccccaacgcgcatctctgctctctgccgcaacatatatatctctcggccccaatcattctcggtccatctgccgtttggctacgtggcacttctggccggccctgttggcatgaatacgtggcccattacagtattgattagagtacggtcagaactaaagaatccacaagatagcacgccgtgcaggttcctgcatgctgggaatctttaggacaaaaaaaaaagtttctcatgtttttgttcacagcggcgattaataaggacaagcttgagccagcgaaaattgacatatgagatcgggcactttgtgcccgttattttggagttcgaaaattgcatttatacggcttgaaaaaattccaaaattttatgtacatgctatagcagtgtagtataacatgccaaaatttcgctaataaacatatgcttgcattgcatgtgagagctaaaaaagacaaaatacatgcaaatttggggccagtttttttcgattttgagctaatttttcgccttttttgtgcagtgtatacaataatgggcacatgcatgcgtattcatgtgttttttttccaaaaattttaagcacttgttttggtgggttcaaaataaagtactccgttgccccatgttctgaaaatccattttattaagtcaggccctgatcgatgataagcctagtcagatgcttattactgtaagaaccgatgcagagatgcctgcagtaaattgttaggaaacaacatccgtatcttgtgaaattatttacaacaagtacgatctgacatggatactctaatttagatcacagtatccgtgcatatgggtactagtgctgcaaaaatctctgctggtttttggcattgttatcttgatccagataacaaatcgacatggcttgtgcaatgggattccattgttgcatcagctctcataattaggctaattttgacaaattaccagtagcatgctgaattccaagcataccactaaattgtctaaacctacaaagtttctccaccggtgcctcctttttctaaactttgagcgactgctggcctgacttcacacgacgcatctttaatatggttcaaaaagactgatgtattgtttcagctggtctgcctcatctcaatttcgggagaatattatacggaaaccaatattaataaaaaattgtgaaaatcatattttaaagttttgaaaaaatctgaaaaaaaattcggaatgttaagagtgtgatgttttattgccacgcaatttcatgttgaatttttgcgtggtaataaaacatcaccctcttaacatcccatatttttttcagattttttgaaacttttaaaatatggtttccacaaagttttcattaaattggtttccgtatgatattcttccctcaacttctgctccaactatatattacccggcatcctccactcttttccttaccctcttacaacacatcaacctgaaacctgcactgcacagtcgagaattctccaagaaaaaaaagttagagcttctagtttgagtatcaataaatggtggacctgtccatgatcgacatggaatggctccaagagccattgagctggctgtttgttgcctcggtcatcttcgtgttgctgcagcggcggcgtggcaaggcgccgccgctgcccccaggaccgtattcaccgccgattgtcggcaatatcttcatgatggaccagctgactcaccggggcttcgcagcgctggccaagcagtatggcggccttctccacctccgccttggcaaggtccacaccttcgccgtgtcgacgccggagtacgcccagcaggtgctgcaggcccaggacgccgccttctcgcaccggccagcaaccatcgccaccacctacctcacctacgaccgtgcggacatggtgttcgcccgctacgggccattctggcgccagatgcgcaagctgtgcgtgatgaagctcttcagccggcgccgtcctgggacctggctggccgtgcgcgacgagtctgcggccctcgtccgtgccgtggcccggcggagcggtgagcccgtcaacctcggcgacctcatcttcaacctcagcatgaacgtcaccttccgcgccgcgtttggcgctgaagccgccggtgacggtgatggccggaagcagcacgagttcatcgctatcatgcaggagttctccaagctcttcggcgcgttcagcatcggcgacttcatcccgtggctcggctgggcggacccacagggcatcaaggtgcgcctccgcgccgcgcgcaccgccctcgacgagttcatcgacaagatcatcgacgagcacataaagaggggcaggaaccctgacgacatggacgccgacatggtagacggcatgctcgcgttcctccctgaagccaagccggacaaggccgcgggcgacgacctgcaccacacgctccgcctcaaccgtgacaacattaaggccatcatcatggtacgtaagcatcaccaaacatccattatgtgttgttagcctctatatatccgtctaccatgtttattatctatgatgttttctttattttcgttctctattgttttctgaattcctttccaacaattgaatcttgaatcaaaaagtggtttggtatgtcatgtgaatatggatattaaattaaagcatatgtttgaaaatattcttgctgacttagtagaatatttatgtctctattgcatatacaccgacaattaattagtaatcaaaatgaatttgtgccatccaacattttttcggaaacaattatggaaccttaacactgttcagccaacaacaccacccattgaactttcgtcatccatgagacttctatgtttttctaagtgacatactccctccgtccggaaatacgtgtcctaagaatggttgtatttggacttgttttagttatagatatatccattttatttatttttaggacaagtatttccggacggagggagtatgatatactgtaagtactatcagactgctctgtttgtgtgtgtgtgtgtgataataaagtaatgtgttgacgcggcctgcctgcaggatgtgatgtttggcgggacggagacggtggcatcggcaatcgaatgggcaatggccgaaatgatgcacagcccaaatgatctcttgcaactgcagcaggagctcgccgacacggtgggtatcgaccgaaatgtggacgagccggacctcaacaagctctcctttctcaaatgtgtgatcaaggagacactccggttacaccctcccatcccgctgctccaccgcgagaatgccgaggactgcgtgcttggcggctactccgtgccccagggctcgagcgtcaatatcaatgtcttcgcaatgggccgcgacaccaaggtgtggaaggacgccgacacattccgtccgtcacggttcatggagggggaaggggaggcagcgggggtcgacttcaagggcgggtgcttccagttcctgccgtttgggtctggtcgtcgttcgtgccccgggatggctctcggcctgtactcgctggagctcgttattgcgcagctcgcccatgggttcaactgggcgctgcccaatggtgagaagccgtcggtgctcgacatgagtgacattttcggcctcaccgcaccacgtgccacaaggctctgggtcgtgcccacgccccgacttacctgccctttggttgttgatgtctgacgccgcgtgccagacatgatatcgtgagcgcacagctgatggtatctaccagctgcgcagggcgtgatggtgtttgatcagcgtgttagttaatctagttgtagctgtagctattttccccttttgtgttagatatttcctagcttcgtttagacacgatcgcacccgtataaatcggctgccctggacgtggaattgtgggcatcgtggagccgggtcacgatcgttcctttcatcatgaataaaaaggaaatgaagaatgacagaaaaacaggaagttgttcgtagcacaaatgtttacaccagcatgttattcttgtcctttgtttctcctccttccgcgaacttggttgataacgatgttgttttcattttcgagagttagttacacttttgatatataaacctgcaattgatgtacatattcatacgtaaacttgaaaaaatgatacaaatcggtgctacaacttgtgtttcagatacattcaagtacatttcgctgatgactctccgttaaattctgcacgttagtttacctacacttcatatctgcgcatgggtcacgcaaagaaagaaagagaaatatatatatagcaaaaccgaaagaaaataagagcgtacgccgcgcattgccgctcatggtaaggcgccccagcccatccacctttgacctaggcagccgccatctccaaaatctaaggttttctgcctcccgccggcgccggcgccggtccgtcccgtctccggtggccttagggccatggagacgtggtggatctcagcccttgccggtgggagggctccgtttttagatcttttttcaagctttgttaggatttgtatcctgctcaggaaggcaagacggcggcggctccctgaagatggaataaatgtctccccgcctagccccccttccggtgatgcgcttagcatcatcggtgggcatgtggaggtgtgtctccggcggatctgtctttggtggatttgctcggatctgttcgtcgttcgtgttctttgtttgtcctcagatttgatccttttgatcgacactcttcatctgcggcggttgctgttctggtgcgttggttctatggacccttagcacgacgacttcccgactgtctactacaacaacgtttgcccggctccggcgagggaggggcgatgacagcggcgcgccttcggcgcttcagtgcttgtagtcgtcgctaggtggtctacgaatctggatgtaattttttattatttttagtgtttattgcactaacatgattgaagatgaatagattgaaagtttttcccgcaaaataaataaataaatatatatagcaaaacccattgcgcacggttggaatcaaactcggaaggttgcccccaacactacacgtgccaaccaattcaccacgtgcagctttctattagcgcgcgttatatatagacattcctttagatgactaggaaacaattatgaagtcaaaacatacactgctagatgagagaagtccttatttaacaccgtgtctaaattttatgccctatttgacatcgacaaataatgtcttccctatctaacaacgagtctaaattttatcccttttataacacttctgtccattttgagccaaatgacacctgaaaagaccattttgccctcacgtggtttgtttgtgtgcgggtgtgcgcgcgcgtacgtgtgttcctgcttagtgtgtgtgtgtgtgtgtgtgcatgtgtgtttgctattgtgtgtgtacgtgtgtgtttgtcactgggtgtgtgcgtgcatgtgtgtgtgtgcgggcgcgctctgttgcgtgcatgtgtgttgtgtgcgtctgtgctgctgtgtgtgctacgtgtgtttgctgcagcagcgtgtgtgtgctgcatgcgtgtgtctgtgtgccgcgtgcgtgtgtgccgctgcatgttgagtgctgcgtgtgtgttgcctcgtgtgtatgtgctgctacgtgtgtgctactgccctcgcacagatgttacgtgtgtgctactgccctggtctgaacaacgtgttttttcttctcttgtttggttttattctgcgcaggaccagacaacctggtttccaaaaaaaatctgaatttctagtagctttttcttcgaaatttccaaacggattttgaattcaaatttcaaacaaaattgagttgaaaaaatatgagtttttgaccgatataaaattgggggcttaccaaaaatattaaatgaaatttaaatacatgcatacaagtttattttacaatatctgaccttttaaattgaaaaaacttaaaattttgaacgatatcaaaaacattgggggctcact

SEQ ID NO: 98 - DNA, 5635 bp, Oryza sativa
  misc_feature 1..5635: note = source = /organism="Oryza sativa"/mol_type="unassigned DNA"
  source 1..5635: mol_type = unassigned DNA
accacatggtattacatggcacgtcaattataatttaggcatttcgagatgaacatgacgtacacacttgtaagatgaagatgttctctccacacaaccataaccagcccattacccctcactatatatactaccaacttcttcatcggtcgtgcattgttgtattctttcgccaactcgtttcttgttttcaaagttgtgtttgagtctgttcaagacgaagatgaagtgcaggtatgtaaaaccagaaagtcgttagcgtatgattaattgggttataattattacaaacttggaaaacatatttatttgatatttctaaaaacaacttccatataaaaatttccgcataaaatgtaccgtttatcagtttaaaaaacgtgctaacaaaaaccaagataaattttgtatcttaatcaagttagaacgtatacccaccaatgtttgatccgtcaatttcatattgtattgctgattaagtttggtataatttgccattctatttgctcttaaggctatggtaaccactgtacatctctttgcatgtttaatcactaaattcatgattttatttgctgatcttgcgcatattactgtgttgtagttgatattttatttgtattcatttgctctgttttggatccaagagaatttaatgtgtatgtactttctgtttagtttcaggtaggataattgaagttggatgatcctacacctccctttaagtatttcattcactcccacaacaactgaattatttgtttggtggctgggtgtccttcttcaaccgtgcatattttcattgtgctaaaaatgttatatttcctcttgagttaaagtggtctcaaatatgaaatacctaataatgttttccctttctaatcctaaataaattaaattcctgaatatttctttgaaaatgtttttttagtacatatgaagtatactgtataccacatatgcttcagtatgtatatgtgtaaagcggcatattaattggatatgaagtgtatggcgtaaaatcgggttagattataattgtaaatttcagttcaatttgcaggtttaagctggttatccttagattgagagtttttaatcagttccatgaatatagaaattattgctgttttttcccggttgtaataaatccacagacaaggttatctgaaccttgcatgaacatactctttaatactatctccattttaaatacatagtattgtttacttttaaacatatatgtccattcgtcttatttaaaacaaatatgctaatatgcaaaaatataaactgtatttaaagtaactttaataataaaataaatgatgttagcatattttctaaataacatgaatgatcaaatatatgtctaaaaatcaacggcattatatactaaagaaaatggagggagtaggaatcagcgtatctgctaaaattaagtcggatacaaccttataatctcaatataacctcgtaaccatttttcctctttgagtgccaggctatctaacaacactagctccaaaagttcttgatgtggcatggtggacatgaatttgtaactgcatgcaggattccagctcagagaattgcaggctgttgatatctcatccctgatttctggtgcccgttattgtatcatctagcacatttagagctgatgcttttgataattaaattaaggagaaaaaaaagagaggaactgatgttttttatcctaatctgaggagaaaaataaataacagttgcttttaagtcactaatctaaggagaaaataaagaactgatctaaccactaatccaaggatgaaaaaaataacagagatgaactgatccttttgccaagaaacccggaagaaatgatccaggaatgaaataatcttccccaaactttcgttgagcaatttatactacgaaatttctacctatataaataggcgcattgcccctctcccatttctcaccagcttcgatcacattccgatcaagctgcaacaatggcgaacggcgtggcagagtacttgctgatggacccgtggcttgttctatggctcgtcctggcctccatggcgttcgcattgctgcacctgcggcggcgcgcacgccgtggggcgccgccgctgccgccgggtccgcggccgctgccgatcatcggaaacatgctcatgatggaccagctgacgcaccgcgggctggcggccatggcggccaggtacggcgggttgctgcacctccgcctcggccgcgtccacatggtggtggtgtcgagcccggagcacgcgcgcgaggtgctccaggtccaggacggcgacttctccaaccggccggcgagcatcgccatcgcctacctcacctacggccgcgccgacatggccttctcccactacggccacttctggcgacaggtgcgcaagctcagcgccgtgcggctcttcagccggcggcgcgcccagtcgtggcgcgccgtccgcgacgagtcggccaagctggtcggcgccattgccaggagagccggcgaggccgtcgacctcggtgagctcatcttcgggctcaccaaggacgtcatctttcgcgccgccttcggcacgcgcgacggcggcggccacggcgagctggaggttctcctgcaggagttctccaagctgttcggcgcgttcaacgtcggggacttcatcccctggctcgcgtggctcgacccgcacggcatcaaccgacggctccgcgcggcgcgcgccgccctcgacagcgtcatcgacaggatcatcgacgagcacgtcagcaaccccgccggcgacgaggacgccgacatggtggacgacatgctcgcgttcctcgacgaggccggacgagaccaaaccggcggcggcggcgagctgcagggcacgctccggctgacgcgcgacaacatcaaggccatcataatggtacgtgccattctgacaccactgccatggcatgcacgtcgcgttgcttgatggagttggcgacgacaagtgcaggacttcgtcttcggcgggacggagacggtggcgtcggcgatcgagtgggcgatggcggagttgctgcacagccccggcgatctccggcggctgcaggcggagctcgccgacgtggtggggctcgggcgcggcgtggaggagggcgacctggagaagctccccttcctcaggtgcgtcgccatggagacgctgcgtctccacccgccgatcccgctgctcctccacgaggcggcggcggactgcgtcgtcggcgggtactccgtgccgaggggcgcccgcgtggtggtcaacgtgtggagcgtcggccgcgacgccggcgcgtggaagggcgacgccggcgcgttccggccggcgaggttcatggcgggcggcgaggcggcggggatggacctcaggggcggatgcttcgagctcctgccgttcgggtcggggcggagggcgtgccccgccatcgtgctcgggatgtacgagctggagctcgtcgtcgcgcgcctcgtccacgcgttcgggtgggcgccgccgggcggcgtggcgccggaggagctcgacatggcggacggcttcggcctcaccgcgccgcgcgccgcgaggctccgcgccgtgccgacgccccggctcacctgcccgatgtgacgcgcgttctcgatttggcgcgagagatgcgcaacgcagctcgaagtgtgactgtgacagtgtgagcgtgtaatctggtgtggacatcgtatgcatcatgtgtgcttatggataattaaatatataaataatatttggatgtggatttcggctgaaattttctgtcatttcgaaaaaaattcgtcctagaattcaaatagaatattttttttctcattattttttatagttgcacattggctacattatctgtagaagcttcttgattcacgagatctagcgggcgtgacaatatggtcaaagaattctttaccctagacatgggcgtcgatatggtcgtaaagctgctcttcagcttagtagccacatccataatttaatcttaggattatttttacagttgattccgatattttttcatcgtaaacatggggttttacatcattaaaaacccacatataaaatcctttcccttaaaagaatgatggtctttgaagcccttatttctttttttaatcttcgagtatgtatgaatagtttgattacgtgcattttaattatgtagagatttggaatgctattcgacaattatattatcttaatgttacattaagtgagatgcttttattatttaaaaatcgattatattttaaaacaaaatctgaagcactccgtggaccgtggttacacgtgccaggtcgacgtgttcttctggaagaaatctatgtctacttttgaaatggaatatactacctccgttaaaaataagtacaaacgtaaataaataagtacaaacgtaaatatccgtatgaacgttcgtcttattccaaaaatttataaaaataaattataaaaaaacagtcacatataaaatattatttatgttttataatctaatagtaacaaaaaaatattaatcataaaagataaagaaaaatttaaataagataaacgctgaatataaataatattttataaagctgtacttattttaagacggaaagagtaaaaaattttggaaaaatttctagtgcagcatgctgctcatttgggaggtgtgtgctgctgctctccagtctcccccaaccaaaatttgaccgagtttggcacgaaagaaaccgcctcgacgccgtccaacggaagccagccgaggcgaccaacccaagggtaggcctaggcgtaaaccctgcacctgaatcgatcgccggcgatggccacgacgctgacgccgacgcagcgctacgcggcgggggcgctgctggcgctcgcgctccgccaggcccagatccaccagtccgtcctcctcggcgcccaccaccaccacgacgacgacgacgaggaacaggggcgcaccagcaccagcagcggcggcggcggcggcagctcctcctcctcctccaactccggcgccggcgccgacgccgacctctggacacacgactcccacggcctcctccgccccgtcttcaggtgttttctcgctcctctccgcttgtaaatttggggggtttgagatgaatgcttccgctttacttggcgatcggggggctctgctaggttcctggagatcgatcccaaggcgtggtcggggctggaggagacggcggcgtcgtccgaggccaagcatcacatcggcgcggtacggcactcactgcattttctgccctcgtattgtaattggccgtgcgttttggtcagtgttgcagtgatggtttggggcttaggtgtttgattatatgcctacaagactgttcaacttcaatttccgcgtgtgcttgaccagtgaaatttgtgttttcgtagcatctcatatcactctcattttgtagtttctaaggataatattcgaagaagatggcgaaagctcctcagacagatccgttcaggagcttgccttggcaaaaggagttgatgtgatggtaatgagtttgggcaatgacagtgaagtgggtaacacaattaaaggtggggatcaagatgctttgcctagcagctctgga

SEQ ID NO: 99 - DNA, 10744 bp, Oryza sativa
  misc_feature 1..10744: note = source = /organism="Oryza sativa"/mol_type="unassigned DNA"
  source 1..10744: mol_type = unassigned DNA
gttgtcgcagaacttgttacaattgtatagtcctttagttttttgtactaatgtcatattggcaacacatattccttgttgagttggtactacctgcatcagcaccttaatctttcctagtttgctctgctaatgatgaacaatgttatcaccaagttagagcagttacgtaactatagagtatttgttgtacaaatttcttagcctacttgccactaataatataatggcaatatattcactcttctttacgagaatacatccatcaaaatcagatatgatctttgtattatcaataaattgtttatctatggggtccatgaatatgtgattgtggcttaggtcgtgttcgattcagccgtctggcagtattattgtttgtaattagtattatctattataaacttaaaaataaatatacttaattttaagaaatttttatataaaattttttacacgaaatgtaccatttaacaattttaaaaagtgtacttactttatgagaaatgagtaataaagctcatccgagaatccaaaaagaacgaaccttaattaggccttgtttgtttcctattgagtttttgtcttcaacttaaagccaacgaaagcttaaggttctcgaaacgaaattgtagcttctctacttctggaagaggctaaaagccaaaatggaggttacaaatgtatctatcagctgtgatcgggcccacatccatacatttctttaaagaaaaagaagaaagagaaaaaactctgtagctagtttttttttttattttttgaacttttaaattttaaaaataaacccttataaaacttgttttcaagatctgaacattttcgtcacgttagccaaactgacttggcaaaacaacattgtcacgatggtttggctgacgtggcagcattatttagtcacgccggtctggctggtatgacaatgttatttgaccactccgccctcgatgacgtagcagaataaaattgtcatgcccattatgtgcgacgtgccagtgttgttttgccacgcaagacagacgggtgttgctaaaagtttcagattctggaaaaaaagttttggcaaggttaatttttaaaattaaaaaataaaagttcaaaaaaaaattgtagctagtatggaaggtgctgagaaagcaactgaatgcaattttccaaaatggaatagttatggatttggcggatacaatgaaaataagttgaaagtgaacatttgtatttgttttcttgatccatcctttaaccgtatatggtgatgtaaacagctagtcttaagaaaactgaaaaatatatggtgatgactgatgaataccaaatgctaatcgtgtacggtcgtttaagcatgtgcactaaatatacaaaaaggaaatgtatctgttcaacatcttggattagactaagcattcatggatcttcagcccaactgtagtattgattactatttgatatgaacaattccagacctcgtgttggacaagttcaccctgttactttggccggctaattgccagtttgccaattgccaccacttgctcagtcagttgtgttactcagctgtgctaacgtaatttccatgctattggcaaacctaatttgctgcaagatatggaagtgtcaataaaaatatattacttttaatagtaacgaaaatataataagagttatgatataggatacttcatatatctaaaaatatactaaattctaacgatgtatttggatatatttatctaaatactagtcagaagttactatattttgagatgtggatatataggtgtcatgaaaaatgattgaaggagaaccttttcgtgatatactgggacccactgcctaaaaatatagaggaaaaaaaaacgaaaatgcatgtatctatggaaatttgagatcctatactagtcttaaaccagatataaaaatatcattcctcacctactcctcccaactctatataaaccctggatcatcctaactttctcctcagctctacaccaagagcgcttataaacataaaaaacatctcaaacacaacccaaaatagggccaaaattctagaatattatttttgttttgaaaggaatttaagtttgtgtggtgtgtcatccatggcggacatggtgaagttcaccatggagtggcttcaggatcctctgagcctggcgatcgtcgtcacggtggccgtcctaatcatgcggatgcagcggcggcgcgcggcgccgttcccgccggggccgaagccgctgccgatcgtcggcaacatggcgatgatggaccagctgacgcaccgcggcctggcagcgctggcgaaggagtacggcggcctgatgcacctccggctcggccggctccacgcgttcgccgtgtcgacgccggagtacgcgcgcgaggtgctccaggcgcaggacggcgcgttctcgaacaggccggcgaccacggcgatcgcctacctcacctacgaccgcgcggacatggcgttcgcgcactacgggcccttctggcgccagatgaggaagctgtgcgtggtgaagctcttcagccggcgccgcgccgagacgtggctcgccgtgcgcgacgagtccgcggcgctcgtgcgcgccgtggcggcgtcgcgcggcgaggccgccgtcaacctcggcgagctcatcttcaacctcaccaagaacgtcatcttccgcgccgcgttcggcacgcgcgacggcgaggggcacgacgagttcatcgccatcctccaggagttctccaagctgttcggcgcgttcaacatcggcgacttcatcccctggctcagctgggcggacaccaacggcatcaacgcccgcctcgtcgcggcgcgcaccgcgctcgacaggttcatcgacaagatcatcgacgagcacatggagcgcggcaagaaccccgacgacgccgacgccgacatggtcgacgacatgctcgcgttcctcgccgaggccaagccgcacgccggcaaggccgccgccgccgccgccggcgccggcgacggggccgacgacctgcagaacacgctccgcctcacccgcgacaacatcaaggccatcatcatggtacgtgcagttgacaacctactagtaattaattactgccaaatttttcaccaacttaatctctctatcacggtaatgattaattaccacttactcctcccggccgatcaagataaaacccgatcgaaattaggcaacaccaaatccgtaaaattactggaattaatgattcaccgatttagagcaagtttgatagtatagccaactattaactctaaatcatctatagcaaatataatagctaatttatacaatagttacttactatactattaatatctggtcccacctgtcatacacacattacgtcttagagttcgtgctgcagctggctatagattttgtagcccgctgctcttttctctcttgctttatctttttaaaatatatttatagttggcttacttatagcctgctattgtaactactcttacacagcacagcagtatggagtagtaatatatatcttaaactctgacaattaaacatgataaattcatctgacgcaaacacaactgtacgctataaatacgcgtacaaatcagcaacagtagtgtacggggccggattattagtcaggtaatatatcattttattttaggcgattaagtcaggcaaattatcttgatccgtgtagattatttgacagggcagagatctctcctggtatagttgacatgaacaccatgtgcagtccatgtgtgtgttcgcctcgtttgcaggtgaatcgatatgatcaaccgttagatggcgccacgtatgcatcatggatcatagattaagccctgcaaattaaagccactcaatgaaatgctagatttgaggagtaattaattttagtgtagaatacagtggtcctccttgatgcatccctcgcaatgtatctgcacttctacaggcagtcaactcacttacataagcattgtcgactgttcccgaggagatcgacaccgtcagatacggcattaacctatgattatttatacccatatttcaatatacgggtagtaaaatattccgagaattttatggagtagcacgtactcggaggaagtagtaaaaatagctttgtacttatccctccgttgtataaaatatccaatccttaccatgtacttcctccgtaaagtcgttttgaacaacgatacggtctccaaaatacaactttgatttcttgtttctataaaaatatctattaaaaagtaatatatgtatacttttatgaaaatatttttcaagacaaatctattcatataatttttacattttcaaactcaacaacttaagagttattcatgatttatattcctaagatttgacttaaaaacagtcctaaacgacttcctttacaagcacagagggagtacccggatatatacgtgtctagttacgtggctagaaattgtctctctctttttcttttttttcttttttgatatagtagtatatcggatacagctagaagtaactatagtgttagccttttttagcgaataaaggtagaagctaatctttaaccacgttctggtaggatgacagctctaattttatttcacatcttttagatgtggatgttttcagcgtgtaatatatatctcctttttcaaaaaaaaaacctatattttaaacgaacttatataggaattttcagtggtagactgaataagtgttttgacttgctggtagaacgactggatatttgcacacacgaataatcttacagtcgcttggcgcaacagccaacctagcttgaggctcaaattaaagtcagagcacgagcctgaccaacgccggccggcggcgggtcttctccggcgccggcggccggtgatcgatttcatttccaggcttattctccgcgtatgcatgatgtgtcaagctactgagctatagctagccgtctctctcgtgtacgagtctcttaccactcttggctagcaaatctgacgactctttgactgaacgaacaactcgtacgtcttaaaagccaagaaaacaaccccggtaggctgtaggtgtgttcaaggggtattttgatttggtacctatatattcttgtctttatcaattaattcgtttgtaataaattgtacttacatgttaactatttgaattagacctaaagtataccaaatcaatagagtggtgtcaaacaggcactaccaaataaaaaaaatgcctactagcaatttttattagggtttgaaaccatacaaatcctaagttatctttgtgatactattgtcgtcggcctcacccatggtttgcatatggttgaatatatctgggattttttttcacttaaacggacatctccttgttattattttaaaaaaaattgacaataaagaatattatgtaatatttcaatctaaaaatatataagtttaaatttaacttttaaaatatattaaaaataataaatattattttgaatgtctgtatactagctatagtttaatttgtttttttaataacttgtggaagttaaacttagcattattcgtgtgcgaagtgatatatatcatattaatttattttatagaaaaagaatttttttataactgtttgagtggcctaagaaaaataatggaacatcctctcgaggaataaaaattcatctccaatatatttactgtgttgatctttcgctggtgtgttatagtgtgcgtccgtcttctttttttgccttgtcaacctattacgtactgtcatgtatgttaacggacttgttagggaagcaaactgtgagtatgtcttgtttatttttcttggttttctacctaactattactatcatgtatgttagtgtacttgttagggaagcaaccatgagttaggagacatacgtaatttgttggactgttgggtgtgtgtggtgtatttttgtggctagagttaatttggatttttattattttagttctttaaaaaaattaaaatttctcattgatttttttggcattaagataattttataaaaccatgaaaatagtgaattcattcatctgtctattaccatgttttaacgattggagagcacatattaacatgaatgcattttgcacttagtacctccattgttttttttttgggatcgaaatacatcacgtaataacacctaggaacgtacgcaacatcattttcaacttttaagacccctttctccaataattgaaaaattaaacatgaaccaacctattgaacttctatgtatgtcaccatcatcgcatctcccttttaacctactccctatgtttcacattataaattgttttcggctagtcaaaattattctaatctaattaagtttataggataaaacgtaaaagcatttttaaaacaaagcaaacatataaaaatatagtaaatgatagatttaatgaaaacaaattttgtattataaatgttgctaatttttttataaacttgactaaatgttaaaaagtttgacttaagataaactctaaatggcttatgatgtgaaacctggatagtaagtccatatagataggctctggccatattggtgcatgtcaccctccaaagtaaaatcttcatttatctggccccaacggccgctcaatactttagaccaaccaaatcatttccttccatctcacggtaacaaaatgaatgatgtttttattgtctcttcatctttgcacattaaaaaagtgaaaactttataacatcattactggtaagcattacgcaacatggcactgacttcttgtctgaatgtacacaagcaacagcttgtttttataggacaaaatgacaacatattgccctgttggaacttagatgtacatttgtcatgtaaggataatattaaaatgaaatctgttgataggaaaagtgaaattttctttctaccactacctaatatgtggtcctacctatgtatacgtaagattggcacaagtaaatttggtatggaaaattgtattattatacttttattaatccctccggtttttttagtacgacgtcatgaatgttggatacatgcttgaacatttatcttatttaaaaaattagtcaattattatttgtgatattgtgatttattttatcactaaatgtatcttgagcataatatttaattcttttttatatttgcacaaatttttgaataaattgaacaatcaaatagttaaatgtatttccaaaagtaccatatactagttatgttttttaataggtaatgccgttgactttcagacacacatttgaccattcatcttatattaaataatatatataataattatcatttattttgttatgagttattttatcagtaaaagtactttaagcatgatttggaacttatgcatttgcacaaaactttaaaagtaaaacggatgatcaaatacgtgtctaaaattcaatagtgtcatctattaaaaacaaaaatagtattaaaaacagtggtggagccaccgcctgggatcatcgcagcgaacacactgtaaaatttgatatattatagaattgaaaacaaaattttcatagcaaatacttgttgtagttagatttacccgagcatcgtaaattttctagtctctgccactgattagaaatggagggaataataagtaaacaacaagtagtgtgagtaataagcaaaggtgatgacgtggcatgcaggacgtgatgttcggggggacggagacggtggcgtcggcgatcgagtgggcgatggcggagatgatgcacagccccgacgacctccgccgcgtgcaggaggagctcgccgccgtggtgggcctcggccgggacgtcgccgagtccgacctcgacaagctccccttcctccgctgcgtcatcaaggagacgctccgcctccacccgcccatccccatcctcctccacgagaccgccgccgactgcctcgtcgccggctactccgtccccaggggctcccgcgtcatggtcaacgtgtgggccatcgcccgcgaccgcgccgcctggggccccgacgccgacgcgttccgcccctcgcggttcgccgccggcgccgccgccgaggggctcgacttcaggggcggctgcttcgagttcctccccttcggctccggccgccgctcgtgcccgggcatggcgctggggctctacgcgctcgagctcgccgtcgcgcggctcgcccacggcttcaactggtcgctccccgacgggatgaagccgtccgagctcgacatgtccgacatcttcggcctcaccgcgccgcgcgccacccgcctctccgccgtcgccacgccccggctcacctgccccttgtactgacggcggccggcgaccgcggcggcgtacggcaagaatggtttgcaggtgatggtgttaaaagttcagacgcgtgtggcgccattggaggagcttggtgtggctagcttagcttcctctgggcctttttttttttgctggtttaattttcttttttttttctcctttgtaatttaatccatgtctccagaacaataagtggattagtagggtataattaagggtgacagtgtgatatatagactataagatcaaagttgtaaatttggtgtccggtgtgcctgtccacataagcacagcctctgaattttcttgtaaaactttgtcatgaaaattgagaaagaaaacacctcgctttccacgcgtacgcttaccaaactattaaatggtgtgtttttttaaaaaaaaattctatggaaaagttgcttcaaaaataatattaattcatttttaaaatttaaaatagttaatactcacctaatcatacgcgctaatgacatacctcgttttgtgtatctttttttttcctctcctctcaaactatcgggttgctatatgttattaaaacgggaaaaaaaaggcaaactccgaatgtttgttgtgaatttacatttctactaaaaatttaggaccgcatgagttgtgatgtagtggcctgagatgtgaatggggagagatcgtacagggccaaatatttccccagcggctgtacagaaagaccacctatacaacttttctcaatgtagtaatattatactccctccgtttctaaatatttaacaccattaactttttagcacatgtttaaccgttcgtcttatttaaaaattttttgtgaaatatgtaaaactatatgtatacatatgagtatatacttatatttaacaataaatcaaatgatatgaaaataattaataattacttaaattttttaataagatgaacggtcaaacatatttaaaaggtcaacggcgttaaatatgtagaaacggagagagtaattttcttaggcatcctaccagcattagtagttagagcacccgcaatggtaaagtaaggtgctatctataaaatatgtacatctcagcaatagactaaattaatagtaaaacacctcaatagtatgtctacataggtatctatagctctctaatcaattgactcatttttctctatagactatctctaagttagtagatagttttactctctcttttcatttaatctctttcaagtaggaaaatatgctgacatagggagcctatagataatcattgcgggtgcccttagtacacctgtagtctagtcacacagttgcattaatgagtagtttataatagccataattaggttacttgtaaccaacagtactgatgacgaacatatatatatatatatatatatatatatatatctgtttttgtttggtgaaacggccttttcttgtgtaagattaattcaaaattagatggcgcaggagcaggagcagaattcgttcaacgaattcactgttcagcaacgtcggccagatcagaaaacgaccctgcagaacagcagaagtggagctttcttaattacaatcttcaggcatcgaggtagctacccgaccctctagtttcatttagctcttcagagttaacattcatcatgcatggagttggttgctagtgattactccatctggtcacaatcaaaaaaaaacatgttgtcttcggcatttcttcttccagtcaagagagaacataatattgttggtgcatcaaatttaaaatggaagacaaagctatcaaccataatgaccggttgttagcttaataaggagtgacgaatacttgaaagctggctaattcagaaccttctttctaggtcggccgaaggattaggctaacctaattatattaagattgagaagtttaatttacagatacttttttaatccttgaaagaaaatcaaagtgtgataaatatgatttttttaaaaaaaaaacaatcaccctcttttctccaaattatctgcggtaaggcatgtccactttgtggccaccctccaatgccgaacctttgctgaatctcaagcaaaaggaacaagcaagaaatctctgctggaaagggaggttgataaaaggcactactccacaaatgtcagtttttttgtctgagcttggtattatttatttaaaagtagctaaatttgtaagttggttacaattgtgcaataatagtaataacttgcaacgtccgagctaatttacaattatgggtgaccatgtttcgcatcactaaggctgtgtttagtccataccaaaattgaaagtttggttaaacttggaacgatgtgacgaaaaagttgaaagtttgtgtgtaggaaagttttg

SEQ ID NO: 100 - DNA, 7710 bp, Zea mays
  misc_feature 7039..7138: note = /note="n = a or t or c or g"
  misc_feature 1..7710: note = source = /organism="Zea mays"/mol_type="unassigned DNA"
  source 1..7710: mol_type = unassigned DNA
tgttattttgcatcactatttttaaccaatttttaagttaccgtaccaatgcacgggcacccatctagtataatatatagaaaccataacgacaaaagggcaactaactagtacacaataaattccatgggaaaattttgacgttggaggaaaacctactagtgttctaccatctaccgagtccatatcttgggtgctctgccagatccatattttgctacaattattatccttcttaatatcaggcacttcagtgttactactccatcacttctcatcgtcagggaagggataccataacctaatttcgaatgcaaatataatataattaaatgtagaaaacgagatcatattcctatcaccagttatctcaacctaatatttaattcttccaaatgaacctttgatccatctagatcatgaaacataagttacataaaaatccaacaagatacaccgatctcatacacaaatctaactggtgggaataaatagccaaaaacaatttgtatagttggcttttttaatagtacagtagtacatccaacaatcactacaagtaactgttcttttggaattatgcagtcaacacgtatggaatttgactatcaacatctctaagaatattaatttcaaatgatattttgaaaatggtatggctagattttcatcaatattaggttcaacatattataaattacaatacgttttaaacatactcatacaaattattcatagtccaagtgtttagttggctatagaaaaacaatattttgtttgttatacaaaagagtcagattctttgaaattaatatttttaaatccatacttataaaccaccggttttcacggttccacaggcgtcatatgtcaccctttcccttcttcttttatgcaaaaataagataaattattcataagtgggagtttgaatcctggttgttggctccacattaacaccatcctaatcaataaaacacacacttttgtgttttattaaaataaaatctctctatgtgatacatagaaaccgtagcaatgcaagaacatctaactagttaatcctttatgcttattagattccacctagtgcatgcaacattaggtgcataaaaacccaagagaagaaaatgtagaaaacggtaatagtcttcaattctgccaaaaaaacttacctatccagtgcatgcaacattagctgcttaaaaatccaagaaatatatgctgacgctcatatataaatccagctggttggaataaatagctgggaaaaaaacaagatgcatagttggctacttaacagtacagcagtacttccaaaaatcacaataactgttgctttggatgtatgcagtcaacatatgtcttgatcttgaccatcactgtgtcgaggaatattatgttcaaatgatgttctgaaaatagtatggctagatttttcatcaaaattaatttgaaattttcttacacaaaattaatctgtactattttttactatcagaacatatttttatgttcttttgtttaagtatctcgattgaagcaccatctcgcaccatttttaggcatgaaaaattcgtatgtcttttctgtttctcttttttcttttactattttgaattttctatacatttcaaacatactgatgcaattttcatagccaaatatatagttcctccgtgatcatttatctgtttgttataggaaagaccgaaagagcagcattccaagaaataaaagtaaataaatagatcattttatttgcttagaaatccccttttcattttcaggctctagaaatccatccgttaattaatttgctccatagtattaataagagatagagagatggggagggagatccttttcgtaaaagagaaaagaagatcctatttctatcacgctattagattatctccagcagtctctcttattctattgtatattttaaaatttactctgtaaaccgtgtctagagtttagaatgtaacatagtatttttgaacagtattagacggcacaactatctcaatttctcagacccaatatggaagtccaggtcctcacctactcctccggctatatatgaaccgtgcactcctccctttcttttcagctctactcgaagagcgttctaccaccacaccacaatctaactaaaacccaaacgaaataaaaggaagaggtcaaaaaataaaaggtgttgtgcaatcgatcatggtgaccgtggccaagatcgccatggagtggctccaagaccctctgagctgggtgttcctgggcacgctggccttggtggtcctgcagctgcgacgacggggcaaagcgccgctgccgcccgggccgaagccgctgccgatcgtgggcaacatggcgatgatggaccagctgacccaccgcgggctggcggcgctggccgagaggtacggcgggctgctgcacctccgcctgggccggctgcacgcgttcgcggtgtcgacgcccgagtacgcgcgcgaggtgctgcaggcgcaggacggcgcgttctcgaaccggccggccactatcgccatcgcgtacctgacgtacgaccgcgccgacatggcgttcgcgcactacgggcccttctggcgccagatgcgcaagctgtgcgtgatgaagctgttcagccggcgccgcgccgagacgtgggtggccgtgcgcgacgagtgcgcggcgctggtccgcgccgtggcgtccggcggcggcggcggcggcgaggccgtgaacctgggcgagctcatcttcaacctgaccaagaacgtgacgttccgcgccgccttcggcacccgcgacggcgaggaccaggaggagttcatcgccatcctgcaggagttctcgaagctgttcggcgccttcaacgtcgtcgacttcctgccgtggctgagctggatggacctgcagggcatcaaccgccgcctccgcgccgcacgatccgcgctggaccggttcatcgacaagatcatcgacgagcacgtgaggcgggggaagaaccccgacgacgccgacgccgacatggtcgacgacatgctcgccttcttcgccgaggccaagccgcccaagaaggggcccgccgccgccgcggacggtgacgacctgcacaacaccctccggctcacgcgcgacaatatcaaggctatcatcatggtacgtacacacgtcatcctctgacctccctgttgtcacgctaatcacgccaaaatcgtccgttacgttcctcgactcgtttttgcgcttaggaaaatcgtgatgtgccagagcaatgaacagtagcaacatgcatcgacgaaagaatttgaaatgataggattcgatatatatatatcctgactgagatatatatagagcatctcacttctcgtgtgtcaggatatataggtgtgatttaattctattattactaactgctatgaatggtagtattacggaatcagacgtgctttctgctggtgacagactgagtaggtgttgtcttgttcctgcatatgtgcgtaggtgctgaacgtccatatgtattcacgagctcaaagtcaaaccacacatagtatactacgccttgtctttcattcagtatactaattgtccatatgtattcacgagctagctacgtaagtaccgccacgatcgacgtgtatataacatacgtatccttttgcgatgatatttttctgtatatatacggctatattaattgtccgccctgtagtttgcacatccagcgagtccaaactaatccggccagccaatctctggtttaaatacatagatatgtcgtaatcgtaatagaatgcgtgcatgtagtcgaccttccgtcatggacacaactggcatgtctctgacgaatgtggcgtggtacagacggcgctccctacaacttttgtagtatgtatatctcattgcattgtcactaagatcttattattattatttattcttgatgaatggtcaaagctgcaaattaaaaaaaatcatagtagcaaagtgcaggtacattgatatggtttccatttataagtatagagtataaaacataatgatatatgtgttacgtggatttggagctcacaaccaaagttcaaatcccacttgtacaataattttagatttttatagatatcgacagcgtcagcgtgcatcatcgtcctgtatcatgcatccagaataaatgatacatgtgttatgttggttagttagttgagtataaatgtggatttggagctcacaaccatatagttcaaatctcacttgtacgataattttagatttttttttatttaaatatgtgggaacaccacgaccgttcatagatattgacagcgtcagagtgcatcatcgtcctgtatcatgcatccagaacaaatgttttatttctctgttagttgttgctggatcgaactatgggcctgtttgttttggcttctggcagcttctggccaccaaaagctgttgcggactgccaagcgctcagcttttcagccagcttctataaaattcgttggagcaaaaaccatccaaaatcaacataaacacataatcggttgagtcgttgtaatagtaggaatccgtcactttctagatcatgagccctataaacaactttatcttcctccacacgtaatcgtaatgatactcagattctccccacagccagattctcagaaaagctggtcaaaaaagctaaaccaaacagcccctataaatgatgcgtctctatcttttggtcgttgatggacgacgcgtgcgcaggacgtgatgtttggcgggacggagacggtggcgtcggcgatcgagtgggcgatggcggagatgatgcacagccccgacgacctgcgccggctgcagcaggagctcgccgacgtcgtgggcctggaccggaacgtgaacgagtcggacctggacaagctccccttcctcaagtgcgtcatcaaggagacgctccggctgcacccgccgatcccgctgctcctgcacgagaccgccggcgactgcgtcgtgggcggctactccgtgcccaggggctcccgcgtcatggtcaacgtgtgggccatcggccgccaccgcgcctcgtggaaggacgccgacgcgttccggccgtcgcgcttcacgcccgagggcgaggccgcggggctcgacttcaagggcggctgcttcgagttcctgcccttcggctccggccgccgctcgtgccccggcacggcgctgggcctgtacgcgctggagctcgccgtcgcccagctcgcgcacggcttcaactggtcgctgcccgacggcatgaagccctcggagctggacatgggcgacgtcttcggcctcaccgcgccgcgcgccacgaggctctacgccgtgcctacgccccggctcaactgccccttgtactgacgccatgcgcgggcgactgccattaccatcgtcccctcgggtgggtgtggggtacgggggtaggagtttggtgcctttctctgtcgtcttttttccctttaaaaaacatgcctggtcgatgttgtagggtgtgttgtagacagccattatcaattttttttattctcaattctcatgttgcgtgtgcctagctaggaagtaaaaggatcaagctgtggctgtacgggctcatcaaactatatgaaaaaaacatgtcagccacagattcctggaactttcatgcacggcaaatttcatagatatttctttggcacctcttatctatcgtgtatcccatatatattaatctctgtattaacaaaattggagcttcctcacgtacgcacccaggcactgctcaccaccgtttaagatacagctcaataatgttcctgcactgtttaatattgtattgtatatcattgttgcccgcgctaaatatatgtttactgtttggcaacatatctagtgcacatggactgctggtgcacatcatacatatattaaatcctaccctttcatctaagatcaagcgtctcacaattatctcacctctctctctctcctgcctttttgctaaaatgccctcccgcctctcccacttcctaccatgtcgttttttagaaagctgcatggcggactgtttatctgcttgatcagaacggctgggagtatgcaggattcagccgtgccagatttttttgcttcctccggtcaatagaatatgaatgtatggtgtagatgaatatatagtggcgttttcatgcaccaacagaatgaacgaacaagatcaatgagcaaagaaactgactacatctagtacatatatcccctattgttctctctccatctttctctgtctctcaaataaaatatgagatatcaataaaaaactaaatatgctctaactttttgaccaatgatttgaagccgcagctcaaaaacaaaaaataatatgcaatagatcagtaaatctttcattgcaccattttcgatcaccatacctatgtaccacagtcttgtatatgatgacattgtaagatacatgtcatccaacaaagagaccgttgattctaaatcgccctggacagaaaatccataaattagaattacaaaattaacaacatacgaaggtcatccgtggtgatcgtgacggcgccaagtgatggtgatcgagcctttgtaggctccctctttggcgagtgtctgctggactagtggtcgacaaagaaggctcccgtggccccctttgctagtctctttgtcgagtgctccaagaggcactcggcaaagtctctctctttgccgagtgcttactcgactaacggtcggcaaagggagcaccagtgggcccctttaccaggccctttgtcgagtgctccaggaggcactcgacaaagattgtttttttgtcaagtgtaacactcgacaaagtgactagaatatctcattttatttgtttttattattccatccaaacaaacaaaagatatatcacgaaattatcacatatatatcatagatatcacaaaaatcatcgcatacatcgtacacgccacatatctcacaaagaccacaaatctcacaaatatcaccatatcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaggtattagatatttcgtcacaagtttcagacacaaacataagtctccatcactcacaaacatttgtctcaagtatctcacaaattattaccaatgacaacaagttcagaccaagttatctcataaagtattaacaacaccaatagacataagatcgtgtgcacaaagtatctcggcaaggtggagggctggactgattcggcgataggttggacgatccatgagggttgtttgatgccaacgtttgtccctatacagagaagagattgcatgtgtgagaccagataaatatatatcatgcagggctggaactttgatactcacataagtatataactgagtagggttagcttgagggaacaacgaggtggtggaacaaaaccatgtgcggcaccaaggctctgcatgtactgaaacatcttcgtcattcttgatccgccaagcgagctgcctgctccgcctcccactccgccctcatcctcgcctccatctccatctgttccctcctctcttcttttagctaggcctgtaatatttcacccaatgttataataatgcaaagagaaggtat

SEQ ID NO: 101 - DNA, 7353 bp, Zea mays
  misc_feature 1..7353: note = source = /organism="Zea mays"/mol_type="unassigned DNA"
  source 1..7353: mol_type = unassigned DNA
taaaaaaaaccgccagtggaaatagcatttccactgacggttttcttaataaaaccgccagtggaaatgctatttccagtggaaatggcgattttactggcggttttagaataaccgccagtggaaatggcgatttccactggcggcactgaaaaacgccagtgcaaacagctctagaaccgccactatagagcctctgtgtactagtgttaaagtaaaatccataattttgtaggatcaatatccttgttcccatagtcaattgtgggagtactccttgtctcctactcaatccactagtagaaaaaggcttaacacctgcgggacgatatatttttacagacggatccggttatccaccgactgtgctatttctagtggcggttttcttaaggaaccaccactagaaatcatttttatccttaatttttcgaatttttcaaacgacctcgtatgacaaaaccaccaaaataaaagttgtagatctctagaagttatgaaactttgtagttgacaacttttttatttgaacttatttcggttctcaaaaattgaatctaagtatgtcaaatttaaaattcaaattttgcaaactacctcggatgaaaaaagtgtcaaaataaaagttgtagaacttcaaaagttatttatctttgtagttgacaacttttttatttgaattcgtttagggtctcaaataagcaatttacactcaaatggttgtaatatgtggagaaaacaactacaaactagacacaaagcatgtcatagacggagtggtagtggagggtacgcgcgaggtcgacgggtcaattactaacaaccgcgtagcgcgcgaaaaatgccgtgacttgcgacttcggagacgggcgggtggggtgggcctaataaaaataaatttttttactatttttaaaatctgttttatgttttctggaaacgatttgcactggcggtattattacgtcgatcgccagtgaaaatcgattttcactggcggttttattatgtcgaccgccagtgaaaatcgattttcccgcctgtaaaaatgatgatttttactagccccctagcactgacggttacgataaacgccactaaaaataggtttacgaccgtcagtatagatcttctctatactagtgatatccctttctcatgtaactccatttcaaatgaaaataaagaacaagtagaaaaaaaaatgaaagagacccaattcttgtcaccattattaggcacagttaactgatcgagttttcaacccaatgtatccctgggtcctcacctagagatggcaatgggtacccgcgacccgatacctgatgggtatttactccattagggtatgtatgtgggctaaatattctacccgtgggtctgttattgggcaaaaatcttcacccaatgggtaaacgggtattagaacgttccgccttcacccatacccgttaacccgtgggtataaaatacccaatataaacttatccgaagcatgaacttggactttagtcatccaacttttttactatttaataacaatttaatgaccatgtaatggaacatgtaatgtgctatgtagtactatcatagtgttactactttatccaattatctttggtgtgttgaatggatggttaaatgatgctagaaaattttgtaatggtatttatatggatatacttgccatttgtatagtataattttttgatagttgtttaatttttgtgggtacgggtgacccgatgggtgacccatacccacatgggtatgggtatgggggtaaatctatacccgccagtgtatatgggtgacccggtggggttatttttgtgtcgtgggtatgggtatggggtagtaatacccggtgggtatttacccattgccatctctatcctcacctactcctccggctatatataaaaaccgtgcacctccccccttgtcctcagctctactcaaagagcgctctaccagtacaactacaaccacactaaaacccaaacaaaaaaaaggaagcaggtgctgccgatatatcgattgatcgatcatggcggcggccgtggccaatatcggcatggagtggctccaagaccctctgagctgggtgttcctgggcacggtgtgcttggtggtcctgcagcagctgcgacgtcggcggggcaaagcgccgcttccgcctgggccgaagccgctgccgatcgtaggcaacatggggatgatggaccagctgacgcaccgcgggctggcggcgctggcggagacgtacggcgggctgctgcacctccggctggggcggctgcacgcgttcgcggtgtcgacgcccgagtacgcgcgcgaggtgctgcaggcgcaggacggcgcgttctcgaaccggcctgcgaccgcggccatcgcgtacctgacgtacgaccgcgcggacatggcgttcgcgcactacgggcccttctggcggcagatgcgcaagctgtgcgtgatgaagctgttcagccggcggcgcgccgagacgtgggcggccgtgcgcgacgagtgcgcggcgctggtccgggccgtggccgtgggcggcgggagcgggggcgaggccgtgaacctgggcgagctcatcttcagcctgacgaagaacgtgacgttccgcgcggcgttcggcacccgcgacggcgagggccaggaggagttcatcgccatcctgcaggagttctccaagctgttcggcgccttcaacgtgggcgacttcctaccgtggctgggctggatggacctgcagggcatcaaccggcgcctgcgcgccgcccgctccgcgctggaccggttcatcgacaagatcatcgacgagcacgtgaggcgcgggaagagccccgacgacgccgacgccgacatggtcgacgacatgctcgcgttcttcgtcgaggccacgcccggcaaggcgacgggcgccgccgccgctgctgacggcggcgacgacctgcacaacaccctccggctcacgcgcgacaacatcaaggccatcatcatggtacgtacgttacgtatacacacacgtcgtgctgtgcaccatccctcgatctgacctgctgtcagtcacgctaatcacgccaaaaccgtccgttccgagttccgacgccgggaggactgggtgccggccggggttcgggcctcgggagcactgaatcaaaacgcttggctgaagaaacccgtaaacaaaaatacttactatatatatacctaaaatgtgtgacgacgtgtgatgtttgacaaccaaccaatcaggagtgctttgcgtctctgcatgctggtgacaggtgtttgtcgtgttcctgcatgcacgcagcacatgtggatctcttcgcccgccacggagctagattaagtcagggccacgtgtgcgtaactcgatctccagtagtgcatttttttcttcggtttggagttctcttctaccccggccggcttctacgttgacaagctccaaccggccggccggccggcttccagaaaagcagtacgtgaacgtgacgttcggtaggtgtacttgttatttgtttgtgtcgtcctccttggtgtctggagatcgagtcgattagccagacagaccagacgtagccgcgcacggtcggcttgcttgtgcatggtgagacacgttttctcttttttgttcatacatgcatgtagtactgtgcgcatttaatttgtggccatgcttatgagtcgtctcacactagtactgtacgcatttttaatttgtgcgttggtggattttttttctccccatatatcacgacacgacgcacaaacatttgtctatagtccaactttgcgcgtgacgtaagtcggtcggtcgtcgtacaaagtttggacggccggataccctgcatcgatcgttctccaccctatatacggagctgaaagtaggtaaacgtgatgaaagcatccgagtttgatcgagctagccaaccattggagaccatgcatgtcacttttcggtgcatttttttcgtctaagatagctctgtccaccttggtgcctcggtgcgtgcatgtcaccttccaaagtatataaaaaaaacacatgcatcacccattttctgcagtatatatataaccgcgctagctgccgtaaatatcttgcacaattaaaccgtcatgatttcgcttgtaattaataatctagcattctagcttccgtcatgggcacaactagctagcatgtgcatatatgactgatatgatcatgtgtggcgtggtgtaattaatattcactaaaaacatcataaataaagggtaatatgtacctcaccatcgcgtcgttatgtattttgttgttttttatgagcagtcaaagctgtaaactaataatgatgtatggacgccgcatgcaggacgtgatgttcggcgggacggagacggtggcgtcggcgatcgagtgggccatggcggagatgatgcacagccccgacgacctgcgccgggtgcagcaggagctcgccgacgtcgtgggcctcgaccgcaacgtgagcgagtcggacctggacaggctccccttcctcaggtgcgtcatcaaggagacgctccggctgcacccgcccatcccgctgctcctccacgagaccgccgacgactgcgtcgtggccgggtactccgtgcccaggggctcccgcgtcatggtcaacgtctgggccatcggccgccaccgcgcctcgtggaaggacgccgacgcgttccgcccgtcgcggttcgcggcgcccgagggggaggccgcggggctcgacttcaagggcgggtgcttcgagttcctgccgttcgggtcgggccgccggtcctgccccgggatggcgctcggcctgtacgcgctggagctcgccgtcgcccagctcgcgcacgccttcaactggtcgctgcccgacggaatgaagccctcggagatggacatgggcgacatcttcggccttaccgcgccgcgcgccacgcggctctacgccgtgcctacgccccggctcaactgccccttgtactgacgccctgcacgtggcgcgcggggactgccattacgcatgcatgcgtttggactttggtgttcatccctggggtggggccgccgtgggggaagttaggagtttggtggctttctagctctgtcttcttgtattctgtttattataaattttcccaacccttccatgcctgatcgatgtgcggtaataattgttagaaaatgtgacattttgtatgtaatcaatctatggggtgcaattgttatctcgtcaaaggacacaccactcgacttgcaccccttcatgtatatatatacatacacgaacatcccctgcaataaagaaatcgctgtcactttctttcgaactcctttccactgttttacaatatgttatcaacatgtgctcgcgagaaaatagtgagaagagaaaaatgctcgctgttttggagaagaacggaggcccggcctcaagatcgacgagaaatatgcatcatatgatcaactcaacggaggatgaattatacattgtggacagttgtaccacaaactctatacttagagagatcaaatattttcagactcttaaaaatgaagatgaaaaagttttgaccatcgctggatgcgatgcggtgatagttagctctggacaactattaccctctccatgggtactaaaattgttatcggagatgcactattgtatcctgatcccactcgtaccctactaagttttagagatatgctatagatgggttctatatttaaacccatgatgaaaatcaagaagagtttctctttttgactaagccggacagatatggcaaacgcatatatgacaaaattttattactcacgtctgggttgtactatacattcattaagtacattgcacatgttgttgcatataaggtgatttttcaaaattggttatcgaggcatagggattatgataaaaatcacaggcaattctattggtcataatttgtccataacaaattttcttaaatcgaaagattttatttgcactacatgtgcaactgggaaattgattttgagaccatcacatctcaaaattaaagttgaaccgctaaaattccttgaaagaatttaagtaaatatttgtggatcaattacaccaactttcgggccattcagatacttcatggtattgattgacgcatctactcgatggtcacatgtgtgtttactatcaacacgcaaccatacatttatcaagataatgtctcaaattatcaaattaaaggcaaattttcctgaacatcagattcaaacaatccggatggacaatgccgctgaatttacatttcaagcattcaataattattgtatggcactggatattcagattcaacactcggtaccatatgtccataaccaaatggtttggatgagtcactaatcaagaggattaaacttattgcaagaccattgttaatgaattgcaaattgtcttcgtcatgttagggtcatgcagttctgcacgctgtcgatcttatcgaactatgacctactgcatattatgctacttcctcaatacaaatggtacatggaaaatctccaagtatttcctatttgtataaatttggatgtcgtgtatacattccaatctcaccacctcaaagaacagtcatgggcccacacaggaaagtggggatctatatgggatttcaatctccatcgatcataaaatatttaggactcttgataggagccctaaggcagctaggagccgttgaagctccatttggtagcaaaagttgccttctgtacgtgggcgcaccggacatgaatagtgcacgatctccttccttatttagtgaagccgaccgttgcagccagggtcctcttggcatactggacagtccggtgcggcttgttgaccgttggcgctggccacgtgtcgcccactgatttcgctgccgaccattggcacgggcgctgctggctcaccggacagtccggtgcacaccggacagtccggtgaattttagccgcagtgtccccaacaaatcccgagagcagcgagttcaccgacgtgctccagtctgggcaccggacactgtccggtgcgtaccggaaagtccggtgcacccgtaggctggtgcaagtctggctttttcagccaaacttcttttgctcctttttgacttgacttaactgagttcctggtgcttagacaagcatgtttagctcataaaacatatgactaagtgtctagaacttaccttcatacttgatacatctagattgcttcatatacttttgctcaacatcatgtcagttctcacatagtgtgttgtgcatctaatcatcaaaacaatatagaaatggtccaagggcac

SEQ ID NO: 102 - DNA, 7394 bp, Glycine max
  misc_feature 1..7394: note = source = /organism="Glycine max"/mol_type="unassigned DNA"
  source 1..7394: mol_type = unassigned DNA
atgatcattcacaattaatcacatgactaattatgataattaatcacattcttggtatgtgaaataaaatagtattcttattttatgagcaagttccacattcataaaataatactccatttttatattaattatattcttggtgctagtaaaaaatataataatatttcactaaaatatttatttgattaaattaaattctctaatttaattatcaaataaattattttcttttgtaaagattagaacactcgtttatatgtaaccccgtaggtttaatactaaactggtagtaaattaatcataattaatttactaattaaggaagacgtctagcagcagtccattaagtccaagcatgaagtaacatcttttaccttcaagaacaattgaagaataatgtaatatttcatttcattgttgtttataactcaaggttaattctaaagtataatattattatcaaactctaataagataatacatttaatcaatgaatgacttaagaaactcttttttctttcattcaattgtcatgtctaagatcttaattctatcatagagctcaaacttgttacctagagttggtggattccttcttgattaataattaattctataagtatttaatcatatccaatattcattaaactagtgccttaagacattaagtgtctaaaatttaaatataacaaataatttgttaactattatgataatctcatgtcaaaggaaactattatatttcttcttgagaactttctattgatatattaagataatattaactattaggaattttcaattaagtcatttcaatgatgacatccatatatatatcatctatatatgcaatttaataaataatatttattaatttttatctaataaagacttttacatatatattaatttatctgaattattgatgtcttattcataataatcttacgattaagaataatttatattaaaattataaaggaattatttatcattatcataatcttgatcatgataacaaacctctaattttaattttggacttgtcaattctactggaatttttttaaaaaagatgagtgcattgcagtatggcagaaaccttctcacaatagctgctatatttttgcatgttagctagtgattttaatagttagttttgggtgttggtagttttctagttgtaatttttgtggattctttttgttgatatattccttaaacatgattttaatagccttatcttttaataattgtgctgtaaatgtactttaaaaaattatatctaaaacggatcgattagaacttttttcattaaattaatttttttaaaaaaatacattctaagacagtttttttaaaaaaaccttcttagaaagtctattttttaagatagtttttggaaaaatcatcttaaaatcttcaaattttttaatttttttgttttaaaaaacacattctaaaatggttttttgaaaaaaatcgtcttagaatccttattaattttttttatttttttaaaaatgattctaagacgattcttttaaaaaccttcttagaaggatatttttttctaagatgatttttaatggaaccgccgtcgaaagtcaagattttcgatgacattcgcttcgaagacaatcttgagtcgtctttgaatgtagttatgaatcgtcgtggaatgatgcttttgcagtaggttacaaaattatttatcatataatattaaaatatataataaatatttactatatttattaaaatctatttattatttaaatatacttatttgttaaatatttataatatatcaaatatgatataatacataagtatatttaataataataaatatttgcctataacatgaacacagcaaattagaatatatttttctttttgttaagaaccaaattagaatttaagaaactaaaatcatgcaaatataaaagtttggaaactaaaattatataattaatggtccagatttttgcaaataggtaatcctgaagctgcaaaggcaggagatcagatccctgtctccgtagtatcttattatatttaatatttaatatataattaattagaacgtccacgtttaataagtatcttgcaggaagaagaaactgaagtgtatatataggtccccatgacactcccctcaactcagcaacaacaacacttacttgaagttgtgctattcaatcccaaagccacagaaataatattattcagattttagagactgaaaacatcatggccaacctcgacctcgacccattccagacaagcatcttaatcctcgtcccaatagcactcctggtggcgttactatctcgtactcgtcgaagagcaccctacccaccaggcccaaagggtttaccaatcataggaaacatgttaatgatggagcagctaacccaccgcggcctcgccaacctggccaaacactacggcggcatcttccacctccgcatggggttcctccacatggtcgccatctccgaccccgtcgccgcgcgtcaggttctccaagtccaagacaacatcttctccaaccgcccagccaccatcgccatcagctaccttacctacgaccgcgccgacatggcttttgcccactacggccccttctggcgccagatgcggaaactctgcgtcatgaagctcttcagccgcaagcgcgccgagtcctggcagtcagtccgcgatgaggtcgacgccgccgttcgcgccgtcgctagcagcgtcggaaagcccgtcaacattggagaattagtgtttaacctcaccaagaacatcatctaccgcgccgcgttcgggtcgagttcccaagaaggccaggacgagttcattaaaatactgcaggagttctccaagctctttggcgcgtttaatattgcggattttataccctatctcgggtgcgtggatccacaaggtttgaactcgagactcgctagggcacgtggcgcgctcgatagcttcattgataagatcatcgatgagcacgtgcataagatgaagaatgataagagcagtgaaattgttgatggagaaacggacatggtggatgagttgctggcgttctacagcgaggaggcgaagttgaacaatgaatcggacgatttgcagaactctatcagactcactaaggataacatcaaggctatcattatggtatggattcgtattcaaattaatatactatcttattcttatcagccacctaatgaaattgataattcgtggcatgaaagtttacgcatttgactatttcattaagagttgttaagaacacggtttttctcaaaaaccacgtaagcacgtacaacgccaatacctcgatgattggatgtcttttggaatcctttctagtttaatgagattttttttaattgagaaatctaatttcttacatgattttttcaatgcaagaagagaagattcagatcaataaggtttatgcatgattatagaaaaattggatgaatttaagcttttaaaaagtttatgtatttttttaaaatttaatgtaatattgacatagagtattactgattttattaatgattaattttcgttaaaaaatttggttaataaaatttcttattttaatcagatttattctgcgtataaaatttaaatttaaaatcttacttaaacttttgcttaattaatttaacaaacacttctaaaaaaaagaaaacatattaagaataacaggtaaaatgctttaaaagtggttctaccttgagtttatagtgaatatgtgtaaaaatatccacttttgtatatcaaattataatagatgcttttataaaataaaatttaactttctagtatattttcttcaaaggatggttaatgttatttttaaaatttgaatttagatctcttttaaattgattgtatcttttttataatttgaatattttttaatttatttaaatgttttttaatttttaccttttagattttatttaaatttcaaaatgtcaacatatgttacatgaaaacttgctaaatacataagtgtttgcatgcccgtcagtaattttggttagtgaagttttaatttgtcaaaagtttatttgcatgtctaagatataaattatctatttaatattttctcaaatcatacaaccacctcacatttacatgtgatcatatttagtttacttattaaactgatcagagttcatttaaaaaacaaattgtcaacactacaaaaaaaaaaaagtctccaaatcaattatgattacttatgccgaaattgaaaaagtaaaagcagatttactatagttattatggactagctggagatgaacaaataaagtagataagatcataagattgagaataagatttgggaacactagccaccaaaaatgcagtgagtgactatcccaacgtgatcatagtgggtttgggtacggttggtgtcatttccccgtccaaatctacagcttcttctctttatttatttttttcaattaaagtttgattttagaatgcaaaattcaatattgtgtcgtatgaaggtggtaagtaaataaagaaaattgatatcggtgatgattgtttgtttgtggtgtgcgttaacattacaatttacacaggacgtgatgttcggaggcacggaaacggtagcgtcagcgatcgagtgggccatggcggagctcatgagaagcccagaagatcaaaagcgggtccaacaagagctggcggatgtagtgggcctggaccgtcgggccgaagagtccgatttcgagaaactcacttatctcaaatgtgccctcaaagagaccctccgcctccaccctccgatcccgctcctcctccacgagacggcggaggacgcgacggtcggcggttacctcgtccccaagaaggcgcgtgtcatgatcaacgcgtgggccattgggagggacaagaacagctgggaggaacccgagagcttcaagcccgcccggtttcttaaaccgggcgtgcccgatttcaaagggagcaacttcgagttcattccattcgggtcgggtcgcagatcctgccccgggatggtgttggggctctacgcgctcgagttggcggtggcgcacctccttcactgcttcacgtgggaattgccagatgggatgaagccaagtgagatggacatgggtgacgtgttcggactcaccgctccaagatccacgcgactcattgctgtgccaaccaagcgcgtggtgtgccctctcttttaaaaaaacaacaatttggatcttttttcttccttttttttgtattttctttttatccctcaattatacataatcatattaaaataatataaagggaaaaaaggcagctacacaataagaatgtgggagtatttggaagaaaaaaaaattgtaaacaatcaattatatgcattttgttatttttgccccatttccgttttgattcttatatacgagtagagtaatgatggagtcattctttttaatactctttcttaaagacttttttatgattaattataattcattaaacaaatattaattttgattgatcttgtttttgatttaatacaaataaggagaatgaatcattaaataagaacaaccaaaaataatatttttaaataaattctaattaatcataaaaaaatcttaaaaaaagagtgtcaaagatagtacatctctcttacatattaacagaaataaaagtgtaccggccattaaaagagtcagtagctatggtggcataatggcatgttaccaataattgttgtgacaaattaagttgcgcgattgtggtaatttagtttcattttttgtcttcgatgcgacagtgaataaattaccacaagtgcgtaccatggggaatttatttttgttgcggggatttcacatcaaatcacgtatttaacagtggtagtattgttttctaagtgggttctagaggcgaagttgatgacttttgagtgtgaaagattgataggaaagtgtagctattattagaaggggcgatacaaatcaacaagtgtcccaaaaatcaacatcgctcaggttgattagtcctgaaaatatatataacgatgttgaaatgcatggaccctgcgctataaattgctttcctaattgagtttgagcgtggcaatggcaatggcaaaagaattttaagaatgcttgctcactgacgctgcttctaatgggaaagactttttctacatgcatatgttttaagaaaaagaatttatcgtataagaaaaaatctagggcgaaaaataagttacattaaaagcttgacatgaattaagggtgcccatgtgagtcagctgttaacatgagatgaaaatttattgaacttatcttgaatttatgtagttaattagatttgtcggtgcgtattccgaatatggaaaagtggcagtaatttgttttatataagagaaatgattgaaggtgcagcgtggaaaagtatatttcaaagcgtatatgtaatggcgtgtaatatcgaaacagaaagatgaatgagtagaagccgaagagtttaggtgagtgttaaaaagaaccaggggccattctctgattttattcaaattcaatatcagaaaatgaccctcgttgggcccttattttgtatccataagcaaagtcaactgaggaactcttccggggttgcaaagctttaactactttgtgataccaacacagactacacttgtatttaaggtccgtttgtttgactattttgcttctgaagaagaaaattctaccactcgtttcacacaatgcttcttggtcagcgtgaacactcgttgttttcagacgtagaatctcaaacaagttaaatcggcagattatcatttgcttatgtgtgtcttgttaaggtgtgaagttattactctttactagacagttagacataaatattttcaatttgctggttcaatcagacaatgtgaacaaattccataaacaatcaaattttgatcaccattaacctaaaaacaaaacaataaatgaggaccgcccaattttgtgtttagtttattagctaaataaacccgtggaaggcctgcttatggagtcatggtcccatgccaagattttcttttgttgaatttagagtagatttgaagagagacagagagagagagagacaaccgaatcttttggagagaatgcataaaatgaaaaaggctttcttattcttgtaaaatgcagtacaaagattttgcatatcatttatatgaatagtagggtcagcccaacaaattttgttggactaatacaaattctatattttaactaaggaacaattaaactaattctttaagacaaaagcacatactagtactagttataattgtcatatgtaacagatgatgaaaaaattataaa

SEQ ID NO: 103 - DNA, 7394 bp, Glycine max; identical to SEQ ID NO: 102
  misc_feature 1..7394: note = source = /organism="Glycine max"/mol_type="unassigned DNA"
  source 1..7394: mol_type = unassigned DNA

SEQ ID NO: 104 - DNA, 7224 bp, Glycine max
  misc_feature 1..7224: note = source = /organism="Glycine max"/mol_type="unassigned DNA"
  source 1..7224: mol_type = unassigned DNA
cttaattgaatttgtcattctcctccataatttactcatagctatttgatggccatcatttgaagagtaaaaatattgatttaaaaattactgatttttttattacaaatatgatcaaatacattaaaaaaacaaaatactggttttgaaaatagtaaataaacaaaaggccttattatttgaagttttatataatgaataagaattattaaatactcttcattttaaaagtataatttatttaattatacttttattttattaacattttgtcattctactagattgacatacaaccgcggtacttttactaaatgctctgaatttaactataataatgactcatttttattaaagcgaaaagtaattcaaataacatgaactttttgaaggaaaacaacatcactttaggtactgtttgtactatttttcaattaaaattaaagtttctcagtaatttatattgtattaaaatattttgtagattatcaagaagaatcattagcaaaaacatacatatgatattgtacctaaggtttctccataggaattcagttttttaaagtcaacaaattttacaatttttttacaactttagagttgacctaatatttatccaataaatgtactacaaactttcttgaattttttattcctgagtgggccaggaactttcttgaaattgtataattttaaaacgtaataaatactttcttggaattaggaatagttgggagtactatgtttttttaaaggaaagttggtaccactatattagattttagttgtttcaaatttactaatttaatttgtttaaaattattataaatgtgtttttaattttaaacttttctatatattataagaaagtttaactaacttctatactaattaaaaaagttaaataatattccttttatctttaaataaatatcacatttgaaaatattatctcaaaataaatgtcatacatgttaattttttactttaatattaattttttattaaggttaattttataaaattattaatttttcatttatttattattttttcttaatttatataaaataatttaaaatgacacttattttaagataaaaaaatataatattatattaatatcatgacattgatttggagaatctcattgataacttttaacttatagaatctcatttccaaccttatcttttaacatacaaggctcaaattaacttgacatctttcacaaggagattgagtttagttattagttaatattaattatttaattttactaattatatttaataaataatttttcttaagagctcttcattggaattgataaaaaaatagttattgaaaatatatattcaattaattgaatgatatttttaggataatattattaaaaaactaaaatagttaaaacttaattatatttgagttcatcatatcttttttattgtttataattgactccggaggtgctaggtactttcttgaaattaggaatagtttgagattaagaaaaataaagtgaaatgtatttaaggaatatttatcaaatatttattataaatattaagtaataaatgtaataagtatttcttataaatattaagtaataaatattcattaaatatattagtattttaatactaaatatgagtaaaatataataaataatcgtaaatatttattatttaaatatacttacttactaaatatttattatataatgcataagcatattttaagtatagttaataataagaaatatttggctataacattaacacaccaaattaaaatttttttttatgagaacgaaattaaaatttaagaaactaaaatcatgcatttataaaatttaaaagagtaaaattagacgtatacaattagtggtcgagtttatttgaatcggtagtatctttgttattatattaaattaattagaaacgtccacgtttaataaatatctgaagagaagaaagtgtatatataagtccacatgagactcccctgaacccagcaacaacaacacttacttgtagttgcgaaatcatccaaacacattccatctattaagattttagagagtgaaaatggattggcaaagcatgatgggcaacctggacccattccaaagaacaatcttaatcctcgtcccactaacactactcctgctgctgttactatctcgtacccgtccaagaccgccctatccaccaggccctaagggcttcccaatcataggaaacatgtttatgatggaccagctaacccaccgcggtctcgccaacctggccaaacactacggcggaatcttccaccttcgcatgggcttcctccacatggtcgccatctccgaccccgatgccgcgcgacaggttctccaagtccaagacaacatcttttccaaccgccccgccaccatcgccatcagctacctcacctacgaccgcgccgacatggccttcgcccactacggccccttctggcgccagatgaggaaactctgcgtcatgaagctcttcagccgcaagcgcgccgagtcctggcagtccgttcgcgacgaggtcgactccgccgtccgcgccgtcgccaacagcgtcggaaaacccgtcaacatcggagaattagtgtttaacctcaccaagaacatcatctaccgcgccgcgttcgggtcgagttcgcaggaaggtcaagacgatttcattaaaatattgcaggagttctctaagctctttggcgcgtttaatattgcggattttataccctacctcgggcgcgtagatccacaaggtttgaactccagactcgctagggcacgtggcgcgctcgatagcttcattgataagatcatcgacgagcacgtgcagaagaagaataattatcagagcagtgaaattggtgatggtgaaacggacatggtggatgagttgctggcgttctacggcgaggaggcgaagttgaacaatgaatcggacgataatttgcagaactctatcagactcactaaggataacatcaaggctatcattatggtatggattcgttttcaaataaatatatcttattcttatcagccacctaatgaaatcgataattattcatggcatgaaagtttaagcattttactatttcatccagagtatttaagataagagcactgtttttttttctcaaaaaccacctaagcacgtacaacttaacgccaatacctcaatgattggattgatcttcagcttatctttggattaatttctactttaatactttaatgggatttttttattgtgaaatctaattgttaaatgttttttagtgcaagaatattaagatctataaagtttaatttattgatgttcatagaaaaattggatgaagaattaaccttttaaaaagtttatttatttcatctcctatacattttatatttttttattggtgtttattaagaagttgtccaaaaagacttttactatttttaattctttatcgtccttcaccttaaaaagaaaaaaagtggctctaccttgagtatgtataatatattgggaatgtgtaaaaatatcaacttttgtttgctaaattataatatattttaaaaaaatttactttctgtaaaggctggttgaattcgtgatattcttaattttcaattttgaatttacattttttaaattagatatatgtatttttaaatgcttttttcaaacattttatacttcatttaaattaaaaatatcaatatattatacataaaatgcgtgttaaatattttttaaaccttttagttgtatagaagtatttgcatgtctgttagtcatcctaaattgctttgtttggaagttttaatttgtcagaagttgatttaagtgcctaagttataaattttctatttatgtgtcatcaatatgtagtttacttattaaactgatttgagttcatttagaaaacaaattttcaacatttacaaaaaaatgtctccaaatcaattatgattatttatgccgaaattgaaaaaagtaaaagcagatttactatgattattatgggctaactggagatgaacaaataaggtagataagatcataagatttggaaacactagccaccaaacatgcagtactagtgagtgactatcccaacgtgtttcatagtgggtttgggttggtgtcatttctccgtccatatctacagttgcttcttcttctctctctctcttttttttttttgtttcaatcaaagttttatttttgaatgcaaaattcaacgttgtgccctatgaaggtggtaagtaaataaagaaaattgatatcggtgatgattgtttcatttgtttgtggtgtgtgttaatattattacactttacacaggacgtgatgttcggaggcacggaaacggtggcgtcagcaatcgagtgggtcatgtcggagctgatgagaagcccagaagaccaaaagcgggtccaacaagagctggcggatgtagtgggcctggaccgtcgggtcgaagagtccgatttcgagaaactcacttatctcaaatgtgccctcaaagagaccctccgcctccaccctccgataccgctcctcctccacgagacggcggaggacgccaccgtcggcggctacttcgtccccaggaaggcgcgtgttatgatcaacgcgtgggccattgggagggacaagaattcctgggaggaacccgaaacttttaagcccgcccggttccttaaaccgggcgtgcccgatttcaaggggagcaacttcgagttcattccattcgggtcgggtcgaagatcctgccccggaatggtgttggggctctacgcgcttgaattggcggtggctcatcttcttcactgcttcacgtgggaattgccagatggaatgaaaccaagtgagatggacatgggtgacgtgttcggactcaccgctccaaggtccacgcgactcattgctgtaccaaccaagcgcgtggtgtgccctctcttttaaaaataaaaaaaaaacacttggatctttctttttttcctctttgtattttctttttatcccttaattatacataatcataataaatgatataaagggaaaaaagggaggtgcgcaataagaatgtgggagtatttggaagaaaaaaatttgtaaaactcttcttgatcattgatgtgagcttttgtgcagaaacaattaattatgtatgtattttgtttctttttgcctcacttccgttttgattaaaaaactgaaataaaagtgaaccggccattgaaagaatcagtggttatggtggcataatggcattaccaataatttgttgtgacaaatttttgtcttcaatgcgacagtgaatatgcaagtgtgtcccatggggaattttatttttgttgcggggatttcacatcaaatcacgtatttaacagtggtagtattgttttctaagtaagtgtattctagagacgaagttgatgactttggagtgtgaaagattgaaaggaaaatgtagccattattagaaggggcgatacaaatcaacaacgagtgtcccaaaaatcaacatcgctcaggttgattagtcctgaaaataaatataatgatgttgaaatgcatgggccctgggctataaattgctttcctaattgagtttgaatacgctgcctctaatgggaaagactttttctacatgtggcagtaatttgttttgtatacgaaagtgcatagtattataatagaattgattgaaggtgcagcgtggaaaagtatatttcaaagcgtatatgtaatggcgtgtaaaatcgaaacaggaagttgagagtagaagtcgaagtgttttagtgagtgttgaaaggaacgtggggccattcttcgattatattgaaattcaatataaaaagttaaaaccgtgaaaatgacccttgttgttgggcccttattttgtatgcataggctaagtcaactgaggaactactccggggttgcaaagctttaactactttgtgataccaacacagactacacttatgtttaaattaaaagccccgtttgtttgactattttgcttctgaaaaacaaaaatctggcactcgattaacacaatgcttgtttgtcagcgtgaacactggttgttttcagacatggaatctcaaacaagtcagcagattatcatttggtgacatgtgtctcgttaaggtgtcaagttattactctttactaggcactagacagtcagacataaatattttcaatttgctagttcaattcaatcagacaatgtgaacaaattgcataaacaatgaaatttgatcaccattaacctaaaaacaaaacaataaatgaggaccgcccaattttgtgtttactttattagctaaatataaacccgtggaaggcctgattatggagtcatggccccatgccaaggttttttttgttgaatttagaatagatttggagagagagagagagagagccgaatcttttggagagaataatatataaaatgaaaaggctttctttcttattcttgtaaattgcagtacagagattttgcatatcatttatatgaatagtagggtcagcgcaacaaattttgttggattaatgcaaattttgaggtggagcgtaattatgtcataaattcgattaatgcctatattttaactaagggacaattaaattaattgtctaagacaaacacatactagtactagctactataattgtcctatgtaacaaatgatgaaaattttatatgaatacattcttgtaaaatgcagtactaagattttgcatgctaccatttatatgaatagtagggtcagcgcaacaaattttgttggactaatacaaattcaggtggagagtaatcatgacataaattcgattaatgtttacattttaactaagggacaattaaattagttctctaagacaaacacatactagtactagttataattgtcccatgttaacaaatgatgaaaaatttataaacactacaaccagcttcccttatttttagatattcaagactaatgggcacaaatattcatatacgaaatgtatcaagtgagagactttttatatatgagaataggagaaatgaatattaataattagatcttgtcatgaatagtcaaaattaaactaaacaattttttgagttttaaaataatcgccttacattaaatctaattcctccacaaataattacatggccttagct

SEQ ID NO: 105 - DNA, 9459 bp, Glycine max
  misc_feature 1..9459: note = source = /organism="Glycine max"/mol_type="unassigned DNA"
  source 1..9459: mol_type = unassigned DNA
aagttggatctataataagagcacgaggaagtattatataagacacattgaattttgatatttatgataatgacaataattatatattaataatttgaatttaatggttatgtactatgtaaaaaaatttatatttttaaaccataaaaaagtattgttggtatgataattaagataattattataaaaataaataaaattattatatataattgttcataattgaatgataatgtaaaactttacataaggtattttctttaataatttttaaaataccatcaaagttgtacactacatatgtaaaaaaaaaaagtaaaaatatttgtacactgcatataattataaatgataagtgaatcaataaataaatatagatggataactttaattcttttttttttttaatttgaaacttttatttttaaaaattaaaactcataaatataattatctaaaaaaactcatcaatataataaatgagtgaataaattaatgaatgcacaaaatcaaattcttattaatgacaccattgaaatttattatttttctcaatattaataatatatgtgtttttatacgcatttttttattaaatatcccattaattatcaactaaaaaggaatttattaagcacactaaataactaaaatgggatatttggttaaataaaagtgtaagtaatttttttttataaaaaaacaccaaacctctatgaaactaattgacactaaagtaatccttaagggggagagagactaaagaatagcattgttaggatgtaattatctttaatttttattcaccttttaatttttttcttatcgatccacctataaaaaattacaccttaaaatgaaaggaatcaactttttattttcgtttatagttttatgccgtcttacatgcatgcatggtacaacacgagcaacacatggactctctattttaatattttgtatattttaaaaatgcatggcttcatttacttgtcttctaatcaacaaaacactcacagtcacatgatccacttgcactagagatatttttgaagtctttttttaatgtataattcttaaccaggtcccaccttcaagaaacatgcgtttatagttttagaatggacaaggatatatgaacatccatgtatttaaacatttatttatcctaccaaattatatcatttacggtatcaatgttttttgtccgatctttatttatttctcttttaaagtgctcaatagaatagtgtccacgttgctttattcttttagaaaaatatacattaacacctgaagtgctagttgtcgcttagggagagaaaaataaattaaaaaatagcaattataattgaacatttatttgttataaatatatgatcaatatttttatttttttctatctactcttaacacatcataaattcttaatacttatatttttttcactagaaattctaaatacttattttttttttcactaaatgtctaataacatttgggatgttcattatttaaaaaaaaaatagaacaatgataaacactaaccattttttatttcaagcattcacaaatacttctcatttttctttatcttttttttccatatgtcatacctacattattttttgagtgaatatttcaactcccacttattttaaacatctcatcaataatttttatttctctccatttctatctttttatcacatcctaaactttatcatatttatattatttcatcttctgttttctttctctagatgttggataacacattggaaaaatataaatataatagataatttgtataataaagaaataagatgtgttaattgaatatttcaaatacgcctgcaaagatttgtcatatgccaaagcacatgggtttgatattggttgtagatataagaattttattagtgaataagatgtaaatatatgagtaaatattctttcaactttgagattaccataatcctaataagtctccatattaataggtgtaatattatcattaatttataatttttttcgtgacagaacagagacatgtgagtatcgtcagagtatatatataaaccccgtgataagtccctgaccttagcaatctatagcagcacttgaaaacacaattcatttccttgtttccttcccaatcaacacatagaaccagagtgcaaagcttttgcagtcagcaagagaaccaaaaaaaatcccacaccatggatttgctcctagaactgaaaaccgcactggaaccgttccgcgaaacactattgttcacaatcccattgactctattgctgctgggcatagtgtcccgaatccgcagaaaaacggcgccgtatccaccgggcccaaaaggcctccccctaataggcaacatgaacatcatgaaccagctcacacacaaaggcctggccaacttagcgaagcaatacggcggcgttttgcacctccgcatagggttcctccacatggtggcgatctccaacgcagaagcggcgcgtgaggttctccaagtccaagacaacatcttctccaaccgccccgcgacaatcgccatcagctatttaacctacgaccgcgccgacatggctttcgcgcactacggtcccttctggcgccagatgcgcaagatctgcgtcatgaagctcttcagccgcaagcgcgccgagtcatggaacaccgtcagagacgaagtcgacttcatcatccgttctgttacgaacaacctcggaagccccgtcaatgtcggagaactcgtcttcaacctaacgaagaacatcatctaccgcgccgcgttcgggtctagttcccaggaggggcaggacgagttcatttcgattcttcaggagttctcgaagctcttcggggcttttaatgttgcagatttcgttccgtttcttgggtgggttgatcctcaggggctcaacaagaggcttgtgaaggcacgtgcctcgctggatagcttcattgacaagatcatcgacgagcacgtgcagaagaggaggagtggccacgacggcgatgaggaaagtgacatggttgatgagttactgaatttttatagccatgaggcgaagctgaatgatgagtccgacgagttgctcaactccatcagcctcaccagggacaacatcaaagccatcatcatggtacgaacctccctgccacactatttattttcaaatgtctaatacatcaaagccatcatcatggtacgaacctccctgccacactatttattttcaaatgtctaatacatcaaagccatcatcatggtacgaacatccctgccacactatttattttcaaatgtctaatacatcaaaatattttattattttaaatttctcatcaccatcttttaaattagaagagaaaattattaaaaaaaaatgagacactgaatcacaaagaaacatcaagaagaaagtgtaagagaaaatatagctaacattcatatatattttaattatatatataaataaaatgaatacaaattgtacttttcttaaaaactattaaaataagatgactacaaattgtacttcacttagaaataagaaatttttgtatacttacggactgtctcgcgtgttcataattttaactttcaatttatcaggaaaaaaagatttcatcaaatatcacaaaaaattaacgggaatcattgataatactgtcctgtttggtatggagaagacaaataaaaattacagtattaatgaaagtaaagagggtgaataaaaatagagaagaaataaaattaaaatagattattttaattaaaattaaaattaaagagggtgaatacaaagatagctaaaaaaaatagctgaaaaataatataatgaattccatttcatcatttttaaaattattttatctcgtttttttttcttttatcaaacaaaaaaaaaatatcattctttcccccttctaatttgtctgcaaccgaataagcactgcatgtgacaataatgcaagaatttttcttactccacaccattaaatcatcacttgtaggaaaaaaaataatcatcaatgaaagtactattcactatggctcaaccaccccctcatttcaattatcaacgagtctatgtaaagtcggaaaaaaaaaaattaaagactttcagacagtctgtacgtgcaaagatgtatcatcatcatgaataattataaattttcttacaatattttttttaacaaatcttcatggttattagatgaaaaaatattaaatatttttatttgtttataagagtttaatggttatatttaaaataattttatattattatttaatcataaattattattataacttttaaaataattattataaaaattaattaaatattatatatataataaattataattaaataatgatataaaattattttagctctaagtatccatccttctcagtctttcgaccgcctctaaaaaaaaagaaaaaaaacttgagttgccaggaactgtataaaaaaggtttgagacttgaaaagagcaagatatgtaagcagtggccattccggttgctattttctctgtttataatatattaaatatatgtattctttcatttattttttttataaagtattaaagatattggtaagaaaataattaatgtgcgggaatataactttacccagtatacatgtcaaatagtaattcactcaaataatatattcttagctaataaactcttgtgccatgatattttcgtctattatttgttttgtgaaaaagttgaggctaggaccgcatattttcgtctctaagtcaaatggagtaatatgtgattatagattgatgtggatggtattacattacaagaaaacataaaaataaaaaataaaaaaatacgccgacaaaacaatttccaaaacttgtttctttgtttagaagaaaacaaagaccattagaattgtatgaaaattatttttgacttgctgtataaggagagattctttaaagattctctccaactggttcgtttgtttaacataacctggattcgtttgtttcataaaaaggatgagatttatgggtaataaaaagggaagaagggggaagagaacaattcttccttcctttggacatggaaaaggaaagtgtgcgaggttagtcctacgtcacttgtatcatttctcagtttatgaatggaaattcagaaacaaaaaacgggttatattacttatattttattttctattttttctttcatttttctaaaaaaagcaacttttaacctttttgtgaattaatgtgtagaaatactattatgcatgttttattatttcatatttcagtaatattttagtactaaatgttataataggatgaagcacattaaattaaaggattgatactatttggtttgtaaaaaatagggtgggtttaggaaagaaggaagtggggggaggtggacgtactatcaaatccttccttccttttttgaacatcgttgactgtgtaataattgtattttttttataactggaaataattatatttgcagctaagtcccacatcattcatataatttgtcaatttatgaacggtagaaaaaaagggctacctgacaaaaatattttaatttatattacatttattttatatttgagatacttatgctattttatattcatgttttgtttcttcactttctatacaactaaacaagtaaaaggaaattgtttatctctctttcatttcactttttcttctgtcgaataatctaaaaattattatactttctacattttttcattctttcacttttttttttcttatatcaaacacaacaaaaatgttaaaacaatattttactttatatacttttatatactttattacttttatcatatacttcagtaataatatgatggattgtcttaagaagaaagtaataatatgatgtgacacatcaaaataaaagattgaaatgtttatagtatccctagtcacatgtatgtgacaaatagcttgtttattatttgtttaaattaatagaaaatgatatttttaatatagttcggatattctggtaatcataacatatattcctgagtaataaaaaatgtgaaccaaacgtgtgcttcaattattcctagacccagtgtccatgcataattttttttaaaaaagattttcatgttacaggcaaaagcatacactataattaactagttaactacgtataaaatatgcaaacttagatcgggagaatgaatggtataaactgtgaagcctcaaagataaaaagtccccattgttttaatttagtggttaatatcggtgccaattatctttcggtccatgtcattgtgatatttcaatttcaagttttgaacaacaacaacaaaataatacaaacaaaaagaaaagaaaaccagaaatgaaactgttgatgtaattattatttttggtttttgtgtacactagtaaccagtaacactattaatgtatagtatttgaaacaagattttggtggatagtaactgacaactatgcaagaccatgcaggacgtgatgtttggaggaaccgaaactgttgcgtcgggaattgagtgggcaatggcggagctaatgagaagcccagacgacctacggcgcgtgcagcaagaactcgccgacgtggtgggcttggaccggcgcgtggaggaatcagacctcgaaaagctcgtatatctaaaatgcgctgtcaaggagacgctgcggctgcacccacctatcccgctgctcctccatgagactgcggaggatgcggcggtgtgtggttaccacgtgccgaagggctcgcgcgtgatgataaacgcgtgggccatagggcgggacaagagcgcgtgggaggacgcggaagcgttcaagccttcgcggtttctgaacccgcacgtgccggatttcaaagggagtaactttgagttcattccattcgggtcgggtcggagatcctgccccggaatgcaactgggtctttacacgctggaactggccatggcacacttgcttcactgcttcacatgggagttaccagatggaatgaaacccagcgagttggacacgagtgacgtgttcggactcaccgcgcccagggcgagtcgactcgtcgcagtcccatttaagcgcgtgttatgccctctctaataaaagaaaaaaaagtatcgcaagtttacgaataacggcctcgaagggcttcaccaaagtataaaaagaacatggcggtgaagtgcttcgggactttaaggttgttttttcttcttctttaattgatgttttttattcctcaattgtacgtgggtgatcgtgtctggaggagaacccagttattgtaaagaagacaaggggatattaggttaagttttctgttgattattgtaaagacgatttgatctacgtgcggaaacatcattattgttgggttcacttggttgtattttttggtcgcactctggttttagttgtttgcaaacttgataaaaccgcagcaaaacggaagtaccttgcaaaagtcttgtccagaaaatttagggttttattaggcaaaatttacatggaggctaaatataaattattaaattcagggggcaaaattattgacaaagttttataatgtagtaattttaacatttcacttttaacaaataatatatatatgactaatttatttaagttattttacaatataattgattccaaataaaatttgagtaattttaattttataaattaaaatatgattaataatattattttagtgatgcttgattacactttcaaaaaaaaaagtaatgcatgcattaacaaatataatcttttcattatacttttttgaaaaattattgtttaattattcaaatcaacaatattttttaaactttaatgtattaaaaatatttttgcaaaaattataccttcttagtcttgtcacgggcgtctaattgtcatgtcattaccttcaatgacttgcatggacgtgtcaatgattgaacatgatgatgtaacactctgtttgtcatatcatcacttaatgaaaagatgactaacggtaggtagtaaaataaaataattttttttaagtatgtatttgataaaaaaaaataaatttttagatagtaatctaaaaacaacctatttttcaggtatgaaaaacttatttaatatatgacatgaaaaatttatttaaattatatataagtggtattttaccgtttataattaatgtacatatgtaacatattatgcattctctcactattccttcactattctctggattcaaaatgtatatcctcattatttgatcttgttggattctcttctaaccttgtccagatattaaaatgtatgcatcttaactctttaattttacctttaatattatcttattaatctagttcaattaattaaatacgataaattgatatatgagttattgtaaatttcaaatagtattttttttaaatgtatttcttattttaaatgaggaattatcttaaaaaaattaaataagataaagtttctcaaatttatataaaattaaattacttatatgtttatcaagttattttaattaatttttaattttttgacaagtttacaaatatttgatcaaataaaagtctatgagaatcgattttttttattgacttataagatttgcttttaaacataacttaaatttactattatcatttttattttcattttcaataaaatgataaaatttatcatttatcattttatctgaaattaaattatttattttgataaaactcctccttatcatttgttaactaaaaaattatttttcatattgacataaataagtttttcatatctaaaaaatagaccaattttagattattacctaaaaatttattttttgtcaaatatttacctgaaaaaaaaatagtttcattttactacttgttgttaatttcttttcgttaagtaatgacgtgtgacagagggagtgccagattatcatgctcaatcactaacacaaggtaataacatgataatgaagtatttgtgataagatgtaatgatatgatactccatctgtcacgtcatcatttaacggaaggggaaattattgtcaagtgcttggttgaaaaaaaataatgttaaatgatatttaaaaaaacctatattccatgtatgaaaaaattatttaaataagttattttgtaatattcgttgtattgtatcaaaagatatttcatacatttgttaactttttattaatgctttttttcaaaattttataacaaagttttataatttatatatattcttttacaatgaccttctatttagatatattaacaaattaagttttatctgaaaaaaaaaattaattaagttttataatttagtctactttttccatttttatccgctccatttctcacattttattctctcttatct

SEQ ID NO: 106 - DNA, 7224 bp, Glycine max; identical to SEQ ID NO: 104
  misc_feature 1..7224: note = source = /organism="Glycine max"/mol_type="unassigned DNA"
  source 1..7224: mol_type = unassigned DNA

SEQ ID NO: 107 - DNA, 6752 bp, Cynara cardunculus var. scolymus
  misc_feature 1..6752: note = source = /organism="Cynara cardunculus var. scolymus"/mol_type="unassigned DNA"
  source 1..6752: mol_type = unassigned DNA
tgttgtgttatgtattgttttaattgtaaggaatataggacaaaaattagtcttctcttcttcaatacaatcatttttatgttatataatatttttggaataacttcaaaattacataacgagttatctcattttaaatgattaagtccttgaactatttttttaaatgaaaaacaccattaatttttttttaatgaaaaaccattaaactttttcatttttattaattaaatccctggacatttaaaaaaataaatcatgcatttttaaaacttggttatctacatcattttaactcaaaaaaattacattactcaactagcaatagttcgatggtttgtttatttaaaaaatcaggtttaatcattcaaaatgagaaagtttaatggtttttttaattaaaaaaatagttccgaaacttaatcattcaaaatgagaaatgtttaatggttttcttcattcaaaaatatagtttaaagactcaatcatttaaaatgagaaagtttgttgagtttttttggacttttctctaatttttctatataaatcaatatatttgtcatttgtgtatgtatttgtaggacattaaatatgattaagtatatgtatattaattacatggaataatttaataaaaagtaacaaaaaaaatacaattgttagtattataaatatatatatatatatatatatatatgttattttccaatatttatttattttttaaatatgttattaaaatttatataaataattaaaaaatctaattaagaagaggttgaaaaaaacgtgaatatagatgcttgaaaatggtttggaaaagtagtctggtgtggcaagactgcaaaacgttttagcacaaattattataatatttttattggagttggttattatacaacttttgatatttgtgtaaccaatataacataatttcaattgtccatttcatttgattaatatgtgatttcataaaaaatttgattgttttctaatataaagtattgatcataattaatgaaccatattcatcttctataaatattcaaatcataataaatgaactcttatcatttccataaatatctcaatttattgggttgtattgattgtataaatacgacgggttgtatttgaaacaaactctatttttatttttaataaagctttgttatgatattatactcacaaaaatatacaagtcattattaaattaaaatgatttgagaataactaataaaattaagtcattattcttttatattatatttcatataattttattaattatttaaaatatattatctaaattatacaatattgttgatataacacattaatggaaattaatttttaaattggtttataaattacagatatatcataaataattacactgatagatgtaggaatgatcttacatgattaacctgctaatataagatgattttggtatattataccattttagattgttatttattcatttaaccagctatctatatcatatttttcagtatttgaatactactgtattttttaaccagattttatataagattggttagaattacctataattaaccttttactcgaaaatattttgaaatctctttatcatgtttccttttaaacaataagagtagaattatgacattattttaattatatttgattttttcctattttttaattgacatatacataaaatatatatgtatatatatatatatatatttatcatatatgtgataagaaaattagcttttttatttttaaaatattatgaaaaaatgaaaattaaagtcaaacaatttgggaattaatttagaaaaatataaataattggaaactggaactcattcaaacaggaaccacccaccttaccctgtatatatacatccccttatttctttcattaacgggcaaccttctctcagttgcacttcgtagtattacacacatacatattccgatacgacggttcttcgccggaaaatatggatgttttacaactccaagggctattacaccctatgcccatcctattttacgttgcactccctctcttaactttcttccttctttccagttttcgccggaaacctcttccgccgggtccaagggggtggccgctgatcgggaacatgttgatgatggatcaactaacccatcgtggtcttgctagtttggctgacaaatacggcggtctgcttcatctcaagatgggcttcagccatacagtcgctgtctcgtcaccggaaatggcgagacaagtactccaggtgcaagataacgtctttgccaaccgtccggccaccatcgccatcacttacctcacctacgaccgggaagacatggcgttcgccaactacggtcccttctggcgccagatgcgtaagctttgtgtcatgaagctattcagccggaaacgagcggaatcatgggattccgttcgggatgaagttgtctccatgatcaaaatcacggcggctagttccggctccgccgttaaccttggtgagctcgtgttcgggctgacccatgacatcatctaccgagccgctttcgggtcgatttcccatgaaggaaaagaagagttcatcagaattcttcaagaatacaccaagctttttggtgctttcaacttggccgatttcattccatggctagggtttatcgaccccgccggactgaacacccgattaccaaaggcccggggtgagttagacgggttcatcgataaaattatcgacgagcacctccggaaagagaagaaaaccggcgacgacgccgacaacgatatggtggacgagatgttggcgttttacagtgaagaaggaaaggtgaacgaaggcgaggatttgcagaacgcgattaggctcacgcgaaacaatatcaaagccattattatggtgagttattttttaatattttatttttgttaaaataccaaaaataacaaagtttatgatcataaacatatttattaattaattaattaattaaatcataaccgtgatgtttgattggtttgagtttggctccaaatatcaaaggtattaggaggtccaaggttcaaatccaagaaggaataaaaattctcttatggtcacggaggttcaaatacggtgactccgggttatctgtaaagtaggtggtcgtcctaggactagtcagctcgcatagtgggttgtatatcctggttaataaaaaaaatattatatatatatattttgcttgaaatataatctaatgaatataaatatgtgttaatccacaaattaagtaacgtttccccttataaaagacctttgatttagagtcaatttggtaacatgtataacaattaaatgaaatcataaattctatttatttataaaaaaattaaatatttaatacatattttatataaaaaaaatatttaatgcaatttatttctttagaagccgtaaagttaagtgaccccatttcattaaactttttcttcattttaataattaatgattttaatttattttaaaataatcatcaaaaaacaaaatttatataacaaccgatgtcatcttcttaattttgttttcacttataaaacaaatatttaatggtgaagatcatgaatgattccatctccatgctcttgttaaccccaattaattgtcttttttttgggtttttttaatctaattaattaattttgtgcttattttatccttcaattagtatattattttgaataaaaacagattgtattagttgaaatgggcaaacatccacaatcttgatcaagtcatcaaaatgggatatcgatagaattatatatatatatatatatatatagatttgggatcatatgagaaaagcatgttttcatgagactcgtgacaccaactttttacatttgaaacttcatcgtgttttttgttgtgaaggttcaacttttttaaaccttcacagtgtttttgctgtgcagtttcacgtgtaaaaatatggtttcacgggtctaacgaaaacagtctggtctgacgtgaacccaaccatatatatatatatatatatatatgaaaagatgacattagttttgcttaataaagaattaacatttggtaaattatggctttcttataggatgtgatgtttggtgggactgaaaccgtggcttcggcgatcgaatgggctttgacggagctcatgcatacaccagaagcactcaaacgtgcacaacaggagctcgccgatgtcgttggccttgaccgccgtgtcgaagagtctgatttcgagaaactgacctacttcaaatgcatcatcaaagaaaccctccgcctccaccctcccatcccggtcctcctccaccagtcatcggaggccacagaagtcgccggctaccacatcccaaagggaacacgagtcatggttaacgcatacgccatcaaccgtgacaagaacgcttggaaagaccccaacacattcaacccatctcgattcttggaaaatggagctccggatttcaaaggaagcaactacgagtttcttccgtttgggtctggccggagatcttgccctgggatgcagcttgggttgtatgcgatggagatggcggtggcccacctcctccattcgttcacatggcagttgccggacgggatgaagccgagcgagatcgatatgactgacgtgttcggactcacggcaccaaaggcgactcggctggtggcagtgccaactcctcgactgttatgcccactgtattaatcgacgaagggtcggatcttgacttccgagacactgaaaaattgttgatgttgcatgaacgaattcgaatggttttaaatttttttttcttttttgtggtcatgattttttttcgtaaagcaagatcgatatacaaaaagataaataaagatttcaatttcatgtacattaattcataaactcgatttcaattccatatattttcattcataaacgatatcgatagcttttgttttgaaaggtacgtagcggagccatccacattgtacggataaggcaagtaggtggtattcggagttaaaagtgcaaaggcctttctttcttgaaggggatgtttggttcggaaaatgaagatggtaggttgtatggtttaagacggatggatacgtaccaagttaaggaataaattatttgaaacaattttatcatgttaaataatttcatgacactaaaatgaacaatgaaattgatggtttttacggtttgtataaatgatatggtttgcaaaatatccctatcccccctatctctcaatctctctctctctatatatatatatagagagagagagagagagggggggggagtgatcaaatacaaaccatttaatttgtacaaaccgtgcaaagctcattcaaccaatcataattttttattttgttaatttttttttgtaaatttgagaggttgattaatcattttgataatacaattaaaatgagaatatggataaagaaagaaagaggttgttaaataattttattaaatacaaaataagattattaaattactaatttactgtttttattttattaaaaaataatattaattattggaagtgatttgctaatattaaatctcacgattatgacaagctatcgagttcttattttttgaatatatatatatatatagagagagagagagagtttactttgcgaaccacgttatttgtacaaatcgtacaaacttcattcaatcaatcataatttttcattttgttcattttttttgtaattatgagaggttgattaatcatttttattgttgagggtgttaatcatggatcttatcatcttcattattttaattttagagaaataatgaaacagtttcattaaaatttaattaagtattatatgaaacaatttcataaccatatataaaacgacttcatcaaaaaaaatattatatgaaacaatttcataataattttttataagattaaacaatgaaatcgacggaataaaatggtttgtatgatttgtataaattgggtagtttacatttgatcgttcatctctctatgaggggatgtatagtgtaacgtcaaaattaagaatttgatttatattgaaaacttgagaatcaatccatcgattagatgaaaagttatatttccaatgttttttatataggttgcaaggcatatatgtaatttcataattatggaaaaagttatatctcttttttttcccttaattgtttatgtaatttacacttgtattcatttacaaaattaaaaaactataattattttatttataaattattttcaagattatttaaataactttaatatatcaaaatatgtagtcaaaaatcaagatgagtgcatttgacaataaaatggtaatcaagacggaaaaaaagtgagacgttcaatctgttaagaagtaaggcatagttctaggagttataaaaaccagaagaaagatgttctggtgcattaaaaacatagtttgtaggtcacgaaaagttgcacgtagggtttgaactttgaagttgaaaaatggcttctggtgctgaaaagtagaagtttgttacattaattgagaaaaaaaagtttgttacattatatagaaactaaaccaaagttgttacattttgtcctaaaaagtaaaaaactaaagttagaagaaagttcggtttgttcaaagtctcattaacgtggttcagatctggcaaggtaagatcgaacgtagacttgtggtctggct

SEQ ID NO: 108 - DNA, 5847 bp, Cynara cardunculus var. scolymus
  misc_feature 1..5847: note = source = /organism="Cynara cardunculus var. scolymus"/mol_type="unassigned DNA"
  source 1..5847: mol_type = unassigned DNA
atcttaactaaagttacttttcaggtcgatctgtatcaggataaaattaataaataatcgaagtttgataacttaacacgttgtttctaacacggaaactaacttctagaatttcatctgggcttccactacaacataacatgacaatcatatttcgaagagtcatataaaagatataatatacatcatttcgcttaacgtgtttttattgatacagtataatacgacatttagtaggacacctttttgtacaacatcttgtagaaatagttaattgaacacttatgaattgtttatgaaatgtttacttggtaagctcttaccggatcacttaccttattggataaacatgacaatgatcgacctagtaaaagggatggcaatgatcagtaggcttacccgataagtaagtttgttgactcttactcagtaagcgtttatcgggtaaaattattgcatatagtgtcgttttagtggcttagaatcttgcttggtaaggtaattgagagtgttgactcttatcagtaagtgcttaccaggtaaaacgtttgggtaaacaacctcttaatttgtaggatctgtagactggtgtcctataaaacagttattgggacactgatatgtattactaaatgtaatagtagttaatcatattttacaactacataaaaagtgtcctacgttatactctagctattaggaatttttagtgttacataaaacatgttattagacccttgaatttatttcataattatagaatttgttaggtcgtttagctttgtcttaacaacttattgtaacacatttagataagacatcaatgttttgatcctataaattaaaggatcctaaaaataggtttgtaaaacactactacacattctaataggtgtattttgttacaattgttcataatatttattccaatcttttagccacaactttttatttatttatgtaaaagtagtattttgaaaatatttgattaatagtcaaataataatgtaaaaagccttcaacgtgtagctcagtaggcatagagtcaggacgactttgtccaaatacatgataggtcatgggttctaactgcccttgttagtctcaaccattaagaaagacaaaaaatatccccttacatcggtgcgtatgtccgtgtgatactgattggtgttatcgataattactttttttgtccacataaaaccattttggggctaaaataatatggtgaacaaggatggaaacaacaaggacattccaacctttttggaggccctgctacatccaaaacttttgattacattgcataaataagatgacagatgatatatgtcaagaataaaaatgggttaaacttaaaacgattaacaagtatatttgggatccaaaatttcggaggccctaatcaaaatctcacatttaacatgttaagggacggccctggaaaacagtatggtacggggcggataaagcatccctcgaccagaccagacccgacctcgaaccagtcctcgactagactagacccgacctcgaacccgactaatctccgacgggtttggagggaaggaatccctagagggtttgggaacccattgggtacccctctaatatatatatatttttttaaattataattaattatcactgcatagtataaaggccgagtctttttatatcacatcggatgcatatacatagagatacaaccaccccaaattaagatgtaggccactctcttgatttgacgttcaatcttatatatcatataataataaataaataaataattaattaatgcatttcaagacaagcggatttagtcaatattttccattcattatttcagaaaaataagactaataataaatagtaaatcactgtacactttcaaacagggtgtgcccgatgaaacctgtatatatacacctcttcatttcactccttaggtagcaacaaccaaccagttgccacttatctctccacttgaatcaataatggattctatccaaatacccatatcaatctacgttatcattgccctcttaaccttttcttttctagcatggctacgccggaaaaacctcccgcccggccctatgggctggccggtcattggcaacatgttgatgatggatcagctcacccaccgtgggttagcccgtttggctgaaaaatacggtggcatcctccatctcaagatgggtttccgtcacaccattgccgtgtcctcgccggagatagctcgccaaattcttcaagtccaagataacatctttgctaaccgtcccgccactattgccatcacctacctcacctacgatcgtgttgatatggcgtttgccgactacggacctttctggcgtcagatgcgtaagctttgtgtgatgaaactgttcagccggaagcgggcggagtcgtgggattccgtgagggatgaggtggattccatggtgaccaccaccgccaccaactccggcttgccggtaaacttgggggagcttgtattcgggttgacccatgatattatttatcgtgcggctttcgggtcgacgtcgcatgaaggtaaagaggagtttattaggatcctacaggaatacaccaagctttttggtgcattcaacttggctgactttatcccatggctcggcttcattgacccggctgggttgaacacccgtttacctgcggctcgagctgcgttggatgggtttattgacaaaatcatcgaccaacacttggcaaaagagaaaaaggtcggcgatgaaaatgtggataacgatatggttgatgagatgttggcgttttacagcgaggaaggaaaaaccaacgaagccgaggatttgcagaacgccatcaagctgacccgagataacatcaaagccataatcatggtaaattctataaactcattttttaataattatgaatgataatttattaaatttattttctattttagtttattttaaataagtatatttacctttaattataaaatggaattactttattataattatcatttacttaattaaaaagtataataattacgtatttataaaattatatgatgatatatgttttgggaggaatgggtatttatttacatcatcttgtattctattaaaagtaaagaaaagcttttgattcatggtaaattattactaacacaaaaaattacatcattttgatgaaaattttgttgaattgtttgcaggacgtaatgtttggtggtacagagacggttgcttctgcgattgaatgggccatgacggagctcatgcatacacccgaagcgctcaagcttgtgcaacaagagttgaccaacgttgttggtcttgaccgtcgtgtcgaagagtccgacttcgagaaactgccctacttcaaatgtgtcatcaaggaaacccttcgtctgcaccctccgatccccgttctgctccaccaatcgtcagaagccacggaggtttccggctaccacatcccaaaagggacacgtgtcatggtcaactcctacgccatcaatcgtgacaagaacgcttggacagaccctaatacttttaatccatctcgttttctgcaaaacggagccccagattttcggggaagcaactacgagtttctcccatttgggtcgggccgaaggtcgtgcccaggaatgcaactcgggttgtacgcaatggaaatggcagtcgtgcaccttcttcattgtttcaactgggaactaccagatggaatgaaaccaagtgaaatcgacatgggcgacgtgttcgggctcacagccccaaaggcgatacgacttgtagccgtgccgactcctcgtctgttgtgtccgttgtattaaaactctggtttttataatattatttttataaaataatgtataagaaatatatataagaaccacaaagatttgattctataccacttgttcataggtcaaacctaatttgtgcatctgtttttttaattgtatttatgactttttaagcaaagaaatttcaaaaggttggcaaaatcatgctttcccaaattaaatatttgaaacaaaatctatgagggagacatagaaaaatgagcacaaacataattggaattatttatgtaatataaaactaaacacttttgaataaatttacaagtacaaagtttaaatacctagctacgtatataaattttaaataaagatacgagaaatcataatttacgttttaaaaaataattgatgtaatatacacgatgttaataatggaacgtgggtgaaatttaacgtgttgtattcttaaattagacgtacggatttttaagtacatgttttaaaaaaataaatttatatgataaaacgtgttttaaaagtgtaaatggaacgaaagtatggttattattgacgtaattagtcaaagttatatatttgtaataaattaatgatgattttcatatctattcaccacaatttttatatgctaattaaaaataacataatcgtttaccacaagaatacaagtttgggatcaatgttttcaaccacatgatgatgatgaggatgatgttgatgttgatgttgatgttgatgatgatgatgatagggatatttgaagtgaatgtttcacttgtgtaatgccattttccatgatgttgttgggatggtaatgtcatccatgatcatccatgtataatatataaaatgtcatttctttaacattataaatctagttttttttttctaaatttaattgaaatttaaaatttttaattcccgacttttctttgaaactaaagataagattgtaatgatatttaatttcaaattctattaaatctcacaaactagacgagttagggatcgtttacttaaatacttatcaagtaagaatttatcggtaaaattaacactatcatttacttaccggacaagattttattcactaaaacgcatcgtatgcaacaatttatcgcggtaagcgctaaccatggtaagagtctttgtcctcacttactggttaagactcgaaattcttcttttcgctcttaccgggtaatattcttagtctttgtcatattcattcgataaagtaaattgctttggtaagcgcttacccaagtaagcgttttgtaaacggcaccttagtgtttgtggatttatgacttaaatctcacaatcacgggcaatttctctcatgctacacacacttgtacataaattgatcgtgaaggcaagtttcagtgttttatgagttttgatgtcgagttgacaatgtgtatgtcatgataaacttccaattaattgttccttacattcgttgtgtaagatttaatatttttgagatgtttgataacgagtttagtcgtagtaaggcccatcaatacatcagtttggtcgatcacattgtatatgtgtcacaaagtaagaaagaaaactcaaatccgagtttcatatttctatctcggcacatctttcatgccaaataataaatgtaacttaggcctacaaagctatgtaaagaatgacaaaaaaaacgaataaaaacaagcctatttcagtttcgtaaatacaaatgttaccaccatttaatttattcaagtttttggaaaacaaaaaccgtacattgcatccaagataagcacattttcaataacttgtcatctcactacaacaaaattgggtttataggacgcccccaatctcgaagtgtcccaatagaccgttttatagaactaaaatataaagtgttctatctaaagacgttgtaataagtttatatgacgcttttggacgtcttaacaagtttttgaggacatttttaatacaaagtgtcacattagcaactttataggacactttga

SEQ ID NO: 109 - DNA, 6260 bp, Raphanus sativus
  misc_feature 1..6260: note = source = /organism="Raphanus sativus"/mol_type="unassigned DNA"
  source 1..6260: mol_type = unassigned DNA
taagaacttggaacagacacaaacaaagacatgaagttggtaaggtcagactattaagagaatcaatatattattcgatttaatctatattattaaaattgaagtacaaattagattttttgaaacgaataacagtttttttttaaaaaaaaaattgtttagaaacatggataacaatttttaaaagtatgtttggaaacatgaatatcagtttgaaaaagtttgtttagaaacatggataagagtattaaaaataatataacgttgtttggaaacatggatagtaatatattaagaaaaaagtaacaaattttgtattattaaaaatactgaaagtctattatattatgataaattatctatttggaagagttttaaaatataagaaaatgagctaatataattaacaattttttttttgtctaaaataatcataaaacaagattaaaattttatatacatatttcaaatcaaaataataatttacacttgatttatatcaaaattgattcaaaaatatacatatattcaaaagcttattttactaaaatagtttccactaaccattataaaaaatattttcaatatatataagaaaaatacaataaaaagtccattaatttcattataccaacttaaattatggttttaatatttcacattaagtttttaaaatacaatatatgtgattatttatatgatggtatgtattaaatactattaattatatgattacttatatgatgatatatatgaaataaaattaattatatgatgacacaaatatgatatataatagtgattaggggttagcgttcggatacccattcgggttcggtttgaatttgtttggatttctggtttctgggatcaacgatttcagtctcatttggatatttctaaattttggttcagattcaattcagatctttgcagattcgtatttgattcaaatctttgcgggtttggttcggattcagataacccatttaaattatttttcaatttttagaatattttatactttaaatttctcaaaatatgtaaatgaaataatatattacatataagtttgaataatataccaaagaaactcatactaatagagcgttaacgattgtgagttacaccatccagttttgagttttgaccatcgtcgttaaagtgaagtggaaaaatagttttctcggatatttttgaaaattctccgattttagtggatcattagacgataatcaaatctctttcgaatatattcggataccaagatcattaaaaactcatgcttttaatatacagattttctattctaaacaccaaaaacgtaaagaatataatgtctccaagaatataacttcaagtttatatattaatatacttttaatatacatttaatagtacattttttattgtgtgcaaaataaccttgcatgtaacaataaaagtaagggaaccttgttagtctatcagtctatttctaatgtctaagctttagagctattaattgatattaataatttatctagaccttgtgctctgtggatatctttagtaatgttggaaagtagaaaaattacaattgttttacacacttgtataaatatagctgtcgaagtcgcggtaattcttataactaattgtttcaaattattatgttattgatggaaaagtatatttattcaacctatttattctaggtaaaaccaagcagtactcgttaatatttcaacagcattttttgtaactcaccttttctctaatttaaaatccactaataccaataatgatccattatatttctatatatgcaagaatatccattaaaaccagtcagtatattatttgtttccatattaaacaaaataatagtaataattaaaaaggaggtgtatgtataattgtatacatatatatgagtcacggaaatgaaaatccccgtagaaaattagaaatatgtcaagaaatgggaccactccttgattataaaacctacctttcgtaacacatcccaaaatggcgaactttgcctgatcaattcttgagaccctccacaaacaaaagaaaaagcatttcccaaaaagaaaaagagactaaatggagtcttctatatcacaaacactaggccaagtattagatcccaccacgggtattctcatagtcgtctcaattttcatcttcattggcctcatcacacggcgacgaaggcctccgtacccacccggtccacgtggttggcccatcataggcaatatgtcaatgatggaccaactcacccaccgtggtttagccaacctagctaaaaagtacggtggcttgtgccatctccgcatgggatttctccacatgtacgccgtttcatcaccagacgtggctaaacaagtccttcaagtccaagacagcgtcttctcaaaccgaccagcaactatagctataagctatttgacttatgaccgagccgacatggcgtttgcccactacggaccgttttggagacagatgaggaaagtttgtgtcatgaaggtgtttagccgtaaacgagccgagtcatgggcttctgttcgtgatgaggtggacaaaatgatccgctctgtttctaataacgttggtaagttcgctcgcgtatttacactcgctatctgcagttgaatatttaaaaatgaataatcatttttgttagtttattatgcatttgttttctttgatatatgtattttaatagtcaaccaaaaaaattgtaggcaagtctatcaacgttggggagcaaattttcgccctgacccgaaacataacttaccgggcagccttcgggtcagcttgcgaaaagggacaagacgagttcataaggatcttacaagagttctctaagctttttggagccttcaacgtagcggatttcataccttattttgggtggatcgatccacaaggaataaacaagcggctcgtgaaggcccgtaatgacctagacgggtttattgacgatatcatcgatgaacacatgaagaagaaagagaatcaaaacacagctgatgatggagaagttgttgataccgatatggttgatgatcttcttgctttttacagtgaagaggcgaaactagtgagcgaggcaacagaacttcagaactccattaaacttacccgtgacaatatcaaagcaatcatcatggtaaatatttttcccgaggcactagtaatagtcattattcttaatgcgtcgttacgtcataaaacttatccatttaactggctgtttttctcttaaagtatatattttctgtttctacaagtagtcaattgatagtagaaaaatacaaaggtttcatatcatacgtaaataaaataactgcaaatatcataacgtcatgtgaattgacttttttttttaaataggacgttatgtttggaggaacggaaacggtagcgtcagcgatagagtgggcattaactgagttattacggagccccgaggatctaaaacgagttcaacaagaactcgctgaagtagtcggacttgacagacatgtggaagaatcagacatcgagaagttgacttttctgaaatgcacactcaaagaaaccctaaggctccacccaccgatcccactcctcctccacgaaaccgcagaggacactgagatcgacggttacttcgttcccaagaaatctcgcgttatgatcaacgcgtttgcgattggacgcgacaagaactcttgggtcgaccccgaaacgtttagaccatctaggtttttggaaccgggcgtaccagatttcaaagggagtaacttcgagtttataccattcgggtcgggtcgtagatcatgcccgggtatgcaactcgggttatacgcacttgaactagccgtggcccatatattacattgcttcacgtggaaattacctgatggcatgaaaccaagcgagcttgatatgagcgacgtgtttggtctgacggctcctaaagccacgcgtctctacgctgtcccgagcacgcgccttatttgtgctgtttaaagtaatggttcgaagcacgtggacggtgaaaggaaaggtggttggtatggttcttggaagtggtgtgagaactcaaaagaagccctgaagatttgtggatgttatataatactatatgtttatgtatttgtgtactaatgaattaaagttatgtatcacgtgtgttctggatgaaacataaagtggctctttgtttcgtctttcattttcttttgggaaatattttccttgcatgaaatgtaaacactgaaaaataagatttttttacaactaatttgcatattacaaaaaagtatactaatgaattaatgatataaggatattgcatttgctggaaatgttaaaacataggcatttccatcactgtgagccacacatgagtttggcaaccggtgaactaagtgaatattcatcaattttaagatttgagaaacctaatgtagagaatatggagatgataacttgtaattttatcctaaattttcgttacattataatgataatgctagttgtcaattaattaattgttatataagaagagttttgttaaaagaacgagtttcacagtggaagttttattgtacatattttcttgttttatttgattttggtaacgcccgtcttctttattgaaaatatttatattttcctttatatcattagtatggaaaaatattgtagtctttatcgaaacgcatctattttacctgaactagaaaattctgaccattatgtcatagacccttcatttggacgctgttgggactaaactaatactctctccgtttcatattatttatcattctaagtttatgcacacaaattaagaaaatatttaattttgcatattttcaaaataaaagcattatcaccaatacatctaaccatatatcaactaataaaaaataaaatagagaatattttcaataaattttgcattgaaaaccgaaaacgaaacttatttttaaacgaaaattctaaaacgacaaataattccaaacggatggagtattatatttgttaattgaaaaaatcacttatacatgtatgtgttgattatctaggggttgactgtggaaacacaatttttgtgattaataacggttgtttccaaatcgtttcaaaccattcaaaatctcttaaaatcaaaggttagtttctgaattacggacagttatggttgaataaataaataaaaaaaatttaaattttttttttaaactttaacttaaattttaaaatgaagatattatgtataaaatatacataaacatttatttttagcttaaatcataaacagtatcataatttattttataaaaactttaaactaagaacattcactataatttttaaatattaatatatatacatataaaaggatatgttcatatataaacgaaacaaatattattttaaaagttttaataaaaatcttcaaaataaatttcattgaaattataacttttatttaaattaaaaaattaaattatcatgtattgttaataattatactatattaataattaatatattttatcatataagtgtgtttctgatatttttataatgttatatatgttaatattggtaatttattattaaatctatacgataaatcataaaaatcagttataaatatttagtgaacacgataattttcaaacattgtaccagtcatataaaattttatatgattagttacatttgaaatggtaactatctatattcacaaatttctgtatccacaaactcatacatccaaccacaatcgctgcatttgaatccgtaagatccttagtttaacaatatagatagatatcaaaatctaattcatgcatccaatcacaatcgctgcttttaaaccagtgagacctttaattttaacaatatagacgatcagatttctagatagatttctaggtagatgtcaaaattgtatctaaactactcacttgcaaaattgtaacaaccagaatgcatcaagcagtgatcgatcccaacttcaactactttgacgaaatcaataaattacaattacttagtctattttccgtagctacacgtagcgcgtccatgcgtggactattgcatatcacaaaataattgcatataatatcatcattatcagtatttaatgtccgagaaaaaaattcaacgatacagaaaatttctagtataaattataaacaaagtctctaaaaactagcggtgcctccttgacataaaacattttttcagtggtg

SEQ ID NO: 110 - DNA, 6354 bp, Raphanus sativus
  misc_feature 1..6354: note = source = /organism="Raphanus sativus"/mol_type="unassigned DNA"
  source 1..6354: mol_type = unassigned DNA
tattgtttattaacatttgtatatttcttataacaataaatttaaaccgttaatcctaaaattttcaatgtgatatttttaatgcaaatttcaaaattaacatatttatttatttttatattgtacatagtttaatttaaactaatatgtatataatatgaatatatattaaatgagaattcatattgaactactttctttaaaacatattgaatgaaatcattacaccgtccaccttaaggcacaacaatatatcactgtattaactcaaatacataatttttgtgattcctcttatacgaaagggtcaaaatatgaaatcatgaatctttgcaccttgtaatatgaaatcaaacaaaaaaaacgtttcccaattaatatatatgggattagagtaatcatataaatcaatatattatctcttgtttatttacaattttttatggtaaatatatcagaataactattttatttgattgggtcagatgagaactatctcaatagtaagatcaccaacctaagagtattgcttccacagttgaatgattttgacattgatctcccacctaaacttgaatggtttcaagtctgaaacggggggaggggagtaaaagaggacaccgaacgtgttctaagaaatctatgtggaccactcacatgttttttttatgtgttgaccttctagcaaaccaaaacaaccttcttatatagtgatttgttcttatttttgtttacaaatcagaatgatcgtaagctttataccagcttcccataaataatactccacaatgactatatggaaaactgaatataaattagcaattttcgtgtatccgtaaaactcattataggaaagtaatgatctcgtgaatatattctctagttaatgtgttgctaacaatgcgcgatgtatattctttgattgactcttcaaaaattaagttaatgtgacagtgaacttggtggtatttatatagaagcgcattttaggaatgcttttaatttgcctttgactaactaaaacctgtatcattagcaagttagtgaaattaggcatatacaaaataatgatacatacaaaaccgttcaaaattccatctacaattctacatataaagaaaaattcagaatgtaccttcaggtgagcatggcaatacagaattattataccctacatagaaaaagaaaaaccacattaaatatatttaaagattagttttacgaaactatcagtaaaatatcaaaacatgatcataaagatacaaatgtatattttaactctatagtcctcactcctcaatgactatatggaagaaaaaaggaaaaaaaatagaggcttctctatagaaaatttaatggttatgttcaaatgtattatcgatagagaaagaataaaatttaaaactatataataaacctcttgtatcaaaatactatgatccaactcaataaaccacaatgactcaaacatcagcaaagaaataaagtttggtttttctataaaaataataatggttacatagaaatacatcgtcgagtgaaaattcatggtcaaaattaatttgtttatagatacaaaaccataaaatagaccctataatatatttcgaaagtacaaccggactcaataatccactatgtctaaaaatccttatagtaaactatgtaataatatgtttccgaaaatggttggaatctttagacacaagtaataaatatatttatgactaaacgttctaacacataacgttacgtatcatagtgggttacattgagttatccaaaggacaattgaataatatatattcaatggcataatatcgtaaatactctagtaatttatgtctacttctataaaagggctgagaatgcttgtgaagatgccacatcagtaatggcatttttgttaatcatacatcaagcaaaggttaattattaaaagtgctcctcaaataataagaaagggatcacacttagaaatatgtcaataaatgggaccacactttgattataaaacccacctctctaatcacatcccaaaatggcgaactttgcctagacctcttacaaagaaaaaagaaaagaaaaatcagtatggagtcttctgtatcacaaacactaagtcaagtattcgatcccacgacgactattctcatcgtcgtctccgttttcatattcatcgacctcatcacacgacggcgaaggtcttacccacccggtccacgtggatggcccatcataggcaatatgttaatgatggatcaactcacccaccgtggtttagcaaacttagctaagaaatacggcggcttgtgccatctccgcatgggcttccttcatatgtatgccgtctcatcacctgatgtagctcgacaagtcctccaagtccaagacagcatcttctcgaaccggccggcaacgatagctattagttatttgacttatgaccgagccgacatggcgttcgctcactacggaccgttttggagacagatgaggaaagtgtgtgtcatgaaggtgtttagccgtaaacgtgccgagtcatgggcttctgttcgagatgaagtggacaaaatgatccggtcggtatctagtaacgttggtaagttagttatatgcggctaaatatgtaaaaactaaaaacaacaaacatttttctctcttttatacgtattttaacaggcaagccaaaataaattgtaggtaagtcaataaacgtcggggagcaaattttcgccctgacccgaaacataacttaccgagcagcgttcgggtcagcttgtgaaaagggacaagacgagttcataagaatcttacaagagttctctaagctttttggagccttcaacgtagcggatttcataccatattttgggtggatcgatccacaagggataaacaagcggctcgtgaaggcccgtaatgatctagacggatttattgacgatatcatcgatgaacatatgaagaagaaagagaatcaaaacactgttgatgatggagatgttgataccgacatggttgatgatcttcttgctttttacagcgaagaggccaaattagtgagcgagacaacggatctccagaattctatcaaacttacccgtgacaatatcaaagcaatcatcatggtattttttttttcaaaagaccactaggcatagtcattattattagtgcgtcacaccacagaagtgatatccatctcaaaccactagtttcatatttttgtctaaatcaggaaggtagctttctgtatttacaagtagtccatatgaattagagatcttgagtaagcattaggggaaatgacgtcatttactgatagtaagaactctagaatattaaatttcagaaaagacctaaaatacaaaacatgcaaaaaatatgtacaaaggtaatataaatcacacgtaaatataaactgaccgtctttgagctaacaatttatttattttaacactttaaaacttaatttgaaatttgatataaaataaacctaatttgaattgattttttgaatttaataggacgttatgttcggcggaacggaaacggtagcgtcggcaatagagtgggccttaacggagttattacggagccccgaggatctaaaacgggtccaacaagaactcgccgaagttgtcggacttgaccggcatgtggaagaatcagacatcgagaagttgacttttctgaagtgcacactcaaagaaaccctaaggctacacccacctatcccactcctcctccacgaaaccgccgaggacactgagatcgacggttacttcgttcccaagaagtcacgcgttatgatcaacgcgtttgcgattggacgtgaccctaagtcttggcctgacgccgaaacgtttagaccgtcgaggtttttagaaccgggagtagcggattttaaaggaagtaacttcgagtttataccattcgggtcgggtcgtagatcgtgcccgggtatgcaactcgggttatacgcgcttgagttagccgttgctcatatattacattgcttcacatggaaattacctgatgggatgaaaccgagcgagcttgacatgaacgacgtgtttggcctcacggctcctagagccactcgtcttttcgccgtgcctagcacacgcctgatttgtgctgtttaagttatggttcgtagcacgtggcgggtgtaacccaacgaaaggtttgtatgggtgtgagaagtcaaatgtagcactgaacatttgtggatgttattatatgtgtgaatgtgtatcacgtgtcgtctggatgaaaacattaatgctctttttcgattttccttttcttttgttgggatttttccttgaatgaaatgtaactggtaaaataaagtttttttttctacaattacctttgcatattacgtaggtaaacaaactattaagagtgcatttgtcagaaattttatagcctaggctcagatttccatcactatgagtctatgacttacaagagtttggcaacatgtaaaaccaagtcgcttttcaaaaaaaaaaaaacatgtaaaaccaagttaaaagctcaattttagatatgaagaagctgctatatagcctttagtcaaaaaaaaaaaaaaaaagaagctgctatatagggcaagttggaaatcataacttgaattttaaattttagttattgtttgtcacttctcagtttatatgcatgttaaaaagccaaatcaccttagttattaaacgaactagtcacttgtaaaatattttatatattatcactacacctaccttgacgaaagcaataaattacaaatcacttaaatacgttagatttgtagtcgatatacacacgtagcacattcatatacatacatacgtacgtatgtatcatacgtggactatcgaatatcacaaaataattgcgtatatcggtatatgtattattctcagtattgaatatgtcacaagcaaaatatataaccaatacagaaaatttagaaaagataattattgccagtaactaacaaacaagcggtgcctccttgacatacattttgtgtgtgtgttggaagcttacgaaaacgacgattttacagacaagcaatgtgttgggaagaattgaatagataccgtaaaagtagctctaagccaaactattgttgaagcttaactatctaagtgacatatttgcaaacagtggcggagccaatgtattggtaaatggatcaattgacccatgtgaatttcttattttaatttcgtttacttgtataattctttaaaatcaagattattattgcaaattttagctaaataatctctatgatctatgttaacttcagagttgactcgcctaaaaagttttcttagctcctccactatcttcaaagaggttggttcgagtagggcttgtctttgtcttctagagaagccagcttctcgacccatcaaccattcaagtcatactgtttcatttttttagagaagccagcttctccttgaccttgcgcttaaattttggcctttatttttttcataaatagttttacttctgattgtagggacaattcagtcaggcgaccccatttcctttcattggtatggatactttaaaaaaatgcagtgatgaggggattggaatagatctatataacagttgaggttgagcaatgcatgcatggttataattgtaatagaagaacataacttgacaccctcgtaaatgagacagaagcgctgtttaaaaacattgttactactaacttatggagttatttttagttattttaacatggtaacacgattaaaagagaaattaatcggacctcaaagaaaaaagtcttctttcagacataaatacaaacatcagaatccacattttcatttttccttttcgactttttttttgtaatttaaccttttctactttttgagtagaaaaggacacgagaatagtagctatgaaagaccagcatggtcagagaaaacggtaatatgtggtggtggactccgcacgacggcgtttcaaaacaataacaacgcataagttgtcgtctttcaccggcgtcgaacgttcagacaaagacgatgacaattaacaaacaaacaaaataaaagaaacccttaatgtctacgtacagttaataacgacgcttttttttgttgtcgtctgtaattcagaacatgtgaacaggataaaaataaatgcgaagacagaaggtcctattatttgtatcatatacgaatctgttaggatctcacatctttgagtttataaatggaatataaacaatatgaagagagatctaatgattgtcaaattttgagtctgaattgcttacataaaatcaccatacctacgtttgtcgat

SEQ ID NO: 111 - DNA, 6051 bp, Raphanus sativus
  misc_feature 1..6051: note = source = /organism="Raphanus sativus"/mol_type="unassigned DNA"
  source 1..6051: mol_type = unassigned DNA
cagattcaccatggccaaagaacaaatgaaacattaagaaagaactcagatttttggcatctaagaacacaaagagagtcaagaacaaagatgaaactttgtccatcacaatttacacagatacaaattagggcatataaaaatatgtttagatcgaaataaacgaggagagagaatttggtacctctcgagtaactcgcggaaggagtagagagaagagaagggtcgggtcttttccaccgtctgctcgttctcagatctattaaaaaaacaaacatatcaatataaataggaagaagaggaggaagacgaaggtcacgaagccaaagaggaggaagacgaagagatgtcgacccgtggtgatggtgaggaggtgaaaccggtgaggagatggagaaatgagttgattgttacggagaaagagatagattggaagaagaacattggaagaagaacaatccaacgaaggagagagggaaaaagaacaagaaaagaagaaagaaacaaaaaaaaacaaaaagagatttaatctggacccttcataattttaatctaatggttaatattgtcaatccacgcagctcattcttattggttaagaacagagaggtttggagaaaataaaaatacagaaataaaaaaatctgtgttttatttatgttgtttagctattattaatgtctatattttttttaaaaaaatagtttatctttttaaaacagtgtatgattcttcatatactttgatcatttattttataagaaaatggttagatttaaatgtaagagtttatggttttgtatgtaagtttaagtttggggtttagggtttgaagtatgatataggatttgagtttatatgatatatggtttgagataaaaatttgaaatgttctatatagcttgaaatgttgtatatagtgagaaatatatttatcttataggaattgattttatttaaaatataagatttcatgttttatgaaatagattaattttgaagtatgtggtttatagttaagatatgtaattaagtatatgatttgatttttttaaaaatattgagtttatagttttatatttaagattttgaattcatataagtttgaaaatattaagtttgtagagtgattgttataatatatgtttgtaagtttatggtttaatgtgtttaaagtgatgaaggttatatatatatatgtggtatgaggttaattttttaaaatctgaaagttgtaaataagtgttggatattagataaagttaaactaggtaaatattaattagatatattgttttgtaaatatatttaagtttaggatatatatttggggtttagggtataaatcgacatagccatggaaaaatagtggctatacagtggctaacgctcttaccacagaaataaatgtggctaaatcgtggctatgatttaacataaaaaaaattatttactttcgaaaatgctttaaccgggaaaatgtaaaaaatgtaagcgggaataaaaaatccagtggctatatatctagccactataatatttggtggcaatacagtggctttttatctagccacgataaaattcagtggctactatcgtggctaattgactttaccgtggctaactgaaaatcgtacctattttgtggcggaatcgtggcttttttaatttagccacgtttttggagtggcactaccgaggctatgcggaagatttctactagtgataatcacaaaatgttcacacgaaatgacaacaaaacaatatatccatacatatactagggcagtattatttttgtatatatgatctaccacaaagttgttcattttgcaaaagtcaactcattgttttatataaaagtccactctttgaaagtttttattttgtttagatgataaaaatatactcatcaatggggccaagttatggttataaaatctagcgctggccttaataatcacatcatctcagattagttgacacttgacagactaataatggagtctctattatcacaaaatctaaaccatgtaatagatcccataccgtcagctcttctcatcaccatctctcttctagttgtagtctacctcatctcacaatggcttaaaccgctctaccctcccggtcccaaaggcttaccggtaattggaaatatgctaatggtgaaccaactcacacaccatggtctagccaagctagccaatcgatacggcggcttgttccatttacgaatgggatttcgtcacgtgtttgccatcacatcacctgacgtggcccgacaagtcctccaagtacaagacatcagcttctcaaaccggcccgtgactgtagccataaactacttaacctacgatctagccgacatggccttcgctccttacggacccttttggagacagatgaggaaagtgtgcgtcatgaaggtgtttagccggaaacgggccgagtcatgggcatcggtccgtgaagaagtaaacaatatggtccggtctttgtctagcaacgacgtcggtaagcccgtaaacgttggagacctcattttcacattgacacggaacataacgtaccgagcggcgtttggtgcggcctgtgagacagaacaagacgagttcataaggatcttgcaagagttctctaagctatttggagcatttaacatcgcggatttcataccgttcctaggctggttggatcttcaagggataaacaagaggcttgttaaggcacgtaatgatcttgacgggttcatcgatgaagttatcgatgagcatatgaagaagagggagactataaacggtgatgaagatacagatatggtggatgatctacttgccttttatagcgaggattcatcacctaatcgtagcaaaaacgccgtaaaactcacgcgtgataatatcaaagccctcgtcatggtaagccaaaacgttaaccttttttatctatatgattttcctcttagtaatcatttattccgttaacatttctataaataaacaactagataatttagttgtaacgatatttcagccaaattttaaatttgaaaaccaatttcgaccaatttaattactaaaagaatttttgaagttgatggtttatatattgggatgaatgatagtttcgaataatattaattagtagcaattatgtattaccgttcttgtttgtgtatacaataccattttggttttggaagtgatatttatctttgtgttacactgttaggaatcatacatttgatatgagatggaatataaagtagtacacaatagatattgttatatttcagttgattatgaatttgaaattcataaattttttggacggttttgataactaaaataaatggtagctcgatatatattaatttactttttttttaacttttattaatttactttcttattactggaaggatgttatgtttggaggaacggagacgatggcgtcagggatcgaatgggctttgacagagctactacgtaacccagctgaactcaaacggctccaacaagaactcaccgaggtcgtgggtcttgaccagcgcgtggatgatactcacctggagaaactaacgttcctaaaatgcacactcaaagaaaccatgagactccacccacccatcccactcatcctccacgaggctatcgaggacacaaagctccaaggcttctctgttcccaaaggctcacgcttgatgataaacgccttcgccatcgcgcgtgacccgaagttgtgggttgacccggaaacgtttcggccttgtaggtttatggaaccgggtatgcctgatttcatggggactaacttcgagtttataccgttcggggcgggtcggagatcgtgtccgggaatgcagcttgggctttatgcgatggaggtggctgtggctaacatcattcactgtttcacgtggaagttgcctgatgggatgaaacctagtgagctcgatatgagcgacgtcatgggtcttaccgccccgagagcaacgcgcttgatcgcagtgcctgacacgcgcctcatatgctctgtttggccctgaccatggagagagaggccttgttgttctgttcaggattaatggtgcaaaagctttcgtcttcttttttaaaatttaaactatgtttttctttttcacttcgtttttctgttcccaagggacttttcttcctcaaaatgtatatgtaaataaatgttgtacaggaagaccaacgtacaaaacgtaaacaatgttgtgtgcagccaaaaaaaatatcacgaagaaacatttgatcatatcttcttcttatgcgccgctcttttctttctttctgatgatcagacaatctctggacactggatgatgtatggtataatgtgtgcccgccctggttatgattctctccggttgaaccgtgcccacagctaattgttagttcttgaaagcatttgtggagctcatagagtcacctttgcttacatcaagtaaaaggctatgcccttcaacgggactgaagctaacgttaatggagtggtggattgataattctcgagggccacggaatgttgaagttggagaaggaagacgtttttggcactccatggcttcgacttccgccaaaagatctgagactgactcgtcacattcgtcaggtccagtcacaatgggttgccaatccgtggtatcattagaggcaaagttgagtttgaaggttggactaaagacatctaggaagtctatttcttggttgtctgtaaacgacaccaaaccagaggtgttcgaatcccacacatttccacttctgttgcaggctgagctctttgtgatgatactgttactgctagttataacactctggtcagtacacactgagttggagctaaagtcaagtactctactctctagtttagtaacagccacgctcctgttttgatcgttagctatagtaattgtctcttcctgcttcaagtcatcgattggtgtattattggagagagcagcagactttacaacgtcatggtttctgctatcggaactggaaggtctaaactaaaacaaaattttttttgtcaaaaacgttatatgcatgggtatattatagatatatgaattatttttattgtcaaaaataaaacaaaaaaattggtaaatggtaatcctatctaattcttgagaccacatccttcaatcagagttttgcagattcactcgactgatcgatgtgacatgagctacctcactagaatgtcattgttaaataaagtctcaaaatactgcatcaagtattcaggttatccttcacattccgtataaattcaaaactacagctagttcttgaactaacgcaccagttttgcttcttgtgttcagatttgactagtgtattaatgtgtgttaactcagatttaccatgaaatgtgaaatgtgtttgaagactcaccttggaagctatattgaacttctctttggttttggagtcaccaagcagctccagcagttacgtacgtatttaggaagcagaatacttccgcagctaaaactcattgtccacatttctcggaaaaataaaaacaaaacaaaacatagcagtgacttgtatctattgcttagactttaggcgtgaatcagagtggagttgggattaatgtatatttttaacacttacaagaaagcaggcttgagaaatttcacacagaatgataatacaagtcagagtcagtaccagggatgtgttattatgaagtcataaagaagaaattgagataatacaatatcttgttttatgtttttgcttgtatcaaaaatcatgcagaattaaaaccacaatagcaaaagcttttttttattattcttctatcttcttcatctttggtcaagaagacaagtaaaaaaaaaaagctggtaacttttgatacatgatagcctttggaggagatcaagcagttctcttaatgatatggactccactgtctgtatccacaatctcgcttatatcaccaaccttgagtgcgtatgttgcctcctcaaacggtttctgcatttgacctctcccaaacggacct

SEQ ID NO: 112 - DNA, 5750 bp, Raphanus sativus
  misc_feature 1..5750: note = source = /organism="Raphanus sativus"/mol_type="unassigned DNA"
  source 1..5750: mol_type = unassigned DNA
caaattgctcaatattaatatttcttaatttttactatatttttctttagaaaatgtttataaaaattcgttaaagaatctatataatatttttaaatacactaaacatcatttgttttctatgactttaaaatgttgttatgtgcttggtctttctttgctttttcttccattactttcttttgaatcttgtactccatctatatataaacaacaacaaaaaaaaaattgtaaattggtgttgcaaataaaatattttgagttcaatcaaccagataaatcatttattttgatctgagttaatggttttcattgagtgacaagaatcaattgttcctttttaaatggaccaagattctcataactgaagattacatgcaaattacatagatctgagttaatggttttcatatgacgttaaaaagaataatataacacgatggatgataattatttcttctttttcatatttaaaataaaacaaaataaacacatatgtgattacttagatgatgacttgtctcgatagatgatgatttcaaagaaaaatggaaaattatataaaattaaaaaatatattaaaatctataataaatttcgtgataaaatcaacacaaatcataaaacacatatttattttaaacataatttattataatattattattaatttcgaataatatattaattactgaaatattgaattgatgttcatttaaacaacaaaggcatcagttgttcttaactgattttaaatatatatatatatatatatatatatttgtatcatttcaaaataagtaattcaaaatcatatcacttatttttgtgatgcaaattaagtgatacaattcaatggttttttatatgaatatggtattttttaaaaagcacaatttgatgtcggaagttaaaaacagagacacatcaacacatttctgcgatttcttcgtaatactaatacaaagtactttaaaaactagagtaagtaattacctctctgaacctagaacatatggacttacatagtatgtagcaccaaagatacaatctagatacaatactcagaaattgttttctttattcatcttttcaatataattatttaacggtttgataccaacgaatcatgctctatatttgcgtatattcatttgtttattgtatttaaactcagaaactgtactatcatgtatctgtcatgtaaatcttgagaaagctacatagttggttttacaaagcctaaccatgcacgttctttttcgtttgattatttccatacgttttttttttgttagattatttccatgtaatttattttttcttcagtactaaataaatataacacgtatactttgactggaaaatctacattcatatcatttacatattaaaaattccaaataaaattttaaatatttgtatataattctcgaacctttagagcagcctcattggctaatactgggtagaggatctcaacaattttattttaaatcaatacaatttatgttaagcaattaaaatcttttattcttttcaacaaaaaaaacaatgataacatgccatctaatgttaaagagttatcacaatatctctactgctataccatctcacaatctttctttctttttaataaattcatcgtgagaacctaatttttatctcgtttaagtcaaaaaaaaaatcttcatctcatcgatggaggtattcttagatctattgatctttattgtaaatctaatttctttcatcggtagattttctattcaaactacatatatattgttatccttattgggatatatattgattattgggatattgactattgacttgtgtttttttttaagaaaacaaatctttcattataccatacatatatctctaagtgctaataaatcgaatacagatacagccactttcatgtgtatacaaccatattaaaaacctcatcaccatctttttactctgtgtattgaaaagaaaaaacataaaagatggattgtttgctttgctcaccaattttatatgtagttcttatatttctttggtatttggttagagttttggtaactcgtggaaatccatttccacctggtccaaaaggctatccaataatcggtaacatgaaactaaagaatcagttgaatcatcgtggtttagccgagttggccaaacaatacggtggtcttctacaccttcaaatgggtaaaattcatattgtggccgcttcaacggctgagatggcccgtgaaattcttcaggtatgcgattttatacggtttagaaattgttttgatattttaagtattcttctaaacatttggataggataacaatgaatttctagtttttttgcaagacaacattcatttcaaacattgagttatgttaggttcaagatgtggttttcgcaaaccggccagctaacgtggcgatttcatatcttacctacaaccgggccgatatggcgtttgcgaactacggtcccctctggcgtcaaatgagaaaggtttgcgtaatgaagctgtttagcagaaaacgggccgaatcatgggcctcggttcgcgacgaaattaatacgatggttcaaactctgactaaacaaaccggttcaccggttaatgttggtgagcttgtatttgctttgacgcggaacataacgtaccgagccgcgtttgggtcgttcgctcgcgacggtcaagacgaattcgtcaagattctacaggagttctcaaaactctttggagcgtttgatatcactgagtttttaccgtggatgaaatggtttagtaaccgcgatttcagcaagcggttggaaaacgccaggaaatcgctggatgggttcatagacagaatcatcgatgcacatatcgagaagaagaactcaagaaaacaagaggatgatgggttggaggacgacatggtggatgaactaatggcgttttatagcggtgagaacggcggcaaatctaacgattccttgtctacatttagactcacgagagataacatcaaagcccttgtcatggtaactagctaatggaatataatttatacatacataaaccataggttaatgtcatattatatttattaatataaaatgatttaaaataggatgtgatgtttggcgggacggaaacggtggcgtctgcgattgaatgggccatgacggagctgatgaagaatcctcacgaactcgtaaagctgcagcaagagctcgctgacgtcatcggattgaaccgtcagtttcacgaatctgatctggagaatcttccttacttcaggtgcgcgatgaaagagacgttgaggctacaccctccgattcctcttctcctccacgaggcggcggcggattccgtcgtctccggctactccattccccgtgactctcgcgtcatgatcaacgtgtacgcgatcgggcgagacggatcggtatggactgaaccggatgcgttcagaccgggtcggtttatggaagacaaagctccggatttcaaaggtagtgacttcgagtttcttccgtttgggtcgggtcgaaggtcgtgcccgggtatgcagttggggctttatgctatggagctcgccgtcgcgcacatgctccattcatttgactggaagttgccggaaggagttagctctggtgatctggatatgaccgacatgtttggtctcacggcgcctagagccaccaggctcatcgctgttccgagctaccggctaaaatgtccgatggtgatttgaaattcgtgttgtttgatactgttttatttcaactgttcgctgttttcatgactccttatacaatgtgttgttgttttgtttttgttgaactgtttgctgtttcgtgatacaatgtgttgttgtggtttaatattccaatctgttttctcaattatccagaatctcggttgggattttcctatttttaaaagtaatttcatgatcatgtcaggagtcataacaaaagcataaatcaactctgcttctcctcagaacactaacttagcaaaagctacttagtacctaaaggggtagtgatggtggtggtggtggtggtggttcactaatctcccagaagggcattcataggcaatgtgtccccttccaccacagtttcgacatatcatcagcctagccctgcagtctctgctcatatgacccacttgccggcagttcctgcaaaccacctcctcctccctgatttgggctctgatccctcgtggtaacacatgggttttagggcatttgatagcaaggtgaccagacaggttacagtgattgcacacgggatcgtctctgcagtcacgagcaaaatgaccggtcttcctgcacttgttgcaggctttctcgttagtgcaatcagctgagaagtgaccttgcttgtaacagttgttgcagagtctaaggtcaccaggagggaggtggcgagccgtgcagtctttagcttggtgtccggcaataccgcagccgtgacaaatgccttcgttggtgcagctattagacatgtgacctggttcacggcagttccaacacagcgacttcgcggagcattctgataaaatgtgcctgccaagtatttttattagaaaaataaaataaaaagacagggatatattatcaagttattacattaatgtaccttgaagattagtgtataaaaaatgtagcaagaacttcagtataacaatctaaaaacaagttaaaacaacgtgatgcaaagagcgtaagattgagaagtagagagaaagaatagggtttacccaggaaggccacagttgtggcagatagagacattgggacactctcgagcaaaatgaccaggttgcttacaattcttgcacatattactttggctgttaatggtagccaaaggaagaagaagcatatcagttaagaaaaaaaaaccaatttgatttccttcttctaagagaaatgccaatttttcgatcttaatggagatgagagaaaaacctgaaagtccgacgagaatctctccggtaaggatcatcacggtaagaaaaacgatcagaagcaatctttctgtccatcggattcctaatcctctccaaactcattatcttagcaacaaaaaaaaaaaacaaaagagagggtatcattcaattgatgtgaataggagatgcaaagtgtgtctaaattgaaagaagcacggaacatcctcctccatccactctagaaaaaaaaaacatgaataatcacactccttaagatgcgcttaaaccctaacaacgattctcacaactaactaaacccaaaaattaaccaaactgcttttcttacaccgtcagtacctccttccttcacgtcgacgacaaccaatattttgacgacaaccaattttccgacaagtaaactgtcagcagttcgacaaatctggcgtgcggcgaaagaacctctgcgacacacatcctcttaacacgctcacaaaacaactcagcagttataaatgggctttaaaagcctttcgtgacccattaactaagcgagacacgcctgttatgtagtaacgaattacggttaagttcggtaatgcgtccacttgctcttttctgatacggtccagtctacaaatacgtacaaacatcaaatttgtaacatcatatgcatggcccatatcccatatgcaatctaaatttctaatgcctacaataaattaatttaacatcatatctgctcatcgtactttgtagtagtaaatataaaagacgatccaattactatca

SEQ ID NO: 113 - DNA, 6153 bp, Raphanus sativus
  misc_feature 1..6153: note = source = /organism="Raphanus sativus"/mol_type="unassigned DNA"
  source 1..6153: mol_type = unassigned DNA
gtgcagcgtctgcgacaaggcgttttcttcttaccaagctctcggcgggcacaaggcgagtcaccggaaaaactcatcgcagactcagtctagcggaggagatgagaaatccacgtcctccgcggtaaccatcgctagccacggcggcggcggcggaggaggaattgtgaagcctcacgtctgcacgatctgtaacaagtctttcgcgacgggtcaagctctcggcggccacaaacggtgccattacgaaggcaaaaacggcggcgttgcaagcagtagcttgtccgtttccgaaggcgtggggtccacaagccacgtcagcagcggcagccatcatcaccaacaccaccaccgtgggtttgacctcaacatcccgccgataccggagttctcgacggtcaacggagaagaagaggtgatgagccccatgccgacgaccaagaaactgaggctcgagtagattcagctagcagtttaaatgaaatttgttatatactgtacatataccaatttttagattccggttcgtgttcttcttcattgaatcagtattccttctttgttatatttgtgtagttttcttgttttctttaagttgtcgcttaaagaaaaagaccctcaaacaattcaaatacgtttcagtttctaaaaaacacatattacaaccttacggcctgtttgttggtccgttggactagcttggtgggctcgtatgacttaacctacaagtctcagcccatgtaaagagattcgtttgaggtagataaactgaagtttccttgcagtataataacaaaatccacatacaagtaattcttttagaccttccttggttataagaacagtgcaaaatataagatgtatttttttcaaccatataatgatataagatgtatttgaaatcctttttttttgaacggcaacgtatttaaactccatacgtataaaatgttatttacattaccaaccaaaagcaccatctttttttaaggagccattccacaacctgacaaatattgacctacctacttagcaaaaaaaaataaaaattgacctaccatataattattatgcgtccaaagagcatacattattatgttatatataattgatatgtacgtcaatgtgataaaattggctaaacatccagtaagatcattggttggttttgtttatcatttttgttgaatatcatgtactcccacaaaagattttgcgtggatatccagctatacaacaatatacatagaggtaagacgtcctcattgcgaatgcgagacaaggcctctaccatcaaaactcaaaacgttaaagaaaaatccaacagtaacatcatactgacaatatattttaactttgataactatggtattaatataatcacagacatggttacagactaaatagaaatatgagatctctcttgaacaaaatattccacagctactgaaatttacgtagctcatttggtgtgtttgaccccttcttgacacccctttcatagacacacctggattccaaaaccccaaattgtaattttctgttacaaaaaaaaaatattctacagcttaattttaaatcgaagttcaatccacaacggattaaccactactgactactaagtttgctccactctgtttcgctctctaaagaatttttgtcgtctacctaatttagtagatagagtgagatgataatgcaaatatacacaatacatgccatacaagctacttacgcctgtcgttttcaccaaaaactatataataaaccctaatgatcgatcaccctgcacgttgtgacttctttaattgttctttgcaccaatcaatcaatcatggaccatggtcacatgaaacacattgataaagaccaatttcaataaaataacagcaaatgtacacaaaagaggtattagagttgaaatagttgttaagtcatatcaatcacattattttcttaacatatataaaatagacccacgcacactaactcgtatcaaaatgtatactccaatgactctcatccttctcgtccctcttctacttttcctttactggcacctcctttcacggcggctaaggttgcggaaatcataccctcccggcccaaaaggcttacctatcattggcaatatactcatgatgaaccaagtcaaccaccgtggcctggccaaactcagccgcacatacggcggattacttcacctccgtctcggactttcccatcttttcgtagtctcctctcctcaaatcgcacgtcaagtcctccaagttcaagaccacgttttctcgaaccgtccaaccacaatagcaatccgttacttgacctacggtcaatccgacttagcattcggcaattatggcccgttctggcgtaggatgagaaaactctacgtcatgatgctctttagccgtaaacgagctgagtcgtgggcctctgtcgacgaagaggtccacaaagcagtccgttttgtagcggccaatgtcggaaaaccactaaacatatgcaaagtcgctttctccttgacaagagatataacgttcggagcagcgttcggctcttcctcgtcaacttctgacgaaggcagactagatgagttccttgagatcatacaagagttctctaagctctttggtgagttcaacgtagcggattacgtcccgtcctggcttagttggatcgacccgcaagggataaacaagcgggtcgagagagctcgaaaatctctagactgtttcattgagtcaatcatcaatgatcatttagacaagaagaagacagaaaacaacgttgacgttgacgaggagaccgatatggttgaccagttacttgcgttctacaaagaagaagtcaaagtcaaggactcggagacaaaaatcaatctcaacaacataaagggcatcatcatggtaaaagctaaacatagtccattccatccgtttcaaatcatacaatcaaatttttaaaatagtagaatgtacaattttatttttatttaactattagaattatgtagcattttaaaaaagaattatgtagcatttattttttaaattttttaattagtgataaaagtgaacaattaataaaaacagcattaaatcatatttatttttaaaaaaatatttagaccgtaatttgatgaacaaaatttggaatatgaatgcatggaatagatcattccatttaactccttgaaaaatacactgttacaagaaaaatattattcataaagcattcctaaaattcttttggaatcaatggaaagagaataatttatttgtaaaatcattacacaacgtaatcggtaaggatcaagtggaatgaatggaatgcatcattttatttttaatttaattaataaatatattttatttctatttcattcctcttcattctatttaattacattcttttagttcataatattcccctaaatgatttccggttacacccttagtttatatatttgaaaagtttttgataacagaatgaaaatgaaatataggatgtgatgttcggaggaactgagacggtggcattagcaatcgagtgggtactaaccgagctactccggagccccgagaacatgaaacgggtccaggaggagctagcgactgtggtcgggcttgagcggtggagcgtagaggacacacaccttgagaagctcactttcctaaaatgtgtactcaaagagaccctccgactccacccgccgttccctctcctcctccacgagacggtggaggacgccgaggtctccggttactccattcccaagggatcacgtgtgatggtcaacacctacgctctcgggcgtaacccgaattcctggtccgacccagaaatatttaacccgagtaggttcttggacccaggtgctcctgatctcaaagggaacagtttcgagttcattccgttcgggtcgggtcggagatcgtgtccgggtatgcaactcgggttgtacgcttttgagcttgcggtggctcatctactacactgcttcacgtggaagttaccaaacggtgtgaaacccggtgatgtggacaccatcgaagggcctggtctcaccgttcctaaggcgaactctcttgtagcagtgccgaccacgcgcctcctctctcccatcgtcttggaaagccacaatgtttaaaaaacttgttttcataagcattgttttacgttgatattttctttcgttatgtatatattgtgttcgtgtttaagttggcatttttgttcgtaaaatgtatatccaaataaatacataaagcttcgaaaatgttttgttttttaaaagatactgacaccaagtagaaagacgaatacattttttcaactactatgtgatatatataataatatttcttaaagaaacaaattaactatgtattttaaataactgttgccaaaaacaggagaaaaataataggttacaaaaaaacaacacaggagaaaaaataataatataaccgatgcttaaatacataaacattacaaataaatggtttaaacataataatttttagttattgttgtaaagtagtcttataaaagggtaaacccccttttaaaatcactgttgcattaacgggcagaaatgaatgtaagtaacgttcacatgtaaaacagatataaacatgtaattcatagtttaattaagcaagatattgtcatcgagtcaaagggttgacttgtgatgtcggagaaacagagaattaaaagcagtacgagattgaatcacctagtggactaatctataatgcacacactaaattagaagtgtagatttattacacaatcgttttaagtagaagaggaagtcaaagagattaccagaaataaaaagaggaagtcgaagataacctttgatatacaaaagcatgaagtaagcttctgaacaacaatcttttgcatattaggagcccaagatgccaagaggagattcagcagtctcaccgcaatttcacctttacgtgagacttctccgtatgattggactttctctgatgcatgtctggatgttcttctcataattcctccggtgagaaaaatgcaagtggtcttagctaacttagacagagaaaaaaaaacaaaacagtatcatgtttcaacaaaccttgcatcatcagcactcaaggtttcagtcacttccggttcttgaaaatcaaacgtttccgtctgttgtctagcagagactctgcccatttatacaagaaattagtgacctctctaacgcattgttttagatggctattagatatttatctaataatgtcagattgtcacggctgaacaaaattgtttacatggtttgtggtttcagtttcccgagttgtaattaagggagtttcagagaattgtgatgtaactgcactaatcattcactatgtaattatgttgttgtgtttaatcatacctcaatgggtcatattgaaaggaataaaaaaaaaacaaaagataagatgatatttatccaccttcttcttagtcaacttcaacttcaggatagagatgtggatgctcaacatgcataaatgtctttctgctttgaaacacctcgagacagatcactgcaccacataaacaatggtaatcgatgatggtcacgaggggatgtcagaaacagtccaaaattgttggaaactctgccattctgcaaaaaagagaagagagtaacaatagagatatacatttgtaattacaacgaactcttatttttgaatgctaaacaacattaaatataaatataaaataatttaaaaaaataagaaacataaaaatataaaccaaatttcttgaaataaaactttaaatgaaagtcaaaactactcaaaatgaattaaaagtttcagaattgaattttacgaattcaaactgaaccaaaccagaattatatgaaaccaaaacaataaacatcacaagttgaaatgttaaatcatggttttggtattttaaataaaatgcacttgtttagtgagatattttactcttcgaatttcatttcaattgccatgtgccattatgtattgtaatttcaaatgtcattagtttccttgtcccactgtactcttctattgaattagttatcatgcattttcgaattattgaactctcgaattattgaactctgcttttgatcaatttacttgt

SEQ ID NO: 114 - DNA, 1825 bp, Allium cepa
  misc_feature 1..1825: note = source = /organism="Allium cepa"/mol_type="unassigned DNA"
  source 1..1825: mol_type = unassigned DNA
actgggaatcaaagaaaaacaaagaagcagactccaacgagggaagctagattcaaacacaagaactcttcccctcgcatacacaaagctcaaagaaacaaaaccaaagccatgatggacatgcaatccatcctcatattcaccctcccattcgtcaccctcctcttcctggtcattaccagccgccgcaggcccaaactcccgcttccgcccggcccgcgcccactacccatcatcggcaacctcaacctcatcgacaaactcacccaccgtggcctggcccatctggccaaccaatacggtggcatcttccacctcaagcttgggtccgtccacaccttctccatctccacacccgaaatcgcaaaggaggtccttcaaacccaggacctggccttctccaaccgccccgccaccatcgccatcacctacctcacctacgaccgggccgacatggccttcacccactacggcccattctggcgccaaatccgcaaactctgcgttatgaagctgttcagtcgaaaaagagcggaatcatgggcctccgtccgagaggagatagagaaggctgtatcgaccgcagccgcgggcgctgggactgtggtcaacgtaggggagctggtgttcaatctgacgaagaatataacgttcagggcggcgtttggagcgaagtcgggcgaggagcaggatgagtttctggggattttgcaggagatatcaaagctgtttggagcgtttaatgtcggggatttcgtaccgggtttgaggtacttggatccacaggggatagggaggaggatgaggaaggtgaggaaggaactggatggatttattgacaggataatagatgagcatgtgcagaataggaaggaggtggatgatgtggaggccgacatggtggatgagatgttggcgtttgttggacaagggaagagcattggtagggattctgatgagttgaggcttactaggaataatatcaaggctattatcatggacgtaatgttcggaggaacagagacagtggcctcggcaatagaatgggcaatggcagagctccttaaaagcccagaagatctcaaaaggctccagcaagaactgacgtcggtagtgggcctggaccgcaaagtccaggactccgacctcgacaaactgccgtacctcaaatgcgtcatcaaggaaacgctgcgacttcatccaccgatcccgcttcttcttcacgaaacagcggaggactgcgagatccagggctactcgatccccaaaaaatcgagggtaatgatcaacgtgtgggcgatcgggcgcgacaagtccgcctggaaggacgcggaccagttcaagccgtcgagattcgtgaagggaggggagtacgagcaggtggattttaaggggaatttcttcgagctgctgccgtttggggcgggaaggaggtcgtgcccggggatgcagctggggctgtacgcgctggacctgacggtggcgaatatggcgcactgtttcgattgggagttgccggacgggatgaagccaggcgagatggatatgagcgacgtgtttgggttgactgcgcctagggctgtgaggcttgctgctgtgccttctcctagattgacttgtcgtatttgatttcaacatgtacatacgatttgtgtttatatatacgttataaagtatacacaacggtgattgtttgtataattgtaatgctgttccatggtgcggactaataaaaatgttcaatgagagggttttgaaagacgtatggtgtatgtgaattgtaaatttatataattgttacttcgtcattaatgaaactttttca

SEQ ID NO: 115 - DNA, 1521 bp, Lactuca sativa
  misc_feature 1..1521: note = source = /organism="Lactuca sativa"/mol_type="unassigned DNA"
  source 1..1521: mol_type = unassigned DNA
atggaatcacttcaaatccccatagcattctacgctataatagctatcttaactttcttctttctttcatgggtccgccggaaaccactcccgccggggccaatggggtggccaatcatcggcaacatgttgatgatggaccaacttacccaccgtggcttagcccgtttggcagaaaaatacggtggtatccttcatctaaagatgggtttcagccacaccattgcagtgtcctcgccggagatggcgaggataatacttcaagaaaaagataacatctttgccaaccgtccggcaaccatcgccatcacttacctgacttacaacggcgtagatttggcttttgctaattatggacctttctggcgacaaatgcgaaagctttgtgtcatgaagctgttcagccggaaacgagcggagtcatgggactccgtcagggatgaggtggacaccatggtgaaagccaccgccattaactccggtacgccggtaaacttgggtgagcttgtttttgggttgacccatgatattatctaccgagcagcttttgggtcgatttcacatgaagggaaagaagagtttatcagaatccttcaagaatacaccaaactttttggcgcattcaatttggctgactttatcccgttcctcgggtttattgatccggcggggttgaacacacgtttaccggcggccagggcggcgttggacggattcattgacaaaatcatcgacgagcatttgcgtaaaggaaagaaaaccggcgatgaaggtttggataacgatatggttgatgagatgttggcgttttacagcgaggaaggaaaagtcaacgaaggtggtgatttgcaaaacgccattaaccttactcgagataacatcaaagccataatcatggatgtaatgttcggtggaactgagacagtggcgtccgccatagaatgggccatgacggagctaatgcatacaccggaggcactaaagcgcgtgcaacaggagatggcaaatgtcgtcggacttgaccggcgcgtggaggagtctgacttggagaagctgacgtacttcaaatgcgtcatcaaggaaaccctccgactacaccctccgatcccagttctcctccaccagtcttcggaggcgacagaagtttccggctaccatatacctaaaggaacacgtgtcatggtgaacgcgtatgctattaatcgtgataagaactcttgggaagatccggatacgtttaacccgtcacgttttttacaaaacggagctccggattttagaggaagcaactatgagtttctgccatttggttctggtcggaggtcgtgtccggggatgcaactagggttgtatgcgatggagatggcggtggcccaccttttgcattgtttcacgtgggaattgccggatggaatgaagccaagtgaaatcgacatgggtgatgtgtttggactcacagcaccaaaagcaataagattggtagcagtgccaactccgcgtttattatgcccattgtattga

SEQ ID NO: 116 - DNA, 1512 bp, Lactuca sativa
  misc_feature 1..1512: note = source = /organism="Lactuca sativa"/mol_type="unassigned DNA"
  source 1..1512: mol_type = unassigned DNA
atggatcctaagtccatcttactttacgttgtactccctctcttaaccttcttccttctctcccgattacgccgaaaacctcttccgcctggtccaagagggtggccgctgatcggtaacatgttaatgatggaccaactcacccaccgtggccttgctcgtttgggagaaaaatacggtggtcttcttcatctgaagatgggtttcagccataccgtcgctgtctcgtcccccgaaatagccaggcaagtactccaagttcaagacaacatcttcgccaaccgcccggccaccatcgccattagttacctcacctacgaccggcaagacatggcgttcgccaactacggtcccttttggcgtcagatgcgtaagctttgcgtcatgaagctgttcagcagaaagcgagctgagtcttgggactccgtcagagacgaagttgtctccatggtcaaaatcaccgctgcaagctccggcaccgctgttaaccttggagagcttgttttcgggttaacccatgatatcatttaccgagcagctttcgggtctatctctcatgaaggaaaagaagaattcatcagaattctacaagaatacacaaagctttttggtgctttcaatttggcagattttgtcccgtggcttggatttatcgaccctgccggactgaatacccgtttaccgaaggccagggcggcgcttgacagattcattgataaaatcatcgacgagcaccttgcaaaagagaggaaaacgggcgatgaggaagataatgatatggtggatgagatgttggctttttacagtgaagaaggaaaggtaaacgaaggcgaggatttgcagaacgcgattagactcacccgaaacaatatcaaagccattattatggatgtaatgtttggtgggactgaaactgttgcttctgctatcgaatgggctttaactgagctaatgcacaccccagaatccttaaaacgtgcacaacaagagctcgctgatgttgttggccttgatcgtcgtgtagaagaatcagatttcgagaagctaacttacttcaaatgtgtcatcaaagaaaccttacgtctccaccctccgatccctgtccttttgcaccaatcatcagaagccacgtcggttgctggctaccacatacctaaagggacacgtgtcatggttaacgcattcgccattaatcgtgataagaactcatggaaggatccacacacgttcaacccatcacgtttcttgcaagatggggcacccgactttaaaggaagcaattatgagtttcttccatttggatctggacgtagatcatgtcctggaatgcaacttggattgtacgcaatggagatggcagtggctcaccttcttcattcattcacatggcagttgcctgatggaatgaaaccaagtgagattgacatgaatgatgtgtttggactcactgcaccaaaagcgattcgacttgttgctgtgccaactcctcggttgttgtgtccgctgtattga

SEQ ID NO: 117 - DNA, 101 bp, Lactuca sativa
  misc_feature 1..101: note = source = /organism="Lactuca sativa"/mol_type="unassigned DNA"
  source 1..101: mol_type = unassigned DNA
cttacaacggcgtagatttggcttttgctaattatggacctttctggcgataaatgcgaaagctttgtgtcatgaagctgttcagccggaaacgagcggag

SEQ ID NO: 118-123 - skipped (6 entries)

SEQ ID NO: 124 - DNA, 101 bp, Lactuca sativa
  misc_feature 1..101: note = source = /organism="Lactuca sativa"/mol_type="unassigned DNA"
  source 1..101: mol_type = unassigned DNA
tgagtcttgggactccgtcagagacgaagttgtctccatggtcaaaatcatcgctgcaagctccggcaccgctgttaaccttggagagcttgttttcgggt

SEQ ID NO: 125 - DNA, 101 bp, Lactuca sativa
  misc_feature 1..101: note = source = /organism="Lactuca sativa"/mol_type="unassigned DNA"
  source 1..101: mol_type = unassigned DNA
ctccatggtcaaaatcaccgctgcaagctccggcaccgctgttaaccttgaagagcttgttttcgggttaacccatgatatcatttaccgagcagctttcg

SEQ ID NO: 126 - DNA, 101 bp, Lactuca sativa
  misc_feature 1..101: note = source = /organism="Lactuca sativa"/mol_type="unassigned DNA"
  source 1..101: mol_type = unassigned DNA
caaagccattattatggatgtaatgtttggtgggactgaaactgttgcttttgctatcgaatgggctttaactgagctaatgcacaccccagaatccttaa

SEQ ID NO: 127 - DNA, 101 bp, Lactuca sativa
  misc_feature 1..101: note = source = /organism="Lactuca sativa"/mol_type="unassigned DNA"
  source 1..101: mol_type = unassigned DNA
agatggggcacccgactttaaaggaagcaattatgagtttcttccatttgaatctggacgtagatcatgtcctggaatgcaacttggattgtacgcaatgg

SEQ ID NO: 128 - DNA, 101 bp, Lactuca sativa
  misc_feature 1..101: note = source = /organism="Lactuca sativa"/mol_type="unassigned DNA"
  source 1..101: mol_type = unassigned DNA
ggcacccgactttaaaggaagcaattatgagtttcttccatttggatctgaacgtagatcatgtcctggaatgcaacttggattgtacgcaatggagatgg

SEQ ID NO: 129-142 - skipped (14 entries)

SEQ ID NO: 143 - DNA, 101 bp, Lactuca sativa
  misc_feature 1..101: note = source = /organism="Lactuca sativa"/mol_type="unassigned DNA"
  source 1..101: mol_type = unassigned DNA
cttacaacggcgtagatttggcttttgctaattatggacctttctggcgacaaatgcgaaagctttgtgtcatgaagctgttcagccggaaacgagcggag

SEQ ID NO: 144-149 - skipped (6 entries)

SEQ ID NO: 150 - DNA, 101 bp, Lactuca sativa
  misc_feature 1..101: note = source = /organism="Lactuca sativa"/mol_type="unassigned DNA"
  source 1..101: mol_type = unassigned DNA
tgagtcttgggactccgtcagagacgaagttgtctccatggtcaaaatcaccgctgcaagctccggcaccgctgttaaccttggagagcttgttttcgggt

SEQ ID NO: 151 - DNA, 101 bp, Lactuca sativa
  misc_feature 1..101: note = source = /organism="Lactuca sativa"/mol_type="unassigned DNA"
  source 1..101: mol_type = unassigned DNA
ctccatggtcaaaatcaccgctgcaagctccggcaccgctgttaaccttggagagcttgttttcgggttaacccatgatatcatttaccgagcagctttcg

SEQ ID NO: 152 - DNA, 101 bp, Lactuca sativa
  misc_feature 1..101: note = source = /organism="Lactuca sativa"/mol_type="unassigned DNA"
  source 1..101: mol_type = unassigned DNA
caaagccattattatggatgtaatgtttggtgggactgaaactgttgcttctgctatcgaatgggctttaactgagctaatgcacaccccagaatccttaa

SEQ ID NO: 153 - DNA, 101 bp, Lactuca sativa
  misc_feature 1..101: note = source = /organism="Lactuca sativa"/mol_type="unassigned DNA"
  source 1..101: mol_type = unassigned DNA
agatggggcacccgactttaaaggaagcaattatgagtttcttccatttggatctggacgtagatcatgtcctggaatgcaacttggattgtacgcaatgg

SEQ ID NO: 154 - DNA, 101 bp, Lactuca sativa
  misc_feature 1..101: note = source = /organism="Lactuca sativa"/mol_type="unassigned DNA"
  source 1..101: mol_type = unassigned DNA
ggcacccgactttaaaggaagcaattatgagtttcttccatttggatctggacgtagatcatgtcctggaatgcaacttggattgtacgcaatggagatgg

SEQ ID NO: 155-168 - skipped (14 entries)

SEQ ID NO: 169 - protein, 50 aa, Lactuca sativa
IFSNRPATIAISYLTYDRADMAFAHYGPFWRQMRKLCVMKLFSRKRAESW

SEQ ID NO: 170 - protein, 50 aa, Lactuca sativa
DFKGSNFEFIPFGSGRRSCPGMQLGLYALEMAVAHLLHCFTWELPDGMKP

SEQ ID NO: 171 - protein, 50 aa, Lactuca sativa
TRDNIKAIIMDVMFGGTETVASAIEWAMTELMHSPEDLKRVQQELADVVG

SEQ ID NO: 172 - protein, 50 aa, Lactuca sativa
YLKCCIKETLRLHPPIPLLLHETAEDCEVAGYHIPKGSRVMINAWAIGRD

SEQ ID NO: 173 - protein, 50 aa, Lactuca sativa
PYPPGPKGWPIIGNMLMMDQLTHRGLAKLAKQYGGICHLRMGFLHMVAVS

SEQ ID NO: 174 - DNA, 1521 bp, Lactuca sativa
  misc_feature 1..1521: note = source = /organism="Lactuca sativa"/mol_type="unassigned DNA"
  source 1..1521: mol_type = unassigned DNA
atggaatcacttcaaatccccatagcattctacgctataatagctatcttaactttcttctttctttcatgggtccgccggaaaccactcccgccggggccaatggggtggccaatcatcggcaacatgttgatgatggaccaacttacccaccgtggcttagcccgtttggcagaaaaatacggtggtatccttcatctaaagatgggtttcagccacaccattgcagtgtcctcgccggagatggcgaggataatacttcaagaaaaagataacatctttgccaaccgtccggcaaccatcgccatcacttacctgacttacaacggcgtagatttggcttttgctaattatggacctttctggcgataaatgcgaaagctttgtgtcatgaagctgttcagccggaaacgagcggagtcatgggactccgtcagggatgaggtggacaccatggtgaaagccaccgccattaactccggtacgccggtaaacttgggtgagcttgtttttgggttgacccatgatattatctaccgagcagcttttgggtcgatttcacatgaagggaaagaagagtttatcagaatccttcaagaatacaccaaactttttggcgcattcaatttggctgactttatcccgttcctcgggtttattgatccggcggggttgaacacacgtttaccggcggccagggcggcgttggacggattcattgacaaaatcatcgacgagcatttgcgtaaaggaaagaaaaccggcgatgaaggtttggataacgatatggttgatgagatgttggcgttttacagcgaggaaggaaaagtcaacgaaggtggtgatttgcaaaacgccattaaccttactcgagataacatcaaagccataatcatggatgtaatgttcggtggaactgagacagtggcgtccgccatagaatgggccatgacggagctaatgcatacaccggaggcactaaagcgcgtgcaacaggagatggcaaatgtcgtcggacttgaccggcgcgtggaggagtctgacttggagaagctgacgtacttcaaatgcgtcatcaaggaaaccctccgactacaccctccgatcccagttctcctccaccagtcttcggaggcgacagaagtttccggctaccatatacctaaaggaacacgtgtcatggtgaacgcgtatgctattaatcgtgataagaactcttgggaagatccggatacgtttaacccgtcacgttttttacaaaacggagctccggattttagaggaagcaactatgagtttctgccatttggttctggtcggaggtcgtgtccggggatgcaactagggttgtatgcgatggagatggcggtggcccaccttttgcattgtttcacgtgggaattgccggatggaatgaagccaagtgaaatcgacatgggtgatgtgtttggactcacagcaccaaaagcaataagattggtagcagtgccaactccgcgtttattatgcccattgtattga

SEQ ID NO: 175 - DNA, 1512 bp, Lactuca sativa
  misc_feature 1..1512: note = source = /organism="Lactuca sativa"/mol_type="unassigned DNA"
  source 1..1512: mol_type = unassigned DNA
atggatcctaagtccatcttactttacgttgtactccctctcttaaccttcttccttctctcccgattacgccgaaaacctcttccgcctggtccaagagggtggccgctgatcggtaacatgttaatgatggaccaactcacccaccgtggccttgctcgtttgggagaaaaatacggtggtcttcttcatctgaagatgggtttcagccataccgtcgctgtctcgtcccccgaaatagccaggcaagtactccaagttcaagacaacatcttcgccaaccgcccggccaccatcgccattagttacctcacctacgaccggcaagacatggcgttcgccaactacggtcccttttggcgtcagatgcgtaagctttgcgtcatgaagctgttcagcagaaagcgagctgagtcttgggactccgtcagagacgaagttgtctccatggtcaaaatcatcgctgcaagctccggcaccgctgttaaccttggagagcttgttttcgggttaacccatgatatcatttaccgagcagctttcgggtctatctctcatgaaggaaaagaagaattcatcagaattctacaagaatacacaaagctttttggtgctttcaatttggcagattttgtcccgtggcttggatttatcgaccctgccggactgaatacccgtttaccgaaggccagggcggcgcttgacagattcattgataaaatcatcgacgagcaccttgcaaaagagaggaaaacgggcgatgaggaagataatgatatggtggatgagatgttggctttttacagtgaagaaggaaaggtaaacgaaggcgaggatttgcagaacgcgattagactcacccgaaacaatatcaaagccattattatggatgtaatgtttggtgggactgaaactgttgcttctgctatcgaatgggctttaactgagctaatgcacaccccagaatccttaaaacgtgcacaacaagagctcgctgatgttgttggccttgatcgtcgtgtagaagaatcagatttcgagaagctaacttacttcaaatgtgtcatcaaagaaaccttacgtctccaccctccgatccctgtccttttgcaccaatcatcagaagccacgtcggttgctggctaccacatacctaaagggacacgtgtcatggttaacgcattcgccattaatcgtgataagaactcatggaaggatccacacacgttcaacccatcacgtttcttgcaagatggggcacccgactttaaaggaagcaattatgagtttcttccatttggatctggacgtagatcatgtcctggaatgcaacttggattgtacgcaatggagatggcagtggctcaccttcttcattcattcacatggcagttgcctgatggaatgaaaccaagtgagattgacatgaatgatgtgtttggactcactgcaccaaaagcgattcgacttgttgctgtgccaactcctcggttgttgtgtccgctgtattga

SEQ ID NO: 176 - DNA, 1512 bp, Lactuca sativa
  misc_feature 1..1512: note = source = /organism="Lactuca sativa"/mol_type="unassigned DNA"
  source 1..1512: mol_type = unassigned DNA
atggatcctaagtccatcttactttacgttgtactccctctcttaaccttcttccttctctcccgattacgccgaaaacctcttccgcctggtccaagagggtggccgctgatcggtaacatgttaatgatggaccaactcacccaccgtggccttgctcgtttgggagaaaaatacggtggtcttcttcatctgaagatgggtttcagccataccgtcgctgtctcgtcccccgaaatagccaggcaagtactccaagttcaagacaacatcttcgccaaccgcccggccaccatcgccattagttacctcacctacgaccggcaagacatggcgttcgccaactacggtcccttttggcgtcagatgcgtaagctttgcgtcatgaagctgttcagcagaaagcgagctgagtcttgggactccgtcagagacgaagttgtctccatggtcaaaatcaccgctgcaagctccggcaccgctgttaaccttgaagagcttgttttcgggttaacccatgatatcatttaccgagcagctttcgggtctatctctcatgaaggaaaagaagaattcatcagaattctacaagaatacacaaagctttttggtgctttcaatttggcagattttgtcccgtggcttggatttatcgaccctgccggactgaatacccgtttaccgaaggccagggcggcgcttgacagattcattgataaaatcatcgacgagcaccttgcaaaagagaggaaaacgggcgatgaggaagataatgatatggtggatgagatgttggctttttacagtgaagaaggaaaggtaaacgaaggcgaggatttgcagaacgcgattagactcacccgaaacaatatcaaagccattattatggatgtaatgtttggtgggactgaaactgttgcttctgctatcgaatgggctttaactgagctaatgcacaccccagaatccttaaaacgtgcacaacaagagctcgctgatgttgttggccttgatcgtcgtgtagaagaatcagatttcgagaagctaacttacttcaaatgtgtcatcaaagaaaccttacgtctccaccctccgatccctgtccttttgcaccaatcatcagaagccacgtcggttgctggctaccacatacctaaagggacacgtgtcatggttaacgcattcgccattaatcgtgataagaactcatggaaggatccacacacgttcaacccatcacgtttcttgcaagatggggcacccgactttaaaggaagcaattatgagtttcttccatttggatctggacgtagatcatgtcctggaatgcaacttggattgtacgcaatggagatggcagtggctcaccttcttcattcattcacatggcagttgcctgatggaatgaaaccaagtgagattgacatgaatgatgtgtttggactcactgcaccaaaagcgattcgacttgttgctgtgccaactcctcggttgttgtgtccgctgtattga

SEQ ID NO: 177 - DNA, 1512 bp, Lactuca sativa
  misc_feature 1..1512: note = source = /organism="Lactuca sativa"/mol_type="unassigned DNA"
  source 1..1512: mol_type = unassigned DNA
atggatcctaagtccatcttactttacgttgtactccctctcttaaccttcttccttctctcccgattacgccgaaaacctcttccgcctggtccaagagggtggccgctgatcggtaacatgttaatgatggaccaactcacccaccgtggccttgctcgtttgggagaaaaatacggtggtcttcttcatctgaagatgggtttcagccataccgtcgctgtctcgtcccccgaaatagccaggcaagtactccaagttcaagacaacatcttcgccaaccgcccggccaccatcgccattagttacctcacctacgaccggcaagacatggcgttcgccaactacggtcccttttggcgtcagatgcgtaagctttgcgtcatgaagctgttcagcagaaagcgagctgagtcttgggactccgtcagagacgaagttgtctccatggtcaaaatcaccgctgcaagctccggcaccgctgttaaccttggagagcttgttttcgggttaacccatgatatcatttaccgagcagctttcgggtctatctctcatgaaggaaaagaagaattcatcagaattctacaagaatacacaaagctttttggtgctttcaatttggcagattttgtcccgtggcttggatttatcgaccctgccggactgaatacccgtttaccgaaggccagggcggcgcttgacagattcattgataaaatcatcgacgagcaccttgcaaaagagaggaaaacgggcgatgaggaagataatgatatggtggatgagatgttggctttttacagtgaagaaggaaaggtaaacgaaggcgaggatttgcagaacgcgattagactcacccgaaacaatatcaaagccattattatggatgtaatgtttggtgggactgaaactgttgcttttgctatcgaatgggctttaactgagctaatgcacaccccagaatccttaaaacgtgcacaacaagagctcgctgatgttgttggccttgatcgtcgtgtagaagaatcagatttcgagaagctaacttacttcaaatgtgtcatcaaagaaaccttacgtctccaccctccgatccctgtccttttgcaccaatcatcagaagccacgtcggttgctggctaccacatacctaaagggacacgtgtcatggttaacgcattcgccattaatcgtgataagaactcatggaaggatccacacacgttcaacccatcacgtttcttgcaagatggggcacccgactttaaaggaagcaattatgagtttcttccatttggatctggacgtagatcatgtcctggaatgcaacttggattgtacgcaatggagatggcagtggctcaccttcttcattcattcacatggcagttgcctgatggaatgaaaccaagtgagattgacatgaatgatgtgtttggactcactgcaccaaaagcgattcgacttgttgctgtgccaactcctcggttgttgtgtccgctgtattga

SEQ ID NO: 178 - DNA, 1512 bp, Lactuca sativa
  misc_feature 1..1512: note = source = /organism="Lactuca sativa"/mol_type="unassigned DNA"
  source 1..1512: mol_type = unassigned DNA
atggatcctaagtccatcttactttacgttgtactccctctcttaaccttcttccttctctcccgattacgccgaaaacctcttccgcctggtccaagagggtggccgctgatcggtaacatgttaatgatggaccaactcacccaccgtggccttgctcgtttgggagaaaaatacggtggtcttcttcatctgaagatgggtttcagccataccgtcgctgtctcgtcccccgaaatagccaggcaagtactccaagttcaagacaacatcttcgccaaccgcccggccaccatcgccattagttacctcacctacgaccggcaagacatggcgttcgccaactacggtcccttttggcgtcagatgcgtaagctttgcgtcatgaagctgttcagcagaaagcgagctgagtcttgggactccgtcagagacgaagttgtctccatggtcaaaatcaccgctgcaagctccggcaccgctgttaaccttggagagcttgttttcgggttaacccatgatatcatttaccgagcagctttcgggtctatctctcatgaaggaaaagaagaattcatcagaattctacaagaatacacaaagctttttggtgctttcaatttggcagattttgtcccgtggcttggatttatcgaccctgccggactgaatacccgtttaccgaaggccagggcggcgcttgacagattcattgataaaatcatcgacgagcaccttgcaaaagagaggaaaacgggcgatgaggaagataatgatatggtggatgagatgttggctttttacagtgaagaaggaaaggtaaacgaaggcgaggatttgcagaacgcgattagactcacccgaaacaatatcaaagccattattatggatgtaatgtttggtgggactgaaactgttgcttctgctatcgaatgggctttaactgagctaatgcacaccccagaatccttaaaacgtgcacaacaagagctcgctgatgttgttggccttgatcgtcgtgtagaagaatcagatttcgagaagctaacttacttcaaatgtgtcatcaaagaaaccttacgtctccaccctccgatccctgtccttttgcaccaatcatcagaagccacgtcggttgctggctaccacatacctaaagggacacgtgtcatggttaacgcattcgccattaatcgtgataagaactcatggaaggatccacacacgttcaacccatcacgtttcttgcaagatggggcacccgactttaaaggaagcaattatgagtttcttccatttgaatctggacgtagatcatgtcctggaatgcaacttggattgtacgcaatggagatggcagtggctcaccttcttcattcattcacatggcagttgcctgatggaatgaaaccaagtgagattgacatgaatgatgtgtttggactcactgcaccaaaagcgattcgacttgttgctgtgccaactcctcggttgttgtgtccgctgtattga

SEQ ID NO: 179 - DNA, 1512 bp, Lactuca sativa
  misc_feature 1..1512: note = source = /organism="Lactuca sativa"/mol_type="unassigned DNA"
  source 1..1512: mol_type = unassigned DNA
atggatcctaagtccatcttactttacgttgtactccctctcttaaccttcttccttctctcccgattacgccgaaaacctcttccgcctggtccaagagggtggccgctgatcggtaacatgttaatgatggaccaactcacccaccgtggccttgctcgtttgggagaaaaatacggtggtcttcttcatctgaagatgggtttcagccataccgtcgctgtctcgtcccccgaaatagccaggcaagtactccaagttcaagacaacatcttcgccaaccgcccggccaccatcgccattagttacctcacctacgaccggcaagacatggcgttcgccaactacggtcccttttggcgtcagatgcgtaagctttgcgtcatgaagctgttcagcagaaagcgagctgagtcttgggactccgtcagagacgaagttgtctccatggtcaaaatcaccgctgcaagctccggcaccgctgttaaccttggagagcttgttttcgggttaacccatgatatcatttaccgagcagctttcgggtctatctctcatgaaggaaaagaagaattcatcagaattctacaagaatacacaaagctttttggtgctttcaatttggcagattttgtcccgtggcttggatttatcgaccctgccggactgaatacccgtttaccgaaggccagggcggcgcttgacagattcattgataaaatcatcgacgagcaccttgcaaaagagaggaaaacgggcgatgaggaagataatgatatggtggatgagatgttggctttttacagtgaagaaggaaaggtaaacgaaggcgaggatttgcagaacgcgattagactcacccgaaacaatatcaaagccattattatggatgtaatgtttggtgggactgaaactgttgcttctgctatcgaatgggctttaactgagctaatgcacaccccagaatccttaaaacgtgcacaacaagagctcgctgatgttgttggccttgatcgtcgtgtagaagaatcagatttcgagaagctaacttacttcaaatgtgtcatcaaagaaaccttacgtctccaccctccgatccctgtccttttgcaccaatcatcagaagccacgtcggttgctggctaccacatacctaaagggacacgtgtcatggttaacgcattcgccattaatcgtgataagaactcatggaaggatccacacacgttcaacccatcacgtttcttgcaagatggggcacccgactttaaaggaagcaattatgagtttcttccatttggatctgaacgtagatcatgtcctggaatgcaacttggattgtacgcaatggagatggcagtggctcaccttcttcattcattcacatggcagttgcctgatggaatgaaaccaagtgagattgacatgaatgatgtgtttggactcactgcaccaaaagcgattcgacttgttgctgtgccaactcctcggttgttgtgtccgctgtattga

(19) United States
(12) Patent Application Publication
(10) Pub. No.: US 2023/0112932 A1
(43) Pub. Date: Apr. 13, 2023

(54) PLANT WITH REDUCED WOUND-INDUCED SURFACE DISCOLORATION PHENOTYPE

(71) Applicant: RIJK ZWAAN ZAADTEELT EN ZAADHANDEL B.V., De Lier (NL)

(72) Inventors: Cornelis Maria Petrus Van Dun, De Lier (NL); Johannes Wilhelmus Schut, De Lier (NL); Silvester De Nooijer, De Lier (NL); Johannis Hendrik De Rooij, De Lier (NL)

(21) Appl. No.: 17/944,033

(22) Filed: Sep. 13, 2022

Related U.S. Application Data
(63) Continuation-in-part of application No. 16/787,231, filed on Feb. 11, 2020.
Application No. 16/787,231 is a continuation of application No. 16/106,684, filed on Aug. 21, 2018, now Pat. No. 10,731,174.
Application No. 16/106,684 is a continuation-in-part of application No. PCT/EP2017/054343, filed on Feb. 24, 2017.
Application No. PCT/EP2017/054343 is a continuation-in-part of application No. PCT/EP2016/053895, filed on Feb. 24, 2016.

(51) Int. Cl. C12N 15/82; A01H 5/12; A01H 6/14; C12N 15/01

(52) U.S. Cl. CPC C12N 15/8271; A01H 5/12; C12Y 114/13; A01H 6/1472; C12N 15/8249; C12N 15/01; C12N 9/0073

(57) ABSTRACT

This disclosure relates to plants having a modified F5H gene which confers the trait of reduced wound-induced surface discoloration. The disclosure further relates to all progeny, seed, and plant parts of said plant. Furthermore, the disclosure relates to a propagation material suitable for producing said plants, and to methods for selecting and producing said plants.

### RELATED APPLICATIONS AND INCORPORATION BY REFERENCE

This application is a continuation-in-part of U.S. application Ser. No. 16/787,231 filed Feb. 11, 2020, which is a continuation of U.S. application Ser. No. 16/106,684 filed Aug. 21, 2018, which is a continuation-in-part application of international patent application Serial No. PCT/EP2017/054343 filed 24 Feb. 2017, which published as PCT Publication No. WO 2017/144669 on 31 Aug. 2017, which claims benefit of European patent application Serial No. PCT/EP2016/053895 filed 24 Feb. 2016 and European patent application Serial No. PCT/EP2016/053999 filed 25 Feb. 2016.

The foregoing applications, and all documents cited therein or during their prosecution (“above cited documents”) and all documents cited or referenced in the above cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated by reference, and may be employed in the practice of the invention. More specifically, all referenced documents are incorporated by reference to the same extent as if each individual document was specifically and individually indicated to be incorporated by reference.

### SUMMARY OF THE INVENTION

The present invention relates to a plant comprising a modified F5H gene which confers the trait of reduced wound-induced surface discoloration. The invention further relates to all progeny, seed, and plant parts of said plant. Furthermore, the invention relates to a propagation material suitable for producing said plant, and to methods for selecting and producing said plant.

It is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 USC § 112, first paragraph) or the EPO (EPC Art. 83). Reserved is the right to disclaim and this disclosure hereby discloses a disclaimer of any previously described product, process of making the product, or method of using the product. All rights to explicitly disclaim any embodiments that are the subject of any granted patent(s) in the lineage of this application or in any other lineage or in any prior filed application of any third party are explicitly reserved.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE FIGURES

FIGS. 1A-1F: amino acid sequences of mutant plants comprising a mutation in the F5H1 protein.

FIGS. 1G-1L: amino acid sequences of mutant plants comprising a mutation in the F5H2 protein.

### DETAILED DESCRIPTION OF THE INVENTION

Regular consumption of fresh (raw) vegetables and fruits has been associated with the prevention of various chronic diseases, such as vasculary diseases and several forms of cancer. In accordance with the dietary advantages, the consumption of fresh vegetables and fruits shows an increasing trend worldwide.

Fresh vegetable and fruit is often sold as processed, ready-to-eat (cut, washed and packaged) product. Various consumer trends, such as convenience, premiumisation, online retailing, and shelf life extension have led to an increased demand for processed vegetable and fruit products worldwide. In the case of fresh leafy vegetables, such as lettuce (Lactuca sativa), the demand for processed products is especially pronounced.

One of the most important and frequently encountered problems during the processing and storage of lettuce is the development of wound-induced surface discoloration, which is visible as a pink discoloration at the wound surface of the plants or parts thereof which gradually turns brown after prolonged storage.

Wound-induced surface discoloration or wound-induced discoloration is caused by a strong wound response at and around the wound and leads to a rapid deterioration of the harvested and optionally processed product. Consumers consider discoloration of vegetables and fruits to be unattractive and to compromise the product quality, thus reducing the product's marketability and/or leading to a waste of harvested and optionally processed products.

The wound response is a means of a plant or part thereof to heal the wound and defend itself against pathogens by creating a new insulation barrier. The response is a complex biological response of a plant to physical injury such as cutting or bruising, and implies the activity of numerous proteins. The local response is mainly aimed at closing the wound surface which is effectuated by the local death of cells at or just behind the wound surface. In addition to these visible effects, other responses like increased respiration or ethylene production are known to be induced.

At the biochemical level, studies have shown that wounding can lead to the induction of the phenylpropanoid pathway which is required for inter alia the production of polyphenols and other compounds important for the plant.

The first step of the phenylpropanoid pathway is the conversion of the amino acid phenylalanine into cinnamic acid by the phenylalanine ammonia-lyase (PAL). PAL is enhanced upon wounding by the induction of gene expression of at least one of its isoforms. This response leads to the formation of polyphenols which are oxidized by the polyphenol oxidase (PPO). PPO is residing in plastids and is released and activated upon wounding. Oxidation of polyphenols leads to the formation of highly reactive quinones, that can react with amino acids or proteins which leads to pink, brown or black discoloration.

In order to reduce the wound-induced surface discoloration in lettuce, many post-harvest and post-processing treatments have been developed and applied. Examples of chemical or physical treatments are the packaging of fresh cut lettuce under a modified atmosphere, the application of edible coatings, heat-shock treatment and the addition of chemicals. Although these treatments prevent the appearance of the wound-induced discoloration, the harvested and eventually processed product is still susceptible to discoloration if the package is damaged or opened. In addition, the use of chemicals and the need for specialized equipment for such treatments significantly increases costs. For these reasons, a more viable genetically-based solution which works to reduce wound-induced surface discoloration in plants is preferred.

In the research that led to the present invention, it was surprisingly found that a reduced or absent ferulate-5-hydroxylase (F5H) protein functionality leads to a reduction of wound-induced surface discoloration in lettuce plants or parts thereof, as compared to lettuce plants or parts thereof in which the F5H protein functionality is not reduced or absent.

The F5H protein belongs to a family of plant cytochrome P450-dependent mono-oxygenases called CYP84. The F5H enzyme is part of the phenylpropanoid pathway, where it is responsible for the hydroxylation of coniferaldehyde and coniferyl alcohol.

The number of F5H gene homologs within a specific species can differ among different plant species. The Lactuca sativa plant genome may comprise two F5H gene homologs, herein named F5H1 and F5H2.

In the publicly available genome assembly of lettuce (Lactuca sativa) Lsat_Salinas_v8 genome assembly, submitted by the Lettuce Genome Resource in 2020 [based on Reyes Chin Wo et al. (2017) Nature Communications 8:14953], the wild type F5H1 gene homolog is located on chromosome 4, between positions 294787413 and 294789017. The wild type F5H2 gene homolog is located on chromosome 3, between positions100871828 and 100869853.

The present invention provides a plant comprising a first, or first and second modified F5H gene homolog, wherein the wild type of the first modified F5H gene homolog (F5H1) has a coding sequence according to SEQ ID No. 1 or a coding sequence that in order of increased preference has 95%, 96%, 97%, 98% or 99% sequence identity to SEQ ID No. 1 [but the 95% or higher sequence identity to SEQ ID No. 1 not including modifications as herein described that result in reduced or absent ferulate-5-hydroxylase (F5H) protein functionality and/or the herein described phenotype of reduced wound-induced surface discoloration], and wherein SEQ ID No. 1 encodes a protein having SEQ ID No. 2, or a protein that in order of increased preference has 95%, 96%, 97%, 98% or 99% sequence similarity to SEQ ID No. 2 [but the 95% or higher sequence similarity to SEQ ID No. 2 not including modifications as herein described that result in reduced or absent ferulate-5-hydroxylase (F5H) protein functionality and/or the herein described phenotype of reduced wound-induced surface discoloration], and the wild type of the second modified F5H gene homolog (F5H2) has a coding sequence according to SEQ ID No. 9 or a coding sequence that in order of increased preference has 95%, 96%, 97%, 98% or 99% sequence identity to SEQ ID No. 9 [but the 95% or higher sequence identity to SEQ ID No. 9 not including modifications as herein described that result in reduced or absent ferulate-5-hydroxylase (F5H) protein functionality and/or the herein described phenotype of reduced wound-induced surface discoloration], and wherein SEQ ID No. 9 encodes a protein having SEQ ID No. 10, or a protein that in order of increased preference has 95%, 96%, 97%, 98% or 99% sequence similarity to SEQ ID No. 10 [but the 95% or higher sequence similarity to SEQ ID No. 10 not including modifications as herein described that result in reduced or absent ferulate-5-hydroxylase (F5H) protein functionality and/or the herein described phenotype of reduced wound-induced surface discoloration], and wherein each of the first and second modified F5H gene homologs comprises at least one mutation, wherein the at least one mutation comprises one or more nucleotides replaced, inserted and/or deleted relative to the wild type, and wherein said one or more replaced, inserted and/or deleted nucleotide results in a reduction or complete absence of protein function, e.g., ferulate-5-hydroxylase (F5H) protein functionality, and wherein the modified gene homolog confers the phenotype of reduced wound-induced surface discoloration. The at least one mutation can be a mutation as herein discussed. Such a plant can be an agronomically elite plant as herein discussed. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the at least one mutation.

As used herein, sequence identity or sequence similarity is the percentage of nucleotides or amino acids, respectively, that is identical or similar between two sequences after proper alignment of those sequences. The person skilled in the art is aware of how to align sequences, for example by using a sequence alignment tool such as BLAST®, which can be used for both nucleotide sequences and protein sequences. To obtain the most significant result, the best possible alignment that gives the highest sequence identity or similarity score should be obtained. The percentage sequence identity or similarity is calculated through comparison over the length of the shortest sequence in the assessment, whereby in the present case a sequence that is included in such assessment represents a gene that at least comprises a start codon and a stop codon, or a complete protein encoded by such a gene. Sequence identity is used for comparison of nucleotide sequences. Sequence similarity is used to compare amino acid sequences, whereby conservative amino acid substitutions are deemed to be similar and is calculated herein based on the BLOSUM62 scoring matrix.

The modified F5H gene of the plant of the invention comprises one or more nucleotides replaced, inserted and/or deleted relative to the wild type, and said one or more replaced, inserted and/or deleted nucleotides results in a reduced functionality (reduction-of-function mutation) or complete nonfunctionality (loss-of-function mutation) of the F5H protein. The reduction in the F5H protein functionality can have but is not limited to one of the following causes: i) the absence of functional F5H protein can be due to the absence of F5H RNA or a significantly decreased level of F5H RNA (reduced expression), resulting in a complete absence or a reduced and biologically inadequate level of F5H protein; ii) the absence of functional F5H protein can also mean an absence of one or more of the functional protein domains, resulting in a truncated F5H protein that cannot perform its function; iii) the absence of functional F5H protein can mean that the modified protein has gained certain amino acids, destroying the wild type functionality of the protein; iv) the absence of functional F5H protein can further mean that the F5H protein has lost a protein-protein and/or protein-DNA interaction site. The reduction or absence of F5H protein functionality in the plant of the invention means the reduction or absence of functionality of at least a first F5H protein (F5H1).

In one embodiment, the present invention provides a plant comprising a modified F5H1 gene homolog wherein the modified gene homolog comprises a deletion of an adenine in SEQ ID No. 1 at position 152, or on a corresponding position of a homologous sequence having in order of increased preference 95%, 96%, 97%, 98%, or 99% sequence identity to SEQ ID No. 1 [but the 95% or higher sequence identity to SEQ ID No. 1 not including modifications as herein described that result in reduced or absent ferulate-5-hydroxylase (F5H) protein functionality and/or the herein described phenotype of reduced wound-induced surface discoloration]. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the deletion.

In one specific embodiment, the modified F5H1 gene homolog of the plant of the invention comprises a coding sequence having SEQ ID No. 3, or a sequence encoding a protein having SEQ ID No. 4. The invention comprehends plants having a coding sequence having, in order of increased preference, 95%, 96%, 97%, 98%, or 99% sequence identity to SEQ ID No. 3, or a sequence encoding a protein having, in order of increased preference, 95%, 96%, 97%, 98%, or 99% sequence similarity to SEQ ID No. 4, with it understood that the sequence having 95% or higher sequence identity to SEQ ID No. 3 and the sequence having 95% or higher sequence similarity to SEQ ID No. 4 maintain the modifications of SEQ ID Nos. 3 and 4 relative to the respective wild type sequences SEQ ID Nos. 1 and 2, said modification(s) providing for reduced or absent ferulate-5-hydroxylase (F5H) protein functionality and/or the herein described phenotype of reduced wound-induced surface discoloration. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the modifications of SEQ ID Nos. 3 and 4 relative to the respective wild type sequences SEQ ID Nos. 1 and 2, said modification(s) providing for reduced or absent ferulate-5-hydroxylase (F5H) protein functionality and/or the herein described phenotype of reduced wound-induced surface discoloration.

In another embodiment, the present invention provides a plant comprising a modified F5H1 gene homolog wherein the modified gene homolog comprises a deletion of an adenine and a cytosine in SEQ ID No. 1 at positions 152 and 153, respectively, or on corresponding positions of a homologous sequence having in order of increased preference 95%, 96%, 97%, 98%, or 99% sequence identity to SEQ ID No. 1 [but the 95% or higher sequence identity to SEQ ID No. 1 not including modifications as herein described that result in reduced or absent ferulate-5-hydroxylase (F5H) protein functionality and/or the herein described phenotype of reduced wound-induced surface discoloration]. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the deletion.

In another specific embodiment, the modified F5H1 gene homolog of the plant of the invention comprises a coding sequence having SEQ ID No. 5, or a sequence encoding a protein having SEQ ID No. 6. The invention comprehends plants having a coding sequence having at least 95% sequence identity to SEQ ID No. 5, or a sequence encoding a protein having in order of increased preference 95%, 96%, 97%, 98%, or 99% sequence similarity to SEQ ID No. 6, with it understood that the sequence having 95% or higher sequence identity to SEQ ID No. 5 and the sequence having 95% or higher sequence similarity to SEQ ID No. 6 maintain the modifications of SEQ ID Nos. 5 and 6 relative to the respective wild type sequences SEQ ID Nos. 1 and 2, said modification(s) providing for reduced or absent ferulate-5-hydroxylase (F5H) protein functionality and/or the herein described phenotype of reduced wound-induced surface discoloration. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the modifications of SEQ ID Nos. 5 and 6 relative to the respective wild type sequences SEQ ID Nos. 1 and 2, said modification(s) providing for reduced or absent ferulate-5-hydroxylase (F5H) protein functionality and/or the herein described phenotype of reduced wound-induced surface discoloration.

In yet another embodiment, the present invention provides a plant comprising a modified F5H1 gene homolog wherein the modified gene homolog comprises an insertion of an additional adenine in SEQ ID No. 1 at position 152, or on corresponding position of a homologous sequence having at least 95% sequence identity to SEQ ID No. 1 [but the 95% sequence identity to SEQ ID No. 1 not including modifications as herein described that result in reduced or absent ferulate-5-hydroxylase (F5H) protein functionality and/or the herein described phenotype of reduced wound-induced surface discoloration]. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the insertion.

In yet another specific embodiment, the modified F5H1 gene homolog of the plant of the invention comprises a coding sequence having SEQ ID No. 7, or a sequence encoding a protein having SEQ ID No. 8. The invention comprehends plants having a coding sequence having in order of increased preference 95%, 96%, 97%, 98%, or 99% sequence identity to SEQ ID No. 7, or a sequence encoding a protein having in order of increased preference 95%, 96%, 97%, 98%, or 99% sequence similarity to SEQ ID No. 8, with it understood that the sequence having 95% or higher sequence identity to SEQ ID No. 7 and the sequence having 95% or higher sequence similarity to SEQ ID No. 8 maintain the modifications of SEQ ID Nos. 7 and 8 relative to the respective wild type sequences SEQ ID Nos. 1 and 2, said modification(s) providing for reduced or absent ferulate-5-hydroxylase (F5H) protein functionality and/or the herein described phenotype of reduced wound-induced surface discoloration. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the modifications of SEQ ID Nos. 7 and 8 relative to the respective wild type sequences SEQ ID Nos. 1 and 2, said modification(s) providing for reduced or absent ferulate-5-hydroxylase (F5H) protein functionality and/or the herein described phenotype of reduced wound-induced surface discoloration.

The invention further relates to a plant comprising a modified F5H1 gene homolog wherein the F5H1 gene homolog comprises a frameshift mutation leading to a premature stop codon, wherein the premature stop codon results in an absence of functional F5H1 protein.

In a further embodiment, the present invention provides a plant comprising a modified F5H1 and a modified F5H2 gene homolog, wherein the modified F5H1 gene homolog comprises a deletion of an adenine in SEQ ID No. 1 at position 152, or on a corresponding position of a homologous sequence having in order of increased preference 95%, 96%, 97%, 98%, or 99% sequence identity to SEQ ID No. 1 [but the 95% or higher sequence identity to SEQ ID No. 1 not including modifications as herein described that result in reduced or absent ferulate-5-hydroxylase (F5H) protein functionality and/or the herein described phenotype of reduced wound-induced surface discoloration], and the modified F5H2 gene homolog comprises a deletion of two cytosins in SEQ ID No. 9 at positions 144 and 145, respectively, or on a corresponding position of a homologous sequence having in order of increased preference 95%, 96%, 97%, 98%, or 99% sequence identity to SEQ ID No. 9 [but the 95% sequence identity to SEQ ID No. 9 not including modifications as herein described that result in reduced or absent ferulate-5-hydroxylase (F5H) protein functionality and/or the herein described phenotype of reduced wound-induced surface discoloration].

In a further specific embodiment, the modified F5H1 gene homolog of the plant of the invention comprises a coding sequence having SEQ ID No. 3, or a sequence encoding a protein having SEQ ID No. 4, and the modified F5H2 gene homolog of the invention comprises a coding sequence having SEQ ID No. 11, or a sequence encoding a protein having SEQ ID No. 12. The invention comprehends plants having a coding sequence having in order of increased preference 95%, 96%, 97%, 98%, or 99% sequence identity to SEQ ID No. 3, or a sequence encoding a protein having in order of increased preference 95%, 96%, 97%, 98%, or 99% sequence similarity to SEQ ID No. 4, and a coding sequence having in order of increased preference 95%, 96%, 97%, 98%, or 99% sequence identity SEQ ID No. 11, or a sequence encoding a protein having in order of increased preference 95%, 96%, 97%, 98%, or 99% sequence similarity to SEQ ID No. 12, with it understood that the sequence having 95% or higher sequence identity to SEQ ID No. 3 and the sequence having 95% or higher sequence similarity to SEQ ID No. 4 maintain the modifications of SEQ ID Nos. 3 and 4 relative to the respective wild type sequences SEQ ID Nos. 1 and 2, and the sequence having 95% or higher sequence identity to SEQ ID No. 11 and the sequence having 95% or higher sequence similarity to SEQ ID No. 12 maintain the modifications of SEQ ID Nos. 11 and 12 relative to the respective wild type sequences SEQ ID Nos. 9 and 10, said modification(s) providing for reduced or absent ferulate-5-hydroxylase (F5H) protein functionality and/or the herein described phenotype of reduced wound-induced surface discoloration. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the modifications of SEQ ID Nos. 3 and 4 and SEQ ID Nos. 11 and 12 relative to the respective wild type sequences SEQ ID Nos. 1 and 2 and SEQ ID Nos. 9 and 10, said modification(s) providing for reduced or absent ferulate-5-hydroxylase (F5H) protein functionality and/or the herein described phenotype of reduced wound-induced surface discoloration.

In another embodiment, the present invention provides a plant comprising a modified F5H1 gene homolog and a modified F5H2 gene homolog, wherein the modified F5H1 gene homolog comprises a deletion of an adenine and a cytosine in SEQ ID No. 1 at positions 152 and 153, respectively, or on corresponding positions of a homologous sequence having in order of increased preference 95%, 96%, 97%, 98%, or 99% sequence identity to SEQ ID No. 1 [but the 95% or higher sequence identity to SEQ ID No. 1 not including modifications as herein described that result in reduced or absent ferulate-5-hydroxylase (F5H) protein functionality and/or the herein described phenotype of reduced wound-induced surface discoloration], and the modified F5H2 gene homolog comprises a deletion of a cytosin in SEQ ID No. 9 at position 144, or on a corresponding position of a homologous sequence having in order of increased preference 95%, 96%, 97%, 98%, or 99% sequence identity to SEQ ID No. 9 [but the 95% sequence identity to SEQ ID No. 9 not including modifications as herein described that result in reduced or absent ferulate-5-hydroxylase (F5H) protein functionality and/or the herein described phenotype of reduced wound-induced surface discoloration], said mutations acting as nonsense mutations. The nonsense mutations result in reduced or absent ferulate-5-hydroxylase (F5H) protein functionality and/or the herein described phenotype of reduced wound-induced surface discoloration. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the nonsense mutation.

In another specific embodiment, the modified F5H1 gene homolog of the plant of the invention comprises a coding sequence having SEQ ID No. 5, or a sequence encoding a protein having SEQ ID No. 6, and the modified F5H2 gene homolog of the invention comprises a coding sequence having SEQ ID No. 13, or a sequence encoding a protein having SEQ ID No. 14. The invention comprehends plants having a coding sequence having in order of increased preference 95%, 96%, 97%, 98%, or 99% sequence identity to SEQ ID No. 5, or a sequence encoding a protein having in order of increased preference 95%, 96%, 97%, 98%, or 99% sequence similarity to SEQ ID No. 6, and a coding sequence having in order of increased preference 95%, 96%, 97%, 98%, or 99% sequence identity SEQ ID No. 13, or a sequence encoding a protein having in order of increased preference 95%, 96%, 97%, 98%, or 99% sequence similarity to SEQ ID No. 14, with it understood that the sequence having 95% or higher sequence identity to SEQ ID No. 5 and the sequence having 95% or higher sequence identity to SEQ ID No. 6 maintain the modifications of SEQ ID Nos. 5 and 6 relative to the respective wild type sequences SEQ ID Nos. 1 and 2, and the sequence having 95% or higher sequence identity to SEQ ID No. 13 and the sequence having 95% or higher sequence similarity to SEQ ID No. 14 maintain the modifications of SEQ ID Nos. 13 and 14 relative to the respective wild type sequences SEQ ID Nos. 9 and 10, said modification(s) providing for reduced or absent ferulate-5-hydroxylase (F5H) protein functionality and/or the herein described phenotype of reduced wound-induced surface discoloration. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the modifications of SEQ ID Nos. 5 and 6 and SEQ ID Nos. 13 and 14 relative to the respective wild type sequences SEQ ID Nos. 1 and 2 and SEQ ID Nos. 9 and 10, said modifications providing for reduced or absent ferulate-5-hydroxylase (F5H) protein functionality and/or the herein described phenotype of reduced wound-induced surface discoloration.

In yet another specific embodiment, the present invention provides a plant comprising a modified F5H1 gene homolog and a modified F5H2 gene homolog, wherein the modified F5H1 gene homolog comprises an insertion of an additional adenine in SEQ ID No. 1 at position 152, or on a corresponding position of a homologous sequence having in order of increased preference 95%, 96%, 97%, 98%, or 99% sequence identity to SEQ ID No. 1 [but the 95% sequence identity to SEQ ID No. 1 not including modifications as herein described that result in reduced or absent ferulate-5-hydroxylase (F5H) protein functionality and/or the herein described phenotype of reduced wound-induced surface discoloration], and the modified F5H2 gene homolog comprises a deletion of an adenine and a cytosin in SEQ ID No. 9 at positions 146 and 147, respectively, or on corresponding positions of a homologous sequence having in order of increased preference 95%, 96%, 97%, 98%, or 99% sequence identity to SEQ ID No. 9 [but the 95% sequence identity to SEQ ID No. 9 not including modifications as herein described that result in reduced or absent ferulate-5-hydroxylase (F5H) protein functionality and/or the herein described phenotype of reduced wound-induced surface discoloration], said mutations acting as nonsense mutations. The nonsense mutations result in reduced or absent ferulate-5-hydroxylase (F5H) protein functionality and/or the herein described phenotype of reduced wound-induced surface discoloration. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the nonsense mutation.

In yet another specific embodiment, the modified F5H1 gene homolog of the plant of the invention comprises a coding sequence having SEQ ID No. 7, or a sequence encoding a protein having SEQ ID No. 8, and the modified F5H2 gene homolog of the invention comprises a coding sequence having SEQ ID No. 15, or a sequence encoding a protein having SEQ ID No. 16. The invention comprehends plants having a coding sequence having in order of increased preference 95%, 96%, 97%, 98%, or 99% sequence identity to SEQ ID No. 7, or a sequence encoding a protein having in order of increased preference 95%, 96%, 97%, 98%, or 99% sequence similarity to SEQ ID No. 8, and a coding sequence having in order of increased preference 95%, 96%, 97%, 98%, or 99% sequence identity SEQ ID No. 15, or a sequence encoding a protein having in order of increased preference 95%, 96%, 97%, 98%, or 99% sequence similarity to SEQ ID No. 16, with it understood that the sequence having 95% or higher sequence identity to SEQ ID No. 7 and the sequence having 95% or higher sequence similarity to SEQ ID No. 8 maintain the modifications of SEQ ID Nos. 7 and 8 relative to the respective wild type sequences SEQ ID Nos. 1 and 2, and the sequence having 95% or higher sequence identity to SEQ ID No. 15 and the sequence having 95% or higher sequence similarity to SEQ ID No. 16 maintain the modifications of SEQ ID Nos. 15 and 16 relative to the respective wild type sequences SEQ ID Nos. 9 and 10, said modification(s) providing for reduced or absent ferulate-5-hydroxylase (F5H) protein functionality and/or the herein described phenotype of reduced wound-induced surface discoloration. The plant can be an agronomically elite plant. The plant can be of the Asterid clan, or the plant can be of the Asteraceae plant family, or the plant can be of the Lactuca genus, or the plant can be a Lactuca sativa plant. As more fully discussed herein, the invention further comprehends cells (e.g., regenerable cells or protoplasts or meristematic cells), tissue (e.g., undifferentiated or differentiated tissue), tissue culture, germplasm, propagation material, leaf, pollen, embryo, cotyledon, hypocotyl, root, root tip, anther, flower, seed, stem, or progeny of (or for producing, e.g., in the case of propagation material) such a plant, having the modifications of SEQ ID Nos. 7 and 8 and SEQ ID Nos. 15 and 16 relative to the respective wild type sequences SEQ ID Nos. 1 and 2 and SEQ ID Nos. 9 and 10, said modification(s) providing for reduced or absent ferulate-5-hydroxylase (F5H) protein functionality and/or the herein described phenotype of reduced wound-induced surface discoloration.

The invention further relates to a plant comprising modified F5H1 and F5H2 gene homologs wherein the modified F5H1 and F5H2 gene homologs comprise frameshift mutations leading to premature stop codons, wherein the premature stop codons lead to an absence of functional F5H1 and F5H2 proteins.

As used herein, the term “homologous genes” refers to two related genes originating from a common ancestral gene. Homologous sequences are termed “homologs” and this term may be applied to both genes and proteins. The terms “homologous” or “homologs” may be used interchangeably. Homologous genes encode homologous proteins.

As used herein, a wild type gene or gene homolog refers to an unmodified F5H gene or gene homolog as it would occur in a plant not exhibiting a reduction in wound-induced surface discoloration. A wild type plant is used as control plant that does not carry a modified F5H gene homolog and therefore does not show the reduced wound-induced surface discoloration. To be comparable, the plant of the invention that comprises a modified F5H gene homolog and the wild type plant should be of the same species, preferably of the same variety, having the same age and being grown under identical conditions.

As used herein, the term “a modified F5H gene” means one or more modified F5H gene homologs. A “plant comprising a modified F5H gene homolog” is a plant which comprises one or more modified F5H gene homologs.

As used herein, the “modified F5H gene homolog of the invention” refers to a F5H gene homolog that may comprise any modification that leads to reduced wound-induced surface discoloration in a plant. Expressions, such as “modified F5H gene homolog of the invention”, “gene of the invention”, “F5H gene of the invention”, “F5H gene homolog of the invention” can be used interchangeably.

The modified F5H1 gene homolog of the invention alone, or in combination with other modified F5H gene homologs, such as F5H2, when present in a plant, confers the phenotype of reduced wound-induced surface discoloration. Modification in a F5H gene homolog may be present homozygously or heterozygously. Preferably, the modification in the F5H1 gene homolog is present homozygously.

In one embodiment, the modified first or first and second F5H gene homolog of this invention is a nucleic acid, advantageously a nucleic acid molecule, more advantageously an isolated nucleic acid molecule.

From the herein-discussion, the invention further relates to a plant, preferably a plant of the Asterid clan, more preferably a plant belonging to the Lactuca genus, even more preferably a Lactuca sativa plant, wherein the plant comprises in its genome a modified F5H gene homolog as described in the present application. A plant conform to the above description is referred to herein as a ‘plant of the invention’.

Thus, from the herein-discussion, the plant of the invention is a plant, which comprises in its genome a modified F5H gene homolog, said modified F5H gene homolog leading to the production of a modified F5H protein, wherein the modified F5H protein shows a reduced functionality or is completely non-functional. That reduced functionality or non-functionality provides the herein described phenotype of reduced wound-induced surface discoloration.

In one embodiment, from the herein-discussion, the plant of the invention is an agronomically elite plant, preferably an agronomically elite plant of the Asterid clan, more preferably an agronomically elite plant of the Asteraceae plant family, most preferably an agronomically elite Lactuca sativa plant.

In the context of this invention, an agronomically elite plant is a plant having a genotype that, as a result of human intervention, comprises an accumulation of distinguishable and desirable agronomic traits which allow a producer to harvest a product of commercial significance. Preferably the agronomically elite plant of the invention is a plant of an inbred line or a hybrid.

As used herein, a plant of an inbred line is a plant of a population of plants that is the result of three or more rounds of selfing, or backcrossing; or which plant is a doubled haploid. An inbred line may e.g., be a parent line used for the production of a commercial hybrid. Plants of the invention as herein-discussed can be a plant of an inbred line.

As used herein, a hybrid plant is a plant which is the result of a cross between two different plants having different genotypes. Advantageously, a hybrid plant is the result of a cross between plants of two different inbred lines, such a hybrid plant may e.g. be a plant of an F1 hybrid variety. Plants of the invention as herein-discussed can be a hybrid plant.

When used with regard to the claimed phenotype, the term “reduced” is always understood in relation to the wound-induced surface discoloration of a control plant or part thereof that has no modifications to any of its F5H gene homologs and is therefore a wild type plant which may comprise a wild type F5H gene homolog and does not exhibit reduced wound-induced surface discoloration. As used herein, a plant exhibiting a “reduced wound-induced surface discoloration” or a “reduction of wound-induced surface discoloration” is a plant having a reduced wound-induced surface discoloration as compared to the wound induced surface discoloration of a wild type plant. An improvement of the wound-induced surface discoloration is defined by a delayed appearance and/or reduced intensity of the discoloration as compared to a plant which does not comprise a modified F5H gene homolog. A reduced intensity of discoloration is visible by a less intense discoloration of the wound surface and/or a discolored surface that is smaller in size as compared to the discoloration and surface of the wound induced surface discoloration of a wild type plant. Ultimately, the wound-induced discoloration is completely absent. A delayed appearance of the discoloration means that the onset of discoloration occurs later in time. The plant thus maintains its fresh appearance longer, which is in fact an increase of shelf life.

In this application, the word “trait” refers to the phenotype of the plant. “Trait of the invention”, “trait”, or “phenotypic trait”, “phenotype”, “characteristic” may be used interchangeably. The trait of the invention as used herein is the reduced wound-induced surface discoloration as a result of the presence of a modified F5H gene homolog and its corresponding F5H protein.

The presence of the phenotype of reduced wound-induced surface discoloration response is established in a leaf disc test. In the leaf disc test, seeds of each genotype to be tested are germinated in trays and grown in glasshouse until the plants reach the 6-leaves stadium (approximately 8 weeks). Leaf disc samples are taken with the help of a hole punch, by choosing well developed, healthy leaves. The obtained samples are placed between two layers of filter paper moistened with MES buffer and incubated in a closed container at 7.5° C. The wound-induced surface discoloration response is visually assessed after 8 days of incubation. The wound-induced surface discoloration appears as a pink colored ring around the edges of the leaf disc. When the color is saturated and the wound-induced surface discoloration is very strong, the discoloration may appear red to very dark red. The presence of the phenotype of reduced wound-induced surface discoloration response is established, if the surface discoloration response of the plant to be tested is less pronounced than the surface discoloration response of the control plant, in the above-described leaf disc test. A less pronounced surface discoloration response in the leaf disc test means that the color appearing at the edges of the leaf discs is lighter and/or the discolored ring at the edges of the leaf discs is thinner.

In one embodiment, the invention relates to a plant comprising in its genome a first modified F5H gene homolog (F5H1), leading to a reduction of wound-induced surface discoloration in said plant or part thereof, in comparison with a plant or part thereof which does not comprise said modified gene homolog in its genome.

In a further embodiment, the invention relates to a plant comprising in its genome a first and second modified F5H gene homolog (F5H1 and F5H2, respectively), leading to a reduction of wound-induced surface discoloration in said plant or part thereof, in comparison with a plant or part thereof which does not comprise said modified gene homologs in its genome.

In a particular embodiment, the invention relates to a plant comprising in its genome a modified F5H1 and a modified F5H2 gene homolog, wherein the modified F5H1 gene homolog is present homozygously, and the F5H2 gene homolog is present either homozygously or heterozygously, and wherein said modification leads to a reduction of wound-induced surface discoloration in said plant or part thereof, in comparison with a plant or part thereof which does not comprise said modified gene homologs in its genome.

The invention also relates to propagation material suitable for producing a plant of the invention, wherein the propagation material is suitable for sexual reproduction, and is advantageously selected from a microspore, a pollen, an ovary, an ovule, an embryo sac and an egg cell, or is suitable for vegetative reproduction, and is advantageously selected from a cutting, a root, a stem a cell, and a protoplast, or is suitable for tissue culture of regenerable cells or protoplasts, and is advantageously selected from a leaf, a pollen, an embryo, a cotyledon, a hypocotyl, a meristematic cell, a root, a root tip, an anther, a flower, a seed and a stem, wherein the propagation material comprises a modified F5H gene homolog of the invention.

The invention further relates to a cell of a plant of the invention. Such a cell may either be in isolated form or a part of the complete plant or parts thereof and still forms a cell of the invention because such a cell comprises the modified F5H gene of the invention. Each cell of a plant of the invention carries the modified F5H gene homolog of the invention. A cell of the invention may also be a regenerable cell that can regenerate into a new plant of the invention.

The invention further relates to plant tissue of a plant of the invention. The tissue can be undifferentiated or differentiated tissue. Undifferentiated tissue is for example a stem tip, an anther, a petal, or pollen, and can be used in micro propagation to obtain new plantlets that are grown into new plants of the invention. The tissue can also be grown from a cell of the invention.

The invention moreover relates to the progeny of a plant, a cell, a tissue, or a seed of the invention, which progeny comprises the modified F5H gene homolog of the invention. Such progeny can in itself be a plant, a cell, a tissue, or a seed. As used herein, a progeny comprises the first and all further descendants from a cross with a plant of the invention, wherein a cross comprises a cross with itself or a cross with another plant, and wherein a descendant that is determined to be progeny, comprises a modified F5H gene homolog of the invention. A progeny also encompasses material that is obtained by vegetative propagation or another form of multiplication.

The invention further relates to the germplasm of a plant of the invention. The germplasm is constituted by all inherited characteristics of an organism and according to the invention, encompasses at least the trait of the invention. The germplasm can be used in a breeding program for the development of plants that exhibit the phenotype of reduced wound-induced surface discoloration. The use of germplasm that comprises a modified F5H gene homolog of the invention in breeding is also part of the present invention.

The invention also relates to the use of a modified F5H gene homolog of the invention for producing a plant that exhibits the phenotype of reduced wound-induced surface discoloration. The plant is preferably a plant that belongs to the Asteraceae plant family, advantageously the Lactuca genus, most preferably a Lactuca sativa plant.

The invention also relates to the use of a plant of the invention as a crop, as a source of seed or as a source of propagation material.

The present invention relates to a method for identification of a plant comprising a modified F5H gene homolog of the invention, which plant can be identified phenotypically and/or genotypically. A plant of the invention can be identified phenotypically, based on the fact that a plant comprising a modified F5H gene homolog may exhibit a reduced wound-induced surface discoloration phenotype. The genotypic identification of a plant of the invention comprises determining the presence of a modification in the F5H gene homolog, or in a homologous sequence thereof. Such genotypic identification can be followed by a phenotypic identification; i.e. analysing if the plant comprising the modification exhibits reduced wound-induced surface discoloration phenotype.

Determining the presence of a modification in the F5H gene homolog of the invention comprises identification of any modification in SEQ ID No. 1 or in SEQ ID No. 1 and SEQ ID No. 9, that leads to modification of protein function. Determining the presence of a modification includes determining the presence of any of the modifications as described herein. Determining the presence of a modification can be done through sequence comparison, which is known to the skilled person. Alternatively, determining the presence of a modification in the modified F5H gene homolog of the invention is done on the protein level and comprises the identification of any modification in SEQ ID No. 2 or in SEQ ID No. 2 and SEQ ID No. 10, including any modification leading to a change in protein function.

The invention relates to the development and use of a molecular marker to identify a modified F5H gene homolog of the invention. A molecular marker is based upon a modification to a F5H gene homolog that underlies the trait of reduced wound-induced surface discoloration. The person skilled in the art is familiar with creating and using a molecular marker for detecting and selecting plants with a modified F5H gene homolog causative of reduced wound-induced surface discoloration.

The invention further relates to a method for selecting a plant that exhibits a reduced wound-induced surface discoloration phenotype, comprising identifying the presence of a modification in a F5H gene homolog of the invention, and selecting a plant comprising a modification in a F5H gene homolog as a plant exhibiting the phenotype of reduced wound-induced surface discoloration. Optionally, the method comprises a further step in which the wound-induced surface discoloration response is investigated in a phenotypic screening test, such as the one described in Example 3. The selected plant obtained by the selection method is also a part of this invention.

The invention further relates to a method for seed production comprising growing a plant from a seed of the invention that comprises the modified F5H gene of the invention, allowing the plant to produce a fruit with seed, harvesting the fruit, and extracting the seed. Production of the seed is suitably done by selfing or by crossing with another plant that is optionally also a plant of the invention or at least comprises the modified gene. Preferably, the plant grown from the seed produced as described herein exhibits the phenotype of reduced wound-induced surface discoloration.

The invention also relates to a method for producing a hybrid seed, comprising crossing a first parent plant with a second parent plant and harvesting the resultant hybrid seed, wherein the first parent plant and/or the second parent plant is a plant of the invention comprising the modified F5H gene of the invention. Preferably, one of the parent plants comprises the modified F5H1 gene homolog of the invention homozygously.

The invention also relates to a hybrid seed produced by the method described herein and a hybrid plant grown from said hybrid seed.

The present invention also relates to a method for producing a plant that comprises the gene of the invention, said method comprising the introduction of a modification in a F5H gene homolog.

A F5H gene homolog can be modified by different means known in the art, including mutagenesis. Mutagenesis comprises the random introduction of at least one modification to DNA by means of one or more chemical compounds, such as ethyl methanesulphonate (EMS), nitrosomethylurea, hydroxylamine, proflavine, N-methyl-N-nitrosoguanidine, N-ethyl-N-nitrosourea, N-methyl-N-nitro-nitrosoguanidine, diethyl sulphate, ethylene imine, sodium azide, formaline, urethane, phenol and ethylene oxide, and/or by physical means, such as UV-irradiation, fast-neutron exposure, X-rays, gamma irradiation, and/or by insertion of genetic elements, such as transposons, T-DNA, retroviral elements. Mutagenesis also comprises the more specific, targeted introduction of at least one modification by means of homologous recombination, oligonucleotide-based mutation induction, zinc-finger nucleases (ZFNs), transcription activator-like effector nucleases (TALENs) or Clustered Regularly Interspaced Short Palindromic Repeat (CRISPR) systems.

Introduction of a modified F5H gene homolog of the invention can be done through introgression from a donor plant comprising said modified F5H gene homolog, advantageously from another plant exhibiting the phenotype of reduced wound-induced surface discoloration and in which the presence of a modified F5H gene homolog of the invention is identified, into a recipient plant that does not carry a modified F5H gene homolog, or which carries a modified F5H gene homolog heterozygously. Breeding methods such as crossing and selection, backcrossing, recombinant selection, or other breeding methods that result in the transfer of a genetic sequence from a donor plant to a recipient plant can be used. A donor plant, which is preferably a plant exhibiting the phenotype of reduced wound-induced surface discoloration, can be of the same species or of a different and/or wild species. Difficulties in crossing between species can be overcome through techniques known in the art such as embryo rescue, or cis-genesis can be applied. A plant produced by such method is also a part of the invention.

A modified F5H gene homolog may be part of a gene construct, which gene construct comprises a selectable marker, a promoter sequence, a F5H gene homolog sequence, and a terminator sequence.

Gene expression may also be prevented or reduced by preventing the transcription of the gene with for example RNA oligonucleotides or DNA oligonucleotides, or preferably by the expression of a negatively acting transcription factor acting on a F5H gene promoter. Other examples of methods to prevent or reduce the gene expression are the destabilization of the F5H mRNA or transcript, preferably by means of nucleic acid molecules that are complementary to the F5H mRNA or transcript selected from the group consisting of antisense RNA, RNAi molecules, Virus-Induced Gene Silencing (VIGS) molecules, co-suppressor molecules, RNA oligonucleotides or DNA oligonucleotides. Such methods for destabilizing mRNA or transcripts are well known to the person skilled in the art.

Examples of modifications leading to the reduction or absence of a F5H protein functionality are modifications leading to premature stop codons, frameshifts or amino acid substitutions. The said reduction or absence of a F5H protein functionality is herein directly responsible for the trait of reduced wound-induced surface discoloration. The reduced or absent functionality of a F5H protein may occur for example by introducing one or more mutations into the coding sequence of a F5H gene homolog. Mutation(s) to a F5H gene homolog may affect the biological function of the encoded protein, as compared to a F5H protein encoded by a wild type F5H gene homolog where no such mutation(s) is present.

Modification in the F5H1 or F5H2 gene homolog may be present in a heterozygous or in a homozygous state. Preferably the modification in the F5H1 gene homolog is present in a homozygous state.

The invention also relates to a method for the production of a plant exhibiting reduced wound-induced surface discoloration, comprising the steps of:
(a) crossing a first parent plant comprising a modified F5H gene homolog of the invention with a second parent plant to obtain an F1 population;
(b) optionally performing one or more rounds of selfing and/or crossing with a plant from the F1 population to obtain a further generation;
(c) selecting a plant that comprises the modified F5H1 gene homolog homozygously and optionally comprising the modified F5H2 gene homolog homo- or heterozygously.

The invention further relates to a method for the production of a plant comprising a modified F5H gene homolog of the invention, by using tissue culture or by using vegetative propagation.

The invention additionally provides for a method of introducing another desired trait into a plant that exhibits reduced wound-induced surface discoloration, comprising:
(a) crossing a plant comprising a F5H gene homolog of the invention with a second plant that comprises the other desired trait to produce F1 progeny;
(b) optionally selecting in the F1 for a plant that comprises the phenotype of the invention and the other desired trait;
(c) crossing the optionally selected F1 progeny with one of the parents for at least three generations, to produce backcross progeny;
(d) selecting a backcross progeny comprising the phenotype of the invention and the other desired trait; and
(e) optionally repeating steps c) and d) one or more times in succession to produce selected fourth or higher backcross progeny that comprises the phenotype of the invention and the other desired trait.

Optionally, selfing steps are performed after any of the crossing or backcrossing steps in the above described methods. Selection of a plant comprising the phenotype of wound-induced surface discoloration and the other desired trait can alternatively be done following any crossing or selfing step of the method. The other desired trait can be selected from, but is not limited to, the following group: resistance to bacterial, fungal or viral diseases, insect or pest resistance, improved germination, plant size, plant type, improved shelf-life, water stress and heat stress tolerance, and male sterility. The invention includes a plant produced by this method and any plant part therefrom.

The present invention is broadly applicable to all plant species and crops that carry at least one functional F5H gene homolog in their genome. The F5H genes present in other plant species are called “gene orthologs” and are coding for F5H proteins having the same or a similar function. Identification of F5H orthologues, i.e. F5H genes in other species, can be performed by methods known in the art.

TABLE 1
Sequence identity numbers of genomic, coding and amino
acid sequences identified in various lettuce plants,
referred to in the present submission, and optionally
carrying a modification in their F5H gene homologs.

| Plant | F5H1 | | SEQ ID No. | | F5H2 | | SEQ ID No. | |
|---|---|---|---|---|---|---|---|---|
| ID | | Pos CDS | CDS | Prot | | Pos CDS | CDS | Prot |
| 11_1 | WT | — | 1 | 2 | WT | — | 9 | 10 |
| 5_1 | del | A 152 | 3 | 4 | del | CC 144, | 11 | 12 |
| | | | | | | 145 | | |
| 5_2 | del | A 152 | 3 | 4 | del | CC 144, | 11 | 12 |
| | | | | | | 145 | | |
| 5_3 | del | AC 152, | 5 | 6 | del | C 144 | 13 | 14 |
| | | 153 | | | | | | |
| 5_6 | del | AC 152, | 5 | 6 | WT | — | 9 | 10 |
| | | 153 | | | | | | |
| 5_7 | del | AC 152, | 5 | 6 | WT | — | 9 | 10 |
| | | 153 | | | | | | |
| 5_8 | del | A 152 | 3 | 4 | del | CC 144, | 11 | 12 |
| | | | | | | 145 | | |
| 5_9 | del | A 152 | 3 | 4 | del | CC 144, | 11 | 12 |
| | | | | | | 145 | | |
| 5_10 | ins | AA 152 | 7 | 8 | del | AC 146, | 15 | 16 |
| | | | | | | 147 | | |
| 5_12 | del | A 152 | 3 | 4 | WT | — | 9 | 10 |

Abbreviations: ID = identifier, SEQ = sequence, WT = wild-type, del = deletion, ins = insertion, CDS = coding sequence, Prot = protein sequence, Pos CDS = position of the modification on the coding DNA sequence, wherein the hyphen signifies no modification.

### SEQUENCES

TABLE 2
Sequences claimed in the present invention.

| SEQ | |
|---|---|
| ID | |
| No | Sequence |
| 1 | >LsF5H(1)LsV3aug48059_wt |
| | ATGGAATCACTTCAAATCCCCATAGCATTCTACGCTATAATAGCTATCTTAACTTTCTTCTTTCTTTCATGGATCCGCCGGAAA |
| | CCACTCCCGCCGGGGCCAATGGGGTGGCCAATCATCGGCAACATGTTGATGATGGACCAACTTACCCACCGTGGCTTAGCCC |
| | GTTTGGCAGAAAAATACGGTGGTATCCTTCATCTAAAGATGGGTTTCAGCCACACCATTGCAGTGTCCTCGCCGGAGATGGC |
| | GAGGATAATACTTCAAGAAAAAGATAACATCTTTGCCAACCGTCCGGCAACCATCGCCATCACTTACCTGACTTACAACGGC |
| | GTAGATTTGGCTTTTGCTAATTATGGACCTTTCTGGCGACAAATGCGAAAGCTTTGTGTCATGAAGCTGTTCAGCCGGAAAC |
| | GAGCGGAGTCATGGGACTCCGTCAGGGATGAGGTGGACACCATGGTGAAAGCCACCGCCATTAACTCCGGTACGCCGGTA |
| | AACTTGGGTGAGCTTGTTTTTGGGTTGACCCATGATATTATCTACCGAGCAGCTTTTGGGTCGATTTCACATGAAGGGAAAG |
| | AAGAGTTTATCAGAATCCTTCAAGAATACACCAAACTTTTTGGCGCATTCAATTTGGCTGACTTTATCCCGTTCCTCGGGTTTA |
| | TTGATCCGGCGGGGTTGAACACTCGTTTACCGGCGGCCAGGGCGGCGTTGGACGGATTCATTGACAAAATCATCGACGAGC |
| | ATTTGAGTAAAGGAAAGAAAACCGGCGATGAAGGTTTGGATAACGATATGGTTGATGAGATGTTGGCGTTTTACAGTGAG |
| | GAAGGAAAAGTCAACGAAGGTGGTGATTTGCAAAACGCCATTAACCTTACCCGAGATAACATCAAAGCCATAATCATGGAT |
| | GTAATGTTCGGTGGAACTGAGACAGTGGCGTCCGCCATAGAATGGGCCATGACGGAGCTAATGCATACACCGGAGGCACT |
| | AAAGCGCGTGCAACAGGAGATGGCAAATGTCGTCGGACTTGACCGGCGCGTGGAGGAGTCTGACTTGGAGAAGCTGACGT |
| | ACTTCAAATGCGTCATCAAGGAAACCCTCCGACTACACCCTCCGATCCCAGTTCTCCTCCACCAGTCTTCGGAGGCGACAGAA |
| | GTTTCCGGCTACCATATACCTAAAGGAACACGTGTCATGGTGAACGCGTATGCTATTAATCGTGATAAGAACTCTTGGGAAG |
| | ATCCGGATACGTTTAACCCGTCACGTTTTTTACAAAACGGAGCTCCGGATTTAGAGGAAGCAACTATGAGTTTLTGCCATTT |
| | GGTTCTGGTCGGAGGTCGTGTCCGGGGATGCAACTAGGGTTGTATGCGATGGAGATGGCGGTGGCCCACCTTTTGCATTGT |
| | TTCACGTGGGAATTGCCGGATGGAATGAAGCCAAGTGAAATCGACATGGGTGATGTGTTTGGACTCACAGCACCAAAAGCA |
| | ATAAGATTGGTAGCAGTGCCAACTCCGCGTTTATTATGCCCATTGTATTGA |
| 2 | >LsF5H(1)LsV3aug48059_wt |
| | MESLQIPIAFYAIIAILTFFFLSWIRRKPLPPGPMGWPIIGNMLMMDQLTHRGLARLAEKYGGILHLKMGFSHTIAVSSPEMARIIL |
| | QEKDNIFANRPATIAITYLTYNGVDLAFANYGPFWRQMRKLCVMKLFSRKRAESWDSVRDEVDTMVKATAINSGTPVNLGELVFG |
| | LTHDHYRAAFGSISHEGKEEFIRILQEYTKLFGAFNLADFIPFLGFIDPAGLNTRLPAARAALDGFIDKIIDEHLSKGKKTGDEGLD |
| | NDMVDEMLAFYSEEGKVNEGGDLQNAINLTRDNIKAIIMDVMFGGTETVASAIEWAMTELMHTPEALKRVQQEMANVVGLDRRV |
| | EESDLEKLTYFKCVIKETLRLHPPIPVLLHQSSEATEVSGYHIPKGTRVMVNAYAINRDKNSWEDPDTFNPSRFLQNGAPDFRGSNY |
| | EFLPFGSGRRSCPGMQLGLYAMEMAVAHLLHCFTWELPDGMKPSEIDMGDVFGLTAPKAIRLVAVPTPRLLCPLY |
| 3 | >LsF5H(1)LsV3aug48O59_5_1 |
| | ATGGAATCACTTCAAATCCCCATAGCATTCTACGCTATAATAGCTATCTTAACTTTCTTCTTTCTTTCATGGATCCGCCGGAAA |
| | CCACTCCCGCCGGGGCCAATGGGGTGGCCAATCATCGGCAACATGTTGATGATGGACCAACTTACCCCCGTGGCTTAGCCC |
| | GTTTGGCAGAAAAATACGGTGGTATCCTTCATCTAAAGATGGGTTTCAGCCACACCATTGCAGTGTCCTCGCCGGAGATGGC |
| | GAGGATAATACTTCAAGAAAAAGATAACATCTTTGCCAACCGTCCGGCAACCATCGCCATCACTTACCTGACTTACAACGGC |
| | GTAGATTTGGCTTTTGCTAATTATGGACLTTTCTGGCGACAAATGCGAAAGCTTTGTGTCATGAAGCTGTTCAGCCGGAAAC |
| | GAGCGGAGTCATGGGACTCCGTCAGGGATGAGGTGGACACCATGGTGAAAGCCACCGCCATTAACTCCGGTACGCCGGTA |
| | AACTTGGGTGAGCTTGTTTTTGGGTTGACCCATGATATTATCTACCGAGCAGCTTTTGGGTCGATTTCACATGAAGGGAAAG |
| | AAGAGTTTATCAGAATCCTTCAAGAATACACCAAACTTTTTGGCGCATTCAATTTGGCTGACTTTATCCCGTTCCTCGGGTTTA |
| | TTGATCCGGCGGGGTTGAACACTCGTTTACCGGCGGCCAGGGCGGCGTTGGACGGATTCATTGACAAAATCATCGACGAGC |
| | ATTTGAGTAAAGGAAAGAAAACCGGCGATGAAGGTTTGGATAACGATATGGTTGATGAGATGTTGGCGTTTTACAGTGAG |
| | GAAGGAAAAGTCAACGAAGGTGGTGATTTGCAAAACGCCATTAACCTTACCCGAGATAACATCAAAGCCATAATCATGGAT |
| | GTAATGTTCGGTGGAACTGAGACAGTGGCGTCCGCCATAGAATGGGCCATGACGGAGCTAATGCATACACCGGAGGCACT |
| | AAAGCGCGTGCAACAGGAGATGGCAAATGTCGTCGGACTTGACCGGCGCGTGGAGGAGTCTGACTTGGAGAAGCTGACGT |
| | ACTTCAAATGCGTCATCAAGGAAACCCTCCGACTACACCCTCCGATCCCAGTTCTCCTCCACCAGTCTTCGGAGGCGACAGAA |
| | GTTTCCGGCTACCATATACCTAAAGGAACACGTGTCATGGTGAACGCGTATGCTATTAATCGTGATAAGAACTCTTGGGAAG |
| | ATCCGGATACGTTTAACCCGTCACGTTTTTTACAAAACGGAGCTCCGGATTTTAGAGGAAGCAACTATGAGTTTCTGCCATTT |
| | GGTTCTGGTCGGAGGTCGTGTCCGGGGATGCAACTAGGGTTGTATGCGATGGAGATGGCGGTGGCCCACCTTTTGCATTGT |
| | TTCACGTGGGAATTGCCGGATGGAATGAAGCCAAGTGAAATCGACATGGGTGATGTGTTTGGACTCACAGCACCAAAAGCA |
| | ATAAGATTGGTAGCAGTGCCAACTCCGCGTTTATTATGCCCATTGTATTGA |
| 4 | >LsF5H(1)LsV3aug48059_5_1_ |
| | MESLQIPIAFYAIIAILTFFFLSWIRRKPLPPGPMGWPIIGNMLMMDQLTPVA |
| 5 | >LsF5H(1)LsV3aug48059_5-3 |
| | ATGGAATCACTTCAAATCCCCATAGCATTCTACGCTATAATAGCTATCTTAALTTTCTTCTTTCTTTCATGGATCCGCCGGAAA |
| | CCACTCCCGCCGGGGCCAATGGGGTGGCCAATCATCGGCAACATGTTGATGATGGACCAACTTACCCCGTGGCTTAGCCCGT |
| | TTGGCAGAAAAATACGGTGGTATCCTTCATCTAAAGATGGGTTTCAGCCACACCATTGCAGTGTCCTCGCCGGAGATGGCGA |
| | GGATAATACTTCAAGAAAAAGATAACATCTTTGCCAACCGTCCGGCAACCATCGCCATCACTTACCTGACTTACAACGGCGTA |
| | GATTTGGCTTTTGCTAATTATGGACCTTTCTGGCGACAAATGCGAAAGCTTTGTGTCATGAAGCTGTTCAGCCGGAAACGAG |
| | CGGAGTCATGGGACTCCGTCAGGGATGAGGTGGACACCATGGTGAAAGCCACCGCCATTAACTCCGGTACGCCGGTAAACT |
| | TGGGTGAGCTTGTTTTTGGGTTGACCCATGATATTATCTACCGAGCAGCTTTTGGGTCGATTTCACATGAAGGGAAAGAAGA |
| | GTTTATCAGAATCCTTCAAGAATACACCAAACTTTTTGGCGCATTCAATTTGGCTGACTTTATCCCGTTCCTCGGGTTTATTGA |
| | TCCGGCGGGGTTGAACACTCGTTTACCGGCGGCCAGGGCGGCGTTGGACGGATTCATTGACAAAATCATCGACGAGCATTT |
| | GAGTAAAGGAAAGAAAACCGGCGATGAAGGTTTGGATAACGATATGGTTGATGAGATGTTGGCGTTTTACAGTGAGGAAG |
| | GAAAAGTCAACGAAGGTGGTGATTTGCAAAACGCCATTAACCTTACCCGAGATAACATCAAAGCCATAATCATGGATGTAAT |
| | GTTCGGTGGAACTGAGACAGTGGCGTCCGCCATAGAATGGGCCATGACGGAGCTAATGCATACACCGGAGGCACTAAAGC |
| | GCGTGCAACAGGAGATGGCAAATGTCGTCGGACTTGACCGGCGCGTGGAGGAGTCTGACTTGGAGAAGCTGACGTACTTC |
| | AAATGCGTCATCAAGGAAACCCTCCGACTACACCCTCCGATCCCAGTTCTCCTCCACCAGTCTTCGGAGGCGACAGAAGTTTC |
| | CGGCTACCATATACCTAAAGGAACACGTGTCATGGTGAACGCGTATGCTATTAATCGTGATAAGAACTCTTGGGAAGATCCG |
| | GATACGTTTAACCCGTCACGTTTTTTAGAAAAGGGAGCTGGGGATTTTAGAGGAAGCAACTATGAGHTCTGCCATTTGGTTC |
| | TGGTCGGAGGTCGTGTCCGGGGATGCAACTAGGGTTGTATGCGATGGAGATGGCGGTGGCCCACCTTTTGCATTGTTTCAC |
| | GTGGGAATTGCCGGATGGAATGAAGCCAAGTGAAATCGACATGGGTGATGTGTTTGGACTCACAGCACCAAAAGCAATAA |
| | GATTGGTAGCAGTGCCAACTCCGCGTTTATTATGCCCATTGTATTGA |
| 6 | >LsF5H(1)LsV3aug48059_5_3 |
| | MESLQIPIAFYAIIAILTFFFLSWIRRKPLPPGPMGWPIIGNMLMMDQLTPWLSPFGRKI |
| 7 | >LsF5H(1)LsV3aug48059_5_10 |
| | ATGGAATCACTTCAAATCCCCATAGCATTCTACGCTATAATAGCTATCTTAACTTTCTTCTTTCTTTCATGGATCCGCCGGAAA |
| | CCACTCCCGCCGGGGCCAATGGGGTGGCCAATCATCGGCAACATGTTGATGATGGACCAACTTACCCAACCGTGGCTTAGC |
| | CCGTTTGGCAGAAAAATACGGTGGTATCCTTCATCTAAAGATGGGTTTCAGCCACACCATTGCAGTGTCCTCGCCGGAGATG |
| | GCGAGGATAATACTTCAAGAAAAAGATAACATCTTTGCCAACCGTCCGGCAACCATCGCCATCACTTACCTGACTTACAACG |
| | GCGTAGATTTGGCTTTTGCTAATTATGGACCTTTCTGGCGACAAATGCGAAAGCTTTGTGTCATGAAGCTGTTCAGCCGGAA |
| | ACGAGCGGAGTCATGGGACTCCGTCAGGGATGAGGTGGACACCATGGTGAAAGCCACCGCCATTAACTCCGGTACGCCGG |
| | TAAACTTGGGTGAGCTTGTTTTTGGGTTGACCCATGATATTATCTACCGAGCAGCTTTTGGGTCGATTTCACATGAAGGGAA |
| | AGAAGAGTTTATCAGAATCCTTCAAGAATACACCAAACTTTTTGGCGCATTCAATTTGGCTGACTTTATCCCGTTCCTCGGGTT |
| | TATTGATCCGGCGGGGTTGAACACTCGTTTACCGGCGGCCAGGGCGGCGTTGGACGGATTCATTGACAAAATCATCGACGA |
| | GCATTTGAGTAAAGGAAAGAAAACCGGCGATGAAGGTTTGGATAACGATATGGTTGATGAGATGTTGGCGTTTTACAGTGA |
| | GGAAGGAAAAGTCAACGAAGGTGGTGATTTGCAAAACGCCATTAACCTTACCCGAGATAACATCAAAGCCATAATCATGGA |
| | TGTAATGTTCGGTGGAACTGAGACAGTGGCGTCCGCCATAGAATGGGCCATGACGGAGCTAATGCATACACCGGAGGCACT |
| | AAAGCGCGTGCAACAGGAGATGGCAAATGTCGTCGGACTTGACCGGCGCGTGGAGGAGTCTGACTTGGAGAAGCTGACGT |
| | ACTTCAAATGCGTCATCAAGGAAACCCTCCGACTACACCCTCCGATCCCAGTTCTCCTCCACCAGTCTTCGGAGGCGACAGAA |
| | GTTTCCGGCTACCATATACCTAAAGGAACACGTGTCATGGTGAACGCGTATGCTATTAATCGTGATAAGAACTCTTGGGAAG |
| | ATCCGGATACGTTTAACCCGTCACGTTTTTTACAAAACGGAGCTCCGGATTTTAGAGGAAGCAACTATGAGTTTCTGCCATTT |
| | GGTTCTGGTCGGAGGTCGTGTCCGGGGATGCAACTAGGGTTGTATGCGATGGAGATGGCGGTGGCCCACCTTTTGCATTGT |
| | TTCACGTGGGAATTGCCGGATGGAATGAAGCCAAGTGAAATCGACATGGGTGATGTGTTTGGACTCACAGCACCAAAAGCA |
| | ATAAGATTGGTAGCAGTGCCAACTCCGCGTTTATTATGCCCATTGTATTGA |
| 8 | >LsF5H(1)LsV3aug48059_5_10 |
| | MESLQIPIAFYAIIAILTFFFLSWIRRKPLPPGPMGWPIIGNMLMMDQLTQPWLSPFGRKIRWYPSSKDGFQPHHCSVLAGDGED |
| | NTSRKR |
| 9 | >LsF5H(2)LsV3aug951033_wt |
| | ATGGATCTTATGTCCATCTTACTTTACGTTGTACTCCCTCTCTTAACCTTCTTCCTTCTCTCCCGATTACGCCGAAAACCTCTTCC |
| | GCCAGGTCCAAGAGGCTGGCCGCTGATCGGTAACATGTTAATGATGGACCAACTCACCCACCGTGGCCTTGCTCGCTTGGG |
| | AGAAAAATACGGTGGTCTTCTTCACCTGAAGATGGGTTTCAGCCATACCGTCGCTGTCTCGTCCCCCGAAATAGCCAGGCAA |
| | GTACTCCAAGTTCAAGATAACATCTTCGCAAACCGTCCGGCCACCATCGCCATTAGTTACCTCACCTACGACCGGCAAGACAT |
| | GGCGTTCGCCAACTACGGTCCCTTCTGGCGTCAGATGCGTAAGCTTTGCGTCATGAAGCTGTTCAGCAGAAAGCGAGCTGA |
| | GTCTTGGGACTCCGTCAGAGACGAAGTTGTCTCCATGGTCAAAATCACCGCTGCAAGCTCCGGCACCGCTGTTAACCTTGGA |
| | GAGCTTGTTTTCGGGTTAACCCATGATATCATTTACCGAGCAGCTTTCGGGTCTATCTCTCATGAAGGAAAAGAAGAATTCAT |
| | CAGAATTCTACAAGAATACACAAAGCTTTTTGGTGCTTTCAATTTGGGAGATTTTGTCCCGTGGCTTGGATTTATCGACCCTG |
| | CCGGACTGAATACCCGTTTACCGAAGGCCAGGGCGGCGCTTGACAGATTCATTGATAAAATCATCGACGAGCACCTTGCAA |
| | AAGAGAGGAAAACGGGCGATGAGGAAGATAATGATATGGTGGATGAGATGTTGGCTTTTTACAGTGAAGAAGGAAAGGT |
| | AAACGAAGGCGAGGATTTGCAGAACGCGATTAGACTCACCCGAAACAATATCAAAGCCATTATTATGGATGTAATGTTTGGT |
| | GGGACTGAAACTGTTGCTTCTGCTATCGAATGGGCTTTAACTGAGCTAATGCACACCCCAGAATCCTTAAAACGTGCACAAC |
| | AAGAGCTCGCTGATGTTGTTGGCCTTGATCGTCGTGTAGAAGAATCAGATTTCGAGAAGCTAACTTACTTCAAATGTGTCAT |
| | CAAAGAAACCTTACGTCTCCACCCTCCGATCCCTGTCCTTTTGCACCAATCATCAGAAGCCACGTCGGTTGCTGGCTACCACA |
| | TACCTAAAGGGACACGTGTCATGGTTAACGCATTCGCCATTAATCGTGATAAGAACTCATGGAAGGATCCACACACGTTCAA |
| | CCCATCACGTTTGTTGGAAGATGGGGGAGGGGAGTTTAAAGGAAGGAATTATGAGTTTGTTCCATTTGGATCTGGACGTAGAT |
| | CATGTCCTGGAATGCAACTTGGATTGTACGCAATGGAGATGGCAGTGGCTCACCTTCTTCATTCATTCACATGGCAGTTGCCT |
| | GATGGAATGAAACCAAGTGAGATTGACATGAATGATGTGTTTGGACTCACTGCACCAAAAGCGATTCGACTTGTTGCTGTGC |
| | CAACTCCTCGGTTGTTGTGTCCGCTGTATTGA |
| 10 | >LsF5H(2)LsV3aug951033_wt_ |
| | MDLMSILLYVVLPLLTFFLLSRLRRKPLPPGPRGWPLIGNMLMMDQLTHRGLARLGEKYGGLLHLKMGFSHTVAVSSPEIARQVL |
| | QVQDNIFANRPATIAISYLTYDRQDMAFANYGPFWRQMRKLCVMKLFSRKRAESWDSVRDEVVSMVKITAASSGTAVNLGELV |
| | FGLTHDHYRAAFGSISHEGKEEFIRILQEYTKLFGAFNLADFVPWLGFIDPAGLNTRLPKARAALDRFIDKIIDEHLAKERKTGDEE |
| | DNDMVDEMLAFYSEEGKVNEGEDLQNAIRLTRNNIKAIIMDVMFGGTETVASAIEWALTELMHTPESLKRAQQELADVVGLDRRVE |
| | ESDFEKLTYFKCVIKETLRLHPPIPVLLHQSSEATSVAGYHIPKGTRVMVNAFAINRDKNSWKDPHTFNPSRFLQDGAPDFKGSNYE |
| | FLPFGSGRRSCPGMQLGLYAMEMAVAHLLHSFTWQLPDGMKPSEIDMNDVFGLTAPKAIRLVAVPTPRLLCPLY |
| 11 | >LsF5H(2)LsV3aug951033_5_1 |
| | ATGGATCTTATGTCCATCTTACTTTACGTTGTACTCCCTCTCTTAACCTTCTTCCTTCTCTCCCGATTACGCCGAAAACCTCTTCC |
| | GCCAGGTCCAAGAGGCTGGCCGCTGATCGGTAACATGTTAATGATGGACCAACTCACACCGTGGCCTTGCTCGCTTGGGAG |
| | AAAAATACGGTGGTCTTCTTCACCTGAAGATGGGTTTCAGCCATACCGTCGCTGTCTCGTCCCCCGAAATAGCCAGGCAAGT |
| | ACTCCAAGTTCAAGATAACATCTTCGCAAACCGTCCGGCCACCATCGCCATTAGTTACCTCACCTACGACCGGCAAGACATGG |
| | CGTTCGCCAACTACGGTCCCTTCTGGCGTCAGATGCGTAAGCTTTGCGTCATGAAGCTGTTCAGCAGAAAGCGAGCTGAGTC |
| | TTGGGACTCCGTCAGAGACGAAGTTGTCTCCATGGTCAAAATCACCGCTGCAAGCTCCGGCACCGCTGTTAACCTTGGAGAG |
| | CTTGTTTTCGGGTTAACCCATGATATCATTTACCGAGCAGCTTTCGGGTCTATCTCTCATGAAGGAAAAGAAGAATTCATCAG |
| | AATTCTACAAGAATACACAAAGGTTTTTGGTGCTTTCAATTTGGCAGATTTTGTCCCGTGGCTTGGATTTATCGACCCTGCCG |
| | GACTGAATACCCGTTTACCGAAGGCCAGGGCGGCGCTTGACAGATTCATTGATAAAATCATCGACGAGCACCTTGCAAAAG |
| | AGAGGAAAACGGGCGATGAGGAAGATAATGATATGGTGGATGAGATGTTGGCTTTTTACAGTGAAGAAGGAAAGGTAAAC |
| | GAAGGCGAGGATTTGCAGAACGCGATTAGACTCACCCGAAACAATATCAAAGCCATTATTATGGATGTAATGTTTGGTGGG |
| | ACTGAAACTGTTGCTTCTGCTATCGAATGGGCTTTAACTGAGCTAATGCACACCCCAGAATCCTTAAAACGTGCACAACAAG |
| | AGCTCGCTGATGTTGTTGGCCTTGATCGTCGTGTAGAAGAATCAGATTTCGAGAAGCTAACTTACTTCAAATGTGTCATCAAA |
| | GAAACCTTACGTCTCCACCCTCCGATCCCTGTCGTTTTGCACCAATCATCAGAAGCCACGTCGGTTGCTGGCTACCACATACCT |
| | AAAGGGACACGTGTCATGGTTAACGCATTCGCCATTAATCGTGATAAGAACTCATGGAAGGATCCACACACGTTCAACCCAT |
| | CACGTTTGTTGGAAGATGGGGGAGGGGAGTTTAAAGGAAGGAATTATGAGTTTGTTCCATTTGGATCTGGACGTAGATCATGT |
| | CCTGGAATGCAACTTGGATTGTACGCAATGGAGATGGCAGTGGCTCACCTTCTTCATTCATTCACATGGCAGTTGCCTGATG |
| | GAATGAAACCAAGTGAGATTGACATGAATGATGTGTTTGGACTCACTGCACCAAAAGCGATTCGACTTGTTGCTGTGCCAAC |
| | TCCTCGGTTGTTGTGTCCGCTGTATTGA |
| 12 | >LsF5H(2)LsV3aug951033_5_1 |
| | MDLMSILLYVVLPLLTFFLLSRLRRKPLPPGPRGWPLIGNMLMMDQLTPWPCSLGRKIRWSSSPEDGFQPYRRCLVPRNSQASTP |
| | SSR |
| 13 | >LsF5H(2)LsV3aug951033_5_3 |
| | ATGGATCTTATGTCCATCTTACTTTACGTTGTACTCCCTCTCTTAACCTTCTTCCTTCTCTCCCGATTACGCCGAAAACCTCTTCC |
| | GCCAGGTCCAAGAGGCTGGCCGCTGATCGGTAACATGTTAATGATGGACCAACTCACCACCGTGGCCTTGCTCGCTTGGGA |
| | GAAAAATACGGTGGTCTTCTTCACCTGAAGATGGGTTTCAGCCATACCGTCGCTGTCTCGTCCCCCGAAATAGCCAGGCAAG |
| | TACTCCAAGTTCAAGATAACATCTTCGCAAACCGTCCGGCCACCATCGCCATTAGTTACCTCACCTACGACCGGCAAGACATG |
| | GCGTTCGCCAACTACGGTCCCTTCTGGCGTCAGATGCGTAAGCTTTGCGTCATGAAGCTGTTCAGCAGAAAGCGAGCTGAGT |
| | CTTGGGACTCCGTCAGAGACGAAGTTGTCTCCATGGTCAAAATCACCGCTGCAAGCTCCGGCACCGCTGTTAACCTTGGAGA |
| | GCTTGTTTTCGGGTTAACCCATGATATCATTTACCGAGCAGCTTTCGGGTCTATCTCTCATGAAGGAAAAGAAGAATTCATCA |
| | GAATTCTACAAGAATACACAAAGCTTTTTGGTGCTTTCAATTTGGCAGATTTTGTCCCGTGGCTTGGATTTATCGACCCTGCC |
| | GGACTGAATACCCGTTTACCGAAGGCCAGGGCGGCGCTTGACAGATTCATTGATAAAATCATCGACGAGCACCTTGCAAAA |
| | GAGAGGAAAACGGGCGATGAGGAAGATAATGATATGGTGGATGAGATGTTGGCTTTTTACAGTGAAGAAGGAAAGGTAA |
| | ACGAAGGCGAGGATTTGCAGAACGCGATTAGACTCACCCGAAACAATATCAAAGCCATTATTATGGATGTAATGTTTGGTG |
| | GGACTGAAACTGTTGCTTCTGCTATCGAATGGGCTTTAACTGAGCTAATGCACACCCCAGAATCCTTAAAACGTGCACAACA |
| | AGAGCTCGCTGATGTTGTTGGCCTTGATCGTCGTGTAGAAGAATCAGATTTCGAGAAGCTAACTTACTTCAAATGTGTCATC |
| | AAAGAAACCTTACGTCTCCACCCTCCGATCCCTGTCCTTTTGCACCAATCATCAGAAGCCACGTCGGTTGCTGGCTACCACAT |
| | ACCTAAAGGGACACGTGTCATGGTTAACGCATTCGCCATTAATCGTGATAAGAACTCATGGAAGGATCCACACACGTTCAAC |
| | CCATCACGTTTGTTGCAAGATGGGGCACCCGACTTTAAAGGAAGCAATTATGAGTTTGTTCCATTTGGATCTGGACGTAGATC |
| | ATGTCCTGGAATGCAACTTGGATTGTACGCAATGGAGATGGCAGTGGCTCACCTTCTTCATTCATTCACATGGCAGTTGCCTG |
| | ATGGAATGAAACCAAGTGAGATTGACATGAATGATGTGTTTGGACTCACTGCACCAAAAGCGATTCGACTTGTTGCTGTGCC |
| | AACTCCTCGGTTGTTGTGTCCGCTGTATTGA |
| 14 | >LsF5H(2)LsV3aug951033_5_3 |
| | MDLMSILLYVVLPLLTFFLLSRLRRKPLPPGPRGWPLIGNMLMMDQLTTVALLAWEKNTVVFFT |
| 15 | >LsF5H(2)LsV3aug951033_5_10 |
| | ATGGATCTTATGTCCATCTTACTTTACGTTGTACTCCCTCTCTTAACCTTCTTCCTTCTCTCCCGATTACGCCGAAAACCTCTTCC |
| | GCCAGGTCCAAGAGGCTGGCCGCTGATCGGTAACATGTTAATGATGGACCAACTCACCCCGTGGCCTTGCTCGCTTGGGAG |
| | AAAAATACGGTGGTCTTCTTCACCTGAAGATGGGTTTCAGCCATACCGTCGCTGTCTCGTCCCCCGAAATAGCCAGGCAAGT |
| | ACTCCAAGTTCAAGATAACATCTTCGCAAACCGTCCGGCCACCATCGCCATTAGTTACCTCACCTACGACCGGCAAGACATGG |
| | CGTTCGCCAACTACGGTCCCTTCTGGCGTCAGATGCGTAAGCTTTGCGTCATGAAGCTGTTCAGCAGAAAGCGAGCTGAGTC |
| | TTGGGACTCCGTCAGAGACGAAGTTGTCTCCATGGTCAAAATCACCGCTGCAAGCTCCGGCACCGCTGTTAACCTTGGAGAG |
| | CTTGTTTTCGGGTTAACCCATGATATCATTTACCGAGCAGCTTTCGGGTCTATCTCTCATGAAGGAAAAGAAGAATTCATCAG |
| | AATTCTACAAGAATACACAAAGGTTTTTGGTGCTTTCAATTTGGCAGATTTTGTCCCGTGGCTTGGATTTATCGACCCTGCCG |
| | GACTGAATACCCGTTTACCGAAGGCCAGGGCGGCGCTTGACAGATTCATTGATAAAATCATCGACGAGCACCTTGCAAAAG |
| | AGAGGAAAACGGGCGATGAGGAAGATAATGATATGGTGGATGAGATGTTGGCTTTTTACAGTGAAGAAGGAAAGGTAAAC |
| | GAAGGCGAGGATTTGCAGAACGCGATTAGACTCACCCGAAACAATATCAAAGCCATTATTATGGATGTAATGTTTGGTGGG |
| | ACTGAAACTGTTGCTTCTGCTATCGAATGGGCTTTAACTGAGCTAATGCACACCCCAGAATCCTTAAAACGTGCACAACAAG |
| | AGCTCGCTGATGTTGTTGGCCTTGATCGTCGTGTAGAAGAATCAGATTTCGAGAAGCTAACTTACTTCAAATGTGTCATCAAA |
| | GAAACCTTACGTCTCCACCCTCCGATCCCTGTCGTTTTGCACCAATCATCAGAAGCCACGTCGGTTGCTGGCTACCACATACCT |
| | AAAGGGACACGTGTCATGGTTAACGCATTCGCCATTAATCGTGATAAGAACTCATGGAAGGATCCACACACGTTCAACCCAT |
| | CACGTTTGTTGGAAGATGGGGGAGGGGAGTTTAAAGGAAGGAATTATGAGTTTGTTCCATTTGGATCTGGACGTAGATCATGT |
| | CCTGGAATGCAACTTGGATTGTACGCAATGGAGATGGCAGTGGCTCACCTTCTTCATTCATTCACATGGCAGTTGCCTGATG |
| | GAATGAAACCAAGTGAGATTGACATGAATGATGTGTTTGGACTCACTGCACCAAAAGCGATTCGACTTGTTGCTGTGCCAAC |
| | TCCTCGGTTGTTGTGTCCGCTGTATTGA |
| 16 | >LsF5H(2)LsV3aug951033_5_10 |
| | MDLMSILLYVVLPLLTFFLLSRLRRKPLPPGPRGWPLIGNMLMMDQLTPWPCSLGRKIRWSSSPEDGFQPYRRCLVPRNSQASTP |
| | SSR |

The present invention will be further illustrated in the following Examples which are given for illustration purposes only and are not intended to limit the invention in any way.

### EXAMPLES

### Example 1: Generation of a Lettuce Plant Carrying the Gene of the Invention

Lettuce plants comprising a modified F5H protein were generated using the CRISPR/Cas9 gene editing technique. Genetic modification was carried out in L. sativa variety Sensai RZ, by PEG transfection of protoplasts, according to the method described in Park et al. Methods Mol Biol. 2019; 1917:337-354. The sgRNAs used in the transfection are listed in Table 3. After transfection, the protoplasts were cultured in vitro in beads, which induced cell division and callus formation. The calli regenerated into plantlets which were cultured until they rooted. Subsequently, the plants were transferred to the greenhouse and grown to maturity.

TABLE 3
Oligonucleotides used for modifying the F5H1 and
F5H2 gene homologs in L. sativa

| | #RGEN Target (5’ to 3’) | |
|---|---|---|
| sgRNA nr | sgRNA-PAM | Gene name |
| 1 | ATGGACCAACTTACCCACCG | F5H1 |
| 2 | GCTGTTCAGCCGGAAACGAG | F5H1 |
| 3 | ATGGACCAACTCACCCACCG | F5H2 |
| 4 | TTAGTTACCTCACCTACGAC | F5H2 |

### Example 2: Identification of a Plant Carrying the Gene of the Invention

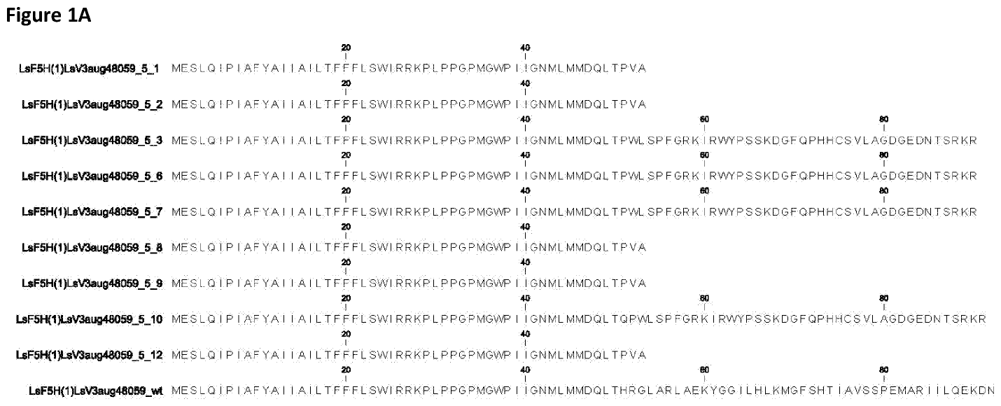

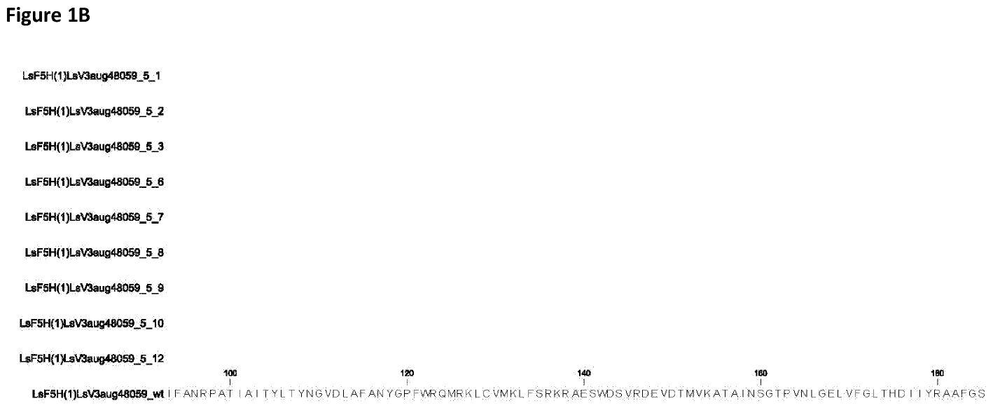

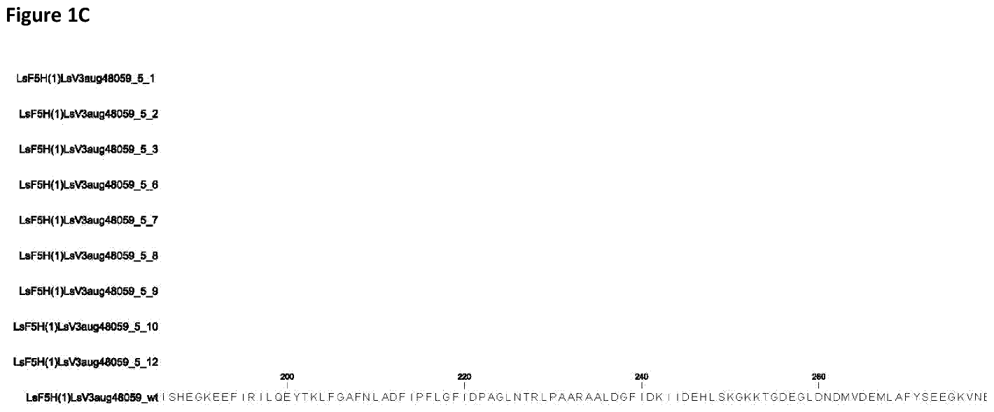

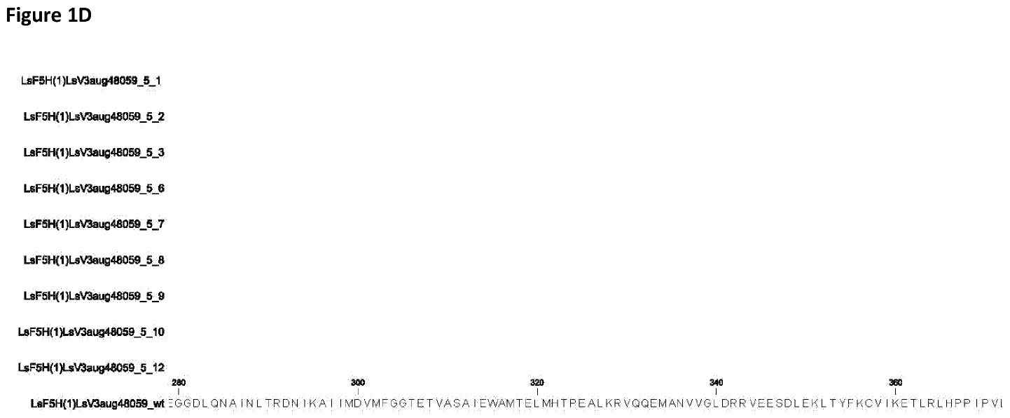

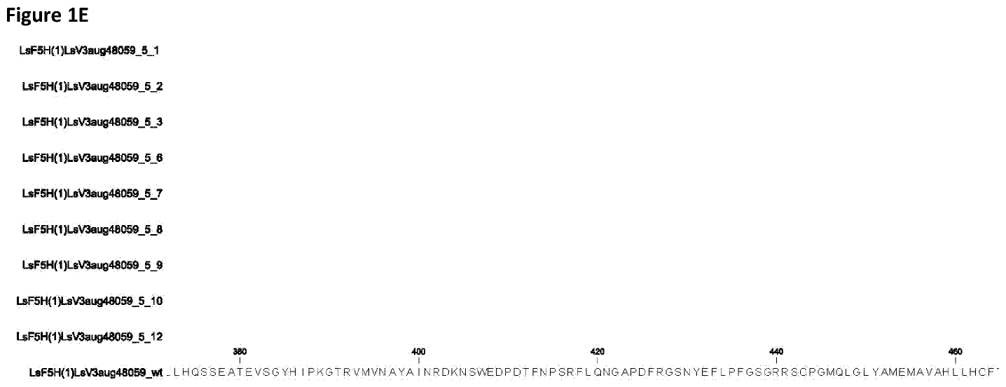

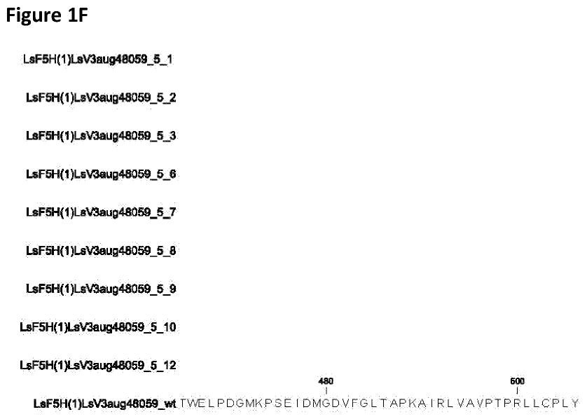

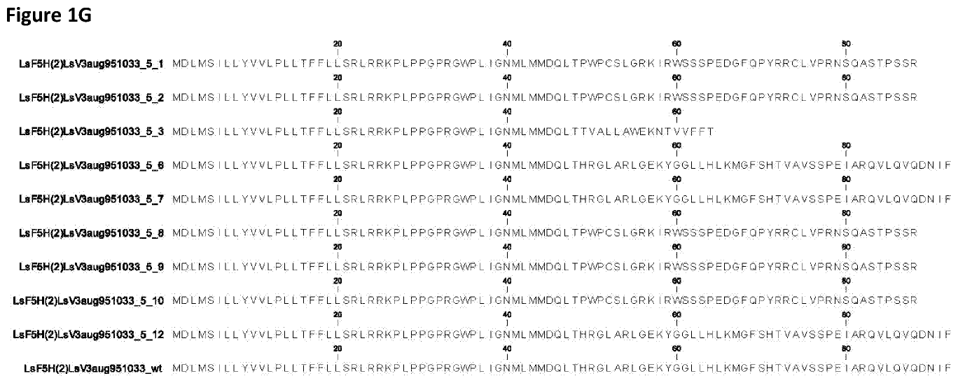

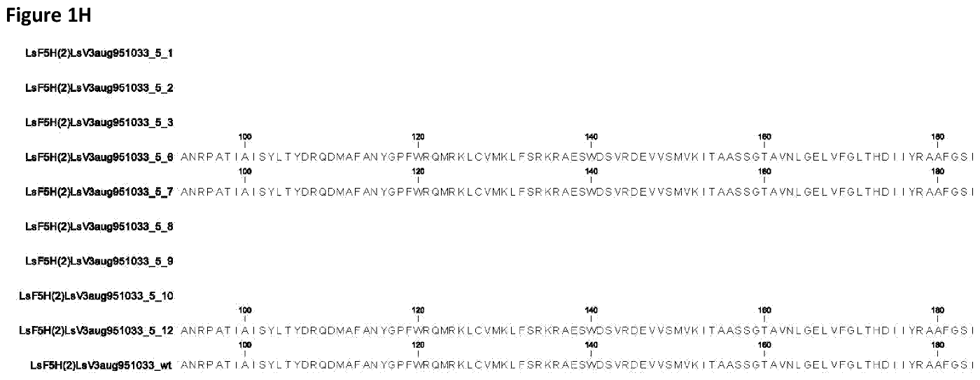

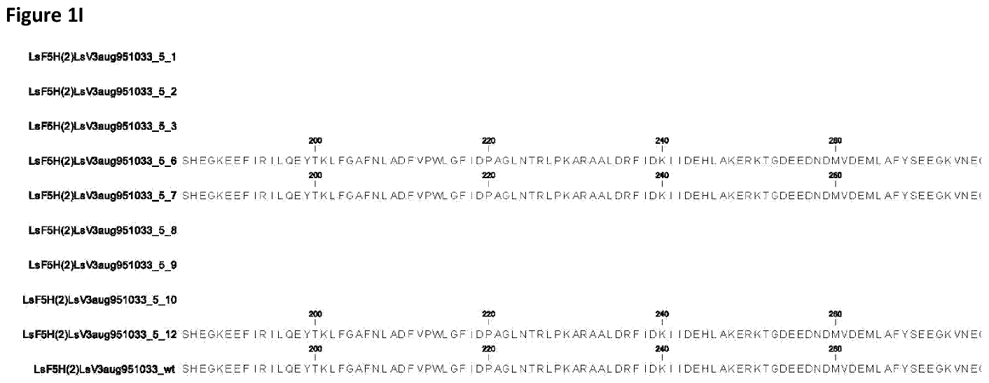

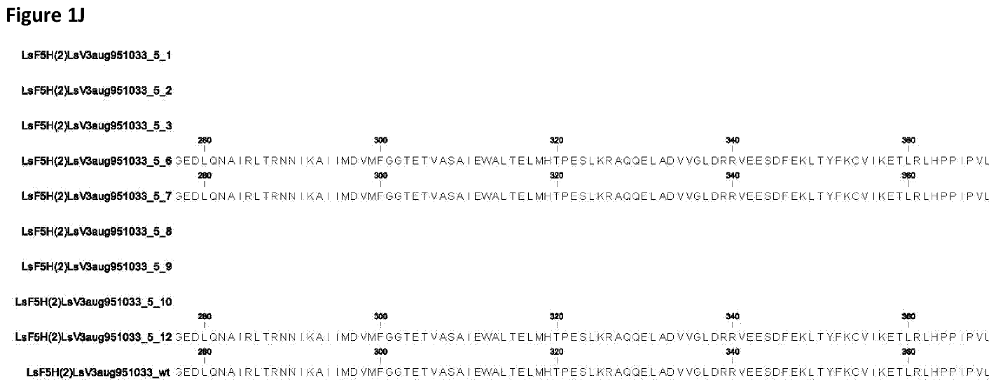

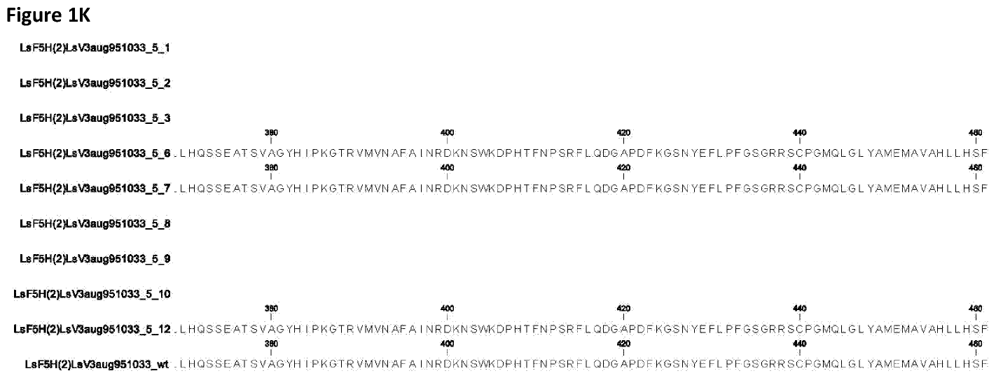

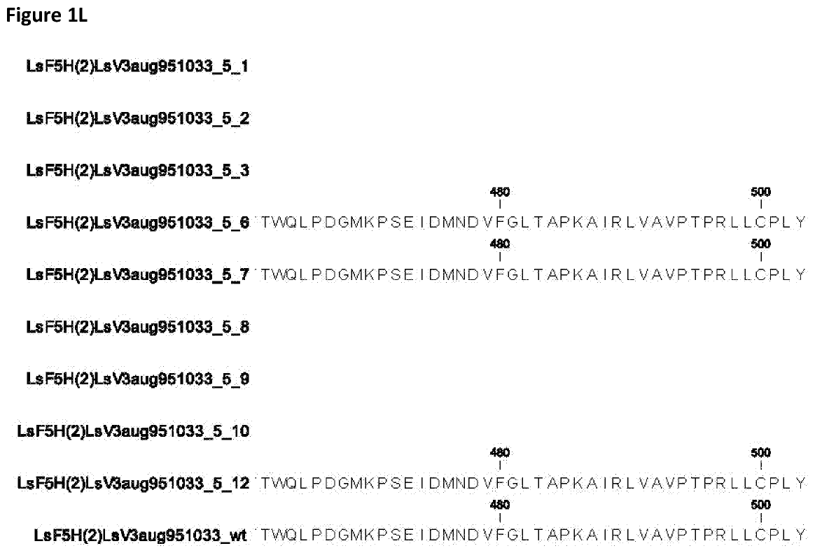

Genetic material of fully grown, healthy individual plants was tested for mutation in the F5H genes, using Sanger sequencing. The results of the sequence analysis are presented in FIG. 1, Table 2 and Table 4. Seeds of mature plants were collected and sown. Mutated lines were phenotyped in the second generation, using the phenotypic test described in Example 3. The results of the phenotypic analysis of selected mutant plants are presented in Table 4.

### Example 3: Phenotypic Identification of a Plant Exhibiting Reduced Wound-Induced Surface Discoloration

Plants were screened for their wound-induced surface discoloration response. Seeds of various genotypes were germinated in trays and grown in glasshouse, where the average conditions were 16 h day time at 20° C. and 8 h night time at 17° C., until the plants reached the 6-leaves stadium (in approximately 8 weeks). Leaf disc samples were taken from plants of each phenotype, taking 8 discs per plant, with the help of a hole punch of 1.2 cm in diameter. The obtained leaf disc samples were placed on a filter paper moistened with MES buffer, with the adaxial (upper) side down, and covered with a second filter paper also moistened with MES buffer. The air bubbles between the two filter papers were removed and the leaf discs were incubated between the wetted filter papers in a closed container at 7.5° C. After eight days of incubation, the wound-induced surface discoloration response of all genotypes was tested visually by a single person. The final results of the phenotypic analysis of some selected genotypes are presented in Table 4.

TABLE 4
Results of genotypic and phenotypic analyses of mutant
lettuce plants comprising a modified F5H gene homolog,
wherein the modification is generated by CRISPR/Cas9-
mediated gene editing and the phenotypic test is
carried out according to Example 3.

| | F5H1 | | F5H2 | | Phenotypic |
|---|---|---|---|---|---|
| Sample | mutation | Pos. CDS | mutation | Pos. CDS | response |
| WT | — | — | — | — | WT |
| 5-1 | deletion | 152 | deletion | 144_145 | reduced |
| 5-2 | deletion | 152 | deletion | 144_145 | reduced |
| 5-3 | deletion | 152_153 | deletion | 144 | reduced |
| 5-6 | deletion | 152_153 | — | — | reduced |
| 5-7 | deletion | 152_153 | — | — | reduced |
| 5-8 | deletion | 152 | deletion | 144_145 | reduced |
| 5-9 | deletion | 152 | deletion | 144_145 | reduced |
| 5-10 | insertion | 152 | deletion | 146_147 | reduced |
| 5-12 | deletion | 152 | — | — | reduced |

The invention is also described by the following numbered paragraphs:

1. A plant discussed herein as a plant of the invention; or a plant comprising a first, or first and second modified F5H gene homolog, wherein the wild type of the first modified F5H gene homolog (F5H1) has a coding sequence according to SEQ ID No. 1 or a coding sequence having in order of increased preference 95%, 96%, 97%, 98%, or 99% sequence identity to SEQ ID No. 1 [but the 95% or higher sequence identity to SEQ ID No. 1 not including modifications as herein described that result in reduced or absent ferulate-5-hydroxylase (F5H) protein functionality and/or the herein described phenotype of reduced wound-induced surface discoloration], and the wild type of the second modified F5H gene homolog (F5H2) has a coding sequence according to SEQ ID No. 9 or a coding sequence having in order of increased preference 95%, 96%, 97%, 98%, or 99% sequence identity to SEQ ID No. 9 [but the 95% or higher sequence identity to SEQ ID No. 9 not including modifications as herein described that result in reduced or absent ferulate-5-hydroxylase (F5H) protein functionality and/or the herein described phenotype of reduced wound-induced surface discoloration] and wherein in each of the first and second modified F5H gene homologs comprises at least one mutation, wherein the at least one mutation comprises one or more nucleotides replaced, inserted and/or deleted relative to the wild type, and wherein said one or more replaced, inserted and/or deleted nucleotide results in a reduction or complete absence of protein function and wherein the modified gene homolog confers the phenotype of reduced wound-induced surface discoloration.

2. The plant of paragraph 1 (or a plant as discussed herein as a plant of the invention), wherein the one or more modified F5H gene homolog comprises a premature stop codon leading to the reduction or absence of F5H protein function.

3. The plant of paragraphs 1 or 2 (or a plant as discussed herein as a plant of the invention), wherein the plant belongs to the Asterid clan.

4. The plant of any of paragraphs 1 to 3 (or a plant as discussed herein as a plant of the invention), wherein the plant is Lactuca sativa.

5. The plant of any of paragraphs 1 to 4 (or a plant as discussed herein as a plant of the invention) wherein the plant is an agronomically elite plant and/or an inbred plant and/or a hybrid plant.

6. A seed capable of growing into a plant as of any of paragraphs 1 to 5 (or a plant as discussed herein as a plant of the invention).

7. Propagation material capable of developing into and/or being derived from a plant of any of paragraphs 1 to 5 (or a plant as discussed herein as a plant of the invention), wherein the propagation material is suitable for sexual reproduction, or the propagration material comprises a microspore, pollen, ovary, ovule, embryo sac or egg cell, or is suitable for vegetative reproduction, or comprises a cutting, root, stem cell, and protoplast, or is suitable for tissue culture of regenerable cells or protoplasts, or comprises regenerable cells or protoplasts or comprises a leaf, pollen, embryo, cotyledon, hypocotyl, meristematic cell, root, root tip, anther, flower or stem.

8. A part of a plant of any of paragraphs 1 to 5 (or a plant as discussed herein as a plant of the invention), wherein the part is a leaf, a whole head of a plant, a fruit, an inflorescence, a seed, a curd, a stem, a tuber, a bulb or a root, optionally in processed form.

9. A food product comprising a part of a plant as in paragraph 8.

10. Use of a modified F5H gene homolog (e.g., an F5H homolog having mutation, addition, deletion, truncation or modification as herein discussed), for producing a plant as of any of paragraphs 1 to 5 (or a plant as discussed herein as a plant of the invention).

11. A method for selecting a plant that exhibits reduced wound-induced surface discoloration phenotype, comprising identifying the presence of a modification in a F5H gene homolog (e.g., identifying the presence of an F5H homolog having mutation, addition, deletion, truncation or modification as herein discussed) that results in a reduced functionality of its protein product, optionally testing the plant for its wound-induced surface discoloration phenotype, and selecting a plant which comprises a modification in its F5H gene homolog or homologs and also exhibits a reduced wound-induced surface discoloration phenotype if the optional phenotypic test is performed.

12. A method for reducing wound-induced surface discoloration in a plant, comprising the step of introducing a mutation in the F5H1 gene homolog (e.g., a mutation, addition, deletion, truncation or modification to the F5H1 gene homolog as herein discussed) and optionally introducing a mutation in the F5H2 gene homolog (e.g., a mutation, addition, deletion, truncation or modification to the F5H2 gene homolog as herein discussed), by random or site-directed mutagenesis, in a way that the resulting plant and/or any plant parts exhibits a reduced wound-induced surface discoloration phenotype.

13. A method for reducing wound-induced surface discoloration in a plant, comprising
(a) crossing a first parent plant comprising a modified F5H gene homolog as in any of paragraphs 1 to 5 (or a plant as discussed herein as a plant of the invention)with a second parent plant;
(b) optionally performing one or more rounds of selfing and/or crossing with a plant from the F1 population in order to obtain a further generation plant; and
(c) selecting a plant that comprises at least a modified F5H1 gene homolog.

14. The method of claim 13, wherein the plant is phenotypically selected and/or selected by use of molecular markers (or said molecular markers comprising, for example, mutation, addition, deletion, truncation or modification to the F5H1 gene homolog and/or F5H2 gene homolog as herein discussed).

15. A method of producing a hybrid plant seed capable of growing into a plant which exhibits reduced wound-induced surface discoloration phenotype, said method comprising the steps of crossing a first parent plant with a second parent plant and harvesting the resultant plant seed, wherein said first parent plant and/or said second parent plant is as defined in any of paragraphs 1 to 5 (or a plant as discussed herein as a plant of the invention).

16. The hybrid seed produced by the method of claim 15 (advantageously having modification as herein discussed).

17. The plant grown from the hybrid seed of claim 16 (advantageously having modification as herein discussed).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A plant comprising a first, or a first and second modified F5H gene homolog, wherein the wild type of the first modified F5H gene homolog (F5H1) has a coding sequence according to SEQ ID No. 1 or a coding sequence that in order of increased preference has 95%, 96%, 97%, 98% or 99% sequence identity to SEQ ID No. 1, and the wild type of the second modified F5H gene homolog (F5H2) has a coding sequence according to SEQ ID No. 9 or a coding sequence that in order of increased preference has 95%, 96%, 97%, 98% or 99% sequence identity to SEQ ID No. 9, and wherein the first or each of the first and second modified F5H gene homologs comprises at least one mutation, wherein the at least one mutation comprises one or more nucleotides replaced, inserted and/or deleted relative to the wild type, and wherein said one or more replaced, inserted and/or deleted nucleotide results in a reduction or complete absence of protein function and wherein the modified gene homolog confers the phenotype of reduced wound-induced surface discoloration; and, wherein the wild type coding sequence having 95% or higher sequence identity to SEQ ID No. 1 and the wild type coding sequence having 95% or higher sequence identity to SEQ ID No. 9 do not include the at least one mutation that results in a reduction or complete absence of protein function that confers the phenotype of reduced wound-induced surface discoloration.

2. The plant as claimed in claim 1, wherein the one or two modified F5H gene homolog comprises a premature stop codon leading to the reduction or absence of F5H protein function.

3. The plant as claimed in claim 1, wherein the plant belongs to the Asterid clan.

4. The plant as claimed in any of the claim 2, wherein the plant belongs to the Asterid clan.

5. The plant as claimed in claim 1, wherein the plant is Lactuca sativa.

6. The plant as claimed in claim 1, wherein the plant is an agronomically elite plant and/or a hybrid plant and/or an inbred plant.

7. A seed capable of growing into a plant as claimed in claim 1.

8. Propagation material capable of developing into and/or being derived from a plant as claimed in claim 1, wherein the propagation material is suitable for sexual reproduction, or the propagration material comprises a microspore, pollen, ovary, ovule, embryo sac or egg cell, or is suitable for vegetative reproduction, or comprises a cutting, root, stem cell, and protoplast, or is suitable for tissue culture of regenerable cells or protoplasts, or comprises regenerable cells or protoplasts or comprises a leaf, pollen, embryo, cotyledon, hypocotyl, meristematic cell, root, root tip, anther, flower or stem.

9. A part of a plant as claimed in claim 1, wherein the part is a leaf, a whole head of a plant, a fruit, an inflorescence, a seed, a curd, a stem, a tuber, a bulb or a root, optionally in processed form.

10. A food product comprising a part of a plant as claimed in claim 9.

11. A method for producing a plant as claimed in claim 1 having a modified F5H gene homolog that gives rise to a reduced wound-induced surface discoloration phenotype, said method comprising plant breeding or plant genetic transformation, thereby producing the plant; optionally said plant breeding or plant genetic transformation comprising screening for the presence of the modified F5H gene homolog, and said plant breeding optionally further comprising crossing a plant that from screening has the modified F5H gene homolog with another plant or with itself and obtaining the plant.

12. A method for selecting a plant that exhibits reduced wound-induced surface discoloration phenotype, comprising identifying the presence of a modification in a F5H gene homolog that results in a reduced functionality of its protein product, optionally testing the plant for its wound-induced surface discoloration phenotype, and selecting a plant which comprises a modification in its F5H gene homolog or homologs and also exhibits a reduced wound-induced surface discoloration phenotype when the optional phenotypic test is performed.

13. A method for reducing wound-induced surface discoloration in a plant, comprising the step of introducing a mutation in the F5H1 gene homolog and optionally introducing a mutation in the F5H2 gene homolog, by random or site-directed mutagenesis, in a way that the resulting plant and/or any plant parts exhibits a reduced wound-induced surface discoloration phenotype.

14. A method for reducing wound-induced surface discoloration in a plant, comprising
(a) crossing a first parent plant comprising a modified F5H gene homolog as claimed in claim 1 with a second parent plant;
(b) optionally performing one or more rounds of selfing and/or crossing with a plant from the F1 population in order to obtain a further generation plant; and
(c) selecting a plant that comprises at least a modified F5H1 gene homolog.

15. The method of claim 14, wherein the plant is phenotypically selected and/or selected by use of molecular markers.

16. A method of producing a hybrid plant seed capable of growing into a plant which exhibits reduced wound-induced surface discoloration phenotype, said method comprising the steps of crossing a first parent plant with a second parent plant and harvesting the resultant plant seed, wherein said first parent plant and/or said second parent plant is as defined in claim 1.

17. The hybrid seed produced by the method of claim 16.

18. The plant grown from the hybrid seed of claim 17.
